(19) United States
(12) Patent Application Publication
(10) Pub. No.: US 2023/0313326 A1
(43) Pub. Date: Oct. 5, 2023

(54) METHOD FOR DETERMINING THE RISK OF COMPLICATION IN A PATIENT

(71) Applicants: BIOMÉRIEUX, Marcy L'Etoile (FR); BIOASTER, Lyon (FR)

(72) Inventors: Laurence VACHOT, Fontaine (FR); François MALLET, Villeurbanne (FR); Guillaume MONNERET, Lyon (FR); Virginie MOUCADEL, Sassenage (FR); Alexandre PACHOT, Sulignat (FR); Estelle PERONNET, Lyon (FR); Thomas RIMMELÉ, Lyon (FR); Julien TEXTORIS, Villeurbanne (FR); Fabienne VENET, Lyon (FR)

(73) Assignees: BIOMÉRIEUX, Marcy L'Etoile (FR); BIOASTER, Lyon (FR)

(21) Appl. No.: 18/014,934

(22) PCT Filed: Jul. 5, 2021
(86) PCT No.: PCT/FR2021/051228

§ 371 Date: Jan. 6, 2023

(30) Foreign Application Priority Data
Jul. 6, 2020 (FR) FR2007131

(51) Int. Cl. C12Q 1/70; C12Q 1/689

(52) U.S. Cl. CPC C12Q 1/702; C12Q 1/689; C12Q 2600/158; C12Q 2600/118

(57) ABSTRACT

An in vitro or ex vivo method for determining the risk of complication in a patient, including a step of measuring the expression of C3AR1 in a biological sample from the patient.

The present invention relates to an in vitro or ex vivo method for determining the risk of complication in a patient, comprising a step of measuring the expression of C3AR1 in a biological sample from said patient.

Some patients may be faced with risks of complications, which it is important to be able to identify as early as possible in order to better treat these patients. Aside from the risk of death, the development of healthcare-associated infections is a significant complication associated with medical care, in particular in medical care facilities such as hospitals (where reference is more specifically made to nosocomial infections). It has been demonstrated that nosocomial infections in intensive care units, which occur in 20 to 40% of patients, were associated with increased morbidity and mortality, a longer requirement for supplemental care for organ failure(s), a longer stay in hospital, higher healthcare costs, and greater use of antibiotics, contributing to antimicrobial resistance. The incidence of healthcare-associated infections has become particularly exacerbated in recent years, due to the increase in multi-drug resistant pathogens. The World Health Organization (WHO) estimates the number of nosocomial infections in European hospitals to be around 5 million, leading to approximately 50 000 deaths and an annual excess cost of 13 to 24 billion Euros. Numerous factors influence the incidence and development of healthcare-associated infections, such as the patient's general state of health, but also factors associated with the patient's treatment (e.g. the administration of antibiotics and/or the use of invasive medical devices), factors associated with the hospital environment (e.g. the ratio of the number of nurses to the number of patients), and the varying use of aseptic techniques by hospital personnel. Recommendations have been published, and the establishment of infection control programs has been encouraged, in particular by the U.S. Department of Health and Human Services, the European Center for Disease Prevention and Control, the WHO and national agencies, for which the prevention and reduction of healthcare-associated infections have become a major priority. It has been shown that programs for controlling healthcare-associated infections have proven particularly effective in reducing serious infections. However, it has been estimated that a maximum of 65 to 70% of cases of blood and urinary tract infections, associated with catheterization, and 55% of cases of pneumonia, associated with mechanical ventilation, and of infections at surgical sites, could be prevented. Moreover, compliance with and application of procedures according to recommendations can be complicated in some hospitals, particularly in low- or middle-income countries. The early identification of patients at risk of developing a healthcare-associated infection would be a key step in preventing these infections and in treating these patients. According to certain models, a biomarker which would reduce the identification time of healthcare-associated infections in a high-risk population would make it possible to reduce mortality in these patients, with a good benefit-cost ratio. However, there is currently no clinical in vitro diagnostic test which makes it possible to identify patients with a high risk of contracting a healthcare-associated infection.

The C3AR1 gene (complement component 3a receptor 1) encodes the C3a complement receptor, a protein released during complement activation. The binding of the anaphylatoxin C3a to its receptor activates chemotaxis and opsonization of bacteria. In the literature, it has been shown that the C3AR1 gene transcript was more greatly expressed in septic patients than in healthy volunteers (Napier et al. (2016), J. Exp. Med. 213(11):2365-2382). Moreover, a list of eleven genes, including C3AR1, made it possible to distinguish between patients suffering from sterile inflammation and patients developing inflammation associated with an infection (Sweeney et al. (2015), Sci. Transl. Med. 7(287):287ra71). These studies therefore suggest the potential usefulness of C3AR1 as a diagnostic biomarker.

It has now been discovered, entirely surprisingly, that measuring the expression of the C3AR1 gene made it possible to determine the risk of complication in a patient. This has never been shown or suggested in the literature. Patients having a high risk of complication, in particular those having a high risk of death and/or of contracting a healthcare-associated infection could advantageously benefit from customized treatment.

Thus, the present invention relates to an in vitro or ex vivo method for determining the risk of complication in a patient, comprising a step of measuring the expression of C3AR1 in a biological sample from said patient.

In the context of the present invention:
The term “patient” denotes an individual (human) who has come into contact with a healthcare professional such as a doctor (for example a family physician) or a medical establishment or a healthcare facility (for example a hospital, and more particularly the emergency department, the resuscitation unit, an intensive care unit or an ongoing care unit, or a medicalized establishment for the elderly, such as an assisted living facility). The patient may for example be an elderly person, in the context of a vaccination protocol (particularly in an assisted living facility or else at a family physician);
The risk of complication corresponds to a risk of an unfavourable change in the state of health of a patient, and includes in particular the risk of incidence of a (primary or secondary) healthcare-associated infection, of viral reactivation, of an increase in the SOFA score by at least one point, of the appearance of at least one organ failure, of needing to be admitted to the emergency department or the resuscitation unit, or else of death.
An infection is said to be “healthcare-associated” if it occurs during, or after, a treatment (diagnostic, therapeutic, palliative, preventative, educational or operating) of a patient by a healthcare professional and if it was not present at the start of treatment. Healthcare-associated infections (HCAI) include infections contracted within a healthcare facility (referred to as nosocomial infections) but also those contracted during care administered outside this context. When the state of infection at the start of treatment is not known precisely, a period of at least 48 hours or a period longer than the incubation period is commonly accepted for defining an HCAI. For infections of the surgical site, infections are usually considered to be healthcare-associated when they occur in the 30 days following surgery or, if an implant, a prosthesis or a prosthetic material has been put in place, in the year following surgery. The infection may be of bacterial, fungal or viral origin. It may also relate to the reactivation of potentially pathogenic latent viruses, such as herpes viruses, for example CMV;
“Biological sample” relates to any sample originating from a patient, which may be of different natures, such as blood or derivatives thereof, sputum, urine, stools, skin, cerebrospinal fluid, bronchoalveolar lavage fluid, abdominal cavity puncture fluid, saliva, gastric secretions, sperm, seminal fluid, tears, spinal cord, trigeminal nerve ganglion, adipose tissue, lymphoid tissue, placental tissue. gastrointestinal tract tissue, genital tract tissue, central nervous system tissue. In particular, this sample may be a biological fluid, such as a blood sample or a blood-derived sample, which may particularly be chosen from total blood (as collected from a vein, i.e. containing white and red blood cells, platelets and plasma), plasma, serum, and any type(s) of cells extracted from the blood, such as peripheral blood mononuclear cells (PBMCs, containing lymphocytes (B, T and NK cells), dendritic cells and monocytes), subsets of B cells, purified monocytes, or neutrophils.

Preferably, in the method as described previously:
the patient is a patient within a healthcare facility, preferably within a hospital, more preferably still within the emergency department, the resuscitation unit, an intensive care unit or an ongoing care unit; particularly preferably, the patient is a patient in a septic state (more particularly in septic shock), a patient suffering from burns (more particularly severe burns), a patient suffering from an injury (more particularly a severe injury), or a patient undergoing surgery (more particularly major surgery); and
the method makes it possible to determine the risk of complication, including the risk of death and/or the risk of incidence of a healthcare-associated infection, more particularly a nosocomial infection, in said patient.

In the case of a patient in a septic state (already infected with a first infection), the method according to the invention particularly makes it possible to determine the risk of incidence of a secondary infection. “Patient in a septic state” (or patient suffering from sepsis) means a patient having at least one life-threatening organ failure caused by an inappropriate host response to infection. “Septic shock” means a sub-type of sepsis in which hypotension persists despite sufficient vascular filling.

Preferably, the method according to the invention, as described previously in all the embodiments thereof, makes it possible:
to determine the risk of death in a patient in the 30 days starting from the day of the immuno-inflammatory attack (i.e. the injury for patients suffering from an injury, the burn for patients suffering from burns, the surgery for patients undergoing surgery, or the sepsis diagnosis for patients in a septic state), i.e. during the 1st, 2nd, 3rd, 4th, 5th, 6th, 7th, 8th, 9th, 10th, 11th, 12th, 13th, 14th, 15th, 16th, 17th, 18th, 20th, 21st, 22nd, 23rd, 24th, 25th, 26th, 27th, 28th, 29th, or 30th day starting from the immuno-inflammatory attack (the 1st day corresponding here to the day on which the immuno-inflammatory attack took place), and/or to determine the risk of incidence of a healthcare-associated infection in a patient:
in the 30 days starting from the day of the immuno-inflammatory attack, more preferably still in the 15 days starting from the day of the immuno-inflammatory attack, i.e. during the 1st, 2nd, 3rd, 4th, 5th, 6th, 7th, 8th, 9th, 10th, 11th, 12th, 13th, 14th, or 15th day starting from the immuno-inflammatory attack (the 1st day corresponding here to the day on which the immuno-inflammatory attack took place); with it being possible for the biological sample to have been taken during the 1st, 2nd, 3rd, 4th, 5th, 6th, 7th, 8th, 9th, 10th, 11th, 12th, 13th, 14th, or 15th day starting from the immuno-inflammatory attack, preferably during the 1st, 2nd, 3rd, 4th, 5th, 6th or 7th day starting from the immuno-inflammatory attack, more preferably still during the 3rd, 4th, 5th, 6th or 7th day starting from the immuno-inflammatory attack; and/or
in the 7 days, 6 days, 5 days, or 4 days following the day on which the biological sample was taken (regardless of the day on which this sample was taken), i.e. during the 1st, 2nd, 3rd, 4th, 5th, 6th, or 7th day following the day on which the biological sample was taken (the 1st day corresponding here to the day after the day on which the biological sample was taken).

Preferably, the method as described previously, in all the embodiments thereof, additionally comprises a step of measuring, in the biological sample from the patient, the expression:
of at least one gene selected from the family of genes encoding molecules involved in the innate immune system, and/or
of at least one gene selected from the family of genes encoding molecules of the cell cycle, and/or
of at least one gene selected from the family of genes encoding cytokines, and/or
of at least one gene selected from the family of genes encoding anti-inflammatory cytokines, and/or
of at least one gene selected from the family of genes encoding pro-inflammatory cytokine receptors, located on chromosome 2 in the region 2q11-2q12, and/or
of at least one gene selected from the family of genes encoding pro-inflammatory cytokines, and/or
of at least one gene selected from the family of genes encoding molecules involved in cytoskeleton formation, and/or
of at least one gene selected from the family of genes encoding molecules involved in gene expression and/or transcription, and/or
of at least one gene selected from the family of genes encoding growth factors, and/or
of at least one gene selected from the family of genes encoding molecules of the metabolism, and/or
of at least one gene selected from the family of genes encoding molecules involved in the adaptive immune system, and/or
of at least one gene selected from the family of genes encoding molecules involved in signal transduction, and/or
of at least one gene selected from the family of genes encoding molecules involved in modulation of the acute phase, and/or
of at least one human endogenous retrovirus (HERV).

As examples of genes encoding molecules involved in the innate immune system, mention may be made of the following genes: GNLY, S100A9, ADGRE3, CD177, CX3CR1, IFIH1, OAS2, OAS3.

As an example of a gene encoding molecules of the cell cycle, mention may be made of the gene CCNB1IP1.

As examples of genes encoding cytokines, mention may be made of the following genes: IL15, IL2, MCP1(CCL2), CXCL10.

As examples of genes encoding anti-inflammatory cytokines, mention may be made of the following genes: IL10, IL1RN.

As examples of genes encoding pro-inflammatory cytokine receptors, located on chromosome 2 in the region 2q11-2q12, mention may be made of the following genes: IL18R1, IL1R2, ILR1 and ILI8RAP.

As examples of genes encoding pro-inflammatory cytokines, mention may be made of the following genes: IFNG, IL1B, IL17A, IL18, IL6, TNF.

As examples of genes encoding molecules involved in cytoskeleton formation, mention may be made of the following genes: ARL14EP, GSN.

As examples of genes encoding molecules involved in gene expression and/or transcription, mention may be made of the following genes: CIITA, DYRK2, GATA3, MDC1, NFKB1, RORC, STAT4, TBX21, TDRD9.

As an example of a gene encoding growth factors, mention may be made of the gene CSF2.

As examples of genes encoding molecules of the metabolism, mention may be made of the following genes: ALOX5, BPGM, TRAP1.

As examples of genes encoding molecules involved in the adaptive immune system, mention may be made of the following genes: CD40LG, CD3D, BTLA, CD274, CTLA4, ICOS, PDCD1, TNFSF4, CD74, FCGR1A, LILRB2, TAP2.

As examples of genes encoding molecules involved in signal transduction, mention may be made of the following genes: FLT1, HAVCR2, IL7R, ZAP70.

As an example of a gene encoding molecules involved in modulation of the acute phase, mention may be made of the gene HP.

As examples of human endogenous retroviruses (HERVs), mention may be made of the following HERVs: 190665001-HERV0376, 220247002-HERV0797, 050286701-HERV0513, 130360601-HERV0808, 021456001-MALR1017, 170369402HE41, 121601801-HERV0492, 011052202-HERV1033, 011052702-MALR1044.

TABLE 1
Chromosomal location of the C3AR1 gene and of the other
biomarkers (genes or HERVs) for which the expression
can be measured in combination with that of C3AR1

| Biomarker (gene or HERV) | Chromosomal location (GRCh38/hg38) |
|---|---|
| C3AR1 | chr12: 8,058,302-8,066,471 |
| ADGRE3 | chr19: 14,619,117-14,690,027 |
| ALOX5 | chr10: 45,374,176-45,446,121 |
| ARL14EP | chr11: 30,323,099-30,338,458 |
| BPGM | chr7: 134,646,779-134,679,816 |
| BTLA | chr3: 112,463,966-112,499,702 |
| CCNB1IP1 | chr14: 20,311,368-20,333,312 |
| CD177 | chr19: 43,353,659-43,363,172 |
| CD40LG | chrX: 136,648,177-136,660,390 |
| CD274 | chr9: 5,450,381-5,470,567 |
| CD3D | chr11: 118,338,954-118,342,744 |
| CD74 | chr5: 150,400,041-150,412,936 |
| CIITA | chr16: 10,866,212-10,943,021 |
| CSF2 | chr5: 132,073,789-132,076,170 |
| CTLA4 | chr2: 203,867,771-203,873,965 |
| CX3CR1 | chr3: 39,263,494-39,281,735 |
| CXCL10 | chr4: 76,021,116-76,023,536 |
| DYRK2 | chr12: 67,648,338-67,665,406 |
| FCGR1A | chr1: 149,781,275-149,792,518 |
| FLT1 | chr13: 28,300,346-28,495,145 |
| GATA3 | chr10: 8,045,378-8,075,201 |
| GNLY | chr2: 85,685,175-85,698,854 |
| GSN | chr9: 121,207,794-121,332,844 |
| HAVCR2 | chr5: 157,085,832-157,142,869 |
| HP | chr16: 72,054,592-72,061,056 |
| ICOS | chr2: 203,936,731-203,961,579 |
| IFIH1 | chr2: 162,267,074-162,318,764 |
| IFNG | chr12: 68,154,768-68,159,741 |
| IL10 | chr1: 206,767,602-206,774,607 |
| IL1B | chr2: 112,829,751-112,836,903 |
| IL15 | chr4: 141,636,583-141,733,987 |
| IL1RN | chr2: 113,099,365-113,134,016 |
| IL1R1 | chr2: 102,064,544-102,179,874 |
| IL1R2 | chr2: 101,991,816-102,028,544 |
| IL17A | chr6: 52,186,375-52,190,638 |
| IL18 | chr11: 112,143,251-112,164,117 |
| IL18RAP | chr2: 102,418,558-102,452,568 |
| IL18R1 | chr2: 102,311,529-102,398,777 |
| IL2 | chr4: 122,451,470-122,456,725 |
| IL6 | chr7: 22,725,442-22,732,002 |
| IL7R | chr5: 35,852,695-35,879,603 |
| LILRB2 | chr19: 54,273,812-54,281,184 |
| MDC1 | chr6: 30,699,807-30,717,966 |
| MCP1 (CCL2) | chr17: 34,255,218-34,257,203 |
| NFKB1 | chr4: 102,501,329-102,617,302 |
| OAS2 | chr12: 112,970,819-113,011,723 |
| OAS3 | chr12: 112,938,433-112,973,251 |
| PDCD1 | chr2: 241,849,881-241,858,908 |
| RORC | chr1: 151,806,071-151,832,238 |
| S100A9 | chr1: 153,357,854-153,361,027 |
| STAT4 | chr2: 191,029,576-191,246,172 |
| TAP2 | chr6: 32,821,833-32,838,823 |
| TBX21 | chr17: 47,733,236-47,746,122 |
| TDRD9 | chr14: 103,928,438-104,052,667 |
| TNF | chr6: 31,575,565-31,578,336 |
| TNFSF4 | chr1: 173,183,731-173,208,072 |
| TRAP1 | chr16: 3,651,639-3,717,597 |
| ZAP70 | chr2: 97,713,560-97,744,327 |
| 190665001-HERV0376 | chr19: 54,891,074-54,891,496 |
| 220247002-HERV0797 | chr22: 36,153,696-36,154,283 |
| 050286701-HERV0513 | chr5: 14,551,189-14,551,685 |
| 130360601-HERV0808 | chr13: 42,884,951-42,886,257 |
| 021456001-MALR1017 | chr2: 102,013,616-102,013,971 |
| 170369402HE41 | chr17: 35,505,737-35,508,365 |
| 121601801-HERV0492 | chr12: 112,971,073-112,971,451 |
| 011052202-HERV1033 | chr1: 78,623,489-78,623,954 |
| 011052702-MALR1044 | chr1: 78,648,318-78,648,697 |

TABLE 2
Classification of biomarkers which can be used in combination with C3AR1, by families and according to
their immune function. The site of expression (blood cell type) and the location are also indicated.

| | | Expression site | | |
|---|---|---|---|---|
| Families | Biomarkers | (type of blood cell) | Location | Immune function |
| Family of molecules | GNLY | gamma delta (γδ) T | Intracellular, secreted | Alarmin/defensin |
| involved in the innate | | lymphocytes, CD4 and CD8 | (different isoforms) | |
| immune system | | T lymphocytes | | |
| | S100A9 | Neutrophils, classical | Intracellular, secreted | Alarmin/defensin |
| | | monocytes | (different isoforms) | |
| | ADGRE3 | Basophils, monocytes | Membrane, secreted | Neutrophil |
| | | | (different isoforms) | migration/activation/degranulation |
| | CD177 | Neutrophils | Intracellular | Neutrophil |
| | | | | migration/activation/degranulation |
| | CX3CR1 | Monocytes, NK | Membrane | Neutrophil |
| | | lymphocytes, CD4 and CD8 | | migration/activation/degranulation |
| | | T lymphocytes | | |
| | IFIH1 | Any cell type (neutrophils) | Intracellular | Interferon pathway |
| | OAS2 | Any cell type (monocytes, | Intracellular | Interferon pathway |
| | | plasmablasts) | | |
| | OAS3 | Neutrophils, myeloid | Intracellular | Interferon pathway |
| | | dendritic cells, monocytes | | |
| Family of molecules of the | CCNB1IP1 | Any cell type (monocytes, | Intracellular | Cell cycle |
| cell cycle | | neutrophils, basophils) | | |
| Family of cytokines | IL15 | Monocytes, SM B | Intracellular, secreted | T cell activation/proliferation |
| | | lymphocytes, MAIT | (different isoforms) | |
| | | lymphocytes | | |
| | IL2 | Th2 and Th17 helper T | Secreted | T cell activation/proliferation |
| | | lymphocytes, CD8 T | | |
| | | lymphocytes, central | | |
| | | memory (CM) T | | |
| | | lymphocytes | | |
| | MCP1 (CCL2) | Monocytes, naive B | Secreted | Monocyte recruitment |
| | | lymphocytes | | |
| | CXCL10 | Monocytes | Secreted | Signal transduction |
| Family of anti- | IL10 | Myeloid dendritic cells, | Membrane, secreted | Anti-inflammatory cytokine |
| inflammatory cytokines | | monocytes, follicular | (different isoforms) | |
| | | helper T lymphocytes, Th1 | | |
| | | helper T lymphocytes, | | |
| | | regulatory T lymphocytes, | | |
| | | gamma delta (γδ) T | | |
| | | lymphocytes | | |
| | IL1RN | Neutrophils, monocytes | Membrane, secreted | Anti-inflammatory cytokine |
| | | | (different isoforms) | |
| Family of pro- | IL1R2 | Neutrophils | Membrane, secreted | Pro-inflammatory cytokine receptor |
| inflammatory cytokine | | | (different isoforms) | |
| receptors | IL18RAP | Neutrophils, NK | Intracellular, secreted | Pro-inflammatory cytokine receptor |
| | | lymphocytes, CD4 T | (different isoforms) | |
| | | lymphocytes, MAIT | | |
| | | lymphocytes, gamma delta | | |
| | | (γδ) T lymphocytes | | |
| | IL18R1 | Myeloid dendritic cells, | Intracellular, secreted | Pro-inflammatory cytokine receptor |
| | | plasmacytoid dendritic | (different isoforms) | |
| | | cells, basophils, | | |
| | | neutrophils, T | | |
| | | lymphocytes, NK | | |
| | | lymphocytes | | |
| | IL1R1 | Neutrophil, CD4 memory T | Membrane, secreted | Pro-inflammatory cytokine receptor |
| | | lymphocytes, memory | (different isoforms) | |
| | | regulatory T lymphocytes | | |
| Family of pro- | IFNG | Gamma delta (γδ) T | Secreted | Interferon pathway |
| inflammatory cytokines | | lymphocytes, CD4 and CD8 | | |
| | | T lymphocytes, NK | | |
| | | lymphocytes | | |
| | IL1B | Myeloid dendritic cells, | Intracellular, secreted | Pro-inflammatory cytokine |
| | | monocytes, neutrophils | (different isoforms) | |
| | IL17A | Follicular helper T | Secreted | Pro-inflammatory cytokine |
| | | lymphocytes, Th17 helper | | |
| | | T lymphocytes, regulatory | | |
| | | T lymphocytes | | |
| | IL18 | Myeloid dendritic cells, | Intracellular, secreted | Pro-inflammatory cytokine |
| | | monocytes, progenitor | (different isoforms) | |
| | | cells, plasmacytoid | | |
| | | dendritic cells, basophils, | | |
| | | neutrophils | | |
| | IL6 | Monocytes, naive B | Intracellular, secreted | Pro-inflammatory cytokine |
| | | lymphocytes | (different isoforms) | |
| | TNF | Monocytes | Intracellular, secreted | Pro-inflammatory cytokine |
| | | | (different isoforms) | |
| Family of molecules | ARL14EP | Any cell type except for | Intracellular | Class II major histocompatibility |
| involved in cytoskeleton | | neutrophils | | complex interaction/export |
| formation | GSN | Any cell type (neutrophils, | Intracellular, secreted | Actin filament regulation |
| | | B lymphocytes) | (different isoforms) | |
| Family of molecules | CIITA | Myeloid dendritic cells, | Intracellular | Antigen presentation |
| involved in gene | | monocytes, B lymphocytes | | |
| expression and/or | DYRK2 | Any cell type (T | Intracellular, secreted | Cell growth |
| transcription | | lymphocytes) | (different isoforms) | |
| | GATA3 | Th2 helper T lymphocytes, | Intracellular | T cell activation/proliferation |
| | | regulatory T lymphocytes | | |
| | MDC1 | Any cell type except for | Intracellular | DNA repair |
| | | neutrophils | | |
| | NFKB1 | Any cell type (myeloid | Intracellular | Gene expression/transcription |
| | | dendritic cells, monocytes, | | |
| | | neutrophils) | | |
| | RORC | MAIT lymphocytes, Th17 | Intracellular | T cell activation/proliferation |
| | | helper T lymphocytes | | |
| | STAT4 | T lymphocytes, NK | Intracellular | T cell activation/proliferation |
| | | lymphocytes | | |
| | TBX21 | NK lymphocytes, CD4 and | Intracellular | T cell activation/proliferation |
| | | CD8 T lymphocytes, MAIT | | |
| | | lymphocytes, gamma delta | | |
| | | (γδ) T lymphocytes | | |
| | TDRD9 | Basophils, classical | Intracellular | Gene expression/transcription |
| | | monocytes | | |
| Family of growth factors | CSF2 | NK lymphocytes, myeloid | Secreted | Cell growth |
| | | cells, lymphoid cells | | |
| Family of molecules of the | ALOX5 | Basophils, neutrophils, | Intracellular | Fatty acid metabolism |
| metabolism | | monocytes, B lymphocytes | | |
| | BPGM | Any cell type | Intracellular | Carbohydrate metabolism |
| | TRAP1 | Any cell type | Intracellular | Mitochondrial respiration |
| Family of molecules | CD40LG | T lymphocytes (except CD8 | Membrane, secreted | T cell activation/proliferation |
| involved in the adaptive | | T lymphocytes) | (different isoforms) | |
| immune system | CD3D | T lymphocytes | Intracellular, secreted | T cell activation/proliferation |
| | | | (different isoforms) | |
| | BTLA | B lymphocytes, naive B | Intracellular, secreted | Co-stimulation by CD28 |
| | | lymphocytes | (different isoforms) | family/checkpoint inhibitor |
| | CD274 | Basophils, neutrophils, T | Membrane | Co-stimulation by CD28 |
| | | lymphocytes | | family/checkpoint inhibitor |
| | CTLA4 | Regulatory T lymphocytes, | Intracellular, secreted | Co-stimulation by CD28 |
| | | CD4 T lymphocytes | (different isoforms) | family/checkpoint inhibitor |
| | ICOS | T lymphocytes | Membrane | Co-stimulation by CD28 |
| | | | | family/checkpoint inhibitor |
| | PDCD1 | CD8 EM T lymphocytes, T | Intracellular, secreted | Co-stimulation by CD28 |
| | | lymphocytes | (different isoforms) | family/checkpoint inhibitor |
| | TNFSF4 | Progenitors | Intracellular | Co-stimulation by CD28 |
| | | | | family/checkpoint inhibitor |
| | CD74 | Myeloid dendritic cells, | Membrane | Antigen presentation |
| | | monocytes, B lymphocytes, | | |
| | | plasmacytoid dendritic | | |
| | | cells | | |
| | FCGR1A | Neutrophils, monocytes | Membrane | Antigen presentation |
| | LILRB2 | Neutrophils, monocytes | Membrane | Antigen presentation |
| | TAP2 | Any cell type (neutrophils) | Membrane | Antigen presentation |
| Family of molecules | FLT1 | Neutrophils, plasmacytoid | Intracellular, membrane, | Tyrosine kinase receptor |
| involved in signal | | dendritic cells | secreted (different | |
| transduction | | | isoforms) | |
| | HAVCR2 | Myeloid dendritic cells, | Intracellular, secreted | Receptor |
| | | monocytes, NK | (different isoforms) | |
| | | lymphocytes | | |
| | IL7R | T lymphocytes | Intracellular, secreted | Cytokine receptor |
| | | | (different isoforms) | |
| | ZAP70 | NK lymphocytes, T | Intracellular | T cell activation/proliferation |
| | | lymphocytes | | |
| Family of molecules | HP | Monocytes, neutrophils, | Intracellular, secreted | Receptor (binding and absorption of |
| involved in modulation of | | basophils | (different isoforms) | ligand by scavenger receptor/ |
| the acute phase | | | | neutrophil degranulation) |

Also preferably, the method as described previously, in all the embodiments thereof, comprises, in addition to the step of measuring the expression of C3AR1, a step of measuring, in the biological sample from the patient, the expression:
of at least one, two, three, four, five, six, seven, eight gene(s) selected from the following genes: GNLY, S100A9, ADGRE3, CD177, CX3CR1, IFIH1, OAS2, OAS3, and/or
of the CCNB1IP1 gene, and/or
of at least one, two, three, four, gene(s) selected from the following genes: IL15, IL2, MCP1(CCL2), CXCL10, and/or
of at least one, two gene(s) selected from the following genes: IL10, IL1RN, and/or
of at least one, two, three, four, gene(s) selected from the following genes: IL18R1, IL1R2, ILR1 and IL18RAP and/or
of at least one, two, three, four, five, six gene(s) selected from the following genes: IFNG, IL1B, IL17A, I-18, IL6, TNF, and/or
of at least one, two gene(s) selected from the following genes: ARL14EP, GSN, and/or
of at least one, two, three, four, five, six, seven, eight, nine gene(s) selected from the following genes: CIITA, DYRK2, GATA3, MDC1, NFKB1, RORC, STAT4, TBX21, TDRD9, and/or
of the CSF2 gene, and/or
of at least one, two, three, four, five gene(s) selected from the following genes: ALOX5, BPGM, TRAP1, and/or
of at least one, two, three, four, five, six, seven, eight, nine, ten, eleven, twelve gene(s) selected from the following genes: CD40LG, CD3D, BTLA, CD274, CTLA4, ICOS, PDCD1, TNFSF4, CD74, FCGR1A, LILRB2, TAP2 and/or
of at least one, two, three, four, gene(s) selected from the following genes: FLT1, HAVCR2, IL7R, ZAP70, and/or
of the HP gene, and/or
of at least one, two, three, four, five, six, seven, eight, nine HERV(s) selected from the following HERVs: 190665001-HERV0376, 220247002-HERV0797, 050286701-HERV0513, 130360601-HERV0808, 021456001-MALR1017, 170369402HE41, 121601801-HERV0492, 011052202-HERV1033, 011052702-MALR1044.

More preferably still, the method as described previously, in all the embodiments thereof, comprises, in addition to the step of measuring the expression of C3AR1, a step of measuring, in the biological sample from the patient, the expression of at least one, two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six gene(s) selected from the following genes: GNLY, S100A9, ADGRE3, CD177, CX3CR1, OAS2, CCNB1IP1, IL10, IL1RN, IL1R2, IFNG, TNF, ARL14EP, CIITA, GATA3, MDC1, TDRD9, BPGM, CD3D, CD274, CTLA4, CD74, TAP2, 1L7R, ZAP70, HP.

More preferably still, the method as described previously, in all the embodiments thereof, comprises, in addition to the step of measuring the expression of C3AR1, a step of measuring, in the biological sample from the patient, the expression of at least one, two, three, four, five, six, seven, eight, nine, ten, eleven gene(s) selected from the following genes: S100A9, CD177, CX3CR1, IL1R2, IFNG, CIITA, CD3D, CTLA4, CD74, TAP2, HP.

Particularly preferred combinations of one or more other gene(s), the expression of which can be measured in combination with the expression of C3AR1, are presented in table 3.

TABLE 3
List of preferred combinations of 1 to 11 other gene(s), the expression
of which can be measured in combination with the expression of C3AR1.

| | |
|---|---|
| Combinations of one gene | S100A9 |
| | TAP2 |
| | CD177 |
| | CX3CR1 |
| | IL1R2 |
| | IFNG |
| | CIITA |
| | CD3D |
| | CTLA4 |
| | CD74 |
| | HP |
| Combinations of two | S100A9, TAP2 |
| genes | S100A9, CD177 |
| | S100A9, CX3CR1 |
| | S100A9, IL1R2 |
| | S100A9, IFNG |
| | S100A9, CIITA |
| | S100A9, CD3D |
| | S100A9, CTLA4 |
| | S100A9, CD74 |
| | S100A9, HP |
| | TAP2, CD177 |
| | TAP2, CX3CR1 |
| | TAP2, IL1R2 |
| | TAP2, IFNG |
| | TAP2, CIITA |
| | TAP2, CD3D |
| | TAP2, CTLA4 |
| | TAP2, CD74 |
| | TAP2, HP |
| | CD177, CX3CR1 |
| | CD177, IL1R2 |
| | CD177, IFNG |
| | CD177, CIITA |
| | CD177, CD3D |
| | CD177, CTLA4 |
| | CD177, CD74 |
| | CD177, HP |
| | CX3CR1, IL1R2 |
| | CX3CR1, IFNG |
| | CX3CR1, CIITA |
| | CX3CR1, CD3D |
| | CX3CR1, CTLA4 |
| | CX3CR1, CD74 |
| | CX3CR1, HP |
| | IL1R2, IFNG |
| | IL1R2, CIITA |
| | IL1R2, CD3D |
| | IL1R2, CTLA4 |
| | IL1R2, CD74 |
| | IL1R2, HP |
| | IFNG, CIITA |
| | IFNG, CD3D |
| | IFNG, CTLA4 |
| | IFNG, CD74 |
| | IFNG, HP |
| | CIITA, CD3D |
| | CIITA, CTLA4 |
| | CIITA, CD74 |
| | CIITA, HP |
| | CD3D, CTLA4 |
| | CD3D, CD74 |
| | CD3D, HP |
| | CTLA4, CD74 |
| | CTLA4, HP |
| | CD74, HP |
| Combinations of three | S100A9, TAP2, CD177 |
| genes | S100A9, TAP2, CX3CR1 |
| | S100A9, TAP2, IL1R2 |
| | S100A9, TAP2, IFNG |
| | S100A9, TAP2, CIITA |
| | S100A9, TAP2, CD3D |
| | S100A9, TAP2, CTLA4 |
| | S100A9, TAP2, CD74 |
| | S100A9, TAP2, HP |
| | S100A9, CD177, CX3CR1 |
| | S100A9, CD177, IL1R2 |
| | S100A9, CD177, IFNG |
| | S100A9, CD177, CIITA |
| | S100A9, CD177, CD3D |
| | S100A9, CD177, CTLA4 |
| | S100A9, CD177, CD74 |
| | S100A9, CD177, HP |
| | S100A9, CX3CR1, IL1R2 |
| | S100A9, CX3CR1, IFNG |
| | S100A9, CX3CR1, CIITA |
| | S100A9, CX3CR1, CD3D |
| | S100A9, CX3CR1, CTLA4 |
| | S100A9, CX3CR1, CD74 |
| | S100A9, CX3CR1, HP |
| | S100A9, IL1R2, IFNG |
| | S100A9, IL1R2, CIITA |
| | S100A9, IL1R2, CD3D |
| | S100A9, IL1R2, CTLA4 |
| | S100A9, IL1R2, CD74 |
| | S100A9, IL1R2, HP |
| | S100A9, IFNG, CIITA |
| | S100A9, IFNG, CD3D |
| | S100A9, IFNG, CTLA4 |
| | S100A9, IFNG, CD74 |
| | S100A9, IFNG, HP |
| | S100A9, CIITA, CD3D |
| | S100A9, CIITA, CTLA4 |
| | S100A9, CIITA, CD74 |
| | S100A9, CIITA, HP |
| | S100A9, CD3D, CTLA4 |
| | S100A9, CD3D, CD74 |
| | S100A9, CD3D, HP |
| | S100A9, CTLA4, CD74 |
| | S100A9, CTLA4, HP |
| | S100A9, CD74, HP |
| | TAP2, CD177, CX3CR1 |
| | TAP2, CD177, IL1R2 |
| | TAP2, CD177, IFNG |
| | TAP2, CD177, CIITA |
| | TAP2, CD177, CD3D |
| | TAP2, CD177, CTLA4 |
| | TAP2, CD177, CD74 |
| | TAP2, CD177, HP |
| | TAP2, CX3CR1, IL1R2 |
| | TAP2, CX3CR1, IFNG |
| | TAP2, CX3CR1, CIITA |
| | TAP2, CX3CR1, CD3D |
| | TAP2, CX3CR1, CTLA4 |
| | TAP2, CX3CR1, CD74 |
| | TAP2, CX3CR1, HP |
| | TAP2, IL1R2, IFNG |
| | TAP2, IL1R2, CIITA |
| | TAP2, IL1R2, CD3D |
| | TAP2, IL1R2, CTLA4 |
| | TAP2, IL1R2, CD74 |
| | TAP2, IL1R2, HP |
| | TAP2, IFNG, CIITA |
| | TAP2, IFNG, CD3D |
| | TAP2, IFNG, CTLA4 |
| | TAP2, IFNG, CD74 |
| | TAP2, IFNG, HP |
| | TAP2, CIITA, CD3D |
| | TAP2, CIITA, CTLA4 |
| | TAP2, CIITA, CD74 |
| | TAP2, CIITA, HP |
| | TAP2, CD3D, CTLA4 |
| | TAP2, CD3D, CD74 |
| | TAP2, CD3D, HP |
| | TAP2, CTLA4, CD74 |
| | TAP2, CTLA4, HP |
| | TAP2, CD74, HP |
| | CD177, CX3CR1, IL1R2 |
| | CD177, CX3CR1, IFNG |
| | CD177, CX3CR1, CIITA |
| | CD177, CX3CR1, CD3D |
| | CD177, CX3CR1, CTLA4 |
| | CD177, CX3CR1, CD74 |
| | CD177, CX3CR1, HP |
| | CD177, IL1R2, IFNG |
| | CD177, IL1R2, CIITA |
| | CD177, IL1R2, CD3D |
| | CD177, IL1R2, CTLA4 |
| | CD177, IL1R2, CD74 |
| | CD177, IL1R2, HP |
| | CD177, IFNG, CIITA |
| | CD177, IFNG, CD3D |
| | CD177, IFNG, CTLA4 |
| | CD177, IFNG, CD74 |
| | CD177, IFNG, HP |
| | CD177, CIITA, CD3D |
| | CD177, CIITA, CTLA4 |
| | CD177, CIITA, CD74 |
| | CD177, CIITA, HP |
| | CD177, CD3D, CTLA4 |
| | CD177, CD3D, CD74 |
| | CD177, CD3D, HP |
| | CD177, CTLA4, CD74 |
| | CD177, CTLA4, HP |
| | CD177, CD74, HP |
| | CX3CR1, IL1R2, IFNG |
| | CX3CR1, IL1R2, CIITA |
| | CX3CR1, IL1R2, CD3D |
| | CX3CR1, IL1R2, CTLA4 |
| | CX3CR1, IL1R2, CD74 |
| | CX3CR1, IL1R2, HP |
| | CX3CR1, IFNG, CIITA |
| | CX3CR1, IFNG, CD3D |
| | CX3CR1, IFNG, CTLA4 |
| | CX3CR1, IFNG, CD74 |
| | CX3CR1, IFNG, HP |
| | CX3CR1, CIITA, CD3D |
| | CX3CR1, CIITA, CTLA4 |
| | CX3CR1, CIITA, CD74 |
| | CX3CR1, CIITA, HP |
| | CX3CR1, CD3D, CTLA4 |
| | CX3CR1, CD3D, CD74 |
| | CX3CR1, CD3D, HP |
| | CX3CR1, CTLA4, CD74 |
| | CX3CR1, CTLA4, HP |
| | CX3CR1, CD74, HP |
| | IL1R2, IFNG, CIITA |
| | IL1R2, IFNG, CD3D |
| | IL1R2, IFNG, CTLA4 |
| | IL1R2, IFNG, CD74 |
| | IL1R2, IFNG, HP |
| | IL1R2, CIITA, CD3D |
| | IL1R2, CIITA, CTLA4 |
| | IL1R2, CIITA, CD74 |
| | IL1R2, CIITA, HP |
| | IL1R2, CD3D, CTLA4 |
| | IL1R2, CD3D, CD74 |
| | IL1R2, CD3D, HP |
| | IL1R2, CTLA4, CD74 |
| | IL1R2, CTLA4, HP |
| | IL1R2, CD74, HP |
| | IFNG, CIITA, CD3D |
| | IFNG, CIITA, CTLA4 |
| | IFNG, CIITA, CD74 |
| | IFNG, CIITA, HP |
| | IFNG, CD3D, CTLA4 |
| | IFNG, CD3D, CD74 |
| | IFNG, CD3D, HP |
| | IFNG, CTLA4, CD74 |
| | IFNG, CTLA4, HP |
| | IFNG, CD74, HP |
| | CIITA, CD3D, CTLA4 |
| | CIITA, CD3D, CD74 |
| | CIITA, CD3D, HP |
| | CIITA, CTLA4, CD74 |
| | CIITA, CTLA4, HP |
| | CIITA, CD74, HP |
| | CD3D, CTLA4, CD74 |
| | CD3D, CTLA4, HP |
| | CD3D, CD74, HP |
| | CTLA4, CD74, HP |
| Combinations of four | S100A9, TAP2, CD177, CX3CR1 |
| genes | S100A9, TAP2, CD177, IL1R2 |
| | S100A9, TAP2, CD177, IFNG |
| | S100A9, TAP2, CD177, CIITA |
| | S100A9, TAP2, CD177, CD3D |
| | S100A9, TAP2, CD177, CTLA4 |
| | S100A9, TAP2, CD177, CD74 |
| | S100A9, TAP2, CD177, HP |
| | S100A9, TAP2, CX3CR1, IL1R2 |
| | S100A9, TAP2, CX3CR1, IFNG |
| | S100A9, TAP2, CX3CR1, CIITA |
| | S100A9, TAP2, CX3CR1, CD3D |
| | S100A9, TAP2, CX3CR1, CTLA4 |
| | S100A9, TAP2, CX3CR1, CD74 |
| | S100A9, TAP2, CX3CR1, HP |
| | S100A9, TAP2, IL1R2, IFNG |
| | S100A9, TAP2, IL1R2, CIITA |
| | S100A9, TAP2, IL1R2, CD3D |
| | S100A9, TAP2, IL1R2, CTLA4 |
| | S100A9, TAP2, IL1R2, CD74 |
| | S100A9, TAP2, IL1R2, HP |
| | S100A9, TAP2, IFNG, CIITA |
| | S100A9, TAP2, IFNG, CD3D |
| | S100A9, TAP2, IFNG, CTLA4 |
| | S100A9, TAP2, IFNG, CD74 |
| | S100A9, TAP2, IFNG, HP |
| | S100A9, TAP2, CIITA, CD3D |
| | S100A9, TAP2, CIITA, CTLA4 |
| | S100A9, TAP2, CIITA, CD74 |
| | S100A9, TAP2, CIITA, HP |
| | S100A9, TAP2, CD3D, CTLA4 |
| | S100A9, TAP2, CD3D, CD74 |
| | S100A9, TAP2, CD3D, HP |
| | S100A9, TAP2, CTLA4, CD74 |
| | S100A9, TAP2, CTLA4, HP |
| | S100A9, TAP2, CD74, HP |
| | S100A9, CD177, CX3CR1, IL1R2 |
| | S100A9, CD177, CX3CR1, IFNG |
| | S100A9, CD177, CX3CR1, CIITA |
| | S100A9, CD177, CX3CR1, CD3D |
| | S100A9, CD177, CX3CR1, CTLA4 |
| | S100A9, CD177, CX3CR1, CD74 |
| | S100A9, CD177, CX3CR1, HP |
| | S100A9, CD177, IL1R2, IFNG |
| | S100A9, CD177, IL1R2, CIITA |
| | S100A9, CD177, IL1R2, CD3D |
| | S100A9, CD177, IL1R2, CTLA4 |
| | S100A9, CD177, IL1R2, CD74 |
| | S100A9, CD177, IL1R2, HP |
| | S100A9, CD177, IFNG, CIITA |
| | S100A9, CD177, IFNG, CD3D |
| | S100A9, CD177, IFNG, CTLA4 |
| | S100A9, CD177, IFNG, CD74 |
| | S100A9, CD177, IFNG, HP |
| | S100A9, CD177, CIITA, CD3D |
| | S100A9, CD177, CIITA, CTLA4 |
| | S100A9, CD177, CIITA, CD74 |
| | S100A9, CD177, CIITA, HP |
| | S100A9, CD177, CD3D, CTLA4 |
| | S100A9, CD177, CD3D, CD74 |
| | S100A9, CD177, CD3D, HP |
| | S100A9, CD177, CTLA4, CD74 |
| | S100A9, CD177, CTLA4, HP |
| | S100A9, CD177, CD74, HP |
| | S100A9, CX3CR1, IL1R2, IFNG |
| | S100A9, CX3CR1, IL1R2, CIITA |
| | S100A9, CX3CR1, IL1R2, CD3D |
| | S100A9, CX3CR1, IL1R2, CTLA4 |
| | S100A9, CX3CR1, IL1R2, CD74 |
| | S100A9, CX3CR1, IL1R2, HP |
| | S100A9, CX3CR1, IFNG, CIITA |
| | S100A9, CX3CR1, IFNG, CD3D |
| | S100A9, CX3CR1, IFNG, CTLA4 |
| | S100A9, CX3CR1, IFNG, CD74 |
| | S100A9, CX3CR1, IFNG, HP |
| | S100A9, CX3CR1, CIITA, CD3D |
| | S100A9, CX3CR1, CIITA, CTLA4 |
| | S100A9, CX3CR1, CIITA, CD74 |
| | S100A9, CX3CR1, CIITA, HP |
| | S100A9, CX3CR1, CD3D, CTLA4 |
| | S100A9, CX3CR1, CD3D, CD74 |
| | S100A9, CX3CR1, CD3D, HP |
| | S100A9, CX3CR1, CTLA4, CD74 |
| | S100A9, CX3CR1, CTLA4, HP |
| | S100A9, CX3CR1, CD74, HP |
| | S100A9, IL1R2, IFNG, CIITA |
| | S100A9, IL1R2, IFNG, CD3D |
| | S100A9, IL1R2, IFNG, CTLA4 |
| | S100A9, IL1R2, IFNG, CD74 |
| | S100A9, IL1R2, IFNG, HP |
| | S100A9, IL1R2, CIITA, CD3D |
| | S100A9, IL1R2, CIITA, CTLA4 |
| | S100A9, IL1R2, CIITA, CD74 |
| | S100A9, IL1R2, CIITA, HP |
| | S100A9, IL1R2, CD3D, CTLA4 |
| | S100A9, IL1R2, CD3D, CD74 |
| | S100A9, IL1R2, CD3D, HP |
| | S100A9, IL1R2, CTLA4, CD74 |
| | S100A9, IL1R2, CTLA4, HP |
| | S100A9, IL1R2, CD74, HP |
| | S100A9, IFNG, CIITA, CD3D |
| | S100A9, IFNG, CIITA, CTLA4 |
| | S100A9, IFNG, CIITA, CD74 |
| | S100A9, IFNG, CIITA, HP |
| | S100A9, IFNG, CD3D, CTLA4 |
| | S100A9, IFNG, CD3D, CD74 |
| | S100A9, IFNG, CD3D, HP |
| | S100A9, IFNG, CTLA4, CD74 |
| | S100A9, IFNG, CTLA4, HP |
| | S100A9, IFNG, CD74, HP |
| | S100A9, CIITA, CD3D, CTLA4 |
| | S100A9, CIITA, CD3D, CD74 |
| | S100A9, CIITA, CD3D, HP |
| | S100A9, CIITA, CTLA4, CD74 |
| | S100A9, CIITA, CTLA4, HP |
| | S100A9, CIITA, CD74, HP |
| | S100A9, CD3D, CTLA4, CD74 |
| | S100A9, CD3D, CTLA4, HP |
| | S100A9, CD3D, CD74, HP |
| | S100A9, CTLA4, CD74, HP |
| | TAP2, CD177, CX3CR1, IL1R2 |
| | TAP2, CD177, CX3CR1, IFNG |
| | TAP2, CD177, CX3CR1, CIITA |
| | TAP2, CD177, CX3CR1, CD3D |
| | TAP2, CD177, CX3CR1, CTLA4 |
| | TAP2, CD177, CX3CR1, CD74 |
| | TAP2, CD177, CX3CR1, HP |
| | TAP2, CD177, IL1R2, IFNG |
| | TAP2, CD177, IL1R2, CIITA |
| | TAP2, CD177, IL1R2, CD3D |
| | TAP2, CD177, IL1R2, CTLA4 |
| | TAP2, CD177, IL1R2, CD74 |
| | TAP2, CD177, IL1R2, HP |
| | TAP2, CD177, IFNG, CIITA |
| | TAP2, CD177, IFNG, CD3D |
| | TAP2, CD177, IFNG, CTLA4 |
| | TAP2, CD177, IFNG, CD74 |
| | TAP2, CD177, IFNG, HP |
| | TAP2, CD177, CIITA, CD3D |
| | TAP2, CD177, CIITA, CTLA4 |
| | TAP2, CD177, CIITA, CD74 |
| | TAP2, CD177, CIITA, HP |
| | TAP2, CD177, CD3D, CTLA4 |
| | TAP2, CD177, CD3D, CD74 |
| | TAP2, CD177, CD3D, HP |
| | TAP2, CD177, CTLA4, CD74 |
| | TAP2, CD177, CTLA4, HP |
| | TAP2, CD177, CD74, HP |
| | TAP2, CX3CR1, IL1R2, IFNG |
| | TAP2, CX3CR1, IL1R2, CIITA |
| | TAP2, CX3CR1, IL1R2, CD3D |
| | TAP2, CX3CR1, IL1R2, CTLA4 |
| | TAP2, CX3CR1, IL1R2, CD74 |
| | TAP2, CX3CR1, IL1R2, HP |
| | TAP2, CX3CR1, IFNG, CIITA |
| | TAP2, CX3CR1, IFNG, CD3D |
| | TAP2, CX3CR1, IFNG, CTLA4 |
| | TAP2, CX3CR1, IFNG, CD74 |
| | TAP2, CX3CR1, IFNG, HP |
| | TAP2, CX3CR1, CIITA, CD3D |
| | TAP2, CX3CR1, CIITA, CTLA4 |
| | TAP2, CX3CR1, CIITA, CD74 |
| | TAP2, CX3CR1, CIITA, HP |
| | TAP2, CX3CR1, CD3D, CTLA4 |
| | TAP2, CX3CR1, CD3D, CD74 |
| | TAP2, CX3CR1, CD3D, HP |
| | TAP2, CX3CR1, CTLA4, CD74 |
| | TAP2, CX3CR1, CTLA4, HP |
| | TAP2, CX3CR1, CD74, HP |
| | TAP2, IL1R2, IFNG, CIITA |
| | TAP2, IL1R2, IFNG, CD3D |
| | TAP2, IL1R2, IFNG, CTLA4 |
| | TAP2, IL1R2, IFNG, CD74 |
| | TAP2, IL1R2, IFNG, HP |
| | TAP2, IL1R2, CIITA, CD3D |
| | TAP2, IL1R2, CIITA, CTLA4 |
| | TAP2, IL1R2, CIITA, CD74 |
| | TAP2, IL1R2, CIITA, HP |
| | TAP2, IL1R2, CD3D, CTLA4 |
| | TAP2, IL1R2, CD3D, CD74 |
| | TAP2, IL1R2, CD3D, HP |
| | TAP2, IL1R2, CTLA4, CD74 |
| | TAP2, IL1R2, CTLA4, HP |
| | TAP2, IL1R2, CD74, HP |
| | TAP2, IFNG, CIITA, CD3D |
| | TAP2, IFNG, CIITA, CTLA4 |
| | TAP2, IFNG, CIITA, CD74 |
| | TAP2, IFNG, CIITA, HP |
| | TAP2, IFNG, CD3D, CTLA4 |
| | TAP2, IFNG, CD3D, CD74 |
| | TAP2, IFNG, CD3D, HP |
| | TAP2, IFNG, CTLA4, CD74 |
| | TAP2, IFNG, CTLA4, HP |
| | TAP2, IFNG, CD74, HP |
| | TAP2, CIITA, CD3D, CTLA4 |
| | TAP2, CIITA, CD3D, CD74 |
| | TAP2, CIITA, CD3D, HP |
| | TAP2, CIITA, CTLA4, CD74 |
| | TAP2, CIITA, CTLA4, HP |
| | TAP2, CIITA, CD74, HP |
| | TAP2, CD3D, CTLA4, CD74 |
| | TAP2, CD3D, CTLA4, HP |
| | TAP2, CD3D, CD74, HP |
| | TAP2, CTLA4, CD74, HP |
| | CD177, CX3CR1, IL1R2, IFNG |
| | CD177, CX3CR1, IL1R2, CIITA |
| | CD177, CX3CR1, IL1R2, CD3D |
| | CD177, CX3CR1, IL1R2, CTLA4 |
| | CD177, CX3CR1, IL1R2, CD74 |
| | CD177, CX3CR1, IL1R2, HP |
| | CD177, CX3CR1, IFNG, CIITA |
| | CD177, CX3CR1, IFNG, CD3D |
| | CD177, CX3CR1, IFNG, CTLA4 |
| | CD177, CX3CR1, IFNG, CD74 |
| | CD177, CX3CR1, IFNG, HP |
| | CD177, CX3CR1, CIITA, CD3D |
| | CD177, CX3CR1, CIITA, CTLA4 |
| | CD177, CX3CR1, CIITA, CD74 |
| | CD177, CX3CR1, CIITA, HP |
| | CD177, CX3CR1, CD3D, CTLA4 |
| | CD177, CX3CR1, CD3D, CD74 |
| | CD177, CX3CR1, CD3D, HP |
| | CD177, CX3CR1, CTLA4, CD74 |
| | CD177, CX3CR1, CTLA4, HP |
| | CD177, CX3CR1, CD74, HP |
| | CD177, IL1R2, IFNG, CIITA |
| | CD177, IL1R2, IFNG, CD3D |
| | CD177, IL1R2, IFNG, CTLA4 |
| | CD177, IL1R2, IFNG, CD74 |
| | CD177, IL1R2, IFNG, HP |
| | CD177, IL1R2, CIITA, CD3D |
| | CD177, IL1R2, CIITA, CTLA4 |
| | CD177, IL1R2, CIITA, CD74 |
| | CD177, IL1R2, CIITA, HP |
| | CD177, IL1R2, CD3D, CTLA4 |
| | CD177, IL1R2, CD3D, CD74 |
| | CD177, IL1R2, CD3D, HP |
| | CD177, IL1R2, CTLA4, CD74 |
| | CD177, IL1R2, CTLA4, HP |
| | CD177, IL1R2, CD74, HP |
| | CD177, IFNG, CIITA, CD3D |
| | CD177, IFNG, CIITA, CTLA4 |
| | CD177, IFNG, CIITA, CD74 |
| | CD177, IFNG, CIITA, HP |
| | CD177, IFNG, CD3D, CTLA4 |
| | CD177, IFNG, CD3D, CD74 |
| | CD177, IFNG, CD3D, HP |
| | CD177, IFNG, CTLA4, CD74 |
| | CD177, IFNG, CTLA4, HP |
| | CD177, IFNG, CD74, HP |
| | CD177, CIITA, CD3D, CTLA4 |
| | CD177, CIITA, CD3D, CD74 |
| | CD177, CIITA, CD3D, HP |
| | CD177, CIITA, CTLA4, CD74 |
| | CD177, CIITA, CTLA4, HP |
| | CD177, CIITA, CD74, HP |
| | CD177, CD3D, CTLA4, CD74 |
| | CD177, CD3D, CTLA4, HP |
| | CD177, CD3D, CD74, HP |
| | CD177, CTLA4, CD74, HP |
| | CX3CR1, IL1R2, IFNG, CIITA |
| | CX3CR1, IL1R2, IFNG, CD3D |
| | CX3CR1, IL1R2, IFNG, CTLA4 |
| | CX3CR1, IL1R2, IFNG, CD74 |
| | CX3CR1, IL1R2, IFNG, HP |
| | CX3CR1, IL1R2, CIITA, CD3D |
| | CX3CR1, IL1R2, CIITA, CTLA4 |
| | CX3CR1, IL1R2, CIITA, CD74 |
| | CX3CR1, IL1R2, CIITA, HP |
| | CX3CR1, IL1R2, CD3D, CTLA4 |
| | CX3CR1, IL1R2, CD3D, CD74 |
| | CX3CR1, IL1R2, CD3D, HP |
| | CX3CR1, IL1R2, CTLA4, CD74 |
| | CX3CR1, IL1R2, CTLA4, HP |
| | CX3CR1, IL1R2, CD74, HP |
| | CX3CR1, IFNG, CIITA, CD3D |
| | CX3CR1, IFNG, CIITA, CTLA4 |
| | CX3CR1, IFNG, CIITA, CD74 |
| | CX3CR1, IFNG, CIITA, HP |
| | CX3CR1, IFNG, CD3D, CTLA4 |
| | CX3CR1, IFNG, CD3D, CD74 |
| | CX3CR1, IFNG, CD3D, HP |
| | CX3CR1, IFNG, CTLA4, CD74 |
| | CX3CR1, IFNG, CTLA4, HP |
| | CX3CR1, IFNG, CD74, HP |
| | CX3CR1, CIITA, CD3D, CTLA4 |
| | CX3CR1, CIITA, CD3D, CD74 |
| | CX3CR1, CIITA, CD3D, HP |
| | CX3CR1, CIITA, CTLA4, CD74 |
| | CX3CR1, CIITA, CTLA4, HP |
| | CX3CR1, CIITA, CD74, HP |
| | CX3CR1, CD3D, CTLA4, CD74 |
| | CX3CR1, CD3D, CTLA4, HP |
| | CX3CR1, CD3D, CD74, HP |
| | CX3CR1, CTLA4, CD74, HP |
| | IL1R2, IFNG, CIITA, CD3D |
| | IL1R2, IFNG, CIITA, CTLA4 |
| | IL1R2, IFNG, CIITA, CD74 |
| | IL1R2, IFNG, CIITA, HP |
| | IL1R2, IFNG, CD3D, CTLA4 |
| | IL1R2, IFNG, CD3D, CD74 |
| | IL1R2, IFNG, CD3D, HP |
| | IL1R2, IFNG, CTLA4, CD74 |
| | IL1R2, IFNG, CTLA4, HP |
| | IL1R2, IFNG, CD74, HP |
| | IL1R2, CIITA, CD3D, CTLA4 |
| | IL1R2, CIITA, CD3D, CD74 |
| | IL1R2, CIITA, CD3D, HP |
| | IL1R2, CIITA, CTLA4, CD74 |
| | IL1R2, CIITA, CTLA4, HP |
| | IL1R2, CIITA, CD74, HP |
| | IL1R2, CD3D, CTLA4, CD74 |
| | IL1R2, CD3D, CTLA4, HP |
| | IL1R2, CD3D, CD74, HP |
| | IL1R2, CTLA4, CD74, HP |
| | IFNG, CIITA, CD3D, CTLA4 |
| | IFNG, CIITA, CD3D, CD74 |
| | IFNG, CIITA, CD3D, HP |
| | IFNG, CIITA, CTLA4, CD74 |
| | IFNG, CIITA, CTLA4, HP |
| | IFNG, CIITA, CD74, HP |
| | IFNG, CD3D, CTLA4, CD74 |
| | IFNG, CD3D, CTLA4, HP |
| | IFNG, CD3D, CD74, HP |
| | IFNG, CTLA4, CD74, HP |
| | CIITA, CD3D, CTLA4, CD74 |
| | CIITA, CD3D, CTLA4, HP |
| | CIITA, CD3D, CD74, HP |
| | CIITA, CTLA4, CD74, HP |
| | CD3D, CTLA4, CD74, HP |
| Combinations of five | S100A9, TAP2, CD177, CX3CR1, IL1R2 |
| genes | S100A9, TAP2, CD177, CX3CR1, IFNG |
| | S100A9, TAP2, CD177, CX3CR1, CIITA |
| | S100A9, TAP2, CD177, CX3CR1, CD3D |
| | S100A9, TAP2, CD177, CX3CR1, CTLA4 |
| | S100A9, TAP2, CD177, CX3CR1, CD74 |
| | S100A9, TAP2, CD177, CX3CR1, HP |
| | S100A9, TAP2, CD177, IL1R2, IFNG |
| | S100A9, TAP2, CD177, IL1R2, CIITA |
| | S100A9, TAP2, CD177, IL1R2, CD3D |
| | S100A9, TAP2, CD177, IL1R2, CTLA4 |
| | S100A9, TAP2, CD177, IL1R2, CD74 |
| | S100A9, TAP2, CD177, IL1R2, HP |
| | S100A9, TAP2, CD177, IFNG, CIITA |
| | S100A9, TAP2, CD177, IFNG, CD3D |
| | S100A9, TAP2, CD177, IFNG, CTLA4 |
| | S100A9, TAP2, CD177, IFNG, CD74 |
| | S100A9, TAP2, CD177, IFNG, HP |
| | S100A9, TAP2, CD177, CIITA, CD3D |
| | S100A9, TAP2, CD177, CIITA, CTLA4 |
| | S100A9, TAP2, CD177, CIITA, CD74 |
| | S100A9, TAP2, CD177, CIITA, HP |
| | S100A9, TAP2, CD177, CD3D, CTLA4 |
| | S100A9, TAP2, CD177, CD3D, CD74 |
| | S100A9, TAP2, CD177, CD3D, HP |
| | S100A9, TAP2, CD177, CTLA4, CD74 |
| | S100A9, TAP2, CD177, CTLA4, HP |
| | S100A9, TAP2, CD177, CD74, HP |
| | S100A9, TAP2, CX3CR1, IL1R2, IFNG |
| | S100A9, TAP2, CX3CR1, IL1R2, CIITA |
| | S100A9, TAP2, CX3CR1, IL1R2, CD3D |
| | S100A9, TAP2, CX3CR1, IL1R2, CTLA4 |
| | S100A9, TAP2, CX3CR1, IL1R2, CD74 |
| | S100A9, TAP2, CX3CR1, IL1R2, HP |
| | S100A9, TAP2, CX3CR1, IFNG, CIITA |
| | S100A9, TAP2, CX3CR1, IFNG, CD3D |
| | S100A9, TAP2, CX3CR1, IFNG, CTLA4 |
| | S100A9, TAP2, CX3CR1, IFNG, CD74 |
| | S100A9, TAP2, CX3CR1, IFNG, HP |
| | S100A9, TAP2, CX3CR1, CIITA, CD3D |
| | S100A9, TAP2, CX3CR1, CIITA, CTLA4 |
| | S100A9, TAP2, CX3CR1, CIITA, CD74 |
| | S100A9, TAP2, CX3CR1, CIITA, HP |
| | S100A9, TAP2, CX3CR1, CD3D, CTLA4 |
| | S100A9, TAP2, CX3CR1, CD3D, CD74 |
| | S100A9, TAP2, CX3CR1, CD3D, HP |
| | S100A9, TAP2, CX3CR1, CTLA4, CD74 |
| | S100A9, TAP2, CX3CR1, CTLA4, HP |
| | S100A9, TAP2, CX3CR1, CD74, HP |
| | S100A9, TAP2, IL1R2, IFNG, CIITA |
| | S100A9, TAP2, IL1R2, IFNG, CD3D |
| | S100A9, TAP2, IL1R2, IFNG, CTLA4 |
| | S100A9, TAP2, IL1R2, IFNG, CD74 |
| | S100A9, TAP2, IL1R2, IFNG, HP |
| | S100A9, TAP2, IL1R2, CIITA, CD3D |
| | S100A9, TAP2, IL1R2, CIITA, CTLA4 |
| | S100A9, TAP2, IL1R2, CIITA, CD74 |
| | S100A9, TAP2, IL1R2, CIITA, HP |
| | S100A9, TAP2, IL1R2, CD3D, CTLA4 |
| | S100A9, TAP2, IL1R2, CD3D, CD74 |
| | S100A9, TAP2, IL1R2, CD3D, HP |
| | S100A9, TAP2, IL1R2, CTLA4, CD74 |
| | S100A9, TAP2, IL1R2, CTLA4, HP |
| | S100A9, TAP2, IL1R2, CD74, HP |
| | S100A9, TAP2, IFNG, CIITA, CD3D |
| | S100A9, TAP2, IFNG, CIITA, CTLA4 |
| | S100A9, TAP2, IFNG, CIITA, CD74 |
| | S100A9, TAP2, IFNG, CIITA, HP |
| | S100A9, TAP2, IFNG, CD3D, CTLA4 |
| | S100A9, TAP2, IFNG, CD3D, CD74 |
| | S100A9, TAP2, IFNG, CD3D, HP |
| | S100A9, TAP2, IFNG, CTLA4, CD74 |
| | S100A9, TAP2, IFNG, CTLA4, HP |
| | S100A9, TAP2, IFNG, CD74, HP |
| | S100A9, TAP2, CIITA, CD3D, CTLA4 |
| | S100A9, TAP2, CIITA, CD3D, CD74 |
| | S100A9, TAP2, CIITA, CD3D, HP |
| | S100A9, TAP2, CIITA, CTLA4, CD74 |
| | S100A9, TAP2, CIITA, CTLA4, HP |
| | S100A9, TAP2, CIITA, CD74, HP |
| | S100A9, TAP2, CD3D, CTLA4, CD74 |
| | S100A9, TAP2, CD3D, CTLA4, HP |
| | S100A9, TAP2, CD3D, CD74, HP |
| | S100A9, TAP2, CTLA4, CD74, HP |
| | S100A9, CD177, CX3CR1, IL1R2, IFNG |
| | S100A9, CD177, CX3CR1, IL1R2, CIITA |
| | S100A9, CD177, CX3CR1, IL1R2, CD3D |
| | S100A9, CD177, CX3CR1, IL1R2, CTLA4 |
| | S100A9, CD177, CX3CR1, IL1R2, CD74 |
| | S100A9, CD177, CX3CR1, IL1R2, HP |
| | S100A9, CD177, CX3CR1, IFNG, CIITA |
| | S100A9, CD177, CX3CR1, IFNG, CD3D |
| | S100A9, CD177, CX3CR1, IFNG, CTLA4 |
| | S100A9, CD177, CX3CR1, IFNG, CD74 |
| | S100A9, CD177, CX3CR1, IFNG, HP |
| | S100A9, CD177, CX3CR1, CIITA, CD3D |
| | S100A9, CD177, CX3CR1, CIITA, CTLA4 |
| | S100A9, CD177, CX3CR1, CIITA, CD74 |
| | S100A9, CD177, CX3CR1, CIITA, HP |
| | S100A9, CD177, CX3CR1, CD3D, CTLA4 |
| | S100A9, CD177, CX3CR1, CD3D, CD74 |
| | S100A9, CD177, CX3CR1, CD3D, HP |
| | S100A9, CD177, CX3CR1, CTLA4, CD74 |
| | S100A9, CD177, CX3CR1, CTLA4, HP |
| | S100A9, CD177, CX3CR1, CD74, HP |
| | S100A9, CD177, IL1R2, IFNG, CIITA |
| | S100A9, CD177, IL1R2, IFNG, CD3D |
| | S100A9, CD177, IL1R2, IFNG, CTLA4 |
| | S100A9, CD177, IL1R2, IFNG, CD74 |
| | S100A9, CD177, IL1R2, IFNG, HP |
| | S100A9, CD177, IL1R2, CIITA, CD3D |
| | S100A9, CD177, IL1R2, CIITA, CTLA4 |
| | S100A9, CD177, IL1R2, CIITA, CD74 |
| | S100A9, CD177, IL1R2, CIITA, HP |
| | S100A9, CD177, IL1R2, CD3D, CTLA4 |
| | S100A9, CD177, IL1R2, CD3D, CD74 |
| | S100A9, CD177, IL1R2, CD3D, HP |
| | S100A9, CD177, IL1R2, CTLA4, CD74 |
| | S100A9, CD177, IL1R2, CTLA4, HP |
| | S100A9, CD177, IL1R2, CD74, HP |
| | S100A9, CD177, IFNG, CIITA, CD3D |
| | S100A9, CD177, IFNG, CIITA, CTLA4 |
| | S100A9, CD177, IFNG, CIITA, CD74 |
| | S100A9, CD177, IFNG, CIITA, HP |
| | S100A9, CD177, IFNG, CD3D, CTLA4 |
| | S100A9, CD177, IFNG, CD3D, CD74 |
| | S100A9, CD177, IFNG, CD3D, HP |
| | S100A9, CD177, IFNG, CTLA4, CD74 |
| | S100A9, CD177, IFNG, CTLA4, HP |
| | S100A9, CD177, IFNG, CD74, HP |
| | S100A9, CD177, CIITA, CD3D, CTLA4 |
| | S100A9, CD177, CIITA, CD3D, CD74 |
| | S100A9, CD177, CIITA, CD3D, HP |
| | S100A9, CD177, CIITA, CTLA4, CD74 |
| | S100A9, CD177, CIITA, CTLA4, HP |
| | S100A9, CD177, CIITA, CD74, HP |
| | S100A9, CD177, CD3D, CTLA4, CD74 |
| | S100A9, CD177, CD3D, CTLA4, HP |
| | S100A9, CD177, CD3D, CD74, HP |
| | S100A9, CD177, CTLA4, CD74, HP |
| | S100A9, CX3CR1, IL1R2, IFNG, CIITA |
| | S100A9, CX3CR1, IL1R2, IFNG, CD3D |
| | S100A9, CX3CR1, IL1R2, IFNG, CTLA4 |
| | S100A9, CX3CR1, IL1R2, IFNG, CD74 |
| | S100A9, CX3CR1, IL1R2, IFNG, HP |
| | S100A9, CX3CR1, IL1R2, CIITA, CD3D |
| | S100A9, CX3CR1, IL1R2, CIITA, CTLA4 |
| | S100A9, CX3CR1, IL1R2, CIITA, CD74 |
| | S100A9, CX3CR1, IL1R2, CIITA, HP |
| | S100A9, CX3CR1, IL1R2, CD3D, CTLA4 |
| | S100A9, CX3CR1, IL1R2, CD3D, CD74 |
| | S100A9, CX3CR1, IL1R2, CD3D, HP |
| | S100A9, CX3CR1, IL1R2, CTLA4, CD74 |
| | S100A9, CX3CR1, IL1R2, CTLA4, HP |
| | S100A9, CX3CR1, IL1R2, CD74, HP |
| | S100A9, CX3CR1, IFNG, CIITA, CD3D |
| | S100A9, CX3CR1, IFNG, CIITA, CTLA4 |
| | S100A9, CX3CR1, IFNG, CIITA, CD74 |
| | S100A9, CX3CR1, IFNG, CIITA, HP |
| | S100A9, CX3CR1, IFNG, CD3D, CTLA4 |
| | S100A9, CX3CR1, IFNG, CD3D, CD74 |
| | S100A9, CX3CR1, IFNG, CD3D, HP |
| | S100A9, CX3CR1, IFNG, CTLA4, CD74 |
| | S100A9, CX3CR1, IFNG, CTLA4, HP |
| | S100A9, CX3CR1, IFNG, CD74, HP |
| | S100A9, CX3CR1, CIITA, CD3D, CTLA4 |
| | S100A9, CX3CR1, CIITA, CD3D, CD74 |
| | S100A9, CX3CR1, CIITA, CD3D, HP |
| | S100A9, CX3CR1, CIITA, CTLA4, CD74 |
| | S100A9, CX3CR1, CIITA, CTLA4, HP |
| | S100A9, CX3CR1, CIITA, CD74, HP |
| | S100A9, CX3CR1, CD3D, CTLA4, CD74 |
| | S100A9, CX3CR1, CD3D, CTLA4, HP |
| | S100A9, CX3CR1, CD3D, CD74, HP |
| | S100A9, CX3CR1, CTLA4, CD74, HP |
| | S100A9, IL1R2, IFNG, CIITA, CD3D |
| | S100A9, IL1R2, IFNG, CIITA, CTLA4 |
| | S100A9, IL1R2, IFNG, CIITA, CD74 |
| | S100A9, IL1R2, IFNG, CIITA, HP |
| | S100A9, IL1R2, IFNG, CD3D, CTLA4 |
| | S100A9, IL1R2, IFNG, CD3D, CD74 |
| | S100A9, IL1R2, IFNG, CD3D, HP |
| | S100A9, IL1R2, IFNG, CTLA4, CD74 |
| | S100A9, IL1R2, IFNG, CTLA4, HP |
| | S100A9, IL1R2, IFNG, CD74, HP |
| | S100A9, IL1R2, CIITA, CD3D, CTLA4 |
| | S100A9, IL1R2, CIITA, CD3D, CD74 |
| | S100A9, IL1R2, CIITA, CD3D, HP |
| | S100A9, IL1R2, CIITA, CTLA4, CD74 |
| | S100A9, IL1R2, CIITA, CTLA4, HP |
| | S100A9, IL1R2, CIITA, CD74, HP |
| | S100A9, IL1R2, CD3D, CTLA4, CD74 |
| | S100A9, IL1R2, CD3D, CTLA4, HP |
| | S100A9, IL1R2, CD3D, CD74, HP |
| | S100A9, IL1R2, CTLA4, CD74, HP |
| | S100A9, IFNG, CIITA, CD3D, CTLA4 |
| | S100A9, IFNG, CIITA, CD3D, CD74 |
| | S100A9, IFNG, CIITA, CD3D, HP |
| | S100A9, IFNG, CIITA, CTLA4, CD74 |
| | S100A9, IFNG, CIITA, CTLA4, HP |
| | S100A9, IFNG, CIITA, CD74, HP |
| | S100A9, IFNG, CD3D, CTLA4, CD74 |
| | S100A9, IFNG, CD3D, CTLA4, HP |
| | S100A9, IFNG, CD3D, CD74, HP |
| | S100A9, IFNG, CTLA4, CD74, HP |
| | S100A9, CIITA, CD3D, CTLA4, CD74 |
| | S100A9, CIITA, CD3D, CTLA4, HP |
| | S100A9, CIITA, CD3D, CD74, HP |
| | S100A9, CIITA, CTLA4, CD74, HP |
| | S100A9, CD3D, CTLA4, CD74, HP |
| | TAP2, CD177, CX3CR1, IL1R2, IFNG |
| | TAP2, CD177, CX3CR1, IL1R2, CIITA |
| | TAP2, CD177, CX3CR1, IL1R2, CD3D |
| | TAP2, CD177, CX3CR1, IL1R2, CTLA4 |
| | TAP2, CD177, CX3CR1, IL1R2, CD74 |
| | TAP2, CD177, CX3CR1, IL1R2, HP |
| | TAP2, CD177, CX3CR1, IFNG, CIITA |
| | TAP2, CD177, CX3CR1, IFNG, CD3D |
| | TAP2, CD177, CX3CR1, IFNG, CTLA4 |
| | TAP2, CD177, CX3CR1, IFNG, CD74 |
| | TAP2, CD177, CX3CR1, IFNG, HP |
| | TAP2, CD177, CX3CR1, CIITA, CD3D |
| | TAP2, CD177, CX3CR1, CIITA, CTLA4 |
| | TAP2, CD177, CX3CR1, CIITA, CD74 |
| | TAP2, CD177, CX3CR1, CIITA, HP |
| | TAP2, CD177, CX3CR1, CD3D, CTLA4 |
| | TAP2, CD177, CX3CR1, CD3D, CD74 |
| | TAP2, CD177, CX3CR1, CD3D, HP |
| | TAP2, CD177, CX3CR1, CTLA4, CD74 |
| | TAP2, CD177, CX3CR1, CTLA4, HP |
| | TAP2, CD177, CX3CR1, CD74, HP |
| | TAP2, CD177, IL1R2, IFNG, CIITA |
| | TAP2, CD177, IL1R2, IFNG, CD3D |
| | TAP2, CD177, IL1R2, IFNG, CTLA4 |
| | TAP2, CD177, IL1R2, IFNG, CD74 |
| | TAP2, CD177, IL1R2, IFNG, HP |
| | TAP2, CD177, IL1R2, CIITA, CD3D |
| | TAP2, CD177, IL1R2, CIITA, CTLA4 |
| | TAP2, CD177, IL1R2, CIITA, CD74 |
| | TAP2, CD177, IL1R2, CIITA, HP |
| | TAP2, CD177, IL1R2, CD3D, CTLA4 |
| | TAP2, CD177, IL1R2, CD3D, CD74 |
| | TAP2, CD177, IL1R2, CD3D, HP |
| | TAP2, CD177, IL1R2, CTLA4, CD74 |
| | TAP2, CD177, IL1R2, CTLA4, HP |
| | TAP2, CD177, IL1R2, CD74, HP |
| | TAP2, CD177, IFNG, CIITA, CD3D |
| | TAP2, CD177, IFNG, CIITA, CTLA4 |
| | TAP2, CD177, IFNG, CIITA, CD74 |
| | TAP2, CD177, IFNG, CIITA, HP |
| | TAP2, CD177, IFNG, CD3D, CTLA4 |
| | TAP2, CD177, IFNG, CD3D, CD74 |
| | TAP2, CD177, IFNG, CD3D, HP |
| | TAP2, CD177, IFNG, CTLA4, CD74 |
| | TAP2, CD177, IFNG, CTLA4, HP |
| | TAP2, CD177, IFNG, CD74, HP |
| | TAP2, CD177, CIITA, CD3D, CTLA4 |
| | TAP2, CD177, CIITA, CD3D, CD74 |
| | TAP2, CD177, CIITA, CD3D, HP |
| | TAP2, CD177, CIITA, CTLA4, CD74 |
| | TAP2, CD177, CIITA, CTLA4, HP |
| | TAP2, CD177, CIITA, CD74, HP |
| | TAP2, CD177, CD3D, CTLA4, CD74 |
| | TAP2, CD177, CD3D, CTLA4, HP |
| | TAP2, CD177, CD3D, CD74, HP |
| | TAP2, CD177, CTLA4, CD74, HP |
| | TAP2, CX3CR1, IL1R2, IFNG, CIITA |
| | TAP2, CX3CR1, IL1R2, IFNG, CD3D |
| | TAP2, CX3CR1, IL1R2, IFNG, CTLA4 |
| | TAP2, CX3CR1, IL1R2, IFNG, CD74 |
| | TAP2, CX3CR1, IL1R2, IFNG, HP |
| | TAP2, CX3CR1, IL1R2, CIITA, CD3D |
| | TAP2, CX3CR1, IL1R2, CIITA, CTLA4 |
| | TAP2, CX3CR1, IL1R2, CIITA, CD74 |
| | TAP2, CX3CR1, IL1R2, CIITA, HP |
| | TAP2, CX3CR1, IL1R2, CD3D, CTLA4 |
| | TAP2, CX3CR1, IL1R2, CD3D, CD74 |
| | TAP2, CX3CR1, IL1R2, CD3D, HP |
| | TAP2, CX3CR1, IL1R2, CTLA4, CD74 |
| | TAP2, CX3CR1, IL1R2, CTLA4, HP |
| | TAP2, CX3CR1, IL1R2, CD74, HP |
| | TAP2, CX3CR1, IFNG, CIITA, CD3D |
| | TAP2, CX3CR1, IFNG, CIITA, CTLA4 |
| | TAP2, CX3CR1, IFNG, CIITA, CD74 |
| | TAP2, CX3CR1, IFNG, CIITA, HP |
| | TAP2, CX3CR1, IFNG, CD3D, CTLA4 |
| | TAP2, CX3CR1, IFNG, CD3D, CD74 |
| | TAP2, CX3CR1, IFNG, CD3D, HP |
| | TAP2, CX3CR1, IFNG, CTLA4, CD74 |
| | TAP2, CX3CR1, IFNG, CTLA4, HP |
| | TAP2, CX3CR1, IFNG, CD74, HP |
| | TAP2, CX3CR1, CIITA, CD3D, CTLA4 |
| | TAP2, CX3CR1, CIITA, CD3D, CD74 |
| | TAP2, CX3CR1, CIITA, CD3D, HP |
| | TAP2, CX3CR1, CIITA, CTLA4, CD74 |
| | TAP2, CX3CR1, CIITA, CTLA4, HP |
| | TAP2, CX3CR1, CIITA, CD74, HP |
| | TAP2, CX3CR1, CD3D, CTLA4, CD74 |
| | TAP2, CX3CR1, CD3D, CTLA4, HP |
| | TAP2, CX3CR1, CD3D, CD74, HP |
| | TAP2, CX3CR1, CTLA4, CD74, HP |
| | TAP2, IL1R2, IFNG, CIITA, CD3D |
| | TAP2, IL1R2, IFNG, CIITA, CTLA4 |
| | TAP2, IL1R2, IFNG, CIITA, CD74 |
| | TAP2, IL1R2, IFNG, CIITA, HP |
| | TAP2, IL1R2, IFNG, CD3D, CTLA4 |
| | TAP2, IL1R2, IFNG, CD3D, CD74 |
| | TAP2, IL1R2, IFNG, CD3D, HP |
| | TAP2, IL1R2, IFNG, CTLA4, CD74 |
| | TAP2, IL1R2, IFNG, CTLA4, HP |
| | TAP2, IL1R2, IFNG, CD74, HP |
| | TAP2, IL1R2, CIITA, CD3D, CTLA4 |
| | TAP2, IL1R2, CIITA, CD3D, CD74 |
| | TAP2, IL1R2, CIITA, CD3D, HP |
| | TAP2, IL1R2, CIITA, CTLA4, CD74 |
| | TAP2, IL1R2, CIITA, CTLA4, HP |
| | TAP2, IL1R2, CIITA, CD74, HP |
| | TAP2, IL1R2, CD3D, CTLA4, CD74 |
| | TAP2, IL1R2, CD3D, CTLA4, HP |
| | TAP2, IL1R2, CD3D, CD74, HP |
| | TAP2, IL1R2, CTLA4, CD74, HP |
| | TAP2, IFNG, CIITA, CD3D, CTLA4 |
| | TAP2, IFNG, CIITA, CD3D, CD74 |
| | TAP2, IFNG, CIITA, CD3D, HP |
| | TAP2, IFNG, CIITA, CTLA4, CD74 |
| | TAP2, IFNG, CIITA, CTLA4, HP |
| | TAP2, IFNG, CIITA, CD74, HP |
| | TAP2, IFNG, CD3D, CTLA4, CD74 |
| | TAP2, IFNG, CD3D, CTLA4, HP |
| | TAP2, IFNG, CD3D, CD74, HP |
| | TAP2, IFNG, CTLA4, CD74, HP |
| | TAP2, CIITA, CD3D, CTLA4, CD74 |
| | TAP2, CIITA, CD3D, CTLA4, HP |
| | TAP2, CIITA, CD3D, CD74, HP |
| | TAP2, CIITA, CTLA4, CD74, HP |
| | TAP2, CD3D, CTLA4, CD74, HP |
| | CD177, CX3CR1, IL1R2, IFNG, CIITA |
| | CD177, CX3CR1, IL1R2, IFNG, CD3D |
| | CD177, CX3CR1, IL1R2, IFNG, CTLA4 |
| | CD177, CX3CR1, IL1R2, IFNG, CD74 |
| | CD177, CX3CR1, IL1R2, IFNG, HP |
| | CD177, CX3CR1, IL1R2, CIITA, CD3D |
| | CD177, CX3CR1, IL1R2, CIITA, CTLA4 |
| | CD177, CX3CR1, IL1R2, CIITA, CD74 |
| | CD177, CX3CR1, IL1R2, CIITA, HP |
| | CD177, CX3CR1, IL1R2, CD3D, CTLA4 |
| | CD177, CX3CR1, IL1R2, CD3D, CD74 |
| | CD177, CX3CR1, IL1R2, CD3D, HP |
| | CD177, CX3CR1, IL1R2, CTLA4, CD74 |
| | CD177, CX3CR1, IL1R2, CTLA4, HP |
| | CD177, CX3CR1, IL1R2, CD74, HP |
| | CD177, CX3CR1, IFNG, CIITA, CD3D |
| | CD177, CX3CR1, IFNG, CIITA, CTLA4 |
| | CD177, CX3CR1, IFNG, CIITA, CD74 |
| | CD177, CX3CR1, IFNG, CIITA, HP |
| | CD177, CX3CR1, IFNG, CD3D, CTLA4 |
| | CD177, CX3CR1, IFNG, CD3D, CD74 |
| | CD177, CX3CR1, IFNG, CD3D, HP |
| | CD177, CX3CR1, IFNG, CTLA4, CD74 |
| | CD177, CX3CR1, IFNG, CTLA4, HP |
| | CD177, CX3CR1, IFNG, CD74, HP |
| | CD177, CX3CR1, CIITA, CD3D, CTLA4 |
| | CD177, CX3CR1, CIITA, CD3D, CD74 |
| | CD177, CX3CR1, CIITA, CD3D, HP |
| | CD177, CX3CR1, CIITA, CTLA4, CD74 |
| | CD177, CX3CR1, CIITA, CTLA4, HP |
| | CD177, CX3CR1, CIITA, CD74, HP |
| | CD177, CX3CR1, CD3D, CTLA4, CD74 |
| | CD177, CX3CR1, CD3D, CTLA4, HP |
| | CD177, CX3CR1, CD3D, CD74, HP |
| | CD177, CX3CR1, CTLA4, CD74, HP |
| | CD177, IL1R2, IFNG, CIITA, CD3D |
| | CD177, IL1R2, IFNG, CIITA, CTLA4 |
| | CD177, IL1R2, IFNG, CIITA, CD74 |
| | CD177, IL1R2, IFNG, CIITA, HP |
| | CD177, IL1R2, IFNG, CD3D, CTLA4 |
| | CD177, IL1R2, IFNG, CD3D, CD74 |
| | CD177, IL1R2, IFNG, CD3D, HP |
| | CD177, IL1R2, IFNG, CTLA4, CD74 |
| | CD177, IL1R2, IFNG, CTLA4, HP |
| | CD177, IL1R2, IFNG, CD74, HP |
| | CD177, IL1R2, CIITA, CD3D, CTLA4 |
| | CD177, IL1R2, CIITA, CD3D, CD74 |
| | CD177, IL1R2, CIITA, CD3D, HP |
| | CD177, IL1R2, CIITA, CTLA4, CD74 |
| | CD177, IL1R2, CIITA, CTLA4, HP |
| | CD177, IL1R2, CIITA, CD74, HP |
| | CD177, IL1R2, CD3D, CTLA4, CD74 |
| | CD177, IL1R2, CD3D, CTLA4, HP |
| | CD177, IL1R2, CD3D, CD74, HP |
| | CD177, IL1R2, CTLA4, CD74, HP |
| | CD177, IFNG, CIITA, CD3D, CTLA4 |
| | CD177, IFNG, CIITA, CD3D, CD74 |
| | CD177, IFNG, CIITA, CD3D, HP |
| | CD177, IFNG, CIITA, CTLA4, CD74 |
| | CD177, IFNG, CIITA, CTLA4, HP |
| | CD177, IFNG, CIITA, CD74, HP |
| | CD177, IFNG, CD3D, CTLA4, CD74 |
| | CD177, IFNG, CD3D, CTLA4, HP |
| | CD177, IFNG, CD3D, CD74, HP |
| | CD177, IFNG, CTLA4, CD74, HP |
| | CD177, CIITA, CD3D, CTLA4, CD74 |
| | CD177, CIITA, CD3D, CTLA4, HP |
| | CD177, CIITA, CD3D, CD74, HP |
| | CD177, CIITA, CTLA4, CD74, HP |
| | CD177, CD3D, CTLA4, CD74, HP |
| | CX3CR1, IL1R2, IFNG, CIITA, CD3D |
| | CX3CR1, IL1R2, IFNG, CIITA, CTLA4 |
| | CX3CR1, IL1R2, IFNG, CIITA, CD74 |
| | CX3CR1, IL1R2, IFNG, CIITA, HP |
| | CX3CR1, IL1R2, IFNG, CD3D, CTLA4 |
| | CX3CR1, IL1R2, IFNG, CD3D, CD74 |
| | CX3CR1, IL1R2, IFNG, CD3D, HP |
| | CX3CR1, IL1R2, IFNG, CTLA4, CD74 |
| | CX3CR1, IL1R2, IFNG, CTLA4, HP |
| | CX3CR1, IL1R2, IFNG, CD74, HP |
| | CX3CR1, IL1R2, CIITA, CD3D, CTLA4 |
| | CX3CR1, IL1R2, CIITA, CD3D, CD74 |
| | CX3CR1, IL1R2, CIITA, CD3D, HP |
| | CX3CR1, IL1R2, CIITA, CTLA4, CD74 |
| | CX3CR1, IL1R2, CIITA, CTLA4, HP |
| | CX3CR1, IL1R2, CIITA, CD74, HP |
| | CX3CR1, IL1R2, CD3D, CTLA4, CD74 |
| | CX3CR1, IL1R2, CD3D, CTLA4, HP |
| | CX3CR1, IL1R2, CD3D, CD74, HP |
| | CX3CR1, IL1R2, CTLA4, CD74, HP |
| | CX3CR1, IFNG, CIITA, CD3D, CTLA4 |
| | CX3CR1, IFNG, CIITA, CD3D, CD74 |
| | CX3CR1, IFNG, CIITA, CD3D, HP |
| | CX3CR1, IFNG, CIITA, CTLA4, CD74 |
| | CX3CR1, IFNG, CIITA, CTLA4, HP |
| | CX3CR1, IFNG, CIITA, CD74, HP |
| | CX3CR1, IFNG, CD3D, CTLA4, CD74 |
| | CX3CR1, IFNG, CD3D, CTLA4, HP |
| | CX3CR1, IFNG, CD3D, CD74, HP |
| | CX3CR1, IFNG, CTLA4, CD74, HP |
| | CX3CR1, CIITA, CD3D, CTLA4, CD74 |
| | CX3CR1, CIITA, CD3D, CTLA4, HP |
| | CX3CR1, CIITA, CD3D, CD74, HP |
| | CX3CR1, CIITA, CTLA4, CD74, HP |
| | CX3CR1, CD3D, CTLA4, CD74, HP |
| | IL1R2, IFNG, CIITA, CD3D, CTLA4 |
| | IL1R2, IFNG, CIITA, CD3D, CD74 |
| | IL1R2, IFNG, CIITA, CD3D, HP |
| | IL1R2, IFNG, CIITA, CTLA4, CD74 |
| | IL1R2, IFNG, CIITA, CTLA4, HP |
| | IL1R2, IFNG, CIITA, CD74, HP |
| | IL1R2, IFNG, CD3D, CTLA4, CD74 |
| | IL1R2, IFNG, CD3D, CTLA4, HP |
| | IL1R2, IFNG, CD3D, CD74, HP |
| | IL1R2, IFNG, CTLA4, CD74, HP |
| | IL1R2, CIITA, CD3D, CTLA4, CD74 |
| | IL1R2, CIITA, CD3D, CTLA4, HP |
| | IL1R2, CIITA, CD3D, CD74, HP |
| | IL1R2, CIITA, CTLA4, CD74, HP |
| | IL1R2, CD3D, CTLA4, CD74, HP |
| | IFNG, CIITA, CD3D, CTLA4, CD74 |
| | IFNG, CIITA, CD3D, CTLA4, HP |
| | IFNG, CIITA, CD3D, CD74, HP |
| | IFNG, CIITA, CTLA4, CD74, HP |
| | IFNG, CD3D, CTLA4, CD74, HP |
| | CIITA, CD3D, CTLA4, CD74, HP |
| Combinations of six genes | S100A9, TAP2, CD177, CX3CR1, IL1R2, IFNG |
| | S100A9, TAP2, CD177, CX3CR1, IL1R2, CIITA |
| | S100A9, TAP2, CD177, CX3CR1, IL1R2, CD3D |
| | S100A9, TAP2, CD177, CX3CR1, IL1R2, CTLA4 |
| | S100A9, TAP2, CD177, CX3CR1, IL1R2, CD74 |
| | S100A9, TAP2, CD177, CX3CR1, IL1R2, HP |
| | S100A9, TAP2, CD177, CX3CR1, IFNG, CIITA |
| | S100A9, TAP2, CD177, CX3CR1, IFNG, CD3D |
| | S100A9, TAP2, CD177, CX3CR1, IFNG, CTLA4 |
| | S100A9, TAP2, CD177, CX3CR1, IFNG, CD74 |
| | S100A9, TAP2, CD177, CX3CR1, IFNG, HP |
| | S100A9, TAP2, CD177, CX3CR1, CIITA, CD3D |
| | S100A9, TAP2, CD177, CX3CR1, CIITA, CTLA4 |
| | S100A9, TAP2, CD177, CX3CR1, CIITA, CD74 |
| | S100A9, TAP2, CD177, CX3CR1, CIITA, HP |
| | S100A9, TAP2, CD177, CX3CR1, CD3D, CTLA4 |
| | S100A9, TAP2, CD177, CX3CR1, CD3D, CD74 |
| | S100A9, TAP2, CD177, CX3CR1, CD3D, HP |
| | S100A9, TAP2, CD177, CX3CR1, CTLA4, CD74 |
| | S100A9, TAP2, CD177, CX3CR1, CTLA4, HP |
| | S100A9, TAP2, CD177, CX3CR1, CD74, HP |
| | S100A9, TAP2, CD177, IL1R2, IFNG, CIITA |
| | S100A9, TAP2, CD177, IL1R2, IFNG, CD3D |
| | S100A9, TAP2, CD177, IL1R2, IFNG, CTLA4 |
| | S100A9, TAP2, CD177, IL1R2, IFNG, CD74 |
| | S100A9, TAP2, CD177, IL1R2, IFNG, HP |
| | S100A9, TAP2, CD177, IL1R2, CIITA, CD3D |
| | S100A9, TAP2, CD177, IL1R2, CIITA, CTLA4 |
| | S100A9, TAP2, CD177, IL1R2, CIITA, CD74 |
| | S100A9, TAP2, CD177, IL1R2, CIITA, HP |
| | S100A9, TAP2, CD177, IL1R2, CD3D, CTLA4 |
| | S100A9, TAP2, CD177, IL1R2, CD3D, CD74 |
| | S100A9, TAP2, CD177, IL1R2, CD3D, HP |
| | S100A9, TAP2, CD177, IL1R2, CTLA4, CD74 |
| | S100A9, TAP2, CD177, IL1R2, CTLA4, HP |
| | S100A9, TAP2, CD177, IL1R2, CD74, HP |
| | S100A9, TAP2, CD177, IFNG, CIITA, CD3D |
| | S100A9, TAP2, CD177, IFNG, CIITA, CTLA4 |
| | S100A9, TAP2, CD177, IFNG, CIITA, CD74 |
| | S100A9, TAP2, CD177, IFNG, CIITA, HP |
| | S100A9, TAP2, CD177, IFNG, CD3D, CTLA4 |
| | S100A9, TAP2, CD177, IFNG, CD3D, CD74 |
| | S100A9, TAP2, CD177, IFNG, CD3D, HP |
| | S100A9, TAP2, CD177, IFNG, CTLA4, CD74 |
| | S100A9, TAP2, CD177, IFNG, CTLA4, HP |
| | S100A9, TAP2, CD177, IFNG, CD74, HP |
| | S100A9, TAP2, CD177, CIITA, CD3D, CTLA4 |
| | S100A9, TAP2, CD177, CIITA, CD3D, CD74 |
| | S100A9, TAP2, CD177, CIITA, CD3D, HP |
| | S100A9, TAP2, CD177, CIITA, CTLA4, CD74 |
| | S100A9, TAP2, CD177, CIITA, CTLA4, HP |
| | S100A9, TAP2, CD177, CIITA, CD74, HP |
| | S100A9, TAP2, CD177, CD3D, CTLA4, CD74 |
| | S100A9, TAP2, CD177, CD3D, CTLA4, HP |
| | S100A9, TAP2, CD177, CD3D, CD74, HP |
| | S100A9, TAP2, CD177, CTLA4, CD74, HP |
| | S100A9, TAP2, CX3CR1, IL1R2, IFNG, CIITA |
| | S100A9, TAP2, CX3CR1, IL1R2, IFNG, CD3D |
| | S100A9, TAP2, CX3CR1, IL1R2, IFNG, CTLA4 |
| | S100A9, TAP2, CX3CR1, IL1R2, IFNG, CD74 |
| | S100A9, TAP2, CX3CR1, IL1R2, IFNG, HP |
| | S100A9, TAP2, CX3CR1, IL1R2, CIITA, CD3D |
| | S100A9, TAP2, CX3CR1, IL1R2, CIITA, CTLA4 |
| | S100A9, TAP2, CX3CR1, IL1R2, CIITA, CD74 |
| | S100A9, TAP2, CX3CR1, IL1R2, CIITA, HP |
| | S100A9, TAP2, CX3CR1, IL1R2, CD3D, CTLA4 |
| | S100A9, TAP2, CX3CR1, IL1R2, CD3D, CD74 |
| | S100A9, TAP2, CX3CR1, IL1R2, CD3D, HP |
| | S100A9, TAP2, CX3CR1, IL1R2, CTLA4, CD74 |
| | S100A9, TAP2, CX3CR1, IL1R2, CTLA4, HP |
| | S100A9, TAP2, CX3CR1, IL1R2, CD74, HP |
| | S100A9, TAP2, CX3CR1, IFNG, CIITA, CD3D |
| | S100A9, TAP2, CX3CR1, IFNG, CIITA, CTLA4 |
| | S100A9, TAP2, CX3CR1, IFNG, CIITA, CD74 |
| | S100A9, TAP2, CX3CR1, IFNG, CIITA, HP |
| | S100A9, TAP2, CX3CR1, IFNG, CD3D, CTLA4 |
| | S100A9, TAP2, CX3CR1, IFNG, CD3D, CD74 |
| | S100A9, TAP2, CX3CR1, IFNG, CD3D, HP |
| | S100A9, TAP2, CX3CR1, IFNG, CTLA4, CD74 |
| | S100A9, TAP2, CX3CR1, IFNG, CTLA4, HP |
| | S100A9, TAP2, CX3CR1, IFNG, CD74, HP |
| | S100A9, TAP2, CX3CR1, CIITA, CD3D, CTLA4 |
| | S100A9, TAP2, CX3CR1, CIITA, CD3D, CD74 |
| | S100A9, TAP2, CX3CR1, CIITA, CD3D, HP |
| | S100A9, TAP2, CX3CR1, CIITA, CTLA4, CD74 |
| | S100A9, TAP2, CX3CR1, CIITA, CTLA4, HP |
| | S100A9, TAP2, CX3CR1, CIITA, CD74, HP |
| | S100A9, TAP2, CX3CR1, CD3D, CTLA4, CD74 |
| | S100A9, TAP2, CX3CR1, CD3D, CTLA4, HP |
| | S100A9, TAP2, CX3CR1, CD3D, CD74, HP |
| | S100A9, TAP2, CX3CR1, CTLA4, CD74, HP |
| | S100A9, TAP2, IL1R2, IFNG, CIITA, CD3D |
| | S100A9, TAP2, IL1R2, IFNG, CIITA, CTLA4 |
| | S100A9, TAP2, IL1R2, IFNG, CIITA, CD74 |
| | S100A9, TAP2, IL1R2, IFNG, CIITA, HP |
| | S100A9, TAP2, IL1R2, IFNG, CD3D, CTLA4 |
| | S100A9, TAP2, IL1R2, IFNG, CD3D, CD74 |
| | S100A9, TAP2, IL1R2, IFNG, CD3D, HP |
| | S100A9, TAP2, IL1R2, IFNG, CTLA4, CD74 |
| | S100A9, TAP2, IL1R2, IFNG, CTLA4, HP |
| | S100A9, TAP2, IL1R2, IFNG, CD74, HP |
| | S100A9, TAP2, IL1R2, CIITA, CD3D, CTLA4 |
| | S100A9, TAP2, IL1R2, CIITA, CD3D, CD74 |
| | S100A9, TAP2, IL1R2, CIITA, CD3D, HP |
| | S100A9, TAP2, IL1R2, CIITA, CTLA4, CD74 |
| | S100A9, TAP2, IL1R2, CIITA, CTLA4, HP |
| | S100A9, TAP2, IL1R2, CIITA, CD74, HP |
| | S100A9, TAP2, IL1R2, CD3D, CTLA4, CD74 |
| | S100A9, TAP2, IL1R2, CD3D, CTLA4, HP |
| | S100A9, TAP2, IL1R2, CD3D, CD74, HP |
| | S100A9, TAP2, IL1R2, CTLA4, CD74, HP |
| | S100A9, TAP2, IFNG, CIITA, CD3D, CTLA4 |
| | S100A9, TAP2, IFNG, CIITA, CD3D, CD74 |
| | S100A9, TAP2, IFNG, CIITA, CD3D, HP |
| | S100A9, TAP2, IFNG, CIITA, CTLA4, CD74 |
| | S100A9, TAP2, IFNG, CIITA, CTLA4, HP |
| | S100A9, TAP2, IFNG, CIITA, CD74, HP |
| | S100A9, TAP2, IFNG, CD3D, CTLA4, CD74 |
| | S100A9, TAP2, IFNG, CD3D, CTLA4, HP |
| | S100A9, TAP2, IFNG, CD3D, CD74, HP |
| | S100A9, TAP2, IFNG, CTLA4, CD74, HP |
| | S100A9, TAP2, CIITA, CD3D, CTLA4, CD74 |
| | S100A9, TAP2, CIITA, CD3D, CTLA4, HP |
| | S100A9, TAP2, CIITA, CD3D, CD74, HP |
| | S100A9, TAP2, CIITA, CTLA4, CD74, HP |
| | S100A9, TAP2, CD3D, CTLA4, CD74, HP |
| | S100A9, CD177, CX3CR1, IL1R2, IFNG, CIITA |
| | S100A9, CD177, CX3CR1, IL1R2, IFNG, CD3D |
| | S100A9, CD177, CX3CR1, IL1R2, IFNG, CTLA4 |
| | S100A9, CD177, CX3CR1, IL1R2, IFNG, CD74 |
| | S100A9, CD177, CX3CR1, IL1R2, IFNG, HP |
| | S100A9, CD177, CX3CR1, IL1R2, CIITA, CD3D |
| | S100A9, CD177, CX3CR1, IL1R2, CIITA, CTLA4 |
| | S100A9, CD177, CX3CR1, IL1R2, CIITA, CD74 |
| | S100A9, CD177, CX3CR1, IL1R2, CIITA, HP |
| | S100A9, CD177, CX3CR1, IL1R2, CD3D, CTLA4 |
| | S100A9, CD177, CX3CR1, IL1R2, CD3D, CD74 |
| | S100A9, CD177, CX3CR1, IL1R2, CD3D, HP |
| | S100A9, CD177, CX3CR1, IL1R2, CTLA4, CD74 |
| | S100A9, CD177, CX3CR1, IL1R2, CTLA4, HP |
| | S100A9, CD177, CX3CR1, IL1R2, CD74, HP |
| | S100A9, CD177, CX3CR1, IFNG, CIITA, CD3D |
| | S100A9, CD177, CX3CR1, IFNG, CIITA, CTLA4 |
| | S100A9, CD177, CX3CR1, IFNG, CIITA, CD74 |
| | S100A9, CD177, CX3CR1, IFNG, CIITA, HP |
| | S100A9, CD177, CX3CR1, IFNG, CD3D, CTLA4 |
| | S100A9, CD177, CX3CR1, IFNG, CD3D, CD74 |
| | S100A9, CD177, CX3CR1, IFNG, CD3D, HP |
| | S100A9, CD177, CX3CR1, IFNG, CTLA4, CD74 |
| | S100A9, CD177, CX3CR1, IFNG, CTLA4, HP |
| | S100A9, CD177, CX3CR1, IFNG, CD74, HP |
| | S100A9, CD177, CX3CR1, CIITA, CD3D, CTLA4 |
| | S100A9, CD177, CX3CR1, CIITA, CD3D, CD74 |
| | S100A9, CD177, CX3CR1, CIITA, CD3D, HP |
| | S100A9, CD177, CX3CR1, CIITA, CTLA4, CD74 |
| | S100A9, CD177, CX3CR1, CIITA, CTLA4, HP |
| | S100A9, CD177, CX3CR1, CIITA, CD74, HP |
| | S100A9, CD177, CX3CR1, CD3D, CTLA4, CD74 |
| | S100A9, CD177, CX3CR1, CD3D, CTLA4, HP |
| | S100A9, CD177, CX3CR1, CD3D, CD74, HP |
| | S100A9, CD177, CX3CR1, CTLA4, CD74, HP |
| | S100A9, CD177, IL1R2, IFNG, CIITA, CD3D |
| | S100A9, CD177, IL1R2, IFNG, CIITA, CTLA4 |
| | S100A9, CD177, IL1R2, IFNG, CIITA, CD74 |
| | S100A9, CD177, IL1R2, IFNG, CIITA, HP |
| | S100A9, CD177, IL1R2, IFNG, CD3D, CTLA4 |
| | S100A9, CD177, IL1R2, IFNG, CD3D, CD74 |
| | S100A9, CD177, IL1R2, IFNG, CD3D, HP |
| | S100A9, CD177, IL1R2, IFNG, CTLA4, CD74 |
| | S100A9, CD177, IL1R2, IFNG, CTLA4, HP |
| | S100A9, CD177, IL1R2, IFNG, CD74, HP |
| | S100A9, CD177, IL1R2, CIITA, CD3D, CTLA4 |
| | S100A9, CD177, IL1R2, CIITA, CD3D, CD74 |
| | S100A9, CD177, IL1R2, CIITA, CD3D, HP |
| | S100A9, CD177, IL1R2, CIITA, CTLA4, CD74 |
| | S100A9, CD177, IL1R2, CIITA, CTLA4, HP |
| | S100A9, CD177, IL1R2, CIITA, CD74, HP |
| | S100A9, CD177, IL1R2, CD3D, CTLA4, CD74 |
| | S100A9, CD177, IL1R2, CD3D, CTLA4, HP |
| | S100A9, CD177, IL1R2, CD3D, CD74, HP |
| | S100A9, CD177, IL1R2, CTLA4, CD74, HP |
| | S100A9, CD177, IFNG, CIITA, CD3D, CTLA4 |
| | S100A9, CD177, IFNG, CIITA, CD3D, CD74 |
| | S100A9, CD177, IFNG, CIITA, CD3D, HP |
| | S100A9, CD177, IFNG, CIITA, CTLA4, CD74 |
| | S100A9, CD177, IFNG, CIITA, CTLA4, HP |
| | S100A9, CD177, IFNG, CIITA, CD74, HP |
| | S100A9, CD177, IFNG, CD3D, CTLA4, CD74 |
| | S100A9, CD177, IFNG, CD3D, CTLA4, HP |
| | S100A9, CD177, IFNG, CD3D, CD74, HP |
| | S100A9, CD177, IFNG, CTLA4, CD74, HP |
| | S100A9, CD177, CIITA, CD3D, CTLA4, CD74 |
| | S100A9, CD177, CIITA, CD3D, CTLA4, HP |
| | S100A9, CD177, CIITA, CD3D, CD74, HP |
| | S100A9, CD177, CIITA, CTLA4, CD74, HP |
| | S100A9, CD177, CD3D, CTLA4, CD74, HP |
| | S100A9, CX3CR1, IL1R2, IFNG, CIITA, CD3D |
| | S100A9, CX3CR1, IL1R2, IFNG, CIITA, CTLA4 |
| | S100A9, CX3CR1, IL1R2, IFNG, CIITA, CD74 |
| | S100A9, CX3CR1, IL1R2, IFNG, CIITA, HP |
| | S100A9, CX3CR1, IL1R2, IFNG, CD3D, CTLA4 |
| | S100A9, CX3CR1, IL1R2, IFNG, CD3D, CD74 |
| | S100A9, CX3CR1, IL1R2, IFNG, CD3D, HP |
| | S100A9, CX3CR1, IL1R2, IFNG, CTLA4, CD74 |
| | S100A9, CX3CR1, IL1R2, IFNG, CTLA4, HP |
| | S100A9, CX3CR1, IL1R2, IFNG, CD74, HP |
| | S100A9, CX3CR1, IL1R2, CIITA, CD3D, CTLA4 |
| | S100A9, CX3CR1, IL1R2, CIITA, CD3D, CD74 |
| | S100A9, CX3CR1, IL1R2, CIITA, CD3D, HP |
| | S100A9, CX3CR1, IL1R2, CIITA, CTLA4, CD74 |
| | S100A9, CX3CR1, IL1R2, CIITA, CTLA4, HP |
| | S100A9, CX3CR1, IL1R2, CIITA, CD74, HP |
| | S100A9, CX3CR1, IL1R2, CD3D, CTLA4, CD74 |
| | S100A9, CX3CR1, IL1R2, CD3D, CTLA4, HP |
| | S100A9, CX3CR1, IL1R2, CD3D, CD74, HP |
| | S100A9, CX3CR1, IL1R2, CTLA4, CD74, HP |
| | S100A9, CX3CR1, IFNG, CIITA, CD3D, CTLA4 |
| | S100A9, CX3CR1, IFNG, CIITA, CD3D, CD74 |
| | S100A9, CX3CR1, IFNG, CIITA, CD3D, HP |
| | S100A9, CX3CR1, IFNG, CIITA, CTLA4, CD74 |
| | S100A9, CX3CR1, IFNG, CIITA, CTLA4, HP |
| | S100A9, CX3CR1, IFNG, CIITA, CD74, HP |
| | S100A9, CX3CR1, IFNG, CD3D, CTLA4, CD74 |
| | S100A9, CX3CR1, IFNG, CD3D, CTLA4, HP |
| | S100A9, CX3CR1, IFNG, CD3D, CD74, HP |
| | S100A9, CX3CR1, IFNG, CTLA4, CD74, HP |
| | S100A9, CX3CR1, CIITA, CD3D, CTLA4, CD74 |
| | S100A9, CX3CR1, CIITA, CD3D, CTLA4, HP |
| | S100A9, CX3CR1, CIITA, CD3D, CD74, HP |
| | S100A9, CX3CR1, CIITA, CTLA4, CD74, HP |
| | S100A9, CX3CR1, CD3D, CTLA4, CD74, HP |
| | S100A9, IL1R2, IFNG, CIITA, CD3D, CTLA4 |
| | S100A9, IL1R2, IFNG, CIITA, CD3D, CD74 |
| | S100A9, IL1R2, IFNG, CIITA, CD3D, HP |
| | S100A9, IL1R2, IFNG, CIITA, CTLA4, CD74 |
| | S100A9, IL1R2, IFNG, CIITA, CTLA4, HP |
| | S100A9, IL1R2, IFNG, CIITA, CD74, HP |
| | S100A9, IL1R2, IFNG, CD3D, CTLA4, CD74 |
| | S100A9, IL1R2, IFNG, CD3D, CTLA4, HP |
| | S100A9, IL1R2, IFNG, CD3D, CD74, HP |
| | S100A9, IL1R2, IFNG, CTLA4, CD74, HP |
| | S100A9, IL1R2, CIITA, CD3D, CTLA4, CD74 |
| | S100A9, IL1R2, CIITA, CD3D, CTLA4, HP |
| | S100A9, IL1R2, CIITA, CD3D, CD74, HP |
| | S100A9, IL1R2, CIITA, CTLA4, CD74, HP |
| | S100A9, IL1R2, CD3D, CTLA4, CD74, HP |
| | S100A9, IFNG, CIITA, CD3D, CTLA4, CD74 |
| | S100A9, IFNG, CIITA, CD3D, CTLA4, HP |
| | S100A9, IFNG, CIITA, CD3D, CD74, HP |
| | S100A9, IFNG, CIITA, CTLA4, CD74, HP |
| | S100A9, IFNG, CD3D, CTLA4, CD74, HP |
| | S100A9, CIITA, CD3D, CTLA4, CD74, HP |
| | TAP2, CD177, CX3CR1, IL1R2, IFNG, CIITA |
| | TAP2, CD177, CX3CR1, IL1R2, IFNG, CD3D |
| | TAP2, CD177, CX3CR1, IL1R2, IFNG, CTLA4 |
| | TAP2, CD177, CX3CR1, IL1R2, IFNG, CD74 |
| | TAP2, CD177, CX3CR1, IL1R2, IFNG, HP |
| | TAP2, CD177, CX3CR1, IL1R2, CIITA, CD3D |
| | TAP2, CD177, CX3CR1, IL1R2, CIITA, CTLA4 |
| | TAP2, CD177, CX3CR1, IL1R2, CIITA, CD74 |
| | TAP2, CD177, CX3CR1, IL1R2, CIITA, HP |
| | TAP2, CD177, CX3CR1, IL1R2, CD3D, CTLA4 |
| | TAP2, CD177, CX3CR1, IL1R2, CD3D, CD74 |
| | TAP2, CD177, CX3CR1, IL1R2, CD3D, HP |
| | TAP2, CD177, CX3CR1, IL1R2, CTLA4, CD74 |
| | TAP2, CD177, CX3CR1, IL1R2, CTLA4, HP |
| | TAP2, CD177, CX3CR1, IL1R2, CD74, HP |
| | TAP2, CD177, CX3CR1, IFNG, CIITA, CD3D |
| | TAP2, CD177, CX3CR1, IFNG, CIITA, CTLA4 |
| | TAP2, CD177, CX3CR1, IFNG, CIITA, CD74 |
| | TAP2, CD177, CX3CR1, IFNG, CIITA, HP |
| | TAP2, CD177, CX3CR1, IFNG, CD3D, CTLA4 |
| | TAP2, CD177, CX3CR1, IFNG, CD3D, CD74 |
| | TAP2, CD177, CX3CR1, IFNG, CD3D, HP |
| | TAP2, CD177, CX3CR1, IFNG, CTLA4, CD74 |
| | TAP2, CD177, CX3CR1, IFNG, CTLA4, HP |
| | TAP2, CD177, CX3CR1, IFNG, CD74, HP |
| | TAP2, CD177, CX3CR1, CIITA, CD3D, CTLA4 |
| | TAP2, CD177, CX3CR1, CIITA, CD3D, CD74 |
| | TAP2, CD177, CX3CR1, CIITA, CD3D, HP |
| | TAP2, CD177, CX3CR1, CIITA, CTLA4, CD74 |
| | TAP2, CD177, CX3CR1, CIITA, CTLA4, HP |
| | TAP2, CD177, CX3CR1, CIITA, CD74, HP |
| | TAP2, CD177, CX3CR1, CD3D, CTLA4, CD74 |
| | TAP2, CD177, CX3CR1, CD3D, CTLA4, HP |
| | TAP2, CD177, CX3CR1, CD3D, CD74, HP |
| | TAP2, CD177, CX3CR1, CTLA4, CD74, HP |
| | TAP2, CD177, IL1R2, IFNG, CIITA, CD3D |
| | TAP2, CD177, IL1R2, IFNG, CIITA, CTLA4 |
| | TAP2, CD177, IL1R2, IFNG, CIITA, CD74 |
| | TAP2, CD177, IL1R2, IFNG, CIITA, HP |
| | TAP2, CD177, IL1R2, IFNG, CD3D, CTLA4 |
| | TAP2, CD177, IL1R2, IFNG, CD3D, CD74 |
| | TAP2, CD177, IL1R2, IFNG, CD3D, HP |
| | TAP2, CD177, IL1R2, IFNG, CTLA4, CD74 |
| | TAP2, CD177, IL1R2, IFNG, CTLA4, HP |
| | TAP2, CD177, IL1R2, IFNG, CD74, HP |
| | TAP2, CD177, IL1R2, CIITA, CD3D, CTLA4 |
| | TAP2, CD177, IL1R2, CIITA, CD3D, CD74 |
| | TAP2, CD177, IL1R2, CIITA, CD3D, HP |
| | TAP2, CD177, IL1R2, CIITA, CTLA4, CD74 |
| | TAP2, CD177, IL1R2, CIITA, CTLA4, HP |
| | TAP2, CD177, IL1R2, CIITA, CD74, HP |
| | TAP2, CD177, IL1R2, CD3D, CTLA4, CD74 |
| | TAP2, CD177, IL1R2, CD3D, CTLA4, HP |
| | TAP2, CD177, IL1R2, CD3D, CD74, HP |
| | TAP2, CD177, IL1R2, CTLA4, CD74, HP |
| | TAP2, CD177, IFNG, CIITA, CD3D, CTLA4 |
| | TAP2, CD177, IFNG, CIITA, CD3D, CD74 |
| | TAP2, CD177, IFNG, CIITA, CD3D, HP |
| | TAP2, CD177, IFNG, CIITA, CTLA4, CD74 |
| | TAP2, CD177, IFNG, CIITA, CTLA4, HP |
| | TAP2, CD177, IFNG, CIITA, CD74, HP |
| | TAP2, CD177, IFNG, CD3D, CTLA4, CD74 |
| | TAP2, CD177, IFNG, CD3D, CTLA4, HP |
| | TAP2, CD177, IFNG, CD3D, CD74, HP |
| | TAP2, CD177, IFNG, CTLA4, CD74, HP |
| | TAP2, CD177, CIITA, CD3D, CTLA4, CD74 |
| | TAP2, CD177, CIITA, CD3D, CTLA4, HP |
| | TAP2, CD177, CIITA, CD3D, CD74, HP |
| | TAP2, CD177, CIITA, CTLA4, CD74, HP |
| | TAP2, CD177, CD3D, CTLA4, CD74, HP |
| | TAP2, CX3CR1, IL1R2, IFNG, CIITA, CD3D |
| | TAP2, CX3CR1, IL1R2, IFNG, CIITA, CTLA4 |
| | TAP2, CX3CR1, IL1R2, IFNG, CIITA, CD74 |
| | TAP2, CX3CR1, IL1R2, IFNG, CIITA, HP |
| | TAP2, CX3CR1, IL1R2, IFNG, CD3D, CTLA4 |
| | TAP2, CX3CR1, IL1R2, IFNG, CD3D, CD74 |
| | TAP2, CX3CR1, IL1R2, IFNG, CD3D, HP |
| | TAP2, CX3CR1, IL1R2, IFNG, CTLA4, CD74 |
| | TAP2, CX3CR1, IL1R2, IFNG, CTLA4, HP |
| | TAP2, CX3CR1, IL1R2, IFNG, CD74, HP |
| | TAP2, CX3CR1, IL1R2, CIITA, CD3D, CTLA4 |
| | TAP2, CX3CR1, IL1R2, CIITA, CD3D, CD74 |
| | TAP2, CX3CR1, IL1R2, CIITA, CD3D, HP |
| | TAP2, CX3CR1, IL1R2, CIITA, CTLA4, CD74 |
| | TAP2, CX3CR1, IL1R2, CIITA, CTLA4, HP |
| | TAP2, CX3CR1, IL1R2, CIITA, CD74, HP |
| | TAP2, CX3CR1, IL1R2, CD3D, CTLA4, CD74 |
| | TAP2, CX3CR1, IL1R2, CD3D, CTLA4, HP |
| | TAP2, CX3CR1, IL1R2, CD3D, CD74, HP |
| | TAP2, CX3CR1, IL1R2, CTLA4, CD74, HP |
| | TAP2, CX3CR1, IFNG, CIITA, CD3D, CTLA4 |
| | TAP2, CX3CR1, IFNG, CIITA, CD3D, CD74 |
| | TAP2, CX3CR1, IFNG, CIITA, CD3D, HP |
| | TAP2, CX3CR1, IFNG, CIITA, CTLA4, CD74 |
| | TAP2, CX3CR1, IFNG, CIITA, CTLA4, HP |
| | TAP2, CX3CR1, IFNG, CIITA, CD74, HP |
| | TAP2, CX3CR1, IFNG, CD3D, CTLA4, CD74 |
| | TAP2, CX3CR1, IFNG, CD3D, CTLA4, HP |
| | TAP2, CX3CR1, IFNG, CD3D, CD74, HP |
| | TAP2, CX3CR1, IFNG, CTLA4, CD74, HP |
| | TAP2, CX3CR1, CIITA, CD3D, CTLA4, CD74 |
| | TAP2, CX3CR1, CIITA, CD3D, CTLA4, HP |
| | TAP2, CX3CR1, CIITA, CD3D, CD74, HP |
| | TAP2, CX3CR1, CIITA, CTLA4, CD74, HP |
| | TAP2, CX3CR1, CD3D, CTLA4, CD74, HP |
| | TAP2, IL1R2, IFNG, CIITA, CD3D, CTLA4 |
| | TAP2, IL1R2, IFNG, CIITA, CD3D, CD74 |
| | TAP2, IL1R2, IFNG, CIITA, CD3D, HP |
| | TAP2, IL1R2, IFNG, CIITA, CTLA4, CD74 |
| | TAP2, IL1R2, IFNG, CIITA, CTLA4, HP |
| | TAP2, IL1R2, IFNG, CIITA, CD74, HP |
| | TAP2, IL1R2, IFNG, CD3D, CTLA4, CD74 |
| | TAP2, IL1R2, IFNG, CD3D, CTLA4, HP |
| | TAP2, IL1R2, IFNG, CD3D, CD74, HP |
| | TAP2, IL1R2, IFNG, CTLA4, CD74, HP |
| | TAP2, IL1R2, CIITA, CD3D, CTLA4, CD74 |
| | TAP2, IL1R2, CIITA, CD3D, CTLA4, HP |
| | TAP2, IL1R2, CIITA, CD3D, CD74, HP |
| | TAP2, IL1R2, CIITA, CTLA4, CD74, HP |
| | TAP2, IL1R2, CD3D, CTLA4, CD74, HP |
| | TAP2, IFNG, CIITA, CD3D, CTLA4, CD74 |
| | TAP2, IFNG, CIITA, CD3D, CTLA4, HP |
| | TAP2, IFNG, CIITA, CD3D, CD74, HP |
| | TAP2, IFNG, CIITA, CTLA4, CD74, HP |
| | TAP2, IFNG, CD3D, CTLA4, CD74, HP |
| | TAP2, CIITA, CD3D, CTLA4, CD74, HP |
| | CD177, CX3CR1, IL1R2, IFNG, CIITA, CD3D |
| | CD177, CX3CR1, IL1R2, IFNG, CIITA, CTLA4 |
| | CD177, CX3CR1, IL1R2, IFNG, CIITA, CD74 |
| | CD177, CX3CR1, IL1R2, IFNG, CIITA, HP |
| | CD177, CX3CR1, IL1R2, IFNG, CD3D, CTLA4 |
| | CD177, CX3CR1, IL1R2, IFNG, CD3D, CD74 |
| | CD177, CX3CR1, IL1R2, IFNG, CD3D, HP |
| | CD177, CX3CR1, IL1R2, IFNG, CTLA4, CD74 |
| | CD177, CX3CR1, IL1R2, IFNG, CTLA4, HP |
| | CD177, CX3CR1, IL1R2, IFNG, CD74, HP |
| | CD177, CX3CR1, IL1R2, CIITA, CD3D, CTLA4 |
| | CD177, CX3CR1, IL1R2, CIITA, CD3D, CD74 |
| | CD177, CX3CR1, IL1R2, CIITA, CD3D, HP |
| | CD177, CX3CR1, IL1R2, CIITA, CTLA4, CD74 |
| | CD177, CX3CR1, IL1R2, CIITA, CTLA4, HP |
| | CD177, CX3CR1, IL1R2, CIITA, CD74, HP |
| | CD177, CX3CR1, IL1R2, CD3D, CTLA4, CD74 |
| | CD177, CX3CR1, IL1R2, CD3D, CTLA4, HP |
| | CD177, CX3CR1, IL1R2, CD3D, CD74, HP |
| | CD177, CX3CR1, IL1R2, CTLA4, CD74, HP |
| | CD177, CX3CR1, IFNG, CIITA, CD3D, CTLA4 |
| | CD177, CX3CR1, IFNG, CIITA, CD3D, CD74 |
| | CD177, CX3CR1, IFNG, CIITA, CD3D, HP |
| | CD177, CX3CR1, IFNG, CIITA, CTLA4, CD74 |
| | CD177, CX3CR1, IFNG, CIITA, CTLA4, HP |
| | CD177, CX3CR1, IFNG, CIITA, CD74, HP |
| | CD177, CX3CR1, IFNG, CD3D, CTLA4, CD74 |
| | CD177, CX3CR1, IFNG, CD3D, CTLA4, HP |
| | CD177, CX3CR1, IFNG, CD3D, CD74, HP |
| | CD177, CX3CR1, IFNG, CTLA4, CD74, HP |
| | CD177, CX3CR1, CIITA, CD3D, CTLA4, CD74 |
| | CD177, CX3CR1, CIITA, CD3D, CTLA4, HP |
| | CD177, CX3CR1, CIITA, CD3D, CD74, HP |
| | CD177, CX3CR1, CIITA, CTLA4, CD74, HP |
| | CD177, CX3CR1, CD3D, CTLA4, CD74, HP |
| | CD177, IL1R2, IFNG, CIITA, CD3D, CTLA4 |
| | CD177, IL1R2, IFNG, CIITA, CD3D, CD74 |
| | CD177, IL1R2, IFNG, CIITA, CD3D, HP |
| | CD177, IL1R2, IFNG, CIITA, CTLA4, CD74 |
| | CD177, IL1R2, IFNG, CIITA, CTLA4, HP |
| | CD177, IL1R2, IFNG, CIITA, CD74, HP |
| | CD177, IL1R2, IFNG, CD3D, CTLA4, CD74 |
| | CD177, IL1R2, IFNG, CD3D, CTLA4, HP |
| | CD177, IL1R2, IFNG, CD3D, CD74, HP |
| | CD177, IL1R2, IFNG, CTLA4, CD74, HP |
| | CD177, IL1R2, CIITA, CD3D, CTLA4, CD74 |
| | CD177, IL1R2, CIITA, CD3D, CTLA4, HP |
| | CD177, IL1R2, CIITA, CD3D, CD74, HP |
| | CD177, IL1R2, CIITA, CTLA4, CD74, HP |
| | CD177, IL1R2, CD3D, CTLA4, CD74, HP |
| | CD177, IFNG, CIITA, CD3D, CTLA4, CD74 |
| | CD177, IFNG, CIITA, CD3D, CTLA4, HP |
| | CD177, IFNG, CIITA, CD3D, CD74, HP |
| | CD177, IFNG, CIITA, CTLA4, CD74, HP |
| | CD177, IFNG, CD3D, CTLA4, CD74, HP |
| | CD177, CIITA, CD3D, CTLA4, CD74, HP |
| | CX3CR1, IL1R2, IFNG, CIITA, CD3D, CTLA4 |
| | CX3CR1, IL1R2, IFNG, CIITA, CD3D, CD74 |
| | CX3CR1, IL1R2, IFNG, CIITA, CD3D, HP |
| | CX3CR1, IL1R2, IFNG, CIITA, CTLA4, CD74 |
| | CX3CR1, IL1R2, IFNG, CIITA, CTLA4, HP |
| | CX3CR1, IL1R2, IFNG, CIITA, CD74, HP |
| | CX3CR1, IL1R2, IFNG, CD3D, CTLA4, CD74 |
| | CX3CR1, IL1R2, IFNG, CD3D, CTLA4, HP |
| | CX3CR1, IL1R2, IFNG, CD3D, CD74, HP |
| | CX3CR1, IL1R2, IFNG, CTLA4, CD74, HP |
| | CX3CR1, IL1R2, CIITA, CD3D, CTLA4, CD74 |
| | CX3CR1, IL1R2, CIITA, CD3D, CTLA4, HP |
| | CX3CR1, IL1R2, CIITA, CD3D, CD74, HP |
| | CX3CR1, IL1R2, CIITA, CTLA4, CD74, HP |
| | CX3CR1, IL1R2, CD3D, CTLA4, CD74, HP |
| | CX3CR1, IFNG, CIITA, CD3D, CTLA4, CD74 |
| | CX3CR1, IFNG, CIITA, CD3D, CTLA4, HP |
| | CX3CR1, IFNG, CIITA, CD3D, CD74, HP |
| | CX3CR1, IFNG, CIITA, CTLA4, CD74, HP |
| | CX3CR1, IFNG, CD3D, CTLA4, CD74, HP |
| | CX3CR1, CIITA, CD3D, CTLA4, CD74, HP |
| | IL1R2, IFNG, CIITA, CD3D, CTLA4, CD74 |
| | IL1R2, IFNG, CIITA, CD3D, CTLA4, HP |
| | IL1R2, IFNG, CIITA, CD3D, CD74, HP |
| | IL1R2, IFNG, CIITA, CTLA4, CD74, HP |
| | IL1R2, IFNG, CD3D, CTLA4, CD74, HP |
| | IL1R2, CIITA, CD3D, CTLA4, CD74, HP |
| | IFNG, CIITA, CD3D, CTLA4, CD74, HP |
| Combinations of seven | S100A9, TAP2, CD177, CX3CR1, IL1R2, IFNG, CIITA |
| genes | S100A9, TAP2, CD177, CX3CR1, IL1R2, IFNG, CD3D |
| | S100A9, TAP2, CD177, CX3CR1, IL1R2, IFNG, CTLA4 |
| | S100A9, TAP2, CD177, CX3CR1, IL1R2, IFNG, CD74 |
| | S100A9, TAP2, CD177, CX3CR1, IL1R2, IFNG, HP |
| | S100A9, TAP2, CD177, CX3CR1, IL1R2, CIITA, CD3D |
| | S100A9, TAP2, CD177, CX3CR1, IL1R2, CIITA, CTLA4 |
| | S100A9, TAP2, CD177, CX3CR1, IL1R2, CIITA, CD74 |
| | S100A9, TAP2, CD177, CX3CR1, IL1R2, CIITA, HP |
| | S100A9, TAP2, CD177, CX3CR1, IL1R2, CD3D, CTLA4 |
| | S100A9, TAP2, CD177, CX3CR1, IL1R2, CD3D, CD74 |
| | S100A9, TAP2, CD177, CX3CR1, IL1R2, CD3D, HP |
| | S100A9, TAP2, CD177, CX3CR1, IL1R2, CTLA4, CD74 |
| | S100A9, TAP2, CD177, CX3CR1, IL1R2, CTLA4, HP |
| | S100A9, TAP2, CD177, CX3CR1, IL1R2, CD74, HP |
| | S100A9, TAP2, CD177, CX3CR1, IFNG, CIITA, CD3D |
| | S100A9, TAP2, CD177, CX3CR1, IFNG, CIITA, CTLA4 |
| | S100A9, TAP2, CD177, CX3CR1, IFNG, CIITA, CD74 |
| | S100A9, TAP2, CD177, CX3CR1, IFNG, CIITA, HP |
| | S100A9, TAP2, CD177, CX3CR1, IFNG, CD3D, CTLA4 |
| | S100A9, TAP2, CD177, CX3CR1, IFNG, CD3D, CD74 |
| | S100A9, TAP2, CD177, CX3CR1, IFNG, CD3D, HP |
| | S100A9, TAP2, CD177, CX3CR1, IFNG, CTLA4, CD74 |
| | S100A9, TAP2, CD177, CX3CR1, IFNG, CTLA4, HP |
| | S100A9, TAP2, CD177, CX3CR1, IFNG, CD74, HP |
| | S100A9, TAP2, CD177, CX3CR1, CIITA, CD3D, CTLA4 |
| | S100A9, TAP2, CD177, CX3CR1, CIITA, CD3D, CD74 |
| | S100A9, TAP2, CD177, CX3CR1, CIITA, CD3D, HP |
| | S100A9, TAP2, CD177, CX3CR1, CIITA, CTLA4, CD74 |
| | S100A9, TAP2, CD177, CX3CR1, CIITA, CTLA4, HP |
| | S100A9, TAP2, CD177, CX3CR1, CIITA, CD74, HP |
| | S100A9, TAP2, CD177, CX3CR1, CD3D, CTLA4, CD74 |
| | S100A9, TAP2, CD177, CX3CR1, CD3D, CTLA4, HP |
| | S100A9, TAP2, CD177, CX3CR1, CD3D, CD74, HP |
| | S100A9, TAP2, CD177, CX3CR1, CTLA4, CD74, HP |
| | S100A9, TAP2, CD177, IL1R2, IFNG, CIITA, CD3D |
| | S100A9, TAP2, CD177, IL1R2, IFNG, CIITA, CTLA4 |
| | S100A9, TAP2, CD177, IL1R2, IFNG, CIITA, CD74 |
| | S100A9, TAP2, CD177, IL1R2, IFNG, CIITA, HP |
| | S100A9, TAP2, CD177, IL1R2, IFNG, CD3D, CTLA4 |
| | S100A9, TAP2, CD177, IL1R2, IFNG, CD3D, CD74 |
| | S100A9, TAP2, CD177, IL1R2, IFNG, CD3D, HP |
| | S100A9, TAP2, CD177, IL1R2, IFNG, CTLA4, CD74 |
| | S100A9, TAP2, CD177, IL1R2, IFNG, CTLA4, HP |
| | S100A9, TAP2, CD177, IL1R2, IFNG, CD74, HP |
| | S100A9, TAP2, CD177, IL1R2, CIITA, CD3D, CTLA4 |
| | S100A9, TAP2, CD177, IL1R2, CIITA, CD3D, CD74 |
| | S100A9, TAP2, CD177, IL1R2, CIITA, CD3D, HP |
| | S100A9, TAP2, CD177, IL1R2, CIITA, CTLA4, CD74 |
| | S100A9, TAP2, CD177, IL1R2, CIITA, CTLA4, HP |
| | S100A9, TAP2, CD177, IL1R2, CIITA, CD74, HP |
| | S100A9, TAP2, CD177, IL1R2, CD3D, CTLA4, CD74 |
| | S100A9, TAP2, CD177, IL1R2, CD3D, CTLA4, HP |
| | S100A9, TAP2, CD177, IL1R2, CD3D, CD74, HP |
| | S100A9, TAP2, CD177, IL1R2, CTLA4, CD74, HP |
| | S100A9, TAP2, CD177, IFNG, CIITA, CD3D, CTLA4 |
| | S100A9, TAP2, CD177, IFNG, CIITA, CD3D, CD74 |
| | S100A9, TAP2, CD177, IFNG, CIITA, CD3D, HP |
| | S100A9, TAP2, CD177, IFNG, CIITA, CTLA4, CD74 |
| | S100A9, TAP2, CD177, IFNG, CIITA, CTLA4, HP |
| | S100A9, TAP2, CD177, IFNG, CIITA, CD74, HP |
| | S100A9, TAP2, CD177, IFNG, CD3D, CTLA4, CD74 |
| | S100A9, TAP2, CD177, IFNG, CD3D, CTLA4, HP |
| | S100A9, TAP2, CD177, IFNG, CD3D, CD74, HP |
| | S100A9, TAP2, CD177, IFNG, CTLA4, CD74, HP |
| | S100A9, TAP2, CD177, CIITA, CD3D, CTLA4, CD74 |
| | S100A9, TAP2, CD177, CIITA, CD3D, CTLA4, HP |
| | S100A9, TAP2, CD177, CIITA, CD3D, CD74, HP |
| | S100A9, TAP2, CD177, CIITA, CTLA4, CD74, HP |
| | S100A9, TAP2, CD177, CD3D, CTLA4, CD74, HP |
| | S100A9, TAP2, CX3CR1, IL1R2, IFNG, CIITA, CD3D |
| | S100A9, TAP2, CX3CR1, IL1R2, IFNG, CIITA, CTLA4 |
| | S100A9, TAP2, CX3CR1, IL1R2, IFNG, CIITA, CD74 |
| | S100A9, TAP2, CX3CR1, IL1R2, IFNG, CIITA, HP |
| | S100A9, TAP2, CX3CR1, IL1R2, IFNG, CD3D, CTLA4 |
| | S100A9, TAP2, CX3CR1, IL1R2, IFNG, CD3D, CD74 |
| | S100A9, TAP2, CX3CR1, IL1R2, IFNG, CD3D, HP |
| | S100A9, TAP2, CX3CR1, IL1R2, IFNG, CTLA4, CD74 |
| | S100A9, TAP2, CX3CR1, IL1R2, IFNG, CTLA4, HP |
| | S100A9, TAP2, CX3CR1, IL1R2, IFNG, CD74, HP |
| | S100A9, TAP2, CX3CR1, IL1R2, CIITA, CD3D, CTLA4 |
| | S100A9, TAP2, CX3CR1, IL1R2, CIITA, CD3D, CD74 |
| | S100A9, TAP2, CX3CR1, IL1R2, CIITA, CD3D, HP |
| | S100A9, TAP2, CX3CR1, IL1R2, CIITA, CTLA4, CD74 |
| | S100A9, TAP2, CX3CR1, IL1R2, CIITA, CTLA4, HP |
| | S100A9, TAP2, CX3CR1, IL1R2, CIITA, CD74, HP |
| | S100A9, TAP2, CX3CR1, IL1R2, CD3D, CTLA4, CD74 |
| | S100A9, TAP2, CX3CR1, IL1R2, CD3D, CTLA4, HP |
| | S100A9, TAP2, CX3CR1, IL1R2, CD3D, CD74, HP |
| | S100A9, TAP2, CX3CR1, IL1R2, CTLA4, CD74, HP |
| | S100A9, TAP2, CX3CR1, IFNG, CIITA, CD3D, CTLA4 |
| | S100A9, TAP2, CX3CR1, IFNG, CIITA, CD3D, CD74 |
| | S100A9, TAP2, CX3CR1, IFNG, CIITA, CD3D, HP |
| | S100A9, TAP2, CX3CR1, IFNG, CIITA, CTLA4, CD74 |
| | S100A9, TAP2, CX3CR1, IFNG, CIITA, CTLA4, HP |
| | S100A9, TAP2, CX3CR1, IFNG, CIITA, CD74, HP |
| | S100A9, TAP2, CX3CR1, IFNG, CD3D, CTLA4, CD74 |
| | S100A9, TAP2, CX3CR1, IFNG, CD3D, CTLA4, HP |
| | S100A9, TAP2, CX3CR1, IFNG, CD3D, CD74, HP |
| | S100A9, TAP2, CX3CR1, IFNG, CTLA4, CD74, HP |
| | S100A9, TAP2, CX3CR1, CIITA, CD3D, CTLA4, CD74 |
| | S100A9, TAP2, CX3CR1, CIITA, CD3D, CTLA4, HP |
| | S100A9, TAP2, CX3CR1, CIITA, CD3D, CD74, HP |
| | S100A9, TAP2, CX3CR1, CIITA, CTLA4, CD74, HP |
| | S100A9, TAP2, CX3CR1, CD3D, CTLA4, CD74, HP |
| | S100A9, TAP2, IL1R2, IFNG, CIITA, CD3D, CTLA4 |
| | S100A9, TAP2, IL1R2, IFNG, CIITA, CD3D, CD74 |
| | S100A9, TAP2, IL1R2, IFNG, CIITA, CD3D, HP |
| | S100A9, TAP2, IL1R2, IFNG, CIITA, CTLA4, CD74 |
| | S100A9, TAP2, IL1R2, IFNG, CIITA, CTLA4, HP |
| | S100A9, TAP2, IL1R2, IFNG, CIITA, CD74, HP |
| | S100A9, TAP2, IL1R2, IFNG, CD3D, CTLA4, CD74 |
| | S100A9, TAP2, IL1R2, IFNG, CD3D, CTLA4, HP |
| | S100A9, TAP2, IL1R2, IFNG, CD3D, CD74, HP |
| | S100A9, TAP2, IL1R2, IFNG, CTLA4, CD74, HP |
| | S100A9, TAP2, IL1R2, CIITA, CD3D, CTLA4, CD74 |
| | S100A9, TAP2, IL1R2, CIITA, CD3D, CTLA4, HP |
| | S100A9, TAP2, IL1R2, CIITA, CD3D, CD74, HP |
| | S100A9, TAP2, IL1R2, CIITA, CTLA4, CD74, HP |
| | S100A9, TAP2, IL1R2, CD3D, CTLA4, CD74, HP |
| | S100A9, TAP2, IFNG, CIITA, CD3D, CTLA4, CD74 |
| | S100A9, TAP2, IFNG, CIITA, CD3D, CTLA4, HP |
| | S100A9, TAP2, IFNG, CIITA, CD3D, CD74, HP |
| | S100A9, TAP2, IFNG, CIITA, CTLA4, CD74, HP |
| | S100A9, TAP2, IFNG, CD3D, CTLA4, CD74, HP |
| | S100A9, TAP2, CIITA, CD3D, CTLA4, CD74, HP |
| | S100A9, CD177, CX3CR1, IL1R2, IFNG, CIITA, CD3D |
| | S100A9, CD177, CX3CR1, IL1R2, IFNG, CIITA, CTLA4 |
| | S100A9, CD177, CX3CR1, IL1R2, IFNG, CIITA, CD74 |
| | S100A9, CD177, CX3CR1, IL1R2, IFNG, CIITA, HP |
| | S100A9, CD177, CX3CR1, IL1R2, IFNG, CD3D, CTLA4 |
| | S100A9, CD177, CX3CR1, IL1R2, IFNG, CD3D, CD74 |
| | S100A9, CD177, CX3CR1, IL1R2, IFNG, CD3D, HP |
| | S100A9, CD177, CX3CR1, IL1R2, IFNG, CTLA4, CD74 |
| | S100A9, CD177, CX3CR1, IL1R2, IFNG, CTLA4, HP |
| | S100A9, CD177, CX3CR1, IL1R2, IFNG, CD74, HP |
| | S100A9, CD177, CX3CR1, IL1R2, CIITA, CD3D, CTLA4 |
| | S100A9, CD177, CX3CR1, IL1R2, CIITA, CD3D, CD74 |
| | S100A9, CD177, CX3CR1, IL1R2, CIITA, CD3D, HP |
| | S100A9, CD177, CX3CR1, IL1R2, CIITA, CTLA4, CD74 |
| | S100A9, CD177, CX3CR1, IL1R2, CIITA, CTLA4, HP |
| | S100A9, CD177, CX3CR1, IL1R2, CIITA, CD74, HP |
| | S100A9, CD177, CX3CR1, IL1R2, CD3D, CTLA4, CD74 |
| | S100A9, CD177, CX3CR1, IL1R2, CD3D, CTLA4, HP |
| | S100A9, CD177, CX3CR1, IL1R2, CD3D, CD74, HP |
| | S100A9, CD177, CX3CR1, IL1R2, CTLA4, CD74, HP |
| | S100A9, CD177, CX3CR1, IFNG, CIITA, CD3D, CTLA4 |
| | S100A9, CD177, CX3CR1, IFNG, CIITA, CD3D, CD74 |
| | S100A9, CD177, CX3CR1, IFNG, CIITA, CD3D, HP |
| | S100A9, CD177, CX3CR1, IFNG, CIITA, CTLA4, CD74 |
| | S100A9, CD177, CX3CR1, IFNG, CIITA, CTLA4, HP |
| | S100A9, CD177, CX3CR1, IFNG, CIITA, CD74, HP |
| | S100A9, CD177, CX3CR1, IFNG, CD3D, CTLA4, CD74 |
| | S100A9, CD177, CX3CR1, IFNG, CD3D, CTLA4, HP |
| | S100A9, CD177, CX3CR1, IFNG, CD3D, CD74, HP |
| | S100A9, CD177, CX3CR1, IFNG, CTLA4, CD74, HP |
| | S100A9, CD177, CX3CR1, CIITA, CD3D, CTLA4, CD74 |
| | S100A9, CD177, CX3CR1, CIITA, CD3D, CTLA4, HP |
| | S100A9, CD177, CX3CR1, CIITA, CD3D, CD74, HP |
| | S100A9, CD177, CX3CR1, CIITA, CTLA4, CD74, HP |
| | S100A9, CD177, CX3CR1, CD3D, CTLA4, CD74, HP |
| | S100A9, CD177, IL1R2, IFNG, CIITA, CD3D, CTLA4 |
| | S100A9, CD177, IL1R2, IFNG, CIITA, CD3D, CD74 |
| | S100A9, CD177, IL1R2, IFNG, CIITA, CD3D, HP |
| | S100A9, CD177, IL1R2, IFNG, CIITA, CTLA4, CD74 |
| | S100A9, CD177, IL1R2, IFNG, CIITA, CTLA4, HP |
| | S100A9, CD177, IL1R2, IFNG, CIITA, CD74, HP |
| | S100A9, CD177, IL1R2, IFNG, CD3D, CTLA4, CD74 |
| | S100A9, CD177, IL1R2, IFNG, CD3D, CTLA4, HP |
| | S100A9, CD177, IL1R2, IFNG, CD3D, CD74, HP |
| | S100A9, CD177, IL1R2, IFNG, CTLA4, CD74, HP |
| | S100A9, CD177, IL1R2, CIITA, CD3D, CTLA4, CD74 |
| | S100A9, CD177, IL1R2, CIITA, CD3D, CTLA4, HP |
| | S100A9, CD177, IL1R2, CIITA, CD3D, CD74, HP |
| | S100A9, CD177, IL1R2, CIITA, CTLA4, CD74, HP |
| | S100A9, CD177, IL1R2, CD3D, CTLA4, CD74, HP |
| | S100A9, CD177, IFNG, CIITA, CD3D, CTLA4, CD74 |
| | S100A9, CD177, IFNG, CIITA, CD3D, CTLA4, HP |
| | S100A9, CD177, IFNG, CIITA, CD3D, CD74, HP |
| | S100A9, CD177, IFNG, CIITA, CTLA4, CD74, HP |
| | S100A9, CD177, IFNG, CD3D, CTLA4, CD74, HP |
| | S100A9, CD177, CIITA, CD3D, CTLA4, CD74, HP |
| | S100A9, CX3CR1, IL1R2, IFNG, CIITA, CD3D, CTLA4 |
| | S100A9, CX3CR1, IL1R2, IFNG, CIITA, CD3D, CD74 |
| | S100A9, CX3CR1, IL1R2, IFNG, CIITA, CD3D, HP |
| | S100A9, CX3CR1, IL1R2, IFNG, CIITA, CTLA4, CD74 |
| | S100A9, CX3CR1, IL1R2, IFNG, CIITA, CTLA4, HP |
| | S100A9, CX3CR1, IL1R2, IFNG, CIITA, CD74, HP |
| | S100A9, CX3CR1, IL1R2, IFNG, CD3D, CTLA4, CD74 |
| | S100A9, CX3CR1, IL1R2, IFNG, CD3D, CTLA4, HP |
| | S100A9, CX3CR1, IL1R2, IFNG, CD3D, CD74, HP |
| | S100A9, CX3CR1, IL1R2, IFNG, CTLA4, CD74, HP |
| | S100A9, CX3CR1, IL1R2, CIITA, CD3D, CTLA4, CD74 |
| | S100A9, CX3CR1, IL1R2, CIITA, CD3D, CTLA4, HP |
| | S100A9, CX3CR1, IL1R2, CIITA, CD3D, CD74, HP |
| | S100A9, CX3CR1, IL1R2, CIITA, CTLA4, CD74, HP |
| | S100A9, CX3CR1, IL1R2, CD3D, CTLA4, CD74, HP |
| | S100A9, CX3CR1, IFNG, CIITA, CD3D, CTLA4, CD74 |
| | S100A9, CX3CR1, IFNG, CIITA, CD3D, CTLA4, HP |
| | S100A9, CX3CR1, IFNG, CIITA, CD3D, CD74, HP |
| | S100A9, CX3CR1, IFNG, CIITA, CTLA4, CD74, HP |
| | S100A9, CX3CR1, IFNG, CD3D, CTLA4, CD74, HP |
| | S100A9, CX3CR1, CIITA, CD3D, CTLA4, CD74, HP |
| | S100A9, IL1R2, IFNG, CIITA, CD3D, CTLA4, CD74 |
| | S100A9, IL1R2, IFNG, CIITA, CD3D, CTLA4, HP |
| | S100A9, IL1R2, IFNG, CIITA, CD3D, CD74, HP |
| | S100A9, IL1R2, IFNG, CIITA, CTLA4, CD74, HP |
| | S100A9, IL1R2, IFNG, CD3D, CTLA4, CD74, HP |
| | S100A9, IL1R2, CIITA, CD3D, CTLA4, CD74, HP |
| | S100A9, IFNG, CIITA, CD3D, CTLA4, CD74, HP |
| | TAP2, CD177, CX3CR1, IL1R2, IFNG, CIITA, CD3D |
| | TAP2, CD177, CX3CR1, IL1R2, IFNG, CIITA, CTLA4 |
| | TAP2, CD177, CX3CR1, IL1R2, IFNG, CIITA, CD74 |
| | TAP2, CD177, CX3CR1, IL1R2, IFNG, CIITA, HP |
| | TAP2, CD177, CX3CR1, IL1R2, IFNG, CD3D, CTLA4 |
| | TAP2, CD177, CX3CR1, IL1R2, IFNG, CD3D, CD74 |
| | TAP2, CD177, CX3CR1, IL1R2, IFNG, CD3D, HP |
| | TAP2, CD177, CX3CR1, IL1R2, IFNG, CTLA4, CD74 |
| | TAP2, CD177, CX3CR1, IL1R2, IFNG, CTLA4, HP |
| | TAP2, CD177, CX3CR1, IL1R2, IFNG, CD74, HP |
| | TAP2, CD177, CX3CR1, IL1R2, CIITA, CD3D, CTLA4 |
| | TAP2, CD177, CX3CR1, IL1R2, CIITA, CD3D, CD74 |
| | TAP2, CD177, CX3CR1, IL1R2, CIITA, CD3D, HP |
| | TAP2, CD177, CX3CR1, IL1R2, CIITA, CTLA4, CD74 |
| | TAP2, CD177, CX3CR1, IL1R2, CIITA, CTLA4, HP |
| | TAP2, CD177, CX3CR1, IL1R2, CIITA, CD74, HP |
| | TAP2, CD177, CX3CR1, IL1R2, CD3D, CTLA4, CD74 |
| | TAP2, CD177, CX3CR1, IL1R2, CD3D, CTLA4, HP |
| | TAP2, CD177, CX3CR1, IL1R2, CD3D, CD74, HP |
| | TAP2, CD177, CX3CR1, IL1R2, CTLA4, CD74, HP |
| | TAP2, CD177, CX3CR1, IFNG, CIITA, CD3D, CTLA4 |
| | TAP2, CD177, CX3CR1, IFNG, CIITA, CD3D, CD74 |
| | TAP2, CD177, CX3CR1, IFNG, CIITA, CD3D, HP |
| | TAP2, CD177, CX3CR1, IFNG, CIITA, CTLA4, CD74 |
| | TAP2, CD177, CX3CR1, IFNG, CIITA, CTLA4, HP |
| | TAP2, CD177, CX3CR1, IFNG, CIITA, CD74, HP |
| | TAP2, CD177, CX3CR1, IFNG, CD3D, CTLA4, CD74 |
| | TAP2, CD177, CX3CR1, IFNG, CD3D, CTLA4, HP |
| | TAP2, CD177, CX3CR1, IFNG, CD3D, CD74, HP |
| | TAP2, CD177, CX3CR1, IFNG, CTLA4, CD74, HP |
| | TAP2, CD177, CX3CR1, CIITA, CD3D, CTLA4, CD74 |
| | TAP2, CD177, CX3CR1, CIITA, CD3D, CTLA4, HP |
| | TAP2, CD177, CX3CR1, CIITA, CD3D, CD74, HP |
| | TAP2, CD177, CX3CR1, CIITA, CTLA4, CD74, HP |
| | TAP2, CD177, CX3CR1, CD3D, CTLA4, CD74, HP |
| | TAP2, CD177, IL1R2, IFNG, CIITA, CD3D, CTLA4 |
| | TAP2, CD177, IL1R2, IFNG, CIITA, CD3D, CD74 |
| | TAP2, CD177, IL1R2, IFNG, CIITA, CD3D, HP |
| | TAP2, CD177, IL1R2, IFNG, CIITA, CTLA4, CD74 |
| | TAP2, CD177, IL1R2, IFNG, CIITA, CTLA4, HP |
| | TAP2, CD177, IL1R2, IFNG, CIITA, CD74, HP |
| | TAP2, CD177, IL1R2, IFNG, CD3D, CTLA4, CD74 |
| | TAP2, CD177, IL1R2, IFNG, CD3D, CTLA4, HP |
| | TAP2, CD177, IL1R2, IFNG, CD3D, CD74, HP |
| | TAP2, CD177, IL1R2, IFNG, CTLA4, CD74, HP |
| | TAP2, CD177, IL1R2, CIITA, CD3D, CTLA4, CD74 |
| | TAP2, CD177, IL1R2, CIITA, CD3D, CTLA4, HP |
| | TAP2, CD177, IL1R2, CIITA, CD3D, CD74, HP |
| | TAP2, CD177, IL1R2, CIITA, CTLA4, CD74, HP |
| | TAP2, CD177, IL1R2, CD3D, CTLA4, CD74, HP |
| | TAP2, CD177, IFNG, CIITA, CD3D, CTLA4, CD74 |
| | TAP2, CD177, IFNG, CIITA, CD3D, CTLA4, HP |
| | TAP2, CD177, IFNG, CIITA, CD3D, CD74, HP |
| | TAP2, CD177, IFNG, CIITA, CTLA4, CD74, HP |
| | TAP2, CD177, IFNG, CD3D, CTLA4, CD74, HP |
| | TAP2, CD177, CIITA, CD3D, CTLA4, CD74, HP |
| | TAP2, CX3CR1, IL1R2, IFNG, CIITA, CD3D, CTLA4 |
| | TAP2, CX3CR1, IL1R2, IFNG, CIITA, CD3D, CD74 |
| | TAP2, CX3CR1, IL1R2, IFNG, CIITA, CD3D, HP |
| | TAP2, CX3CR1, IL1R2, IFNG, CIITA, CTLA4, CD74 |
| | TAP2, CX3CR1, IL1R2, IFNG, CIITA, CTLA4, HP |
| | TAP2, CX3CR1, IL1R2, IFNG, CIITA, CD74, HP |
| | TAP2, CX3CR1, IL1R2, IFNG, CD3D, CTLA4, CD74 |
| | TAP2, CX3CR1, IL1R2, IFNG, CD3D, CTLA4, HP |
| | TAP2, CX3CR1, IL1R2, IFNG, CD3D, CD74, HP |
| | TAP2, CX3CR1, IL1R2, IFNG, CTLA4, CD74, HP |
| | TAP2, CX3CR1, IL1R2, CIITA, CD3D, CTLA4, CD74 |
| | TAP2, CX3CR1, IL1R2, CIITA, CD3D, CTLA4, HP |
| | TAP2, CX3CR1, IL1R2, CIITA, CD3D, CD74, HP |
| | TAP2, CX3CR1, IL1R2, CIITA, CTLA4, CD74, HP |
| | TAP2, CX3CR1, IL1R2, CD3D, CTLA4, CD74, HP |
| | TAP2, CX3CR1, IFNG, CIITA, CD3D, CTLA4, CD74 |
| | TAP2, CX3CR1, IFNG, CIITA, CD3D, CTLA4, HP |
| | TAP2, CX3CR1, IFNG, CIITA, CD3D, CD74, HP |
| | TAP2, CX3CR1, IFNG, CIITA, CTLA4, CD74, HP |
| | TAP2, CX3CR1, IFNG, CD3D, CTLA4, CD74, HP |
| | TAP2, CX3CR1, CIITA, CD3D, CTLA4, CD74, HP |
| | TAP2, IL1R2, IFNG, CIITA, CD3D, CTLA4, CD74 |
| | TAP2, IL1R2, IFNG, CIITA, CD3D, CTLA4, HP |
| | TAP2, IL1R2, IFNG, CIITA, CD3D, CD74, HP |
| | TAP2, IL1R2, IFNG, CIITA, CTLA4, CD74, HP |
| | TAP2, IL1R2, IFNG, CD3D, CTLA4, CD74, HP |
| | TAP2, IL1R2, CIITA, CD3D, CTLA4, CD74, HP |
| | TAP2, IFNG, CIITA, CD3D, CTLA4, CD74, HP |
| | CD177, CX3CR1, IL1R2, IFNG, CIITA, CD3D, CTLA4 |
| | CD177, CX3CR1, IL1R2, IFNG, CIITA, CD3D, CD74 |
| | CD177, CX3CR1, IL1R2, IFNG, CIITA, CD3D, HP |
| | CD177, CX3CR1, IL1R2, IFNG, CIITA, CTLA4, CD74 |
| | CD177, CX3CR1, IL1R2, IFNG, CIITA, CTLA4, HP |
| | CD177, CX3CR1, IL1R2, IFNG, CIITA, CD74, HP |
| | CD177, CX3CR1, IL1R2, IFNG, CD3D, CTLA4, CD74 |
| | CD177, CX3CR1, IL1R2, IFNG, CD3D, CTLA4, HP |
| | CD177, CX3CR1, IL1R2, IFNG, CD3D, CD74, HP |
| | CD177, CX3CR1, IL1R2, IFNG, CTLA4, CD74, HP |
| | CD177, CX3CR1, IL1R2, CIITA, CD3D, CTLA4, CD74 |
| | CD177, CX3CR1, IL1R2, CIITA, CD3D, CTLA4, HP |
| | CD177, CX3CR1, IL1R2, CIITA, CD3D, CD74, HP |
| | CD177, CX3CR1, IL1R2, CIITA, CTLA4, CD74, HP |
| | CD177, CX3CR1, IL1R2, CD3D, CTLA4, CD74, HP |
| | CD177, CX3CR1, IFNG, CIITA, CD3D, CTLA4, CD74 |
| | CD177, CX3CR1, IFNG, CIITA, CD3D, CTLA4, HP |
| | CD177, CX3CR1, IFNG, CIITA, CD3D, CD74, HP |
| | CD177, CX3CR1, IFNG, CIITA, CTLA4, CD74, HP |
| | CD177, CX3CR1, IFNG, CD3D, CTLA4, CD74, HP |
| | CD177, CX3CR1, CIITA, CD3D, CTLA4, CD74, HP |
| | CD177, IL1R2, IFNG, CIITA, CD3D, CTLA4, CD74 |
| | CD177, IL1R2, IFNG, CIITA, CD3D, CTLA4, HP |
| | CD177, IL1R2, IFNG, CIITA, CD3D, CD74, HP |
| | CD177, IL1R2, IFNG, CIITA, CTLA4, CD74, HP |
| | CD177, IL1R2, IFNG, CD3D, CTLA4, CD74, HP |
| | CD177, IL1R2, CIITA, CD3D, CTLA4, CD74, HP |
| | CD177, IFNG, CIITA, CD3D, CTLA4, CD74, HP |
| | CX3CR1, IL1R2, IFNG, CIITA, CD3D, CTLA4, CD74 |
| | CX3CR1, IL1R2, IFNG, CIITA, CD3D, CTLA4, HP |
| | CX3CR1, IL1R2, IFNG, CIITA, CD3D, CD74, HP |
| | CX3CR1, IL1R2, IFNG, CIITA, CTLA4, CD74, HP |
| | CX3CR1, IL1R2, IFNG, CD3D, CTLA4, CD74, HP |
| | CX3CR1, IL1R2, CIITA, CD3D, CTLA4, CD74, HP |
| | CX3CR1, IFNG, CIITA, CD3D, CTLA4, CD74, HP |
| | IL1R2, IFNG, CIITA, CD3D, CTLA4, CD74, HP |
| Combinations of eight | S100A9, TAP2, CD177, CX3CR1, IL1R2, IFNG, CIITA, CD3D |
| genes | S100A9, TAP2, CD177, CX3CR1, IL1R2, IFNG, CIITA, CTLA4 |
| | S100A9, TAP2, CD177, CX3CR1, IL1R2, IFNG, CIITA, CD74 |
| | S100A9, TAP2, CD177, CX3CR1, IL1R2, IFNG, CIITA, HP |
| | S100A9, TAP2, CD177, CX3CR1, IL1R2, IFNG, CD3D, CTLA4 |
| | S100A9, TAP2, CD177, CX3CR1, IL1R2, IFNG, CD3D, CD74 |
| | S100A9, TAP2, CD177, CX3CR1, IL1R2, IFNG, CD3D, HP |
| | S100A9, TAP2, CD177, CX3CR1, IL1R2, IFNG, CTLA4, CD74 |
| | S100A9, TAP2, CD177, CX3CR1, IL1R2, IFNG, CTLA4, HP |
| | S100A9, TAP2, CD177, CX3CR1, IL1R2, IFNG, CD74, HP |
| | S100A9, TAP2, CD177, CX3CR1, IL1R2, CIITA, CD3D, CTLA4 |
| | S100A9, TAP2, CD177, CX3CR1, IL1R2, CIITA, CD3D, CD74 |
| | S100A9, TAP2, CD177, CX3CR1, IL1R2, CIITA, CD3D, HP |
| | S100A9, TAP2, CD177, CX3CR1, IL1R2, CIITA, CTLA4, CD74 |
| | S100A9, TAP2, CD177, CX3CR1, IL1R2, CIITA, CTLA4, HP |
| | S100A9, TAP2, CD177, CX3CR1, IL1R2, CIITA, CD74, HP |
| | S100A9, TAP2, CD177, CX3CR1, IL1R2, CD3D, CTLA4, CD74 |
| | S100A9, TAP2, CD177, CX3CR1, IL1R2, CD3D, CTLA4, HP |
| | S100A9, TAP2, CD177, CX3CR1, IL1R2, CD3D, CD74, HP |
| | S100A9, TAP2, CD177, CX3CR1, IL1R2, CTLA4, CD74, HP |
| | S100A9, TAP2, CD177, CX3CR1, IFNG, CIITA, CD3D, CTLA4 |
| | S100A9, TAP2, CD177, CX3CR1, IFNG, CIITA, CD3D, CD74 |
| | S100A9, TAP2, CD177, CX3CR1, IFNG, CIITA, CD3D, HP |
| | S100A9, TAP2, CD177, CX3CR1, IFNG, CIITA, CTLA4, CD74 |
| | S100A9, TAP2, CD177, CX3CR1, IFNG, CIITA, CTLA4, HP |
| | S100A9, TAP2, CD177, CX3CR1, IFNG, CIITA, CD74, HP |
| | S100A9, TAP2, CD177, CX3CR1, IFNG, CD3D, CTLA4, CD74 |
| | S100A9, TAP2, CD177, CX3CR1, IFNG, CD3D, CTLA4, HP |
| | S100A9, TAP2, CD177, CX3CR1, IFNG, CD3D, CD74, HP |
| | S100A9, TAP2, CD177, CX3CR1, IFNG, CTLA4, CD74, HP |
| | S100A9, TAP2, CD177, CX3CR1, CIITA, CD3D, CTLA4, CD74 |
| | S100A9, TAP2, CD177, CX3CR1, CIITA, CD3D, CTLA4, HP |
| | S100A9, TAP2, CD177, CX3CR1, CIITA, CD3D, CD74, HP |
| | S100A9, TAP2, CD177, CX3CR1, CIITA, CTLA4, CD74, HP |
| | S100A9, TAP2, CD177, CX3CR1, CD3D, CTLA4, CD74, HP |
| | S100A9, TAP2, CD177, IL1R2, IFNG, CIITA, CD3D, CTLA4 |
| | S100A9, TAP2, CD177, IL1R2, IFNG, CIITA, CD3D, CD74 |
| | S100A9, TAP2, CD177, IL1R2, IFNG, CIITA, CD3D, HP |
| | S100A9, TAP2, CD177, IL1R2, IFNG, CIITA, CTLA4, CD74 |
| | S100A9, TAP2, CD177, IL1R2, IFNG, CIITA, CTLA4, HP |
| | S100A9, TAP2, CD177, IL1R2, IFNG, CIITA, CD74, HP |
| | S100A9, TAP2, CD177, IL1R2, IFNG, CD3D, CTLA4, CD74 |
| | S100A9, TAP2, CD177, IL1R2, IFNG, CD3D, CTLA4, HP |
| | S100A9, TAP2, CD177, IL1R2, IFNG, CD3D, CD74, HP |
| | S100A9, TAP2, CD177, IL1R2, IFNG, CTLA4, CD74, HP |
| | S100A9, TAP2, CD177, IL1R2, CIITA, CD3D, CTLA4, CD74 |
| | S100A9, TAP2, CD177, IL1R2, CIITA, CD3D, CTLA4, HP |
| | S100A9, TAP2, CD177, IL1R2, CIITA, CD3D, CD74, HP |
| | S100A9, TAP2, CD177, IL1R2, CIITA, CTLA4, CD74, HP |
| | S100A9, TAP2, CD177, IL1R2, CD3D, CTLA4, CD74, HP |
| | S100A9, TAP2, CD177, IFNG, CIITA, CD3D, CTLA4, CD74 |
| | S100A9, TAP2, CD177, IFNG, CIITA, CD3D, CTLA4, HP |
| | S100A9, TAP2, CD177, IFNG, CIITA, CD3D, CD74, HP |
| | S100A9, TAP2, CD177, IFNG, CIITA, CTLA4, CD74, HP |
| | S100A9, TAP2, CD177, IFNG, CD3D, CTLA4, CD74, HP |
| | S100A9, TAP2, CD177, CIITA, CD3D, CTLA4, CD74, HP |
| | S100A9, TAP2, CX3CR1, IL1R2, IFNG, CIITA, CD3D, CTLA4 |
| | S100A9, TAP2, CX3CR1, IL1R2, IFNG, CIITA, CD3D, CD74 |
| | S100A9, TAP2, CX3CR1, IL1R2, IFNG, CIITA, CD3D, HP |
| | S100A9, TAP2, CX3CR1, IL1R2, IFNG, CIITA, CTLA4, CD74 |
| | S100A9, TAP2, CX3CR1, IL1R2, IFNG, CIITA, CTLA4, HP |
| | S100A9, TAP2, CX3CR1, IL1R2, IFNG, CIITA, CD74, HP |
| | S100A9, TAP2, CX3CR1, IL1R2, IFNG, CD3D, CTLA4, CD74 |
| | S100A9, TAP2, CX3CR1, IL1R2, IFNG, CD3D, CTLA4, HP |
| | S100A9, TAP2, CX3CR1, IL1R2, IFNG, CD3D, CD74, HP |
| | S100A9, TAP2, CX3CR1, IL1R2, IFNG, CTLA4, CD74, HP |
| | S100A9, TAP2, CX3CR1, IL1R2, CIITA, CD3D, CTLA4, CD74 |
| | S100A9, TAP2, CX3CR1, IL1R2, CIITA, CD3D, CTLA4, HP |
| | S100A9, TAP2, CX3CR1, IL1R2, CIITA, CD3D, CD74, HP |
| | S100A9, TAP2, CX3CR1, IL1R2, CIITA, CTLA4, CD74, HP |
| | S100A9, TAP2, CX3CR1, IL1R2, CD3D, CTLA4, CD74, HP |
| | S100A9, TAP2, CX3CR1, IFNG, CIITA, CD3D, CTLA4, CD74 |
| | S100A9, TAP2, CX3CR1, IFNG, CIITA, CD3D, CTLA4, HP |
| | S100A9, TAP2, CX3CR1, IFNG, CIITA, CD3D, CD74, HP |
| | S100A9, TAP2, CX3CR1, IFNG, CIITA, CTLA4, CD74, HP |
| | S100A9, TAP2, CX3CR1, IFNG, CD3D, CTLA4, CD74, HP |
| | S100A9, TAP2, CX3CR1, CIITA, CD3D, CTLA4, CD74, HP |
| | S100A9, TAP2, IL1R2, IFNG, CIITA, CD3D, CTLA4, CD74 |
| | S100A9, TAP2, IL1R2, IFNG, CIITA, CD3D, CTLA4, HP |
| | S100A9, TAP2, IL1R2, IFNG, CIITA, CD3D, CD74, HP |
| | S100A9, TAP2, IL1R2, IFNG, CIITA, CTLA4, CD74, HP |
| | S100A9, TAP2, IL1R2, IFNG, CD3D, CTLA4, CD74, HP |
| | S100A9, TAP2, IL1R2, CIITA, CD3D, CTLA4, CD74, HP |
| | S100A9, TAP2, IFNG, CIITA, CD3D, CTLA4, CD74, HP |
| | S100A9, CD177, CX3CR1, IL1R2, IFNG, CIITA, CD3D, CTLA4 |
| | S100A9, CD177, CX3CR1, IL1R2, IFNG, CIITA, CD3D, CD74 |
| | S100A9, CD177, CX3CR1, IL1R2, IFNG, CIITA, CD3D, HP |
| | S100A9, CD177, CX3CR1, IL1R2, IFNG, CIITA, CTLA4, CD74 |
| | S100A9, CD177, CX3CR1, IL1R2, IFNG, CIITA, CTLA4, HP |
| | S100A9, CD177, CX3CR1, IL1R2, IFNG, CIITA, CD74, HP |
| | S100A9, CD177, CX3CR1, IL1R2, IFNG, CD3D, CTLA4, CD74 |
| | S100A9, CD177, CX3CR1, IL1R2, IFNG, CD3D, CTLA4, HP |
| | S100A9, CD177, CX3CR1, IL1R2, IFNG, CD3D, CD74, HP |
| | S100A9, CD177, CX3CR1, IL1R2, IFNG, CTLA4, CD74, HP |
| | S100A9, CD177, CX3CR1, IL1R2, CIITA, CD3D, CTLA4, CD74 |
| | S100A9, CD177, CX3CR1, IL1R2, CIITA, CD3D, CTLA4, HP |
| | S100A9, CD177, CX3CR1, IL1R2, CIITA, CD3D, CD74, HP |
| | S100A9, CD177, CX3CR1, IL1R2, CIITA, CTLA4, CD74, HP |
| | S100A9, CD177, CX3CR1, IL1R2, CD3D, CTLA4, CD74, HP |
| | S100A9, CD177, CX3CR1, IFNG, CIITA, CD3D, CTLA4, CD74 |
| | S100A9, CD177, CX3CR1, IFNG, CIITA, CD3D, CTLA4, HP |
| | S100A9, CD177, CX3CR1, IFNG, CIITA, CD3D, CD74, HP |
| | S100A9, CD177, CX3CR1, IFNG, CIITA, CTLA4, CD74, HP |
| | S100A9, CD177, CX3CR1, IFNG, CD3D, CTLA4, CD74, HP |
| | S100A9, CD177, CX3CR1, CIITA, CD3D, CTLA4, CD74, HP |
| | S100A9, CD177, IL1R2, IFNG, CIITA, CD3D, CTLA4, CD74 |
| | S100A9, CD177, IL1R2, IFNG, CIITA, CD3D, CTLA4, HP |
| | S100A9, CD177, IL1R2, IFNG, CIITA, CD3D, CD74, HP |
| | S100A9, CD177, IL1R2, IFNG, CIITA, CTLA4, CD74, HP |
| | S100A9, CD177, IL1R2, IFNG, CD3D, CTLA4, CD74, HP |
| | S100A9, CD177, IL1R2, CIITA, CD3D, CTLA4, CD74, HP |
| | S100A9, CD177, IFNG, CIITA, CD3D, CTLA4, CD74, HP |
| | S100A9, CX3CR1, IL1R2, IFNG, CIITA, CD3D, CTLA4, CD74 |
| | S100A9, CX3CR1, IL1R2, IFNG, CIITA, CD3D, CTLA4, HP |
| | S100A9, CX3CR1, IL1R2, IFNG, CIITA, CD3D, CD74, HP |
| | S100A9, CX3CR1, IL1R2, IFNG, CIITA, CTLA4, CD74, HP |
| | S100A9, CX3CR1, IL1R2, IFNG, CD3D, CTLA4, CD74, HP |
| | S100A9, CX3CR1, IL1R2, CIITA, CD3D, CTLA4, CD74, HP |
| | S100A9, CX3CR1, IFNG, CIITA, CD3D, CTLA4, CD74, HP |
| | S100A9, IL1R2, IFNG, CIITA, CD3D, CTLA4, CD74, HP |
| | TAP2, CD177, CX3CR1, IL1R2, IFNG, CIITA, CD3D, CTLA4 |
| | TAP2, CD177, CX3CR1, IL1R2, IFNG, CIITA, CD3D, CD74 |
| | TAP2, CD177, CX3CR1, IL1R2, IFNG, CIITA, CD3D, HP |
| | TAP2, CD177, CX3CR1, IL1R2, IFNG, CIITA, CTLA4, CD74 |
| | TAP2, CD177, CX3CR1, IL1R2, IFNG, CIITA, CTLA4, HP |
| | TAP2, CD177, CX3CR1, IL1R2, IFNG, CIITA, CD74, HP |
| | TAP2, CD177, CX3CR1, IL1R2, IFNG, CD3D, CTLA4, CD74 |
| | TAP2, CD177, CX3CR1, IL1R2, IFNG, CD3D, CTLA4, HP |
| | TAP2, CD177, CX3CR1, IL1R2, IFNG, CD3D, CD74, HP |
| | TAP2, CD177, CX3CR1, IL1R2, IFNG, CTLA4, CD74, HP |
| | TAP2, CD177, CX3CR1, IL1R2, CIITA, CD3D, CTLA4, CD74 |
| | TAP2, CD177, CX3CR1, IL1R2, CIITA, CD3D, CTLA4, HP |
| | TAP2, CD177, CX3CR1, IL1R2, CIITA, CD3D, CD74, HP |
| | TAP2, CD177, CX3CR1, IL1R2, CIITA, CTLA4, CD74, HP |
| | TAP2, CD177, CX3CR1, IL1R2, CD3D, CTLA4, CD74, HP |
| | TAP2, CD177, CX3CR1, IFNG, CIITA, CD3D, CTLA4, CD74 |
| | TAP2, CD177, CX3CR1, IFNG, CIITA, CD3D, CTLA4, HP |
| | TAP2, CD177, CX3CR1, IFNG, CIITA, CD3D, CD74, HP |
| | TAP2, CD177, CX3CR1, IFNG, CIITA, CTLA4, CD74, HP |
| | TAP2, CD177, CX3CR1, IFNG, CD3D, CTLA4, CD74, HP |
| | TAP2, CD177, CX3CR1, CIITA, CD3D, CTLA4, CD74, HP |
| | TAP2, CD177, IL1R2, IFNG, CIITA, CD3D, CTLA4, CD74 |
| | TAP2, CD177, IL1R2, IFNG, CIITA, CD3D, CTLA4, HP |
| | TAP2, CD177, IL1R2, IFNG, CIITA, CD3D, CD74, HP |
| | TAP2, CD177, IL1R2, IFNG, CIITA, CTLA4, CD74, HP |
| | TAP2, CD177, IL1R2, IFNG, CD3D, CTLA4, CD74, HP |
| | TAP2, CD177, IL1R2, CIITA, CD3D, CTLA4, CD74, HP |
| | TAP2, CD177, IFNG, CIITA, CD3D, CTLA4, CD74, HP |
| | TAP2, CX3CR1, IL1R2, IFNG, CIITA, CD3D, CTLA4, CD74 |
| | TAP2, CX3CR1, IL1R2, IFNG, CIITA, CD3D, CTLA4, HP |
| | TAP2, CX3CR1, IL1R2, IFNG, CIITA, CD3D, CD74, HP |
| | TAP2, CX3CR1, IL1R2, IFNG, CIITA, CTLA4, CD74, HP |
| | TAP2, CX3CR1, IL1R2, IFNG, CD3D, CTLA4, CD74, HP |
| | TAP2, CX3CR1, IL1R2, CIITA, CD3D, CTLA4, CD74, HP |
| | TAP2, CX3CR1, IFNG, CIITA, CD3D, CTLA4, CD74, HP |
| | TAP2, IL1R2, IFNG, CIITA, CD3D, CTLA4, CD74, HP |
| | CD177, CX3CR1, IL1R2, IFNG, CIITA, CD3D, CTLA4, CD74 |
| | CD177, CX3CR1, IL1R2, IFNG, CIITA, CD3D, CTLA4, HP |
| | CD177, CX3CR1, IL1R2, IFNG, CIITA, CD3D, CD74, HP |
| | CD177, CX3CR1, IL1R2, IFNG, CIITA, CTLA4, CD74, HP |
| | CD177, CX3CR1, IL1R2, IFNG, CD3D, CTLA4, CD74, HP |
| | CD177, CX3CR1, IL1R2, CIITA, CD3D, CTLA4, CD74, HP |
| | CD177, CX3CR1, IFNG, CIITA, CD3D, CTLA4, CD74, HP |
| | CD177, IL1R2, IFNG, CIITA, CD3D, CTLA4, CD74, HP |
| | CX3CR1, IL1R2, IFNG, CIITA, CD3D, CTLA4, CD74, HP |
| Combinations of nine | S100A9, TAP2, CD177, CX3CR1, IL1R2, IFNG, CIITA, CD3D, CTLA4 |
| genes | S100A9, TAP2, CD177, CX3CR1, IL1R2, IFNG, CIITA, CD3D, CD74 |
| | S100A9, TAP2, CD177, CX3CR1, IL1R2, IFNG, CIITA, CD3D, HP |
| | S100A9, TAP2, CD177, CX3CR1, IL1R2, IFNG, CIITA, CTLA4, CD74 |
| | S100A9, TAP2, CD177, CX3CR1, IL1R2, IFNG, CIITA, CTLA4, HP |
| | S100A9, TAP2, CD177, CX3CR1, IL1R2, IFNG, CIITA, CD74, HP |
| | S100A9, TAP2, CD177, CX3CR1, IL1R2, IFNG, CD3D, CTLA4, CD74 |
| | S100A9, TAP2, CD177, CX3CR1, IL1R2, IFNG, CD3D, CTLA4, HP |
| | S100A9, TAP2, CD177, CX3CR1, IL1R2, IFNG, CD3D, CD74, HP |
| | S100A9, TAP2, CD177, CX3CR1, IL1R2, IFNG, CTLA4, CD74, HP |
| | S100A9, TAP2, CD177, CX3CR1, IL1R2, CIITA, CD3D, CTLA4, CD74 |
| | S100A9, TAP2, CD177, CX3CR1, IL1R2, CIITA, CD3D, CTLA4, HP |
| | S100A9, TAP2, CD177, CX3CR1, IL1R2, CIITA, CD3D, CD74, HP |
| | S100A9, TAP2, CD177, CX3CR1, IL1R2, CIITA, CTLA4, CD74, HP |
| | S100A9, TAP2, CD177, CX3CR1, IL1R2, CD3D, CTLA4, CD74, HP |
| | S100A9, TAP2, CD177, CX3CR1, IFNG, CIITA, CD3D, CTLA4, CD74 |
| | S100A9, TAP2, CD177, CX3CR1, IFNG, CIITA, CD3D, CTLA4, HP |
| | S100A9, TAP2, CD177, CX3CR1, IFNG, CIITA, CD3D, CD74, HP |
| | S100A9, TAP2, CD177, CX3CR1, IFNG, CIITA, CTLA4, CD74, HP |
| | S100A9, TAP2, CD177, CX3CR1, IFNG, CD3D, CTLA4, CD74, HP |
| | S100A9, TAP2, CD177, CX3CR1, CIITA, CD3D, CTLA4, CD74, HP |
| | S100A9, TAP2, CD177, IL1R2, IFNG, CIITA, CD3D, CTLA4, CD74 |
| | S100A9, TAP2, CD177, IL1R2, IFNG, CIITA, CD3D, CTLA4, HP |
| | S100A9, TAP2, CD177, IL1R2, IFNG, CIITA, CD3D, CD74, HP |
| | S100A9, TAP2, CD177, IL1R2, IFNG, CIITA, CTLA4, CD74, HP |
| | S100A9, TAP2, CD177, IL1R2, IFNG, CD3D, CTLA4, CD74, HP |
| | S100A9, TAP2, CD177, IL1R2, CIITA, CD3D, CTLA4, CD74, HP |
| | S100A9, TAP2, CD177, IFNG, CIITA, CD3D, CTLA4, CD74, HP |
| | S100A9, TAP2, CX3CR1, IL1R2, IFNG, CIITA, CD3D, CTLA4, CD74 |
| | S100A9, TAP2, CX3CR1, IL1R2, IFNG, CIITA, CD3D, CTLA4, HP |
| | S100A9, TAP2, CX3CR1, IL1R2, IFNG, CIITA, CD3D, CD74, HP |
| | S100A9, TAP2, CX3CR1, IL1R2, IFNG, CIITA, CTLA4, CD74, HP |
| | S100A9, TAP2, CX3CR1, IL1R2, IFNG, CD3D, CTLA4, CD74, HP |
| | S100A9, TAP2, CX3CR1, IL1R2, CIITA, CD3D, CTLA4, CD74, HP |
| | S100A9, TAP2, CX3CR1, IFNG, CIITA, CD3D, CTLA4, CD74, HP |
| | S100A9, TAP2, IL1R2, IFNG, CIITA, CD3D, CTLA4, CD74, HP |
| | S100A9, CD177, CX3CR1, IL1R2, IFNG, CIITA, CD3D, CTLA4, CD74 |
| | S100A9, CD177, CX3CR1, IL1R2, IFNG, CIITA, CD3D, CTLA4, HP |
| | S100A9, CD177, CX3CR1, IL1R2, IFNG, CIITA, CD3D, CD74, HP |
| | S100A9, CD177, CX3CR1, IL1R2, IFNG, CIITA, CTLA4, CD74, HP |
| | S100A9, CD177, CX3CR1, IL1R2, IFNG, CD3D, CTLA4, CD74, HP |
| | S100A9, CD177, CX3CR1, IL1R2, CIITA, CD3D, CTLA4, CD74, HP |
| | S100A9, CD177, CX3CR1, IFNG, CIITA, CD3D, CTLA4, CD74, HP |
| | S100A9, CD177, IL1R2, IFNG, CIITA, CD3D, CTLA4, CD74, HP |
| | S100A9, CX3CR1, IL1R2, IFNG, CIITA, CD3D, CTLA4, CD74, HP |
| | TAP2, CD177, CX3CR1, IL1R2, IFNG, CIITA, CD3D, CTLA4, CD74 |
| | TAP2, CD177, CX3CR1, IL1R2, IFNG, CIITA, CD3D, CTLA4, HP |
| | TAP2, CD177, CX3CR1, IL1R2, IFNG, CIITA, CD3D, CD74, HP |
| | TAP2, CD177, CX3CR1, IL1R2, IFNG, CIITA, CTLA4, CD74, HP |
| | TAP2, CD177, CX3CR1, IL1R2, IFNG, CD3D, CTLA4, CD74, HP |
| | TAP2, CD177, CX3CR1, IL1R2, CIITA, CD3D, CTLA4, CD74, HP |
| | TAP2, CD177, CX3CR1, IFNG, CIITA, CD3D, CTLA4, CD74, HP |
| | TAP2, CD177, IL1R2, IFNG, CIITA, CD3D, CTLA4, CD74, HP |
| | TAP2, CX3CR1, IL1R2, IFNG, CIITA, CD3D, CTLA4, CD74, HP |
| | CD177, CX3CR1, IL1R2, IFNG, CIITA, CD3D, CTLA4, CD74, HP |
| Combinations of ten | S100A9, TAP2, CD177, CX3CR1, IL1R2, IFNG, CIITA, CD3D, CTLA4, CD74 |
| genes | S100A9, TAP2, CD177, CX3CR1, IL1R2, IFNG, CIITA, CD3D, CTLA4, HP |
| | S100A9, TAP2, CD177, CX3CR1, IL1R2, IFNG, CIITA, CD3D, CD74, HP |
| | S100A9, TAP2, CD177, CX3CR1, IL1R2, IFNG, CIITA, CTLA4, CD74, HP |
| | S100A9, TAP2, CD177, CX3CR1, IL1R2, IFNG, CD3D, CTLA4, CD74, HP |
| | S100A9, TAP2, CD177, CX3CR1, IL1R2, CIITA, CD3D, CTLA4, CD74, HP |
| | S100A9, TAP2, CD177, CX3CR1, IFNG, CIITA, CD3D, CTLA4, CD74, HP |
| | S100A9, TAP2, CD177, IL1R2, IFNG, CIITA, CD3D, CTLA4, CD74, HP |
| | S100A9, TAP2, CX3CR1, IL1R2, IFNG, CIITA, CD3D, CTLA4, CD74, HP |
| | S100A9, CD177, CX3CR1, IL1R2, IFNG, CIITA, CD3D, CTLA4, CD74, HP |
| | TAP2, CD177, CX3CR1, IL1R2, IFNG, CIITA, CD3D, CTLA4, CD74, HP |
| Combinations of eleven | S100A9, TAP2, CD177, CX3CR1, IL1R2, IFNG, CIITA, CD3D, CTLA4, |
| genes | CD74, HP |

Preferably, in the method as described previously, in all the embodiments thereof, the biological sample is a blood sample, preferably a blood sample or a blood-derived sample (e.g. PBMCs which can be obtained using the Ficoll method, well known to those skilled in the art, or purified monocytes).

The measurement of the expression (or expression level) of a gene consists in quantifying at least one expression product of the gene. For the purposes of the present invention, the expression product of a gene is any biological molecule resulting from the expression of said gene. More particularly, the expression product of the gene may be an RNA transcript. “Transcript” means RNA, and in particular messenger RNA (mRNA) resulting from the transcription of the gene. More specifically, the transcripts are RNAs produced by the transcription of a gene followed by post-transcriptional modifications of the pre-RNA forms. In the context of the present invention, the expression level of one or more RNA transcripts of the same gene can be measured. Thus, preferably, in the method as described previously, in all the embodiments thereof, the expression of the gene(s) (i.e. the expression of C3AR1, and optionally of one or more other biomarkers of interest, as described previously) is measured at the RNA or mRNA transcript level. In the case of an mRNA transcript, the detection may be performed by a direct method, by any process known to those skilled in the art which makes it possible to determine the presence of said transcript in the sample, or by indirect detection of the transcript after conversion of the latter into DNA, or after amplification of said transcript or after amplification of the DNA obtained after conversion of said transcript into DNA. Numerous methods exist for the detection of nucleic acids (see for example Kricka et al., Clinical Chemistry, 1999, no. 45(4), p. 453-458; Relier G. H. et al., DNA Probes, 2nd Ed., Stockton Press, 1993, sections 5 and 6, p. 173-249). The expression of the genes may particularly be measured by Reverse Transcription-Polymerase Chain Reaction or RT-PCR, preferably by quantitative RT-PCR or RT-qPCR (for example using the FilmArray® technology or the Biomark™ platform from Fluidigm), by sequencing (preferably by high-throughput sequencing) or by hybridization techniques (for example with hybridization microarrays or by techniques of the NanoString® nCounter® type).

The measurement of the expression level of a gene makes it possible in particular to determine the quantity of one or more transcripts present in the biological sample or to give a value derived therefrom. A value derived from the quantity may for example be the absolute concentration, calculated by virtue of a calibration curve obtained from successive dilutions of a solution of amplicons having a given concentration. It may also correspond to the value of the normalized and calibrated quantity, such as the CNRQ (Calibrated Normalized Relative Quantity, (Hellemans et al (2007), Genome biology 8(2):R19), which integrates the values of a reference sample (or of a calibrator) and of one or more housekeeping genes (also referred to as reference genes). By way of example of housekeeping genes, mentioned may be made of the genes DECR1, HPRT1, PPIB, RPLP0, PPIA, GLYR1, RANBP3, 18S, GAPDH and ACTB.

Thus, preferably, in the method as described previously, in all the embodiments thereof, the expression of the gene(s) of interest is normalized in relation to the expression of one or more housekeeping genes (or reference genes), as is known to those skilled in the art; more preferably still, using one or more of the following housekeeping genes: DECR1 (chromosomal location of the gene according to GRCh38/hg38: chr8:90,001,352-90,053,633), HPRT1 (chromosomal location of the gene according to GRCh38/hg38: chrX:134,452,842-134,520,513) and PPIB (chromosomal location of the gene according to GRCh38/hg38: chr15:64,155,812-64,163,205).

Preferably, in the method as described previously, in all the embodiments thereof, the expression of the gene(s) of interest (preferably the normalized expression) in the biological sample from the patient is compared to a reference value or to the expression of the same gene(s) of interest (preferably the normalized expression) in a reference biological sample (this data being used to calculate the CNRQ, as mentioned above). The reference sample may for example be a sample originating from a volunteer (healthy individual), from a patient, or from a mixture of samples from several volunteers (on the one hand) or from several patients (on the other hand). The reference sample can also be a sample taken from a volunteer (or a mixture of samples taken from several volunteers) then treated ex vivo by an immune system-stimulating agent (such as LPS or lipopolysaccharide). The reference sample can also be a mixture of untreated sample(s) and sample(s) treated ex vivo by an immune system-stimulating agent.

Preferably, the method for determining the risk of complication, in particular a risk of death and/or a risk of incidence of a healthcare-associated infection, as described previously, in all the embodiments thereof, also comprises a step of managing the healthcare provisions in order to reduce the risk of complication. A patient identified as having an increased risk of complication may have adapted management of healthcare provisions with the aim of reducing the risk of complication and, for example, in order to reduce the risk of developing sepsis, septic shock or even the risk of death. By way of examples of healthcare provision management, mention may be made of an immunomodulatory treatment adapted to the patient, or else a prophylactic antibiotic treatment, it being possible for the two treatments to be combined, and/or admittance to an ongoing care unit or resuscitation unit in order to reduce the risk of complication, for example to reduce the risk of developing sepsis, septic shock or even the risk of death, in the days following the measurement of the expression of the biomarker(s). Preferably, the immunomodulatory treatment is an immunostimulant treatment if it is determined that the individual is immunosuppressed, or an anti-inflammatory treatment if it is determined that the individual has an inflammatory state. Among the immunostimulant treatments which may be selected, mention may for example be made of the group of the interleukins, in particular IL-7, IL-15 or IL-3, growth factors, in particular GM-CSF, interferons, in particular IFNγ, Toll agonists, antibodies, in particular anti-PD1, anti-PDL1, anti-LAG3, anti-TIM3, anti-IL-10 or anti-CTLA4 antibodies transferrins and apoptosis inhibitor molecules, FLT3L, Thymosin α1, adrenergic antagonists. Among the anti-inflammatory treatments, mention may particularly be made of the group of glucocorticoids, cytostatic agents, molecules acting on immunophilins and cytokines, molecules which block the IL-1 receptor and anti-TNF treatments. Examples of prophylactic antibiotic treatments suitable for preventing pneumonia are described in particular in Annales Françaises d'Anesthésie et de Réanimation [French Annals of Anaesthesia and Resuscitation] (30; 2011; 168-190). Conversely, a patient not at risk of incidence of complication could be quickly admitted to an outpatient hospital service, for example an infectious diseases unit, rather than remaining in a closely-monitored unit which they do not need.

Another subject of the invention is a kit comprising means for amplifying (e.g. primers) and/or means for detecting (e.g. probes) the expression of C3AR1 and of one or more other biomarker(s), as indicated previously, in all the embodiments, and particularly preferably one, two, three, four, five, six, seven, eight, nine, ten or eleven genes selected from the group consisting of: CD74, CIITA, IFNG, IL1R2, TAP2, CD177, HP, CX3R1, S100A9, CTLA4 and CD3D); said kit being characterized in that all the amplification and/or detection means of said kit enable the detection and/or amplification of at most 100, 90, 80, 70, 60, 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, 2 biomarkers in total. “Biomarker” (or “marker”) means a biological characteristic which can be objectively measured and which is indicative of normal or pathological biological processes or of a pharmacological response to a therapeutic intervention. This biomarker may in particular be detectable at the mRNA level. More particularly, the biomarker may be an endogenous biomarker or loci (such as a gene or an HERV/Human Endogenous RetroVirus, found in an individual's chromosomal material) or an exogenous biomarker (such as a virus).

Thus, said kit may for example also comprise means for amplifying and/or detecting one or more housekeeping genes (preferably selected from the list consisting of: DECR1, HPRT1 and PPIB). The kit may also comprise positive control means, making it possible to qualify the quality of RNA extraction, the quality of any amplification and/or hybridization process.

“Primer” or “amplification primer” means a nucleotide fragment which may consist of 5 to 100 nucleotides, preferably 15 to 30 nucleotides, and having hybridization specificity with a target nucleotide sequence under conditions determined for the initiation of enzymatic polymerization, for example in a reaction for the enzymatic amplification of the target nucleotide sequence. Generally use is made of “primer pairs” consisting of two primers. When it is desired to amplify several different biomarkers (e.g. from different genes or HERVs), several different pairs of primers are preferably used, each preferentially having the ability to hybridize specifically with a different biomarker.

“Probe” or “hybridization probe” means a nucleotide fragment typically consisting of 5 to 100 nucleotides, preferably 15 to 90 nucleotides, even more preferably 15 to 35 nucleotides, having hybridization specificity under conditions determined for forming a hybridization complex with a target nucleotide sequence. The probe also comprises a reporter (such as a fluorophore, an enzyme or any other detection system) which will enable the detection of the target nucleotide sequence. In the present invention, the target nucleotide sequence may be a nucleotide sequence contained in a messenger RNA (mRNA) or a nucleotide sequence contained in a complementary DNA (cDNA) obtained by reverse transcription of said mRNA. When it is desired to target several different biomarkers (e.g. from different genes or HERVs), several different probes are preferably used, each preferentially having the ability to hybridize specifically with a different biomarker.

“Hybridization” means the process during which, under suitable conditions, two nucleotide fragments, for example a hybridization probe and a target nucleotide fragment, having sufficiently complementary sequences, are able to form a double strand with stable and specific hydrogen bonds. A nucleotide fragment which is “able to hybridize” with a polynucleotide is a fragment which can hybridize with said polynucleotide under hybridization conditions, which can be determined in each case, as is known. The hybridization conditions are determined by stringency, i.e. the strictness of the operating conditions. Hybridization is more specific when it is carried out at higher stringency levels. The stringency is defined particularly on the basis of the base composition of a probe/target duplex, and also by the degree of mismatch between two nucleic acids. The stringency can also be based on the reaction parameters, such as the concentration and type of the ionic species present in the hybridization solution, the nature and the concentration of denaturing agents, and/or the hybridization temperature. The stringency of the conditions under which a hybridization reaction must be carried out will chiefly depend on the hybridization probes used. All this information is well known and the suitable conditions can be determined by those skilled in the art. In general, depending on the length of the hybridization probes used, the temperature for the hybridization reaction is between approximately 20 and 70° C., in particular between 35 and 65° C. in a saline solution at a concentration of approximately 0.5 to 1 M. A step of detecting the hybridization reaction is subsequently carried out.

“Enzymatic amplification reaction” is intended to mean a process which generates multiple copies of a target nucleotide fragment by the action of at least one enzyme. Such amplification reactions are well known to those skilled in the art and mention may particularly be made of the following techniques: PCR (Polymerase Chain Reaction), LCR (Ligase Chain Reaction), RCR (Repair Chain Reaction), 3SR (Self Sustained Sequence Replication) with patent application WO-A-90/06995, NASBA (Nucleic Acid Sequence-Based Amplification), TMA (Transcription Mediated Amplification) with U.S. Pat. No. 5,399,491, and LAMP (Loop mediated isothermal amplification) with U.S. Pat. No. 6,410,278. When the enzymatic amplification reaction is PCR, reference will more particularly be made to RT-PCR (RT for reverse transcription) when the amplification step is preceded by a step of reverse transcription of messenger RNA (mRNA) to give complementary DNA (cDNA), and to qPCR or RT-qPCR when the PCR is quantitative.

Another subject of the invention is the use:
of means for amplifying (e.g. primers) and/or means for detecting (e.g. probes) the expression of C3AR1 and optionally also the expression of one or more other gene(s), as indicated previously, in all the embodiments (and particularly preferably one, two, three, four, five, six, seven, eight, nine, ten or eleven genes selected from the group consisting of: CD74, CIITA, IFNG, IL1R2, TAP2, CD177, HP, CX3R1, S100A9, CTLA4 and CD3D); or
of a kit comprising such amplification and/or detection means, and preferably all the amplification and/or detection means of said kit enable the detection and/or amplification of at most 100, 90, 80, 70, 60, 50, 40, 30, 29, 28, 27, 26, 25, 24, 23, 22, 21, 20, 19, 18, 17, 16, 15, 14, 13, 12, 11, 10, 9, 8, 7, 6, 5, 4, 3, 2 biomarkers in total, and optionally said kit also comprises means for amplifying and/or for detecting the expression of one or more housekeeping genes (preferably selected from the list consisting of: DECR1, HPRT1 and PPIB),
in order to determine the risk of complication, in particular a risk of death or a risk of incidence of a healthcare-associated infection, preferably of a nosocomial infection, in a patient, preferably a patient within a healthcare facility, more preferably still within a hospital, more preferably still within the emergency department, the resuscitation unit, an intensive care unit or an ongoing care unit; particularly preferably, the patient is a patient in a septic state (more particularly in septic shock), a patient suffering from burns (more particularly severe burns), a patient suffering from an injury (more particularly a severe injury), or a patient undergoing surgery (more particularly major surgery).

The present invention is illustrated, non-limitingly, by the following examples.

### EXAMPLE 1: MEASUREMENT OF THE EXPRESSION OF C3AR1 MAKES IT POSSIBLE TO PREDICT THE RISK OF COMPLICATION IN A PATIENT

### Materials and Methods

A prospective, longitudinal, single-center observational clinical study was carried out at Ho̊pital Edouard Herriot (Lyon, France). The design of this clinical study was published in Rol et al (2017), BMJ Open 7(6): e015734. The clinical study was approved by the Agence Nationale de Sécurité du Médicament et des produits de santé (ANSM) [French Agency for the Safety of Drugs and Health Products] and the Comité de Protection des Personnes Sud-Est II [South-East II Independent Ethics Committee] in December 2015. Amendments to the protocol were made in July 2016 and January 2017. In brief, a total of 377 patients, in a septic state (n=35) or in septic shock (n=72), suffering from severe burns (n=24), from severe injury (n=137) or hospitalized in a resuscitation unit or an intensive care unit following major surgery (n=109), and 175 healthy volunteers, were included between December 2015 and March 2018.
Patients in a septic state/in septic shock: according to the first clinical protocol, only those patients in septic shock were included, on the basis of a suspected infected site, treatment with catecholamimes was started within the 48 h following admission to resuscitation, and treatment with catecholamines (noradrenalin)>0.25 μg/kg/min for at least 2 hours. Subsequently, the eligibility criteria were modified in August 2016, following the publication of a new definition of septic shock, Sepsis 3 (Singer et al (2016), JAMA 810-801:(8)315). Patients in septic shock were therefore included on the basis of a suspected infected site, treatment with catecholamimes was started within the 48 h following admission to resuscitation, along with vasopressive therapy required to maintain arterial pressure of 65 mm Hg and lactate concentration of >2 mmol/l (18 mg/dl), despite correction of hypovolemia. In 2017, the possibility of including patients in a septic state (according to the definition of Sepsis 3) was added, namely a suspected infected site and an increase in the SOFA score 2 points compared to the baseline SOFA in the 48 h following admission to resuscitation. For this population, day 1 corresponds to the day of diagnosis of sepsis or septic shock.
Severe injury: in the first protocol, only those patients with a sever injury were included (Injury Severity Score (ISS)≥25). In August 2016, the possibility of also including less sever injuries (16<ISS<24) was added. For this population, day 1 corresponds to the day of admission to the resuscitation unit or to the intensive care unit (˜day of the injury).
Major surgery: in the first protocol, only esogastrectomy, Bricker-type bladder resection, cephalic duodenopancreatectomy and abdominal aorta surgery by laparotomy were considered. Other types of surgery with a high risk of complications were added in January 2017: pancreatectomy (total or caudal), neuroendocrine tumors, hepatectomy (on the right-hand side), extended colectomy (laparotomy), abdoperineal resection, nephrectomy (laparotomy, PKD), ilio-femoral bypass (Scarpa). For this population, day 1 corresponds to the day of surgery.
Severe burns: the patients were selected on the basis of a total burn surface area of greater than 30%. For this population, day 1 corresponds to the day of admission to the resuscitation unit or to the intensive care unit (˜day of the burn).

The exclusion criteria related essentially to factors which could have impacted the immune status and could have biased the results (for example: severe neutropenia, corticosteroid treatments, an onco-haematological disease, etc.). Each event leading to a suspected healthcare-associated infection, occurring within the hospital before D30, was reviewed independently by three physicians who were not involved in patient recruitment. Twenty-six percent of the patients developed at least one healthcare-associated infection before D30, or before leaving hospital.

Blood samples were collected in PAXgene® tubes (ref. 762165, PreAnalytiX GmbH Hombrechtikon Switzerland), once for healthy volunteers and several times for the patients, i.e. 3-4 times in the first week (on days 1 or 2: D1/2, on days 3 or 4: D3/4, and on days 5, 6 or 7: D5/7), then 3 times at later times (around D14, D28 and D60).

The expression level of C3AR1 was measured in these samples by RT-qPCR. In brief, the RNA was extracted from the total blood samples using the Maxwell HT Simply RNA kit (ref. AX2420, Promega) and the EVO (TECAN) automated platform, following the kit manufacturer's instructions. Next, 10 ng of total RNA were reverse transcribed to give complementary DNA (cDNA) using the Fluidigm Reverse transcription master mix (ref. PN100-6472 A1, Fluidigm), following the manufacturer's instructions. C3AR1 expression was then quantified by qPCR, using the Biomark HD real-time PCR system from Fluidigm. First, a step of pre-amplification of the cDNA was carried out according to the manufacturer's recommendations, using the PreAmp master mix (Ref. PN100-5875 C1, Fluidigm). Next, a step of treatment by exonuclease I (ref. PN100-5875 C1, Fluidigm) was performed, and the qPCRs were carried out in 192.24 integrated fluidic circuits (Ref. PN100-7222 C1) for detection by EvaGreen, as recommended by the manufacturer. The cycle thresholds (Ct) were then determined. The normalization of the C3AR1 expression in CNRQ (Calibrated Normalized Relative Quantity) was carried out using the Ct of the 3 reference genes (DECR1, HPRT1, PPIB) and a calibrator (corresponding to a mixture of samples treated ex vivo with LPS (immune system-stimulating agent) (50%) and samples which were not treated ex vivo (50%), originating from healthy individuals/volunteers) in each 192.24 integrated fluidic circuit, as described in Hellemans et al (2007), Genome biology 8(2):R19.

Regarding data analysis, associations between the expression of C3AR1, measured at different times during the first week, and the incidence of death or of a healthcare-associated infection before D30 from inclusion in the study were evaluated. The results were calculated in the form of Hazard Ratios expressed as the inter-quartile distance with the associated 95% confidence interval (HR IQR). Next, univariate logistic regression was implemented in order to predict the risk of incidence of a healthcare-associated infection before D15. The power of the values predicted by logistic regression to distinguish between healthcare-associated infection and lack of healthcare-associated infection was quantified by the area under the curve (AUC) of the ROC curve (Receiver Operating Characteristic), and 95% confidence intervals were estimated.

Next, the association between the expression of C3AR1 and the incidence of a healthcare-associated infection was evaluated for different time intervals of incidence of the infection (i.e. periods between taking the sample and the 1st incidence of an infection). The different periods considered were: a healthcare-associated infection in the 4 days and in the 7 days following the sample being taken, regardless of when the sample was taken. For each patient who developed a healthcare-associated infection, the sample considered corresponds to the sample taken closest to the incidence of the first episode of healthcare-associated infection.

For patients who did not develop a healthcare-associated infection (i.e. control patients), a matching method was used to select, for each case, a control patient whose sample was taken on the same day and with close SOFA and Charlson scores. Finally, a single control was selected for each unique case. Univariate logistic regressions were implemented. The power of the values predicted by logistic regression to distinguish between healthcare-associated infection and lack of healthcare-associated infection was quantified by the area under the curve (AUC) of the ROC curve, and 95% confidence intervals were estimated.

Results

An increase in the expression of C3AR1 at the mRNA level, measured on D3/4 or on D5/7 from inclusion in the cohort, was associated with a greater risk of incidence of a healthcare-associated infection before D30 in the overall patient population (univariate analysis: D3/4: HR IQR=1.17 [1.01-1.35], p=0.0316; D5/7: HR IQR=1.70 [1.38-2.08], p=0.0000004). This association was always significant for the measurement time on D5/7 after adjustment with the SOFA and Charlson scores (multivariate analysis: HR IQR=1.68 [1.26-2.25], p=0.0005).

An increase in the expression of C3AR1 at the mRNA level, measured on D1/2, D3/4 or D5/7 from inclusion in the cohort, was associated with a greater risk of death before D30 in the overall patient population (univariate analysis: D1/2: HR IQR=1.42 [1.10-1.83], p=0.0072; D3/4: HR IQR=1.54 [1.27-1.85], p=0.000007; D5/7: HR IQR=2.09 [1.37-3.20], p=0.0007).

Moreover, the prediction models showed that the expression of C3AR1 at the mRNA level, measured on D3/4 or D5.7 from inclusion in the cohort, made it possible to predict the incidence of a healthcare-associated infection before D15 from inclusion in the cohort (table 4).

TABLE 4
Table 4 Performance (AUC and 95% confidence intervals, with AUC2.5 and AUC 97.5
limits) of the measurement of the expression of C3AR1, measured on D3/4 or D5/7
from inclusion in the cohort, in predicting the incidence of a healthcare-associated
infection before D15 from inclusion in the cohort in patients in a septic state/in
septic shock, suffering from severe injuries, or hospitalized following major surgery.

| Day on which sample was taken | AUC | AUC.2.5 | AUC.97.5 |
|---|---|---|---|
| D3/4 | 0.70 | 0.62 | 0.78 |
| D5/7 | 0.75 | 0.66 | 0.84 |

The prediction models also showed that the expression of C3AR1 at the mRNA level made it possible to predict the incidence of a healthcare-associated infection in the 4 days or 7 days following the sample being taken (table 5).

TABLE 5
Table 5 Performance (AUC and 95% confidence intervals, with AUC2.5
and AUC 97.5 limits) of the measurement of the expression of C3AR1
in predicting the incidence of a healthcare-associated infection in the
4 days or in the 7 days following the sample being taken.

| Maximum time interval | | | |
|---|---|---|---|
| between the sample being taken and | | | |
| the potential incidence of first | | | |
| healthcare-associated infection | AUC | AUC.2.5 | AUC.97.5 |
| 4 days | 0.66 | 0.56 | 0.76 |
| 7 days | 0.63 | 0.54 | 0.72 |

Thus, the results obtained show that the measurement of the expression of C3AR1 alone makes it possible to predict the risk of complication, and in particular the incidence of healthcare-associated infection(s) in the 15 days starting from the immuno-inflammatory attack, in the 4 days following the sample being taken or in the 7 days following the sample being taken.

### EXAMPLE 2: THE MEASUREMENT OF THE EXPRESSION OF ONE OR MORE OTHER BIOMARKER(S) IN COMBINATION WITH THE MEASUREMENT OF THE EXPRESSION OF C3AR1 MAKES IT POSSIBLE TO IMPROVE THE PERFORMANCE IN PREDICTING THE RISK OF COMPLICATION

In this example, the expression level of C3AR1 and also that of other biomarkers (genes or HERVs) was measured by RT-qPCR. Multivariate logistic regressions (combination of the measurement of the expression of C3AR1 and of one or more other gene(s) or HERV(s)) were then carried out. The measurement of the expression of one or more of these other biomarkers, in addition to the measurement of the expression of C3AR1, makes it possible to improve the performance (compared to the measurement of the expression of C3AR1 alone) in predicting the risk of complication, in particular the risk of incidence of a healthcare-associated infection, whether before D15 starting from inclusion in the cohort (table 6) or in the 4 days or in the 7 days following the sample being taken (table 7).

TABLE 6
Table 6 Performance (AUC and 95% confidence interval, with AUC2.5 and AUC 97.5 limits) of the measurement of the expression of
C3AR1, in combination with one or more other biomarker(s) (multivariate analysis) measured on D3/4 or D5/7 from inclusion in the
cohort, in predicting the incidence of a healthcare-associated infection before D15 from inclusion in the cohort in patients in a
septic state/in septic shock, suffering from severe injuries, or hospitalized following major surgery.

| Day on which | | | | |
|---|---|---|---|---|
| sample was taken | Biomarkers | AUC | AUC.2.5 | AUC.97.5 |
| D3/4 | C3AR1 + 190665001-HERV0376 | 0.7 | 0.62 | 0.78 |
| D3/4 | C3AR1 + 220247002-HERV0797 | 0.7 | 0.62 | 0.78 |
| D3/4 | C3AR1 + IL10 | 0.7 | 0.62 | 0.78 |
| D3/4 | C3AR1 + 050286701-HERV0513 | 0.7 | 0.62 | 0.78 |
| D3/4 | C3AR1 + 130360601-HERV0808 | 0.7 | 0.61 | 0.79 |
| D3/4 | C3AR1 + IL18R1 | 0.7 | 0.62 | 0.78 |
| D3/4 | C3AR1 + IL1R2 | 0.71 | 0.63 | 0.78 |
| D3/4 | C3AR1 + CD74 | 0.71 | 0.62 | 0.79 |
| D3/4 | C3AR1 + 021456001-MALR1017 | 0.71 | 0.63 | 0.79 |
| D3/4 | C3AR1 + PDCD1 | 0.71 | 0.62 | 0.8 |
| D3/4 | C3AR1 + IL1RN | 0.71 | 0.63 | 0.79 |
| D3/4 | C3AR1 + CX3CR1 | 0.71 | 0.63 | 0.79 |
| D3/4 | C3AR1 + IL15 | 0.73 | 0.65 | 0.8 |
| D3/4 | C3AR1 + IL1R2 | 0.705 | 0.628 | 0.782 |
| D3/4 | C3AR1 + IL1R2 + CD74 | 0.707 | 0.621 | 0.793 |
| D5/7 | C3AR1 + CTLA4 | 0.751 | 0.662 | 0.839 |
| D5/7 | C3AR1 + IL18 | 0.753 | 0.668 | 0.837 |
| D5/7 | C3AR1 + OAS3 | 0.753 | 0.667 | 0.839 |
| D5/7 | C3AR1 + IFIH1 | 0.758 | 0.646 | 0.87 |
| D5/7 | C3AR1 + IL1RN | 0.762 | 0.677 | 0.847 |
| D5/7 | C3AR1 + 050286701-HERV0513 | 0.764 | 0.686 | 0.842 |
| D5/7 | C3AR1 + 170369402HE41 | 0.767 | 0.688 | 0.846 |
| D5/7 | C3AR1 + 190665001-HERV0376 | 0.767 | 0.688 | 0.845 |
| D5/7 | C3AR1 + HP | 0.768 | 0.683 | 0.853 |
| D5/7 | C3AR1 + IL7R | 0.772 | 0.701 | 0.843 |
| D5/7 | C3AR1 + IL18RAP | 0.777 | 0.699 | 0.855 |
| D5/7 | C3AR1 + IL15 | 0.782 | 0.682 | 0.881 |
| D5/7 | C3AR1 + ICOS | 0.782 | 0.713 | 0.85 |
| D5/7 | C3AR1 + CD40LG | 0.784 | 0.717 | 0.852 |
| D5/7 | C3AR1 + CD3D | 0.784 | 0.715 | 0.854 |
| D5/7 | C3AR1 + STAT4 | 0.785 | 0.708 | 0.861 |
| D5/7 | C3AR1 + BTLA | 0.787 | 0.716 | 0.857 |
| D5/7 | C3AR1 + IL18R1 | 0.787 | 0.709 | 0.865 |
| D5/7 | C3AR1 + PDCD1 | 0.787 | 0.711 | 0.864 |
| D5/7 | C3AR1 + S100A9 | 0.793 | 0.707 | 0.88 |
| D5/7 | C3AR1 + CD74 | 0.799 | 0.719 | 0.879 |
| D5/7 | C3AR1 + IFNG | 0.799 | 0.729 | 0.87 |
| D5/7 | C3AR1 + CX3CR1 | 0.802 | 0.722 | 0.882 |
| D5/7 | C3AR1 + IL1R2 | 0.817 | 0.756 | 0.879 |
| D5/7 | C3AR1 + 021456001-MALR1017 | 0.839 | 0.784 | 0.893 |
| D5/7 | C3AR1 + CD177 + CIITA | 0.756 | 0.662 | 0.851 |
| D5/7 | C3AR1 + CD177 + CIITA + TAP2 | 0.775 | 0.683 | 0.867 |
| D5/7 | C3AR1 + CD177 + CIITA + TAP2 + CD3D | 0.775 | 0.686 | 0.864 |
| D5/7 | C3AR1 + CD177 + CIITA + TAP2 + CD3D + CTLA4 | 0.772 | 0.682 | 0.862 |
| D5/7 | C3AR1 + CD177 + CIITA + TAP2 + CTLA4 | 0.773 | 0.679 | 0.866 |
| D5/7 | C3AR1 + CD177 + CX3CR1 | 0.76 | 0.659 | 0.862 |
| D5/7 | C3AR1 + CD177 + CX3CR1 + CIITA | 0.767 | 0.674 | 0.859 |
| D5/7 | C3AR1 + CD177 + CX3CR1 + CIITA + CD3D | 0.763 | 0.666 | 0.86 |
| D5/7 | C3AR1 + CD177 + CX3CR1 + CIITA + CD3D + CTLA4 | 0.754 | 0.647 | 0.86 |
| D5/7 | C3AR1 + CD177 + CX3CR1 + CIITA + CTLA4 | 0.756 | 0.65 | 0.862 |
| D5/7 | C3AR1 + CD177 + CX3CR1 + CIITA + TAP2 | 0.778 | 0.688 | 0.868 |
| D5/7 | C3AR1 + CD177 + CX3CR1 + CIITA + TAP2 + CD3D | 0.777 | 0.689 | 0.866 |
| D5/7 | C3AR1 + CD177 + CX3CR1 + CIITA + TAP2 + CD3D + CTLA4 | 0.774 | 0.683 | 0.864 |
| D5/7 | C3AR1 + CD177 + CX3CR1 + CIITA + TAP2 + CTLA4 | 0.774 | 0.682 | 0.865 |
| D5/7 | C3AR1 + CD177 + CX3CR1 + TAP2 | 0.772 | 0.68 | 0.863 |
| D5/7 | C3AR1 + CD177 + CX3CR1 + TAP2 + CD3D | 0.772 | 0.68 | 0.863 |
| D5/7 | C3AR1 + CD177 + CX3CR1 + TAP2 + CD3D + CTLA4 | 0.769 | 0.676 | 0.862 |
| D5/7 | C3AR1 + CD177 + CX3CR1 + TAP2 + CTLA4 | 0.768 | 0.674 | 0.863 |
| D5/7 | C3AR1 + CD177 + S100A9 | 0.756 | 0.661 | 0.851 |
| D5/7 | C3AR1 + CD177 + S100A9 + CD3D | 0.763 | 0.656 | 0.871 |
| D5/7 | C3AR1 + CD177 + S100A9 + CD3D + CTLA4 | 0.76 | 0.648 | 0.871 |
| D5/7 | C3AR1 + CD177 + S100A9 + CIITA | 0.762 | 0.671 | 0.854 |
| D5/7 | C3AR1 + CD177 + S100A9 + CIITA + CD3D | 0.769 | 0.667 | 0.872 |
| D5/7 | C3AR1 + CD177 + S100A9 + CIITA + CD3D + CTLA4 | 0.762 | 0.655 | 0.868 |
| D5/7 | C3AR1 + CD177 + S100A9 + CIITA + TAP2 | 0.789 | 0.704 | 0.873 |
| D5/7 | C3AR1 + CD177 + S100A9 + CIITA + TAP2 + CD3D | 0.779 | 0.691 | 0.867 |
| D5/7 | C3AR1 + CD177 + S100A9 + CIITA + TAP2 + CD3D + CTLA4 | 0.781 | 0.695 | 0.868 |
| D5/7 | C3AR1 + CD177 + S100A9 + CIITA + TAP2 + CTLA4 | 0.785 | 0.699 | 0.871 |
| D5/7 | C3AR1 + CD177 + S100A9 + CX3CR1 | 0.787 | 0.69 | 0.885 |
| D5/7 | C3AR1 + CD177 + S100A9 + CX3CR1 + CD3D | 0.78 | 0.675 | 0.886 |
| D5/7 | C3AR1 + CD177 + S100A9 + CX3CR1 + CD3D + CTLA4 | 0.775 | 0.664 | 0.887 |
| D5/7 | C3AR1 + CD177 + S100A9 + CX3CR1 + CIITA | 0.786 | 0.691 | 0.882 |
| D5/7 | C3AR1 + CD177 + S100A9 + CX3CR1 + CIITA + CD3D | 0.782 | 0.68 | 0.884 |
| D5/7 | C3AR1 + CD177 + S100A9 + CX3CR1 + CIITA + CD3D + CTLA4 | 0.774 | 0.665 | 0.883 |
| D5/7 | C3AR1 + CD177 + S100A9 + CX3CR1 + CIITA + CTLA4 | 0.778 | 0.668 | 0.889 |
| D5/7 | C3AR1 + CD177 + S100A9 + CX3CR1 + CIITA + TAP2 | 0.79 | 0.704 | 0.875 |
| D5/7 | C3AR1 + CD177 + S100A9 + CX3CR1 + CIITA + TAP2 + CD3D | 0.776 | 0.684 | 0.868 |
| D5/7 | C3AR1 + CD177 + S100A9 + CX3CR1 + CIITA + TAP2 + CD3D + CTLA4 | 0.775 | 0.684 | 0.867 |
| D5/7 | C3AR1 + CD177 + S100A9 + CX3CR1 + CIITA + TAP2 + CTLA4 | 0.789 | 0.7 | 0.878 |
| D5/7 | C3AR1 + CD177 + S100A9 + CX3CR1 + CTLA4 | 0.778 | 0.666 | 0.89 |
| D5/7 | C3AR1 + CD177 + S100A9 + CX3CR1 + TAP2 | 0.792 | 0.707 | 0.876 |
| D5/7 | C3AR1 + CD177 + S100A9 + CX3CR1 + TAP2 + CD3D | 0.777 | 0.688 | 0.865 |
| D5/7 | C3AR1 + CD177 + S100A9 + CX3CR1 + TAP2 + CD3D + CTLA4 | 0.777 | 0.688 | 0.866 |
| D5/7 | C3AR1 + CD177 + S100A9 + CX3CR1 + TAP2 + CTLA4 | 0.79 | 0.704 | 0.875 |
| D5/7 | C3AR1 + CD177 + S100A9 + TAP2 | 0.786 | 0.703 | 0.869 |
| D5/7 | C3AR1 + CD177 + S100A9 + TAP2 + CD3D | 0.778 | 0.69 | 0.865 |
| D5/7 | C3AR1 + CD177 + S100A9 + TAP2 + CD3D + CTLA4 | 0.778 | 0.691 | 0.864 |
| D5/7 | C3AR1 + CD177 + S100A9 + TAP2 + CTLA4 | 0.788 | 0.703 | 0.873 |
| D5/7 | C3AR1 + CD177 + TAP2 | 0.769 | 0.676 | 0.862 |
| D5/7 | C3AR1 + CD177 + TAP2 + CD3D | 0.771 | 0.68 | 0.862 |
| D5/7 | C3AR1 + CD177 + TAP2 + CD3D + CTLA4 | 0.769 | 0.676 | 0.863 |
| D5/7 | C3AR1 + CD177 + TAP2 + CTLA4 | 0.769 | 0.676 | 0.863 |
| D5/7 | C3AR1 + CD74 + CD177 | 0.759 | 0.646 | 0.872 |
| D5/7 | C3AR1 + CD74 + CD177 + CD3D | 0.758 | 0.646 | 0.869 |
| D5/7 | C3AR1 + CD74 + CD177 + CD3D + CTLA4 | 0.752 | 0.641 | 0.863 |
| D5/7 | C3AR1 + CD74 + CD177 + CIITA + CD3D | 0.75 | 0.636 | 0.865 |
| D5/7 | C3AR1 + CD74 + CD177 + CIITA + CD3D + CTLA4 | 0.75 | 0.634 | 0.866 |
| D5/7 | C3AR1 + CD74 + CD177 + CIITA + TAP2 | 0.768 | 0.661 | 0.875 |
| D5/7 | C3AR1 + CD74 + CD177 + CIITA + TAP2 + CD3D | 0.763 | 0.658 | 0.868 |
| D5/7 | C3AR1 + CD74 + CD177 + CIITA + TAP2 + CD3D + CTLA4 | 0.767 | 0.66 | 0.873 |
| D5/7 | C3AR1 + CD74 + CD177 + CIITA + TAP2 + CTLA4 | 0.769 | 0.664 | 0.875 |
| D5/7 | C3AR1 + CD74 + CD177 + CTLA4 | 0.753 | 0.636 | 0.87 |
| D5/7 | C3AR1 + CD74 + CD177 + CX3CR1 | 0.767 | 0.658 | 0.876 |
| D5/7 | C3AR1 + CD74 + CD177 + CX3CR1 + CD3D | 0.761 | 0.649 | 0.873 |
| D5/7 | C3AR1 + CD74 + CD177 + CX3CR1 + CD3D + CTLA4 | 0.757 | 0.643 | 0.872 |
| D5/7 | C3AR1 + CD74 + CD177 + CX3CR1 + CIITA | 0.754 | 0.636 | 0.872 |
| D5/7 | C3AR1 + CD74 + CD177 + CX3CR1 + CIITA + CD3D | 0.755 | 0.638 | 0.872 |
| D5/7 | C3AR1 + CD74 + CD177 + CX3CR1 + CIITA + CD3D + CTLA4 | 0.754 | 0.634 | 0.873 |
| D5/7 | C3AR1 + CD74 + CD177 + CX3CR1 + CIITA + CTLA4 | 0.75 | 0.627 | 0.874 |
| D5/7 | C3AR1 + CD74 + CD177 + CX3CR1 + CIITA + TAP2 | 0.769 | 0.661 | 0.876 |
| D5/7 | C3AR1 + CD74 + CD177 + CX3CR1 + CIITA + TAP2 + CD3D | 0.763 | 0.658 | 0.869 |
| D5/7 | C3AR1 + CD74 + CD177 + CX3CR1 + CIITA + TAP2 + CD3D + CTLA4 | 0.765 | 0.659 | 0.872 |
| D5/7 | C3AR1 + CD74 + CD177 + CX3CR1 + CIITA + TAP2 + CTLA4 | 0.767 | 0.658 | 0.876 |
| D5/7 | C3AR1 + CD74 + CD177 + CX3CR1 + CTLA4 | 0.764 | 0.649 | 0.879 |
| D5/7 | C3AR1 + CD74 + CD177 + CX3CR1 + TAP2 | 0.778 | 0.678 | 0.879 |
| D5/7 | C3AR1 + CD74 + CD177 + CX3CR1 + TAP2 + CD3D | 0.773 | 0.673 | 0.874 |
| D5/7 | C3AR1 + CD74 + CD177 + CX3CR1 + TAP2 + CD3D + CTLA4 | 0.769 | 0.667 | 0.872 |
| D5/7 | C3AR1 + CD74 + CD177 + CX3CR1 + TAP2 + CTLA4 | 0.771 | 0.668 | 0.874 |
| D5/7 | C3AR1 + CD74 + CD177 + S100A9 | 0.768 | 0.665 | 0.87 |
| D5/7 | C3AR1 + CD74 + CD177 + S100A9 + CD3D | 0.774 | 0.68 | 0.868 |
| D5/7 | C3AR1 + CD74 + CD177 + S100A9 + CD3D + CTLA4 | 0.771 | 0.677 | 0.865 |
| D5/7 | C3AR1 + CD74 + CD177 + S100A9 + CIITA | 0.762 | 0.654 | 0.87 |
| D5/7 | C3AR1 + CD74 + CD177 + S100A9 + CIITA + CD3D | 0.772 | 0.673 | 0.871 |
| D5/7 | C3AR1 + CD74 + CD177 + S100A9 + CIITA + CD3D + CTLA4 | 0.772 | 0.669 | 0.874 |
| D5/7 | C3AR1 + CD74 + CD177 + S100A9 + CIITA + CTLA4 | 0.753 | 0.637 | 0.869 |
| D5/7 | C3AR1 + CD74 + CD177 + S100A9 + CIITA + TAP2 | 0.785 | 0.688 | 0.882 |
| D5/7 | C3AR1 + CD74 + CD177 + S100A9 + CIITA + TAP2 + CD3D | 0.783 | 0.689 | 0.876 |
| D5/7 | C3AR1 + CD74 + CD177 + S100A9 + CIITA + TAP2 + CD3D + CTLA4 | 0.785 | 0.692 | 0.878 |
| D5/7 | C3AR1 + CD74 + CD177 + S100A9 + CIITA + TAP2 + CTLA4 | 0.782 | 0.683 | 0.881 |
| D5/7 | C3AR1 + CD74 + CD177 + S100A9 + CTLA4 | 0.763 | 0.659 | 0.868 |
| D5/7 | C3AR1 + CD74 + CD177 + S100A9 + CX3CR1 | 0.788 | 0.698 | 0.878 |
| D5/7 | C3AR1 + CD74 + CD177 + S100A9 + CX3CR1 + CD3D | 0.784 | 0.688 | 0.881 |
| D5/7 | C3AR1 + CD74 + CD177 + S100A9 + CX3CR1 + CD3D + CTLA4 | 0.782 | 0.684 | 0.88 |
| D5/7 | C3AR1 + CD74 + CD177 + S100A9 + CX3CR1 + CIITA | 0.782 | 0.68 | 0.884 |
| D5/7 | C3AR1 + CD74 + CD177 + S100A9 + CX3CR1 + CIITA + CD3D | 0.783 | 0.682 | 0.883 |
| D5/7 | C3AR1 + CD74 + CD177 + S100A9 + CX3CR1 + CIITA + CD3D + CTLA4 | 0.775 | 0.671 | 0.879 |
| D5/7 | C3AR1 + CD74 + CD177 + S100A9 + CX3CR1 + CIITA + CTLA4 | 0.771 | 0.657 | 0.885 |
| D5/7 | C3AR1 + CD74 + CD177 + S100A9 + CX3CR1 + CIITA + TAP2 | 0.793 | 0.698 | 0.888 |
| D5/7 | C3AR1 + CD74 + CD177 + S100A9 + CX3CR1 + CIITA + TAP2 + CD3D | 0.79 | 0.696 | 0.884 |
| D5/7 | C3AR1 + CD74 + CD177 + S100A9 + CX3CR1 + CIITA + TAP2 + CD3D + CTLA4 | 0.791 | 0.698 | 0.884 |
| D5/7 | C3AR1 + CD74 + CD177 + S100A9 + CX3CR1 + CIITA + TAP2 + CTLA4 | 0.791 | 0.694 | 0.889 |
| D5/7 | C3AR1 + CD74 + CD177 + S100A9 + CX3CR1 + CTLA4 | 0.781 | 0.684 | 0.879 |
| D5/7 | C3AR1 + CD74 + CD177 + S100A9 + CX3CR1 + TAP2 | 0.792 | 0.704 | 0.879 |
| D5/7 | C3AR1 + CD74 + CD177 + S100A9 + CX3CR1 + TAP2 + CD3D | 0.789 | 0.7 | 0.877 |
| D5/7 | C3AR1 + CD74 + CD177 + S100A9 + CX3CR1 + TAP2 + CD3D + CTLA4 | 0.792 | 0.706 | 0.879 |
| D5/7 | C3AR1 + CD74 + CD177 + S100A9 + CX3CR1 + TAP2 + CTLA4 | 0.791 | 0.7 | 0.881 |
| D5/7 | C3AR1 + CD74 + CD177 + S100A9 + TAP2 | 0.792 | 0.703 | 0.882 |
| D5/7 | C3AR1 + CD74 + CD177 + S100A9 + TAP2 + CD3D | 0.785 | 0.697 | 0.872 |
| D5/7 | C3AR1 + CD74 + CD177 + S100A9 + TAP2 + CD3D + CTLA4 | 0.785 | 0.698 | 0.872 |
| D5/7 | C3AR1 + CD74 + CD177 + S100A9 + TAP2 + CTLA4 | 0.789 | 0.696 | 0.881 |
| D5/7 | C3AR1 + CD74 + CD177 + TAP2 | 0.773 | 0.671 | 0.875 |
| D5/7 | C3AR1 + CD74 + CD177 + TAP2 + CD3D | 0.773 | 0.672 | 0.873 |
| D5/7 | C3AR1 + CD74 + CD177 + TAP2 + CD3D + CTLA4 | 0.77 | 0.668 | 0.871 |
| D5/7 | C3AR1 + CD74 + CD177 + TAP2 + CTLA4 | 0.769 | 0.665 | 0.873 |
| D5/7 | C3AR1 + CD74 + CD3D | 0.767 | 0.663 | 0.87 |
| D5/7 | C3AR1 + CD74 + CD3D + CTLA4 | 0.769 | 0.66 | 0.877 |
| D5/7 | C3AR1 + CD74 + CIITA | 0.754 | 0.636 | 0.872 |
| D5/7 | C3AR1 + CD74 + CIITA + CD3D | 0.755 | 0.64 | 0.87 |
| D5/7 | C3AR1 + CD74 + CIITA + CD3D + CTLA4 | 0.752 | 0.633 | 0.87 |
| D5/7 | C3AR1 + CD74 + CIITA + CTLA4 | 0.749 | 0.629 | 0.87 |
| D5/7 | C3AR1 + CD74 + CIITA + TAP2 | 0.773 | 0.665 | 0.88 |
| D5/7 | C3AR1 + CD74 + CIITA + TAP2 + CD3D | 0.771 | 0.664 | 0.879 |
| D5/7 | C3AR1 + CD74 + CIITA + TAP2 + CD3D + CTLA4 | 0.769 | 0.655 | 0.883 |
| D5/7 | C3AR1 + CD74 + CIITA + TAP2 + CTLA4 | 0.766 | 0.654 | 0.878 |
| D5/7 | C3AR1 + CD74 + CTLA4 | 0.767 | 0.656 | 0.877 |
| D5/7 | C3AR1 + CD74 + CX3CR1 | 0.766 | 0.658 | 0.874 |
| D5/7 | C3AR1 + CD74 + CX3CR1 + CD3D | 0.767 | 0.664 | 0.871 |
| D5/7 | C3AR1 + CD74 + CX3CR1 + CD3D + CTLA4 | 0.769 | 0.661 | 0.878 |
| D5/7 | C3AR1 + CD74 + CX3CR1 + CIITA | 0.756 | 0.637 | 0.875 |
| D5/7 | C3AR1 + CD74 + CX3CR1 + CIITA + CD3D | 0.755 | 0.639 | 0.87 |
| D5/7 | C3AR1 + CD74 + CX3CR1 + CIITA + CD3D + CTLA4 | 0.754 | 0.633 | 0.874 |
| D5/7 | C3AR1 + CD74 + CX3CR1 + CIITA + CTLA4 | 0.753 | 0.633 | 0.873 |
| D5/7 | C3AR1 + CD74 + CX3CR1 + CIITA + TAP2 | 0.773 | 0.664 | 0.883 |
| D5/7 | C3AR1 + CD74 + CX3CR1 + CIITA + TAP2 + CD3D | 0.767 | 0.659 | 0.875 |
| D5/7 | C3AR1 + CD74 + CX3CR1 + CIITA + TAP2 + CD3D + CTLA4 | 0.768 | 0.654 | 0.882 |
| D5/7 | C3AR1 + CD74 + CX3CR1 + CIITA + TAP2 + CTLA4 | 0.767 | 0.653 | 0.881 |
| D5/7 | C3AR1 + CD74 + CX3CR1 + CTLA4 | 0.768 | 0.659 | 0.878 |
| D5/7 | C3AR1 + CD74 + CX3CR1 + TAP2 | 0.776 | 0.674 | 0.878 |
| D5/7 | C3AR1 + CD74 + CX3CR1 + TAP2 + CD3D | 0.776 | 0.678 | 0.873 |
| D5/7 | C3AR1 + CD74 + CX3CR1 + TAP2 + CD3D + CTLA4 | 0.777 | 0.675 | 0.88 |
| D5/7 | C3AR1 + CD74 + CX3CR1 + TAP2 + CTLA4 | 0.777 | 0.674 | 0.88 |
| D5/7 | C3AR1 + CD74 + IFNG | 0.77 | 0.653 | 0.886 |
| D5/7 | C3AR1 + CD74 + IFNG + CD177 | 0.755 | 0.634 | 0.877 |
| D5/7 | C3AR1 + CD74 + IFNG + CD177 + CD3D | 0.754 | 0.625 | 0.882 |
| D5/7 | C3AR1 + CD74 + IFNG + CD177 + CD3D + CTLA4 | 0.75 | 0.62 | 0.881 |
| D5/7 | C3AR1 + CD74 + IFNG + CD177 + CIITA | 0.756 | 0.633 | 0.879 |
| D5/7 | C3AR1 + CD74 + IFNG + CD177 + CIITA + CD3D | 0.754 | 0.628 | 0.881 |
| D5/7 | C3AR1 + CD74 + IFNG + CD177 + CIITA + CD3D + CTLA4 | 0.751 | 0.622 | 0.879 |
| D5/7 | C3AR1 + CD74 + IFNG + CD177 + CIITA + TAP2 | 0.778 | 0.67 | 0.886 |
| D5/7 | C3AR1 + CD74 + IFNG + CD177 + CIITA + TAP2 + CD3D | 0.769 | 0.653 | 0.885 |
| D5/7 | C3AR1 + CD74 + IFNG + CD177 + CIITA + TAP2 + CD3D + CTLA4 | 0.77 | 0.655 | 0.885 |
| D5/7 | C3AR1 + CD74 + IFNG + CD177 + CIITA + TAP2 + CTLA4 | 0.776 | 0.665 | 0.887 |
| D5/7 | C3AR1 + CD74 + IFNG + CD177 + CX3CR1 | 0.754 | 0.628 | 0.881 |
| D5/7 | C3AR1 + CD74 + IFNG + CD177 + CX3CR1 + CD3D | 0.759 | 0.63 | 0.888 |
| D5/7 | C3AR1 + CD74 + IFNG + CD177 + CX3CR1 + CD3D + CTLA4 | 0.751 | 0.616 | 0.885 |
| D5/7 | C3AR1 + CD74 + IFNG + CD177 + CX3CR1 + CIITA | 0.754 | 0.629 | 0.88 |
| D5/7 | C3AR1 + CD74 + IFNG + CD177 + CX3CR1 + CIITA + CD3D | 0.758 | 0.629 | 0.886 |
| D5/7 | C3AR1 + CD74 + IFNG + CD177 + CX3CR1 + CIITA + CD3D + CTLA4 | 0.754 | 0.621 | 0.886 |
| D5/7 | C3AR1 + CD74 + IFNG + CD177 + CX3CR1 + CIITA + CTLA4 | 0.752 | 0.621 | 0.882 |
| D5/7 | C3AR1 + CD74 + IFNG + CD177 + CX3CR1 + CIITA + TAP2 | 0.78 | 0.671 | 0.889 |
| D5/7 | C3AR1 + CD74 + IFNG + CD177 + CX3CR1 + CIITA + TAP2 + CD3D | 0.774 | 0.659 | 0.889 |
| D5/7 | C3AR1 + CD74 + IFNG + CD177 + CX3CR1 + CIITA + TAP2 + CD3D + CTLA4 | 0.772 | 0.656 | 0.888 |
| D5/7 | C3AR1 + CD74 + IFNG + CD177 + CX3CR1 + CIITA + TAP2 + CTLA4 | 0.778 | 0.665 | 0.891 |
| D5/7 | C3AR1 + CD74 + IFNG + CD177 + CX3CR1 + CTLA4 | 0.751 | 0.62 | 0.883 |
| D5/7 | C3AR1 + CD74 + IFNG + CD177 + CX3CR1 + TAP2 | 0.778 | 0.667 | 0.888 |
| D5/7 | C3AR1 + CD74 + IFNG + CD177 + CX3CR1 + TAP2 + CD3D | 0.772 | 0.656 | 0.887 |
| D5/7 | C3AR1 + CD74 + IFNG + CD177 + CX3CR1 + TAP2 + CD3D + CTLA4 | 0.77 | 0.656 | 0.885 |
| D5/7 | C3AR1 + CD74 + IFNG + CD177 + CX3CR1 + TAP2 + CTLA4 | 0.778 | 0.664 | 0.891 |
| D5/7 | C3AR1 + CD74 + IFNG + CD177 + S100A9 | 0.773 | 0.668 | 0.879 |
| D5/7 | C3AR1 + CD74 + IFNG + CD177 + S100A9 + CD3D | 0.789 | 0.683 | 0.894 |
| D5/7 | C3AR1 + CD74 + IFNG + CD177 + S100A9 + CD3D + CTLA4 | 0.791 | 0.687 | 0.896 |
| D5/7 | C3AR1 + CD74 + IFNG + CD177 + S100A9 + CIITA | 0.767 | 0.657 | 0.877 |
| D5/7 | C3AR1 + CD74 + IFNG + CD177 + S100A9 + CIITA + CD3D | 0.79 | 0.682 | 0.898 |
| D5/7 | C3AR1 + CD74 + IFNG + CD177 + S100A9 + CIITA + CD3D + CTLA4 | 0.788 | 0.68 | 0.896 |
| D5/7 | C3AR1 + CD74 + IFNG + CD177 + S100A9 + CIITA + CTLA4 | 0.763 | 0.644 | 0.882 |
| D5/7 | C3AR1 + CD74 + IFNG + CD177 + S100A9 + CIITA + TAP2 | 0.801 | 0.707 | 0.894 |
| D5/7 | C3AR1 + CD74 + IFNG + CD177 + S100A9 + CIITA + TAP2 + CD3D | 0.8 | 0.704 | 0.896 |
| D5/7 | C3AR1 + CD74 + IFNG + CD177 + S100A9 + CIITA + TAP2 + CD3D + CTLA4 | 0.806 | 0.711 | 0.901 |
| D5/7 | C3AR1 + CD74 + IFNG + CD177 + S100A9 + CIITA + TAP2 + CTLA4 | 0.797 | 0.701 | 0.892 |
| D5/7 | C3AR1 + CD74 + IFNG + CD177 + S100A9 + CTLA4 | 0.777 | 0.668 | 0.885 |
| D5/7 | C3AR1 + CD74 + IFNG + CD177 + S100A9 + CX3CR1 | 0.807 | 0.709 | 0.904 |
| D5/7 | C3AR1 + CD74 + IFNG + CD177 + S100A9 + CX3CR1 + CD3D | 0.802 | 0.695 | 0.909 |
| D5/7 | C3AR1 + CD74 + IFNG + CD177 + S100A9 + CX3CR1 + CD3D + CTLA4 | 0.802 | 0.695 | 0.909 |
| D5/7 | C3AR1 + CD74 + IFNG + CD177 + S100A9 + CX3CR1 + CIITA | 0.802 | 0.7 | 0.903 |
| D5/7 | C3AR1 + CD74 + IFNG + CD177 + S100A9 + CX3CR1 + CIITA + CD3D | 0.8 | 0.691 | 0.909 |
| D5/7 | C3AR1 + CD74 + IFNG + CD177 + S100A9 + CX3CR1 + CIITA + CD3D + CTLA4 | 0.8 | 0.691 | 0.909 |
| D5/7 | C3AR1 + CD74 + IFNG + CD177 + S100A9 + CX3CR1 + CIITA + CTLA4 | 0.795 | 0.681 | 0.908 |
| D5/7 | C3AR1 + CD74 + IFNG + CD177 + S100A9 + CX3CR1 + CIITA + TAP2 | 0.814 | 0.719 | 0.909 |
| D5/7 | C3AR1 + CD74 + IFNG + CD177 + S100A9 + CX3CR1 + CIITA + TAP2 + CD3D | 0.808 | 0.709 | 0.906 |
| D5/7 | C3AR1 + CD74 + IFNG + CD177 + S100A9 + CX3CR1 + CIITA + TAP2 + CD3D + | 0.81 | 0.713 | 0.907 |
| | CTLA4 | | | |
| D5/7 | C3AR1 + CD74 + IFNG + CD177 + S100A9 + CX3CR1 + CIITA + TAP2 + CTLA4 | 0.813 | 0.715 | 0.912 |
| D5/7 | C3AR1 + CD74 + IFNG + CD177 + S100A9 + CX3CR1 + CTLA4 | 0.799 | 0.693 | 0.906 |
| D5/7 | C3AR1 + CD74 + IFNG + CD177 + S100A9 + CX3CR1 + TAP2 | 0.82 | 0.729 | 0.912 |
| D5/7 | C3AR1 + CD74 + IFNG + CD177 + S100A9 + CX3CR1 + TAP2 + CD3D | 0.812 | 0.715 | 0.909 |
| D5/7 | C3AR1 + CD74 + IFNG + CD177 + S100A9 + CX3CR1 + TAP2 + CD3D + CTLA4 | 0.81 | 0.713 | 0.906 |
| D5/7 | C3AR1 + CD74 + IFNG + CD177 + S100A9 + CX3CR1 + TAP2 + CTLA4 | 0.819 | 0.725 | 0.913 |
| D5/7 | C3AR1 + CD74 + IFNG + CD177 + S100A9 + TAP2 | 0.801 | 0.709 | 0.893 |
| D5/7 | C3AR1 + CD74 + IFNG + CD177 + S100A9 + TAP2 + CD3D | 0.799 | 0.703 | 0.896 |
| D5/7 | C3AR1 + CD74 + IFNG + CD177 + S100A9 + TAP2 + CD3D + CTLA4 | 0.809 | 0.715 | 0.903 |
| D5/7 | C3AR1 + CD74 + IFNG + CD177 + S100A9 + TAP2 + CTLA4 | 0.801 | 0.709 | 0.894 |
| D5/7 | C3AR1 + CD74 + IFNG + CD177 + TAP2 | 0.778 | 0.67 | 0.886 |
| D5/7 | C3AR1 + CD74 + IFNG + CD177 + TAP2 + CD3D | 0.769 | 0.654 | 0.884 |
| D5/7 | C3AR1 + CD74 + IFNG + CD177 + TAP2 + CD3D + CTLA4 | 0.769 | 0.654 | 0.884 |
| D5/7 | C3AR1 + CD74 + IFNG + CD177 + TAP2 + CTLA4 | 0.775 | 0.664 | 0.887 |
| D5/7 | C3AR1 + CD74 + IFNG + CD3D | 0.761 | 0.641 | 0.881 |
| D5/7 | C3AR1 + CD74 + IFNG + CD3D + CTLA4 | 0.758 | 0.636 | 0.881 |
| D5/7 | C3AR1 + CD74 + IFNG + CIITA | 0.766 | 0.646 | 0.885 |
| D5/7 | C3AR1 + CD74 + IFNG + CIITA + CD3D | 0.761 | 0.64 | 0.882 |
| D5/7 | C3AR1 + CD74 + IFNG + CIITA + CD3D + CTLA4 | 0.757 | 0.633 | 0.882 |
| D5/7 | C3AR1 + CD74 + IFNG + CIITA + CTLA4 | 0.757 | 0.63 | 0.884 |
| D5/7 | C3AR1 + CD74 + IFNG + CIITA + TAP2 | 0.783 | 0.677 | 0.889 |
| D5/7 | C3AR1 + CD74 + IFNG + CIITA + TAP2 + CD3D | 0.778 | 0.668 | 0.888 |
| D5/7 | C3AR1 + CD74 + IFNG + CIITA + TAP2 + CD3D + CTLA4 | 0.772 | 0.659 | 0.886 |
| D5/7 | C3AR1 + CD74 + IFNG + CIITA + TAP2 + CTLA4 | 0.772 | 0.657 | 0.887 |
| D5/7 | C3AR1 + CD74 + IFNG + CTLA4 | 0.762 | 0.641 | 0.884 |
| D5/7 | C3AR1 + CD74 + IFNG + CX3CR1 | 0.765 | 0.649 | 0.881 |
| D5/7 | C3AR1 + CD74 + IFNG + CX3CR1 + CD3D | 0.762 | 0.642 | 0.882 |
| D5/7 | C3AR1 + CD74 + IFNG + CX3CR1 + CD3D + CTLA4 | 0.761 | 0.636 | 0.885 |
| D5/7 | C3AR1 + CD74 + IFNG + CX3CR1 + CIITA | 0.764 | 0.646 | 0.883 |
| D5/7 | C3AR1 + CD74 + IFNG + CX3CR1 + CIITA + CD3D | 0.758 | 0.637 | 0.879 |
| D5/7 | C3AR1 + CD74 + IFNG + CX3CR1 + CIITA + CD3D + CTLA4 | 0.759 | 0.632 | 0.885 |
| D5/7 | C3AR1 + CD74 + IFNG + CX3CR1 + CIITA + CTLA4 | 0.76 | 0.633 | 0.887 |
| D5/7 | C3AR1 + CD74 + IFNG + CX3CR1 + CIITA + TAP2 | 0.785 | 0.679 | 0.89 |
| D5/7 | C3AR1 + CD74 + IFNG + CX3CR1 + CIITA + TAP2 + CD3D | 0.776 | 0.665 | 0.886 |
| D5/7 | C3AR1 + CD74 + IFNG + CX3CR1 + CIITA + TAP2 + CD3D + CTLA4 | 0.77 | 0.656 | 0.885 |
| D5/7 | C3AR1 + CD74 + IFNG + CX3CR1 + CIITA + TAP2 + CTLA4 | 0.774 | 0.66 | 0.887 |
| D5/7 | C3AR1 + CD74 + IFNG + CX3CR1 + CTLA4 | 0.762 | 0.638 | 0.885 |
| D5/7 | C3AR1 + CD74 + IFNG + CX3CR1 + TAP2 | 0.79 | 0.687 | 0.892 |
| D5/7 | C3AR1 + CD74 + IFNG + CX3CR1 + TAP2 + CD3D | 0.778 | 0.669 | 0.887 |
| D5/7 | C3AR1 + CD74 + IFNG + CX3CR1 + TAP2 + CD3D + CTLA4 | 0.776 | 0.665 | 0.888 |
| D5/7 | C3AR1 + CD74 + IFNG + CX3CR1 + TAP2 + CTLA4 | 0.778 | 0.667 | 0.89 |
| D5/7 | C3AR1 + CD74 + IFNG + S100A9 | 0.782 | 0.681 | 0.883 |
| D5/7 | C3AR1 + CD74 + IFNG + S100A9 + CD3D | 0.797 | 0.699 | 0.895 |
| D5/7 | C3AR1 + CD74 + IFNG + S100A9 + CD3D + CTLA4 | 0.795 | 0.695 | 0.894 |
| D5/7 | C3AR1 + CD74 + IFNG + S100A9 + CIITA | 0.78 | 0.672 | 0.888 |
| D5/7 | C3AR1 + CD74 + IFNG + S100A9 + CIITA + CD3D | 0.788 | 0.681 | 0.895 |
| D5/7 | C3AR1 + CD74 + IFNG + S100A9 + CIITA + CD3D + CTLA4 | 0.784 | 0.675 | 0.893 |
| D5/7 | C3AR1 + CD74 + IFNG + S100A9 + CIITA + CTLA4 | 0.773 | 0.655 | 0.891 |
| D5/7 | C3AR1 + CD74 + IFNG + S100A9 + CIITA + TAP2 | 0.801 | 0.706 | 0.897 |
| D5/7 | C3AR1 + CD74 + IFNG + S100A9 + CIITA + TAP2 + CD3D | 0.796 | 0.696 | 0.895 |
| D5/7 | C3AR1 + CD74 + IFNG + S100A9 + CIITA + TAP2 + CD3D + CTLA4 | 0.796 | 0.697 | 0.895 |
| D5/7 | C3AR1 + CD74 + IFNG + S100A9 + CIITA + TAP2 + CTLA4 | 0.795 | 0.695 | 0.895 |
| D5/7 | C3AR1 + CD74 + IFNG + S100A9 + CTLA4 | 0.785 | 0.68 | 0.89 |
| D5/7 | C3AR1 + CD74 + IFNG + S100A9 + CX3CR1 | 0.804 | 0.71 | 0.898 |
| D5/7 | C3AR1 + CD74 + IFNG + S100A9 + CX3CR1 + CD3D | 0.803 | 0.705 | 0.901 |
| D5/7 | C3AR1 + CD74 + IFNG + S100A9 + CX3CR1 + CD3D + CTLA4 | 0.799 | 0.696 | 0.902 |
| D5/7 | C3AR1 + CD74 + IFNG + S100A9 + CX3CR1 + CIITA | 0.796 | 0.692 | 0.9 |
| D5/7 | C3AR1 + CD74 + IFNG + S100A9 + CX3CR1 + CIITA + CD3D | 0.798 | 0.692 | 0.905 |
| D5/7 | C3AR1 + CD74 + IFNG + S100A9 + CX3CR1 + CIITA + CD3D + CTLA4 | 0.802 | 0.694 | 0.91 |
| D5/7 | C3AR1 + CD74 + IFNG + S100A9 + CX3CR1 + CIITA + CTLA4 | 0.796 | 0.685 | 0.907 |
| D5/7 | C3AR1 + CD74 + IFNG + S100A9 + CX3CR1 + CIITA + TAP2 | 0.804 | 0.708 | 0.9 |
| D5/7 | C3AR1 + CD74 + IFNG + S100A9 + CX3CR1 + CIITA + TAP2 + CD3D | 0.797 | 0.696 | 0.897 |
| D5/7 | C3AR1 + CD74 + IFNG + S100A9 + CX3CR1 + CIITA + TAP2 + CD3D + CTLA4 | 0.795 | 0.693 | 0.896 |
| D5/7 | C3AR1 + CD74 + IFNG + S100A9 + CX3CR1 + CIITA + TAP2 + CTLA4 | 0.795 | 0.692 | 0.899 |
| D5/7 | C3AR1 + CD74 + IFNG + S100A9 + CX3CR1 + CTLA4 | 0.801 | 0.699 | 0.904 |
| D5/7 | C3AR1 + CD74 + IFNG + S100A9 + CX3CR1 + TAP2 | 0.811 | 0.722 | 0.9 |
| D5/7 | C3AR1 + CD74 + IFNG + S100A9 + CX3CR1 + TAP2 + CD3D | 0.808 | 0.716 | 0.9 |
| D5/7 | C3AR1 + CD74 + IFNG + S100A9 + CX3CR1 + TAP2 + CD3D + CTLA4 | 0.809 | 0.717 | 0.901 |
| D5/7 | C3AR1 + CD74 + IFNG + S100A9 + CX3CR1 + TAP2 + CTLA4 | 0.807 | 0.713 | 0.901 |
| D5/7 | C3AR1 + CD74 + IFNG + S100A9 + TAP2 | 0.808 | 0.719 | 0.898 |
| D5/7 | C3AR1 + CD74 + IFNG + S100A9 + TAP2 + CD3D | 0.805 | 0.713 | 0.897 |
| D5/7 | C3AR1 + CD74 + IFNG + S100A9 + TAP2 + CD3D + CTLA4 | 0.806 | 0.714 | 0.897 |
| D5/7 | C3AR1 + CD74 + IFNG + S100A9 + TAP2 + CTLA4 | 0.809 | 0.717 | 0.901 |
| D5/7 | C3AR1 + CD74 + IFNG + TAP2 | 0.789 | 0.685 | 0.892 |
| D5/7 | C3AR1 + CD74 + IFNG + TAP2 + CD3D | 0.778 | 0.669 | 0.888 |
| D5/7 | C3AR1 + CD74 + IFNG + TAP2 + CD3D + CTLA4 | 0.777 | 0.666 | 0.888 |
| D5/7 | C3AR1 + CD74 + IFNG + TAP2 + CTLA4 | 0.777 | 0.665 | 0.889 |
| D5/7 | C3AR1 + CD74 + S100A9 | 0.783 | 0.686 | 0.88 |
| D5/7 | C3AR1 + CD74 + S100A9 + CD3D | 0.791 | 0.703 | 0.878 |
| D5/7 | C3AR1 + CD74 + S100A9 + CD3D + CTLA4 | 0.783 | 0.69 | 0.876 |
| D5/7 | C3AR1 + CD74 + S100A9 + CIITA | 0.772 | 0.662 | 0.882 |
| D5/7 | C3AR1 + CD74 + S100A9 + CIITA + CD3D | 0.771 | 0.666 | 0.877 |
| D5/7 | C3AR1 + CD74 + S100A9 + CIITA + CD3D + CTLA4 | 0.767 | 0.655 | 0.879 |
| D5/7 | C3AR1 + CD74 + S100A9 + CIITA + CTLA4 | 0.758 | 0.637 | 0.879 |
| D5/7 | C3AR1 + CD74 + S100A9 + CIITA + TAP2 | 0.786 | 0.686 | 0.885 |
| D5/1 | C3AR1 + CD74 + S100A9 + CIITA + TAP2 + CD3D | 0.784 | 0.686 | 0.882 |
| D5/7 | C3AR1 + CD74 + S100A9 + CIITA + TAP2 + CD3D + CTLA4 | 0.783 | 0.678 | 0.887 |
| D5/7 | C3AR1 + CD74 + S100A9 + CIITA + TAP2 + CTLA4 | 0.782 | 0.677 | 0.887 |
| D5/7 | C3AR1 + CD74 + S100A9 + CTLA4 | 0.778 | 0.677 | 0.879 |
| D5/7 | C3AR1 + CD74 + S100A9 + CX3CR1 | 0.788 | 0.696 | 0.88 |
| D5/7 | C3AR1 + CD74 + S100A9 + CX3CR1 + CD3D | 0.79 | 0.701 | 0.878 |
| D5/7 | C3AR1 + CD74 + S100A9 + CX3CR1 + CD3D + CTLA4 | 0.786 | 0.692 | 0.88 |
| D5/7 | C3AR1 + CD74 + S100A9 + CX3CR1 + CIITA | 0.78 | 0.671 | 0.888 |
| D5/7 | C3AR1 + CD74 + S100A9 + CX3CR1 + CIITA + CD3D | 0.772 | 0.667 | 0.878 |
| D5/7 | C3AR1 + CD74 + S100A9 + CX3CR1 + CIITA + CD3D + CTLA4 | 0.767 | 0.655 | 0.88 |
| D5/7 | C3AR1 + CD74 + S100A9 + CX3CR1 + CIITA + CTLA4 | 0.766 | 0.649 | 0.883 |
| D5/7 | C3AR1 + CD74 + S100A9 + CX3CR1 + CIITA + TAP2 | 0.785 | 0.685 | 0.884 |
| D5/7 | C3AR1 + CD74 + S100A9 + CX3CR1 + CIITA + TAP2 + CD3D | 0.785 | 0.688 | 0.881 |
| D5/7 | C3AR1 + CD74 + S100A9 + CX3CR1 + CIITA + TAP2 + CD3D + CTLA4 | 0.781 | 0.676 | 0.886 |
| D5/7 | C3AR1 + CD74 + S100A9 + CX3CR1 + CIITA + TAP2 + CTLA4 | 0.782 | 0.677 | 0.887 |
| D5/7 | C3AR1 + CD74 + S100A9 + CX3CR1 + CTLA4 | 0.786 | 0.687 | 0.884 |
| D5/7 | C3AR1 + CD74 + S100A9 + CX3CR1 + TAP2 | 0.79 | 0.704 | 0.875 |
| D5/7 | C3AR1 + CD74 + S100A9 + CX3CR1 + TAP2 + CD3D | 0.788 | 0.703 | 0.873 |
| D5/7 | C3AR1 + CD74 + S100A9 + CX3CR1 + TAP2 + CD3D + CTLA4 | 0.785 | 0.697 | 0.872 |
| D5/7 | C3AR1 + CD74 + S100A9 + CX3CR1 + TAP2 + CTLA4 | 0.785 | 0.696 | 0.875 |
| D5/7 | C3AR1 + CD74 + S100A9 + TAP2 | 0.792 | 0.706 | 0.878 |
| D5/7 | C3AR1 + CD74 + S100A9 + TAP2 + CD3D | 0.788 | 0.702 | 0.874 |
| D5/7 | C3AR1 + CD74 + S100A9 + TAP2 + CD3D + CTLA4 | 0.787 | 0.698 | 0.875 |
| D5/7 | C3AR1 + CD74 + S100A9 + TAP2 + CTLA4 | 0.786 | 0.696 | 0.875 |
| D5/7 | C3AR1 + CD74 + TAP2 | 0.779 | 0.677 | 0.881 |
| D5/7 | C3AR1 + CD74 + TAP2 + CD3D | 0.78 | 0.683 | 0.878 |
| D5/7 | C3AR1 + CD74 + TAP2 + CD3D + CTLA4 | 0.778 | 0.673 | 0.882 |
| D5/7 | C3AR1 + CD74 + TAP2 + CTLA4 | 0.778 | 0.676 | 0.881 |
| D5/7 | C3AR1 + CIITA | 0.778 | 0.686 | 0.87 |
| D5/7 | C3AR1 + CIITA + CD3D | 0.77 | 0.682 | 0.859 |
| D5/7 | C3AR1 + CIITA + CD3D + CTLA4 | 0.76 | 0.659 | 0.86 |
| D5/7 | C3AR1 + CIITA + CTLA4 | 0.756 | 0.653 | 0.859 |
| D5/7 | C3AR1 + CIITA + TAP2 | 0.786 | 0.697 | 0.875 |
| D5/7 | C3AR1 + CIITA + TAP2 + CD3D | 0.789 | 0.701 | 0.876 |
| D5/7 | C3AR1 + CIITA + TAP2 + CD3D + CTLA4 | 0.784 | 0.689 | 0.878 |
| D5/7 | C3AR1 + CIITA + TAP2 + CTLA4 | 0.784 | 0.693 | 0.876 |
| D5/7 | C3AR1 + CX3CR1 | 0.774 | 0.673 | 0.874 |
| D5/7 | C3AR1 + CX3CR1 + CD3D | 0.76 | 0.652 | 0.867 |
| D5/7 | C3AR1 + CX3CR1 + CD3D + CTLA4 | 0.755 | 0.638 | 0.871 |
| D5/7 | C3AR1 + CX3CR1 + CIITA | 0.794 | 0.709 | 0.878 |
| D5/7 | C3AR1 + CX3CR1 + CIITA + CD3D | 0.778 | 0.689 | 0.867 |
| D5/7 | C3AR1 + CX3CR1 + CIITA + CD3D + CTLA4 | 0.77 | 0.665 | 0.874 |
| D5/7 | C3AR1 + CX3CR1 + CIITA + CTLA4 | 0.768 | 0.665 | 0.872 |
| D5/7 | C3AR1 + CX3CR1 + CIITA + TAP2 | 0.783 | 0.692 | 0.874 |
| D5/7 | C3AR1 + CX3CR1 + CIITA + TAP2 + CD3D | 0.786 | 0.698 | 0.873 |
| D5/7 | C3AR1 + CX3CR1 + CIITA + TAP2 + CD3D + CTLA4 | 0.782 | 0.687 | 0.877 |
| D5/7 | C3AR1 + CX3CR1 + CIITA + TAP2 + CTLA4 | 0.783 | 0.691 | 0.874 |
| D5/7 | C3AR1 + CX3CR1 + CTLA4 | 0.753 | 0.637 | 0.869 |
| D5/7 | C3AR1 + CX3CR1 + TAP2 | 0.783 | 0.69 | 0.876 |
| D5/7 | C3AR1 + CX3CR1 + TAP2 + CD3D | 0.784 | 0.694 | 0.874 |
| D5/7 | C3AR1 + CX3CR1 + TAP2 + CD3D + CTLA4 | 0.775 | 0.678 | 0.873 |
| D5/7 | C3AR1 + CX3CR1 + TAP2 + CTLA4 | 0.776 | 0.681 | 0.871 |
| D5/7 | C3AR1 + IFNG + CD177 + CIITA + CD3D | 0.751 | 0.637 | 0.865 |
| D5/7 | C3AR1 + IFNG + CD177 + CIITA + TAP2 | 0.773 | 0.672 | 0.874 |
| D5/7 | C3AR1 + IFNG + CD177 + CIITA + TAP2 + CD3D | 0.769 | 0.667 | 0.872 |
| D5/7 | C3AR1 + IFNG + CD177 + CIITA + TAP2 + CD3D + CTLA4 | 0.77 | 0.668 | 0.872 |
| D5/7 | C3AR1 + IFNG + CD177 + CIITA + TAP2 + CTLA4 | 0.775 | 0.675 | 0.875 |
| D5/7 | C3AR1 + IFNG + CD177 + CX3CR1 + CIITA | 0.766 | 0.664 | 0.867 |
| D5/7 | C3AR1 + IFNG + CD177 + CX3CR1 + CIITA + CD3D | 0.759 | 0.644 | 0.874 |
| D5/7 | C3AR1 + IFNG + CD177 + CX3CR1 + CIITA + CD3D + CTLA4 | 0.758 | 0.642 | 0.875 |
| D5/7 | C3AR1 + IFNG + CD177 + CX3CR1 + CIITA + CTLA4 | 0.755 | 0.641 | 0.868 |
| D5/7 | C3AR1 + IFNG + CD177 + CX3CR1 + CIITA + TAP2 | 0.784 | 0.686 | 0.881 |
| D5/7 | C3AR1 + IFNG + CD177 + CX3CR1 + CIITA + TAP2 + CD3D | 0.776 | 0.674 | 0.878 |
| D5/7 | C3AR1 + IFNG + CD177 + CX3CR1 + CIITA + TAP2 + CD3D + CTLA4 | 0.776 | 0.674 | 0.878 |
| D5/7 | C3AR1 + IFNG + CD177 + CX3CR1 + CIITA + TAP2 + CTLA4 | 0.78 | 0.678 | 0.881 |
| D5/7 | C3AR1 + IFNG + CD177 + CX3CR1 + TAP2 | 0.76 | 0.653 | 0.866 |
| D5/7 | C3AR1 + IFNG + CD177 + CX3CR1 + TAP2 + CD3D | 0.757 | 0.649 | 0.864 |
| D5/7 | C3AR1 + IFNG + CD177 + CX3CR1 + TAP2 + CD3D + CTLA4 | 0.756 | 0.646 | 0.866 |
| D5/7 | C3AR1 + IFNG + CD177 + CX3CR1 + TAP2 + CTLA4 | 0.757 | 0.647 | 0.868 |
| D5/7 | C3AR1 + IFNG + CD177 + S100A9 + CD3D | 0.763 | 0.645 | 0.881 |
| D5/7 | C3AR1 + IFNG + CD177 + S100A9 + CD3D + CTLA4 | 0.762 | 0.644 | 0.88 |
| D5/7 | C3AR1 + IFNG + CD177 + S100A9 + CIITA | 0.758 | 0.663 | 0.853 |
| D5/7 | C3AR1 + IFNG + CD177 + S100A9 + CIITA + CD3D | 0.77 | 0.658 | 0.882 |
| D5/7 | C3AR1 + IFNG + CD177 + S100A9 + CIITA + CD3D + CTLA4 | 0.767 | 0.654 | 0.88 |
| D5/7 | C3AR1 + IFNG + CD177 + S100A9 + CIITA + TAP2 | 0.796 | 0.705 | 0.886 |
| D5/7 | C3AR1 + IFNG + CD177 + S100A9 + CIITA + TAP2 + CD3D | 0.795 | 0.696 | 0.893 |
| D5/7 | C3AR1 + IFNG + CD177 + S100A9 + CIITA + TAP2 + CD3D + CTLA4 | 0.795 | 0.698 | 0.891 |
| D5/7 | C3AR1 + IFNG + CD177 + S100A9 + CIITA + TAP2 + CTLA4 | 0.795 | 0.702 | 0.887 |
| D5/7 | C3AR1 + IFNG + CD177 + S100A9 + CX3CR1 | 0.791 | 0.683 | 0.899 |
| D5/7 | C3AR1 + IFNG + CD177 + S100A9 + CX3CR1 + CD3D | 0.781 | 0.658 | 0.904 |
| D5/7 | C3AR1 + IFNG + CD177 + S100A9 + CX3CR1 + CD3D + CTLA4 | 0.783 | 0.659 | 0.907 |
| D5/7 | C3AR1 + IFNG + CD177 + S100A9 + CX3CR1 + CIITA | 0.803 | 0.705 | 0.9 |
| D5/7 | C3AR1 + IFNG + CD177 + S100A9 + CX3CR1 + CIITA + CD3D | 0.791 | 0.678 | 0.903 |
| D5/7 | C3AR1 + IFNG + CD177 + S100A9 + CX3CR1 + CIITA + CD3D + CTLA4 | 0.789 | 0.676 | 0.902 |
| D5/7 | C3AR1 + IFNG + CD177 + S100A9 + CX3CR1 + CIITA + CTLA4 | 0.792 | 0.681 | 0.903 |
| D5/7 | C3AR1 + IFNG + CD177 + S100A9 + CX3CR1 + CIITA + TAP2 | 0.808 | 0.714 | 0.902 |
| D5/7 | C3AR1 + IFNG + CD177 + S100A9 + CX3CR1 + CIITA + TAP2 + CD3D | 0.808 | 0.708 | 0.907 |
| D5/7 | C3AR1 + IFNG + CD177 + S100A9 + CX3CR1 + CIITA + TAP2 + CD3D + CTLA4 | 0.806 | 0.709 | 0.903 |
| D5/7 | C3AR1 + IFNG + CD177 + S100A9 + CX3CR1 + CIITA + TAP2 + CTLA4 | 0.809 | 0.716 | 0.903 |
| D5/7 | C3AR1 + IFNG + CD177 + S100A9 + CX3CR1 + CTLA4 | 0.783 | 0.663 | 0.902 |
| D5/7 | C3AR1 + IFNG + CD177 + S100A9 + CX3CR1 + TAP2 | 0.808 | 0.713 | 0.902 |
| D5/7 | C3AR1 + IFNG + CD177 + S100A9 + CX3CR1 + TAP2 + CD3D | 0.804 | 0.701 | 0.908 |
| D5/7 | C3AR1 + IFNG + CD177 + S100A9 + CX3CR1 + TAP2 + CD3D + CTLA4 | 0.802 | 0.703 | 0.902 |
| D5/7 | C3AR1 + IFNG + CD177 + S100A9 + CX3CR1 + TAP2 + CTLA4 | 0.805 | 0.71 | 0.901 |
| D5/7 | C3AR1 + IFNG + CD177 + S100A9 + TAP2 | 0.795 | 0.704 | 0.886 |
| D5/7 | C3AR1 + IFNG + CD177 + S100A9 + TAP2 + CD3D | 0.798 | 0.7 | 0.896 |
| D5/7 | C3AR1 + IFNG + CD177 + S100A9 + TAP2 + CD3D + CTLA4 | 0.798 | 0.702 | 0.894 |
| D5/7 | C3AR1 + IFNG + CD177 + S100A9 + TAP2 + CTLA4 | 0.794 | 0.702 | 0.886 |
| D5/7 | C3AR1 + IFNG + CD177 + TAP2 | 0.758 | 0.653 | 0.863 |
| D5/7 | C3AR1 + IFNG + CD177 + TAP2 + CD3D | 0.753 | 0.646 | 0.86 |
| D5/7 | C3AR1 + IFNG + CD177 + TAP2 + CD3D + CTLA4 | 0.755 | 0.649 | 0.862 |
| D5/7 | C3AR1 + IFNG + CD177 + TAP2 + CTLA4 | 0.76 | 0.653 | 0.868 |
| D5/7 | C3AR1 + IFNG + CIITA | 0.759 | 0.66 | 0.857 |
| D5/7 | C3AR1 + IFNG + CIITA + CD3D | 0.762 | 0.656 | 0.869 |
| D5/7 | C3AR1 + IFNG + CIITA + CD3D + CTLA4 | 0.755 | 0.644 | 0.866 |
| D5/7 | C3AR1 + IFNG + CIITA + CTLA4 | 0.752 | 0.641 | 0.862 |
| D5/7 | C3AR1 + IFNG + CIITA + TAP2 | 0.788 | 0.694 | 0.881 |
| D5/7 | C3AR1 + IFNG + CIITA + TAP2 + CD3D | 0.785 | 0.69 | 0.88 |
| D5/7 | C3AR1 + IFNG + CIITA + TAP2 + CD3D + CTLA4 | 0.777 | 0.678 | 0.877 |
| D5/7 | C3AR1 + IFNG + CIITA + TAP2 + CTLA4 | 0.784 | 0.687 | 0.881 |
| D5/7 | C3AR1 + IFNG + CX3CR1 + CIITA | 0.776 | 0.682 | 0.87 |
| D5/7 | C3AR1 + IFNG + CX3CR1 + CIITA + CD3D | 0.765 | 0.659 | 0.87 |
| D5/7 | C3AR1 + IFNG + CX3CR1 + CIITA + CD3D + CTLA4 | 0.762 | 0.652 | 0.872 |
| D5/7 | C3AR1 + IFNG + CX3CR1 + CIITA + CTLA4 | 0.762 | 0.653 | 0.871 |
| D5/7 | C3AR1 + IFNG + CX3CR1 + CIITA + TAP2 | 0.792 | 0.699 | 0.885 |
| D5/7 | C3AR1 + IFNG + CX3CR1 + CIITA + TAP2 + CD3D | 0.785 | 0.69 | 0.88 |
| D5/7 | C3AR1 + IFNG + CX3CR1 + CIITA + TAP2 + CD3D + CTLA4 | 0.779 | 0.681 | 0.878 |
| D5/7 | C3AR1 + IFNG + CX3CR1 + CIITA + TAP2 + CTLA4 | 0.784 | 0.686 | 0.882 |
| D5/7 | C3AR1 + IFNG + CX3CR1 + TAP2 | 0.77 | 0.671 | 0.868 |
| D5/7 | C3AR1 + IFNG + CX3CR1 + TAP2 + CD3D | 0.765 | 0.664 | 0.866 |
| D5/7 | C3AR1 + IFNG + CX3CR1 + TAP2 + CD3D + CTLA4 | 0.769 | 0.666 | 0.872 |
| D5/7 | C3AR1 + IFNG + CX3CR1 + TAP2 + CTLA4 | 0.769 | 0.665 | 0.872 |
| D5/7 | C3AR1 + IFNG + S100A9 | 0.757 | 0.656 | 0.859 |
| D5/7 | C3AR1 + IFNG + S100A9 + CD3D | 0.774 | 0.665 | 0.883 |
| D5/7 | C3AR1 + IFNG + S100A9 + CD3D + CTLA4 | 0.775 | 0.666 | 0.885 |
| D5/7 | C3AR1 + IFNG + S100A9 + CIITA | 0.769 | 0.675 | 0.864 |
| D5/7 | C3AR1 + IFNG + S100A9 + CIITA + CD3D | 0.778 | 0.674 | 0.882 |
| D5/7 | C3AR1 + IFNG + S100A9 + CIITA + CD3D + CTLA4 | 0.78 | 0.674 | 0.886 |
| D5/7 | C3AR1 + IFNG + S100A9 + CIITA + CTLA4 | 0.762 | 0.652 | 0.872 |
| D5/7 | C3AR1 + IFNG + S100A9 + CIITA + TAP2 | 0.802 | 0.713 | 0.891 |
| D5/7 | C3AR1 + IFNG + S100A9 + CIITA + TAP2 + CD3D | 0.801 | 0.705 | 0.897 |
| D5/7 | C3AR1 + IFNG + S100A9 + CIITA + TAP2 + CD3D + CTLA4 | 0.801 | 0.706 | 0.896 |
| D5/7 | C3AR1 + IFNG + S100A9 + CIITA + TAP2 + CTLA4 | 0.802 | 0.71 | 0.893 |
| D5/7 | C3AR1 + IFNG + S100A9 + CTLA4 | 0.757 | 0.643 | 0.871 |
| D5/7 | C3AR1 + IFNG + S100A9 + CX3CR1 | 0.8 | 0.702 | 0.899 |
| D5/7 | C3AR1 + IFNG + S100A9 + CX3CR1 + CD3D | 0.79 | 0.681 | 0.899 |
| D5/7 | C3AR1 + IFNG + S100A9 + CX3CR1 + CD3D + CTLA4 | 0.792 | 0.682 | 0.902 |
| D5/7 | C3AR1 + IFNG + S100A9 + CX3CR1 + CIITA | 0.805 | 0.71 | 0.901 |
| D5/7 | C3AR1 + IFNG + S100A9 + CX3CR1 + CIITA + CD3D | 0.797 | 0.694 | 0.899 |
| D5/7 | C3AR1 + IFNG + S100A9 + CX3CR1 + CIITA + CD3D + CTLA4 | 0.795 | 0.686 | 0.903 |
| D5/7 | C3AR1 + IFNG + S100A9 + CX3CR1 + CIITA + CTLA4 | 0.792 | 0.684 | 0.899 |
| D5/7 | C3AR1 + IFNG + S100A9 + CX3CR1 + CIITA + TAP2 | 0.807 | 0.716 | 0.898 |
| D5/7 | C3AR1 + IFNG + S100A9 + CX3CR1 + CIITA + TAP2 + CD3D | 0.805 | 0.709 | 0.902 |
| D5/7 | C3AR1 + IFNG + S100A9 + CX3CR1 + CIITA + TAP2 + CD3D + CTLA4 | 0.803 | 0.705 | 0.901 |
| D5/7 | C3AR1 + IFNG + S100A9 + CX3CR1 + CIITA + TAP2 + CTLA4 | 0.804 | 0.71 | 0.899 |
| D5/7 | C3AR1 + IFNG + S100A9 + CX3CR1 + CTLA4 | 0.788 | 0.676 | 0.899 |
| D5/7 | C3AR1 + IFNG + S100A9 + CX3CR1 + TAP2 | 0.812 | 0.723 | 0.902 |
| D5/7 | C3AR1 + IFNG + S100A9 + CX3CR1 + TAP2 + CD3D | 0.807 | 0.714 | 0.901 |
| D5/7 | C3AR1 + IFNG + S100A9 + CX3CR1 + TAP2 + CD3D + CTLA4 | 0.807 | 0.714 | 0.901 |
| D5/7 | C3AR1 + IFNG + S100A9 + CX3CR1 + TAP2 + CTLA4 | 0.804 | 0.711 | 0.898 |
| D5/7 | C3AR1 + IFNG + S100A9 + TAP2 | 0.806 | 0.719 | 0.893 |
| D5/7 | C3AR1 + IFNG + S100A9 + TAP2 + CD3D | 0.803 | 0.709 | 0.897 |
| D5/7 | C3AR1 + IFNG + S100A9 + TAP2 + CD3D + CTLA4 | 0.804 | 0.71 | 0.898 |
| D5/7 | C3AR1 + IFNG + S100A9 + TAP2 + CTLA4 | 0.805 | 0.715 | 0.895 |
| D5/7 | C3AR1 + IFNG + TAP2 | 0.769 | 0.669 | 0.869 |
| D5/7 | C3AR1 + IFNG + TAP2 + CD3D | 0.765 | 0.664 | 0.866 |
| D5/7 | C3AR1 + IFNG + TAP2 + CD3D + CTLA4 | 0.768 | 0.666 | 0.871 |
| D5/7 | C3AR1 + IFNG + TAP2 + CTLA4 | 0.768 | 0.666 | 0.871 |
| D5/7 | C3AR1 + IL1R2 + CD177 | 0.773 | 0.673 | 0.872 |
| D5/7 | C3AR1 + IL1R2 + CD177 + CD3D | 0.755 | 0.637 | 0.873 |
| D5/7 | C3AR1 + IL1R2 + CD177 + CD3D + CTLA4 | 0.75 | 0.63 | 0.87 |
| D5/7 | C3AR1 + IL1R2 + CD177 + CIITA | 0.769 | 0.673 | 0.866 |
| D5/7 | C3AR1 + IL1R2 + CD177 + CIITA + CD3D | 0.76 | 0.652 | 0.867 |
| D5/7 | C3AR1 + IL1R2 + CD177 + CIITA + CD3D + CTLA4 | 0.752 | 0.641 | 0.862 |
| D5/7 | C3AR1 + IL1R2 + CD177 + CIITA + CTLA4 | 0.752 | 0.643 | 0.861 |
| D5/7 | C3AR1 + IL1R2 + CD177 + CIITA + TAP2 | 0.788 | 0.694 | 0.882 |
| D5/7 | C3AR1 + IL1R2 + CD177 + CIITA + TAP2 + CD3D | 0.784 | 0.69 | 0.878 |
| D5/7 | C3AR1 + IL1R2 + CD177 + CIITA + TAP2 + CD3D + CTLA4 | 0.784 | 0.69 | 0.878 |
| D5/7 | C3AR1 + IL1R2 + CD177 + CIITA + TAP2 + CTLA4 | 0.787 | 0.691 | 0.883 |
| D5/7 | C3AR1 + IL1R2 + CD177 + CX3CR1 | 0.775 | 0.67 | 0.879 |
| D5/7 | C3AR1 + IL1R2 + CD177 + CX3CR1 + CD3D | 0.766 | 0.65 | 0.882 |
| D5/7 | C3AR1 + IL1R2 + CD177 + CX3CR1 + CD3D + CTLA4 | 0.754 | 0.634 | 0.874 |
| D5/7 | C3AR1 + IL1R2 + CD177 + CX3CR1 + CIITA | 0.776 | 0.68 | 0.872 |
| D5/7 | C3AR1 + IL1R2 + CD177 + CX3CR1 + CIITA + CD3D | 0.769 | 0.666 | 0.871 |
| D5/1 | C3AR1 + IL1R2 + CD177 + CX3CR1 + CIITA + CD3D + CTLA4 | 0.76 | 0.649 | 0.87 |
| D5/7 | C3AR1 + IL1R2 + CD177 + CX3CR1 + CIITA + CTLA4 | 0.761 | 0.651 | 0.872 |
| D5/7 | C3AR1 + IL1R2 + CD177 + CX3CR1 + CIITA + TAP2 | 0.788 | 0.693 | 0.883 |
| D5/7 | C3AR1 + IL1R2 + CD177 + CX3CR1 + CIITA + TAP2 + CD3D | 0.786 | 0.691 | 0.88 |
| D5/7 | C3AR1 + IL1R2 + CD177 + CX3CR1 + CIITA + TAP2 + CD3D + CTLA4 | 0.785 | 0.691 | 0.88 |
| D5/7 | C3AR1 + IL1R2 + CD177 + CX3CR1 + CIITA + TAP2 + CTLA4 | 0.786 | 0.69 | 0.882 |
| D5/7 | C3AR1 + IL1R2 + CD177 + CX3CR1 + CTLA4 | 0.758 | 0.638 | 0.879 |
| D5/7 | C3AR1 + IL1R2 + CD177 + CX3CR1 + TAP2 | 0.786 | 0.691 | 0.882 |
| D5/7 | C3AR1 + IL1R2 + CD177 + CX3CR1 + TAP2 + CD3D | 0.783 | 0.689 | 0.877 |
| D5/7 | C3AR1 + IL1R2 + CD177 + CX3CR1 + TAP2 + CD3D + CTLA4 | 0.783 | 0.689 | 0.878 |
| D5/7 | C3AR1 + IL1R2 + CD177 + CX3CR1 + TAP2 + CTLA4 | 0.787 | 0.691 | 0.883 |
| D5/7 | C3AR1 + IL1R2 + CD177 + S100A9 | 0.785 | 0.694 | 0.876 |
| D5/7 | C3AR1 + IL1R2 + CD177 + S100A9 + CD3D | 0.768 | 0.657 | 0.88 |
| D5/7 | C3AR1 + IL1R2 + CD177 + S100A9 + CD3D + CTLA4 | 0.764 | 0.649 | 0.88 |
| D5/7 | C3AR1 + IL1R2 + CD177 + S100A9 + CIITA | 0.785 | 0.695 | 0.875 |
| D5/7 | C3AR1 + IL1R2 + CD177 + S100A9 + CIITA + CD3D | 0.772 | 0.665 | 0.879 |
| D5/7 | C3AR1 + IL1R2 + CD177 + S100A9 + CIITA + CD3D + CTLA4 | 0.765 | 0.654 | 0.876 |
| D5/7 | C3AR1 + IL1R2 + CD177 + S100A9 + CIITA + CTLA4 | 0.763 | 0.65 | 0.876 |
| D5/7 | C3AR1 + IL1R2 + CD177 + S100A9 + CIITA + TAP2 | 0.799 | 0.714 | 0.885 |
| D5/7 | C3AR1 + IL1R2 + CD177 + S100A9 + CIITA + TAP2 + CD3D | 0.789 | 0.699 | 0.878 |
| D5/7 | C3AR1 + IL1R2 + CD177 + S100A9 + CIITA + TAP2 + CD3D + CTLA4 | 0.79 | 0.701 | 0.878 |
| D5/7 | C3AR1 + IL1R2 + CD177 + S100A9 + CIITA + TAP2 + CTLA4 | 0.799 | 0.712 | 0.886 |
| D5/7 | C3AR1 + IL1R2 + CD177 + S100A9 + CTLA4 | 0.763 | 0.65 | 0.876 |
| D5/7 | C3AR1 + IL1R2 + CD177 + S100A9 + CX3CR1 | 0.793 | 0.695 | 0.891 |
| D5/7 | C3AR1 + IL1R2 + CD177 + S100A9 + CX3CR1 + CD3D | 0.781 | 0.673 | 0.89 |
| D5/7 | C3AR1 + IL1R2 + CD177 + S100A9 + CX3CR1 + CD3D + CTLA4 | 0.775 | 0.662 | 0.888 |
| D5/7 | C3AR1 + IL1R2 + CD177 + S100A9 + CX3CR1 + CIITA | 0.792 | 0.697 | 0.887 |
| D5/7 | C3AR1 + IL1R2 + CD177 + S100A9 + CX3CR1 + CIITA + CD3D | 0.781 | 0.675 | 0.886 |
| D5/7 | C3AR1 + IL1R2 + CD177 + S100A9 + CX3CR1 + CIITA + CD3D + CTLA4 | 0.778 | 0.667 | 0.889 |
| D5/7 | C3AR1 + IL1R2 + CD177 + S100A9 + CX3CR1 + CIITA + CTLA4 | 0.782 | 0.669 | 0.895 |
| D5/7 | C3AR1 + IL1R2 + CD177 + S100A9 + CX3CR1 + CIITA + TAP2 | 0.803 | 0.718 | 0.889 |
| D5/7 | C3AR1 + IL1R2 + CD177 + S100A9 + CX3CR1 + CIITA + TAP2 + CD3D | 0.787 | 0.697 | 0.878 |
| D5/7 | C3AR1 + IL1R2 + CD177 + S100A9 + CX3CR1 + CIITA + TAP2 + CD3D + CTLA4 | 0.79 | 0.701 | 0.878 |
| D5/7 | C3AR1 + IL1R2 + CD177 + S100A9 + CX3CR1 + CIITA + TAP2 + CTLA4 | 0.797 | 0.709 | 0.885 |
| D5/7 | C3AR1 + IL1R2 + CD177 + S100A9 + CX3CR1 + CTLA4 | 0.781 | 0.666 | 0.896 |
| D5/7 | C3AR1 + IL1R2 + CD177 + S100A9 + CX3CR1 + TAP2 | 0.798 | 0.713 | 0.883 |
| D5/7 | C3AR1 + IL1R2 + CD177 + S100A9 + CX3CR1 + TAP2 + CD3D | 0.787 | 0.699 | 0.876 |
| D5/7 | C3AR1 + IL1R2 + CD177 + S100A9 + CX3CR1 + TAP2 + CD3D + CTLA4 | 0.79 | 0.703 | 0.876 |
| D5/7 | C3AR1 + IL1R2 + CD177 + S100A9 + CX3CR1 + TAP2 + CTLA4 | 0.798 | 0.711 | 0.884 |
| D5/7 | C3AR1 + IL1R2 + CD177 + S100A9 + TAP2 | 0.793 | 0.707 | 0.879 |
| D5/7 | C3AR1 + IL1R2 + CD177 + S100A9 + TAP2 + CD3D | 0.785 | 0.696 | 0.873 |
| D5/7 | C3AR1 + IL1R2 + CD177 + S100A9 + TAP2 + CD3D + CTLA4 | 0.791 | 0.704 | 0.877 |
| D5/7 | C3AR1 + IL1R2 + CD177 + S100A9 + TAP2 + CTLA4 | 0.793 | 0.708 | 0.879 |
| D5/7 | C3AR1 + IL1R2 + CD177 + TAP2 | 0.786 | 0.691 | 0.882 |
| D5/7 | C3AR1 + IL1R2 + CD177 + TAP2 + CD3D | 0.785 | 0.69 | 0.879 |
| D5/7 | C3AR1 + IL1R2 + CD177 + TAP2 + CD3D + CTLA4 | 0.785 | 0.69 | 0.88 |
| D5/7 | C3AR1 + IL1R2 + CD177 + TAP2 + CTLA4 | 0.786 | 0.689 | 0.882 |
| D5/7 | C3AR1 + IL1R2 + CD3D | 0.783 | 0.677 | 0.889 |
| D5/7 | C3AR1 + IL1R2 + CD3D + CTLA4 | 0.775 | 0.663 | 0.887 |
| D5/7 | C3AR1 + IL1R2 + CD74 | 0.805 | 0.728 | 0.881 |
| D5/7 | C3AR1 + IL1R2 + CD74 + CD177 | 0.766 | 0.655 | 0.876 |
| D5/7 | C3AR1 + IL1R2 + CD74 + CD177 + CD3D | 0.765 | 0.655 | 0.875 |
| D5/7 | C3AR1 + IL1R2 + CD74 + CD177 + CD3D + CTLA4 | 0.764 | 0.652 | 0.876 |
| D5/7 | C3AR1 + IL1R2 + CD74 + CD177 + CIITA | 0.758 | 0.636 | 0.881 |
| D5/7 | C3AR1 + IL1R2 + CD74 + CD177 + CIITA + CD3D | 0.758 | 0.639 | 0.877 |
| D5/7 | C3AR1 + IL1R2 + CD74 + CD177 + CIITA + CD3D + CTLA4 | 0.758 | 0.638 | 0.879 |
| D5/7 | C3AR1 + IL1R2 + CD74 + CD177 + CIITA + TAP2 | 0.783 | 0.674 | 0.891 |
| D5/7 | C3AR1 + IL1R2 + CD74 + CD177 + CIITA + TAP2 + CD3D | 0.773 | 0.663 | 0.883 |
| D5/7 | C3AR1 + IL1R2 + CD74 + CD177 + CIITA + TAP2 + CD3D + CTLA4 | 0.775 | 0.665 | 0.885 |
| D5/7 | C3AR1 + IL1R2 + CD74 + CD177 + CIITA + TAP2 + CTLA4 | 0.778 | 0.667 | 0.889 |
| D5/7 | C3AR1 + IL1R2 + CD74 + CD177 + CTLA4 | 0.766 | 0.652 | 0.88 |
| D5/7 | C3AR1 + IL1R2 + CD74 + CD177 + CX3CR1 | 0.769 | 0.66 | 0.879 |
| D5/7 | C3AR1 + IL1R2 + CD74 + CD177 + CX3CR1 + CD3D | 0.767 | 0.655 | 0.88 |
| D5/7 | C3AR1 + IL1R2 + CD74 + CD177 + CX3CR1 + CD3D + CTLA4 | 0.762 | 0.648 | 0.876 |
| D5/7 | C3AR1 + IL1R2 + CD74 + CD177 + CX3CR1 + CIITA | 0.763 | 0.643 | 0.883 |
| D5/7 | C3AR1 + IL1R2 + CD74 + CD177 + CX3CR1 + CIITA + CD3D | 0.758 | 0.639 | 0.877 |
| D5/7 | C3AR1 + IL1R2 + CD74 + CD177 + CX3CR1 + CIITA + CD3D + CTLA4 | 0.76 | 0.638 | 0.882 |
| D5/7 | C3AR1 + IL1R2 + CD74 + CD177 + CX3CR1 + CIITA + CTLA4 | 0.751 | 0.623 | 0.88 |
| D5/7 | C3AR1 + IL1R2 + CD74 + CD177 + CX3CR1 + CIITA + TAP2 | 0.779 | 0.668 | 0.89 |
| D5/7 | C3AR1 + IL1R2 + CD74 + CD177 + CX3CR1 + CIITA + TAP2 + CD3D | 0.771 | 0.66 | 0.882 |
| D5/7 | C3AR1 + IL1R2 + CD74 + CD177 + CX3CR1 + CIITA + TAP2 + CD3D + CTLA4 | 0.771 | 0.66 | 0.882 |
| D5/7 | C3AR1 + IL1R2 + CD74 + CD177 + CX3CR1 + CIITA + TAP2 + CTLA4 | 0.778 | 0.666 | 0.889 |
| D5/7 | C3AR1 + IL1R2 + CD74 + CD177 + CX3CR1 + CTLA4 | 0.767 | 0.653 | 0.881 |
| D5/7 | C3AR1 + IL1R2 + CD74 + CD177 + CX3CR1 + TAP2 | 0.78 | 0.679 | 0.881 |
| D5/7 | C3AR1 + IL1R2 + CD74 + CD177 + CX3CR1 + TAP2 + CD3D | 0.777 | 0.675 | 0.88 |
| D5/7 | C3AR1 + IL1R2 + CD74 + CD177 + CX3CR1 + TAP2 + CD3D + CTLA4 | 0.777 | 0.674 | 0.879 |
| D5/7 | C3AR1 + IL1R2 + CD74 + CD177 + CX3CR1 + TAP2 + CTLA4 | 0.78 | 0.677 | 0.883 |
| D5/7 | C3AR1 + IL1R2 + CD74 + CD177 + S100A9 | 0.77 | 0.67 | 0.871 |
| D5/7 | C3AR1 + IL1R2 + CD74 + CD177 + S100A9 + CD3D | 0.777 | 0.681 | 0.873 |
| D5/7 | C3AR1 + IL1R2 + CD74 + CD177 + S100A9 + CD3D + CTLA4 | 0.779 | 0.683 | 0.874 |
| D5/7 | C3AR1 + IL1R2 + CD74 + CD177 + S100A9 + CIITA | 0.775 | 0.665 | 0.885 |
| D5/7 | C3AR1 + IL1R2 + CD74 + CD177 + S100A9 + CIITA + CD3D | 0.781 | 0.677 | 0.886 |
| D5/7 | C3AR1 + IL1R2 + CD74 + CD177 + S100A9 + CIITA + CD3D + CTLA4 | 0.777 | 0.67 | 0.884 |
| D5/7 | C3AR1 + IL1R2 + CD74 + CD177 + S100A9 + CIITA + CTLA4 | 0.766 | 0.649 | 0.884 |
| D5/7 | C3AR1 + IL1R2 + CD74 + CD177 + S100A9 + CIITA + TAP2 | 0.79 | 0.691 | 0.889 |
| D5/7 | C3AR1 + IL1R2 + CD74 + CD177 + S100A9 + CIITA + TAP2 + CD3D | 0.789 | 0.693 | 0.884 |
| D5/7 | C3AR1 + IL1R2 + CD74 + CD177 + S100A9 + CIITA + TAP2 + CD3D + CTLA4 | 0.79 | 0.695 | 0.885 |
| D5/7 | C3AR1 + IL1R2 + CD74 + CD177 + S100A9 + CIITA + TAP2 + CTLA4 | 0.792 | 0.693 | 0.89 |
| D5/7 | C3AR1 + IL1R2 + CD74 + CD177 + S100A9 + CTLA4 | 0.773 | 0.669 | 0.876 |
| D5/7 | C3AR1 + IL1R2 + CD74 + CD177 + S100A9 + CX3CR1 | 0.791 | 0.7 | 0.882 |
| D5/7 | C3AR1 + IL1R2 + CD74 + CD177 + S100A9 + CX3CR1 + CD3D | 0.786 | 0.689 | 0.883 |
| D5/7 | C3AR1 + IL1R2 + CD74 + CD177 + S100A9 + CX3CR1 + CD3D + CTLA4 | 0.781 | 0.681 | 0.881 |
| D5/7 | C3AR1 + IL1R2 + CD74 + CD177 + S100A9 + CX3CR1 + CIITA | 0.785 | 0.681 | 0.889 |
| D5/7 | C3AR1 + IL1R2 + CD74 + CD177 + S100A9 + CX3CR1 + CIITA + CD3D | 0.784 | 0.681 | 0.888 |
| D5/7 | C3AR1 + IL1R2 + CD74 + CD177 + S100A9 + CX3CR1 + CIITA + CD3D + CTLA4 | 0.779 | 0.672 | 0.886 |
| D5/7 | C3AR1 + IL1R2 + CD74 + CD177 + S100A9 + CX3CR1 + CIITA + CTLA4 | 0.777 | 0.663 | 0.89 |
| D5/7 | C3AR1 + IL1R2 + CD74 + CD177 + S100A9 + CX3CR1 + CIITA + TAP2 | 0.793 | 0.695 | 0.892 |
| D5/7 | C3AR1 + IL1R2 + CD74 + CD177 + S100A9 + CX3CR1 + CIITA + TAP2 + CD3D | 0.787 | 0.692 | 0.883 |
| D5/7 | C3AR1 + IL1R2 + CD74 + CD177 + S100A9 + CX3CR1 + CIITA + TAP2 + CD3D + | 0.791 | 0.698 | 0.885 |
| | CTLA4 | | | |
| D5/7 | C3AR1 + IL1R2 + CD74 + CD177 + S100A9 + CX3CR1 + CIITA + TAP2 + CTLA4 | 0.793 | 0.694 | 0.892 |
| D5/7 | C3AR1 + IL1R2 + CD74 + CD177 + S100A9 + CX3CR1 + CTLA4 | 0.782 | 0.682 | 0.882 |
| D5/7 | C3AR1 + IL1R2 + CD74 + CD177 + S100A9 + CX3CR1 + TAP2 | 0.793 | 0.703 | 0.884 |
| D5/7 | C3AR1 + IL1R2 + CD74 + CD177 + S100A9 + CX3CR1 + TAP2 + CD3D | 0.792 | 0.704 | 0.881 |
| D5/7 | C3AR1 + IL1R2 + CD74 + CD177 + S100A9 + CX3CR1 + TAP2 + CD3D + CTLA4 | 0.795 | 0.707 | 0.883 |
| D5/7 | C3AR1 + IL1R2 + CD74 + CD177 + S100A9 + CX3CR1 + TAP2 + CTLA4 | 0.791 | 0.699 | 0.883 |
| D5/7 | C3AR1 + IL1R2 + CD74 + CD177 + S100A9 + TAP2 | 0.791 | 0.699 | 0.883 |
| D5/7 | C3AR1 + IL1R2 + CD74 + CD177 + S100A9 + TAP2 + CD3D | 0.792 | 0.703 | 0.881 |
| D5/7 | C3AR1 + IL1R2 + CD74 + CD177 + S100A9 + TAP2 + CD3D + CTLA4 | 0.795 | 0.707 | 0.883 |
| D5/7 | C3AR1 + IL1R2 + CD74 + CD177 + S100A9 + TAP2 + CTLA4 | 0.792 | 0.7 | 0.885 |
| D5/7 | C3AR1 + IL1R2 + CD74 + CD177 + TAP2 | 0.78 | 0.679 | 0.881 |
| D5/7 | C3AR1 + IL1R2 + CD74 + CD177 + TAP2 + CD3D | 0.779 | 0.677 | 0.881 |
| D5/7 | C3AR1 + IL1R2 + CD74 + CD177 + TAP2 + CD3D + CTLA4 | 0.779 | 0.677 | 0.882 |
| D5/7 | C3AR1 + IL1R2 + CD74 + CD177 + TAP2 + CTLA4 | 0.78 | 0.677 | 0.883 |
| D5/7 | C3AR1 + IL1R2 + CD74 + CD3D | 0.779 | 0.679 | 0.879 |
| D5/7 | C3AR1 + IL1R2 + CD74 + CD3D + CTLA4 | 0.775 | 0.668 | 0.882 |
| D5/7 | C3AR1 + IL1R2 + CD74 + CIITA | 0.771 | 0.651 | 0.891 |
| D5/7 | C3AR1 + IL1R2 + CD74 + CIITA + CD3D | 0.773 | 0.655 | 0.891 |
| D5/7 | C3AR1 + IL1R2 + CD74 + CIITA + CD3D + CTLA4 | 0.762 | 0.637 | 0.886 |
| D5/7 | C3AR1 + IL1R2 + CD74 + CIITA + CTLA4 | 0.762 | 0.636 | 0.887 |
| D5/7 | C3AR1 + IL1R2 + CD74 + CIITA + TAP2 | 0.785 | 0.675 | 0.894 |
| D5/7 | C3AR1 + IL1R2 + CD74 + CIITA + TAP2 + CD3D | 0.783 | 0.671 | 0.894 |
| D5/7 | C3AR1 + IL1R2 + CD74 + CIITA + TAP2 + CD3D + CTLA4 | 0.781 | 0.666 | 0.896 |
| D5/7 | C3AR1 + IL1R2 + CD74 + CIITA + TAP2 + CTLA4 | 0.779 | 0.666 | 0.893 |
| D5/7 | C3AR1 + IL1R2 + CD74 + CTLA4 | 0.776 | 0.668 | 0.885 |
| D5/7 | C3AR1 + IL1R2 + CD74 + CX3CR1 | 0.777 | 0.67 | 0.884 |
| D5/7 | C3AR1 + IL1R2 + CD74 + CX3CR1 + CD3D | 0.78 | 0.683 | 0.878 |
| D5/7 | C3AR1 + IL1R2 + CD74 + CX3CR1 + CD3D + CTLA4 | 0.775 | 0.669 | 0.882 |
| D5/7 | C3AR1 + IL1R2 + CD74 + CX3CR1 + CIITA | 0.772 | 0.652 | 0.893 |
| D5/7 | C3AR1 + IL1R2 + CD74 + CX3CR1 + CIITA + CD3D | 0.767 | 0.648 | 0.886 |
| D5/7 | C3AR1 + IL1R2 + CD74 + CX3CR1 + CIITA + CD3D + CTLA4 | 0.761 | 0.636 | 0.886 |
| D5/7 | C3AR1 + IL1R2 + CD74 + CX3CR1 + CIITA + CTLA4 | 0.762 | 0.636 | 0.888 |
| D5/7 | C3AR1 + IL1R2 + CD74 + CX3CR1 + CIITA + TAP2 | 0.79 | 0.681 | 0.898 |
| D5/7 | C3AR1 + IL1R2 + CD74 + CX3CR1 + CIITA + TAP2 + CD3D | 0.788 | 0.68 | 0.895 |
| D5/7 | C3AR1 + IL1R2 + CD74 + CX3CR1 + CIITA + TAP2 + CD3D + CTLA4 | 0.786 | 0.674 | 0.898 |
| D5/7 | C3AR1 + IL1R2 + CD74 + CX3CR1 + CIITA + TAP2 + CTLA4 | 0.785 | 0.673 | 0.898 |
| D5/7 | C3AR1 + IL1R2 + CD74 + CX3CR1 + CTLA4 | 0.776 | 0.667 | 0.885 |
| D5/7 | C3AR1 + IL1R2 + CD74 + CX3CR1 + TAP2 | 0.793 | 0.694 | 0.892 |
| D5/7 | C3AR1 + IL1R2 + CD74 + CX3CR1 + TAP2 + CD3D | 0.792 | 0.697 | 0.887 |
| D5/7 | C3AR1 + IL1R2 + CD74 + CX3CR1 + TAP2 + CD3D + CTLA4 | 0.794 | 0.696 | 0.892 |
| D5/7 | C3AR1 + IL1R2 + CD74 + CX3CR1 + TAP2 + CTLA4 | 0.793 | 0.693 | 0.893 |
| D5/7 | C3AR1 + IL1R2 + CD74 + IFNG | 0.774 | 0.66 | 0.889 |
| D5/7 | C3AR1 + IL1R2 + CD74 + IFNG + CD177 | 0.759 | 0.638 | 0.88 |
| D5/7 | C3AR1 + IL1R2 + CD74 + IFNG + CD177 + CD3D | 0.758 | 0.629 | 0.886 |
| D5/7 | C3AR1 + IL1R2 + CD74 + IFNG + CD177 + CD3D + CTLA4 | 0.756 | 0.627 | 0.886 |
| D5/7 | C3AR1 + IL1R2 + CD74 + IFNG + CD177 + CIITA | 0.752 | 0.624 | 0.879 |
| D5/7 | C3AR1 + IL1R2 + CD74 + IFNG + CD177 + CIITA + CD3D | 0.758 | 0.629 | 0.887 |
| D5/7 | C3AR1 + IL1R2 + CD74 + IFNG + CD177 + CIITA + CD3D + CTLA4 | 0.756 | 0.625 | 0.886 |
| D5/7 | C3AR1 + IL1R2 + CD74 + IFNG + CD177 + CIITA + CTLA4 | 0.753 | 0.621 | 0.885 |
| D5/7 | C3AR1 + IL1R2 + CD74 + IFNG + CD177 + CIITA + TAP2 | 0.783 | 0.669 | 0.896 |
| D5/7 | C3AR1 + IL1R2 + CD74 + IFNG + CD177 + CIITA + TAP2 + CD3D | 0.778 | 0.664 | 0.892 |
| D5/7 | C3AR1 + IL1R2 + CD74 + IFNG + CD177 + CIITA + TAP2 + CD3D + CTLA4 | 0.782 | 0.67 | 0.894 |
| D5/7 | C3AR1 + IL1R2 + CD74 + IFNG + CD177 + CIITA + TAP2 + CTLA4 | 0.779 | 0.664 | 0.894 |
| D5/7 | C3AR1 + IL1R2 + CD74 + IFNG + CD177 + CTLA4 | 0.755 | 0.628 | 0.883 |
| D5/7 | C3AR1 + IL1R2 + CD74 + IFNG + CD177 + CX3CR1 | 0.756 | 0.629 | 0.883 |
| D5/7 | C3AR1 + IL1R2 + CD74 + IFNG + CD177 + CX3CR1 + CD3D | 0.76 | 0.631 | 0.889 |
| D5/7 | C3AR1 + IL1R2 + CD74 + IFNG + CD177 + CX3CR1 + CD3D + CTLA4 | 0.755 | 0.62 | 0.889 |
| D5/7 | C3AR1 + IL1R2 + CD74 + IFNG + CD177 + CX3CR1 + CIITA | 0.757 | 0.63 | 0.884 |
| D5/7 | C3AR1 + IL1R2 + CD74 + IFNG + CD177 + CX3CR1 + CIITA + CD3D | 0.76 | 0.632 | 0.889 |
| D5/7 | C3AR1 + IL1R2 + CD74 + IFNG + CD177 + CX3CR1 + CIITA + CD3D + CTLA4 | 0.754 | 0.621 | 0.887 |
| D5/7 | C3AR1 + IL1R2 + CD74 + IFNG + CD177 + CX3CR1 + CIITA + CTLA4 | 0.758 | 0.628 | 0.889 |
| D5/7 | C3AR1 + IL1R2 + CD74 + IFNG + CD177 + CX3CR1 + CIITA + TAP2 | 0.784 | 0.67 | 0.897 |
| D5/7 | C3AR1 + IL1R2 + CD74 + IFNG + CD177 + CX3CR1 + CIITA + TAP2 + CD3D | 0.783 | 0.67 | 0.895 |
| D5/7 | C3AR1 + IL1R2 + CD74 + IFNG + CD177 + CX3CR1 + CIITA + TAP2 + CD3D + | 0.781 | 0.668 | 0.895 |
| | CTLA4 | | | |
| D5/7 | C3AR1 + IL1R2 + CD74 + IFNG + CD177 + CX3CR1 + CIITA + TAP2 + CTLA4 | 0.78 | 0.665 | 0.894 |
| D5/7 | C3AR1 + IL1R2 + CD74 + IFNG + CD177 + CX3CR1 + CTLA4 | 0.758 | 0.628 | 0.888 |
| D5/7 | C3AR1 + IL1R2 + CD74 + IFNG + CD177 + CX3CR1 + TAP2 | 0.783 | 0.67 | 0.896 |
| D5/7 | C3AR1 + IL1R2 + CD74 + IFNG + CD177 + CX3CR1 + TAP2 + CD3D | 0.783 | 0.67 | 0.895 |
| D5/7 | C3AR1 + IL1R2 + CD74 + IFNG + CD177 + CX3CR1 + TAP2 + CD3D + CTLA4 | 0.781 | 0.668 | 0.895 |
| D5/7 | C3AR1 + IL1R2 + CD74 + IFNG + CD177 + CX3CR1 + TAP2 + CTLA4 | 0.782 | 0.668 | 0.896 |
| D5/7 | C3AR1 + IL1R2 + CD74 + IFNG + CD177 + S100A9 | 0.781 | 0.677 | 0.886 |
| D5/7 | C3AR1 + IL1R2 + CD74 + IFNG + CD177 + S100A9 + CD3D | 0.791 | 0.685 | 0.896 |
| D5/7 | C3AR1 + IL1R2 + CD74 + IFNG + CD177 + S100A9 + CD3D + CTLA4 | 0.791 | 0.686 | 0.896 |
| D5/7 | C3AR1 + IL1R2 + CD74 + IFNG + CD177 + S100A9 + CIITA | 0.776 | 0.665 | 0.887 |
| D5/7 | C3AR1 + IL1R2 + CD74 + IFNG + CD177 + S100A9 + CIITA + CD3D | 0.792 | 0.684 | 0.9 |
| D5/7 | C3AR1 + IL1R2 + CD74 + IFNG + CD177 + S100A9 + CIITA + CD3D + CTLA4 | 0.789 | 0.68 | 0.897 |
| D5/7 | C3AR1 + IL1R2 + CD74 + IFNG + CD177 + S100A9 + CIITA + CTLA4 | 0.769 | 0.649 | 0.89 |
| D5/7 | C3AR1 + IL1R2 + CD74 + IFNG + CD177 + S100A9 + CIITA + TAP2 | 0.798 | 0.701 | 0.895 |
| D5/7 | C3AR1 + IL1R2 + CD74 + IFNG + CD177 + S100A9 + CIITA + TAP2 + CD3D | 0.8 | 0.703 | 0.897 |
| D5/7 | C3AR1 + IL1R2 + CD74 + IFNG + CD177 + S100A9 + CIITA + TAP2 + CD3D + | 0.807 | 0.712 | 0.902 |
| | CTLA4 | | | |
| D5/7 | C3AR1 + IL1R2 + CD74 + IFNG + CD177 + S100A9 + CIITA + TAP2 + CTLA4 | 0.799 | 0.701 | 0.897 |
| D5/7 | C3AR1 + IL1R2 + CD74 + IFNG + CD177 + S100A9 + CTLA4 | 0.779 | 0.67 | 0.887 |
| D5/7 | C3AR1 + IL1R2 + CD74 + IFNG + CD177 + S100A9 + CX3CR1 | 0.8 | 0.702 | 0.898 |
| D5/7 | C3AR1 + IL1R2 + CD74 + IFNG + CD177 + S100A9 + CX3CR1 + CD3D | 0.801 | 0.694 | 0.907 |
| D5/7 | C3AR1 + IL1R2 + CD74 + IFNG + CD177 + S100A9 + CX3CR1 + CD3D + CTLA4 | 0.801 | 0.694 | 0.907 |
| D5/7 | C3AR1 + IL1R2 + CD74 + IFNG + CD177 + S100A9 + CX3CR1 + CIITA | 0.798 | 0.699 | 0.898 |
| D5/7 | C3AR1 + IL1R2 + CD74 + IFNG + CD177 + S100A9 + CX3CR1 + CIITA + CD3D | 0.8 | 0.692 | 0.908 |
| D5/7 | C3AR1 + IL1R2 + CD74 + IFNG + CD177 + S100A9 + CX3CR1 + CIITA + CD3D + | 0.801 | 0.693 | 0.908 |
| | CTLA4 | | | |
| D5/7 | C3AR1 + IL1R2 + CD74 + IFNG + CD177 + S100A9 + CX3CR1 + CIITA + CTLA4 | 0.795 | 0.683 | 0.907 |
| D5/7 | C3AR1 + IL1R2 + CD74 + IFNG + CD177 + S100A9 + CX3CR1 + CIITA + TAP2 | 0.814 | 0.719 | 0.909 |
| D5/7 | C3AR1 + IL1R2 + CD74 + IFNG + CD177 + S100A9 + CX3CR1 + CIITA + TAP2 + | 0.809 | 0.711 | 0.908 |
| | CD3D | | | |
| D5/7 | C3AR1 + IL1R2 + CD74 + IFNG + CD177 + S100A9 + CX3CR1 + CIITA + TAP2 + | 0.81 | 0.714 | 0.907 |
| | CD3D + CTLA4 | | | |
| D5/7 | C3AR1 + IL1R2 + CD74 + IFNG + CD177 + S100A9 + CX3CR1 + CIITA + TAP2 + | 0.813 | 0.715 | 0.911 |
| | CTLA4 | | | |
| D5/7 | C3AR1 + IL1R2 + CD74 + IFNG + CD177 + S100A9 + CX3CR1 + CTLA4 | 0.797 | 0.692 | 0.903 |
| D5/7 | C3AR1 + IL1R2 + CD74 + IFNG + CD177 + S100A9 + CX3CR1 + TAP2 | 0.82 | 0.728 | 0.911 |
| D5/7 | C3AR1 + IL1R2 + CD74 + IFNG + CD177 + S100A9 + CX3CR1 + TAP2 + CD3D | 0.812 | 0.715 | 0.909 |
| D5/7 | C3AR1 + IL1R2 + CD74 + IFNG + CD177 + S100A9 + CX3CR1 + TAP2 + CD3D + | 0.809 | 0.713 | 0.906 |
| | CTLA4 | | | |
| D5/7 | C3AR1 + IL1R2 + CD74 + IFNG + CD177 + S100A9 + CX3CR1 + TAP2 + CTLA4 | 0.819 | 0.725 | 0.912 |
| D5/7 | C3AR1 + IL1R2 + CD74 + IFNG + CD177 + S100A9 + TAP2 | 0.809 | 0.718 | 0.899 |
| D5/7 | C3AR1 + IL1R2 + CD74 + IFNG + CD177 + S100A9 + TAP2 + CD3D | 0.803 | 0.707 | 0.898 |
| D5/7 | C3AR1 + IL1R2 + CD74 + IFNG + CD177 + S100A9 + TAP2 + CD3D + CTLA4 | 0.81 | 0.715 | 0.904 |
| D5/7 | C3AR1 + IL1R2 + CD74 + IFNG + CD177 + S100A9 + TAP2 + CTLA4 | 0.808 | 0.716 | 0.9 |
| D5/7 | C3AR1 + IL1R2 + CD74 + IFNG + CD177 + TAP2 | 0.788 | 0.679 | 0.897 |
| D5/7 | C3AR1 + IL1R2 + CD74 + IFNG + CD177 + TAP2 + CD3D | 0.779 | 0.665 | 0.892 |
| D5/7 | C3AR1 + IL1R2 + CD74 + IFNG + CD177 + TAP2 + CD3D + CTLA4 | 0.782 | 0.67 | 0.894 |
| D5/7 | C3AR1 + IL1R2 + CD74 + IFNG + CD177 + TAP2 + CTLA4 | 0.785 | 0.675 | 0.896 |
| D5/7 | C3AR1 + IL1R2 + CD74 + IFNG + CD3D | 0.771 | 0.652 | 0.889 |
| D5/7 | C3AR1 + IL1R2 + CD74 + IFNG + CD3D + CTLA4 | 0.768 | 0.647 | 0.889 |
| D5/7 | C3AR1 + IL1R2 + CD74 + IFNG + CIITA | 0.765 | 0.642 | 0.889 |
| D5/7 | C3AR1 + IL1R2 + CD74 + IFNG + CIITA + CD3D | 0.768 | 0.645 | 0.89 |
| D5/7 | C3AR1 + IL1R2 + CD74 + IFNG + CIITA + CD3D + CTLA4 | 0.766 | 0.64 | 0.892 |
| D5/7 | C3AR1 + IL1R2 + CD74 + IFNG + CIITA + CTLA4 | 0.768 | 0.64 | 0.895 |
| D5/7 | C3AR1 + IL1R2 + CD74 + IFNG + CIITA + TAP2 | 0.795 | 0.684 | 0.905 |
| D5/7 | C3AR1 + IL1R2 + CD74 + IFNG + CIITA + TAP2 + CD3D | 0.786 | 0.673 | 0.899 |
| D5/7 | C3AR1 + IL1R2 + CD74 + IFNG + CIITA + TAP2 + CD3D + CTLA4 | 0.785 | 0.668 | 0.902 |
| D5/7 | C3AR1 + IL1R2 + CD74 + IFNG + CIITA + TAP2 + CTLA4 | 0.787 | 0.669 | 0.905 |
| D5/7 | C3AR1 + IL1R2 + CD74 + IFNG + CTLA4 | 0.77 | 0.648 | 0.892 |
| D5/7 | C3AR1 + IL1R2 + CD74 + IFNG + CX3CR1 | 0.773 | 0.654 | 0.892 |
| D5/7 | C3AR1 + IL1R2 + CD74 + IFNG + CX3CR1 + CD3D | 0.766 | 0.645 | 0.887 |
| D5/7 | C3AR1 + IL1R2 + CD74 + IFNG + CX3CR1 + CD3D + CTLA4 | 0.767 | 0.644 | 0.89 |
| D5/7 | C3AR1 + IL1R2 + CD74 + IFNG + CX3CR1 + CIITA | 0.771 | 0.646 | 0.896 |
| D5/7 | C3AR1 + IL1R2 + CD74 + IFNG + CX3CR1 + CIITA + CD3D | 0.765 | 0.643 | 0.888 |
| D5/7 | C3AR1 + IL1R2 + CD74 + IFNG + CX3CR1 + CIITA + CD3D + CTLA4 | 0.765 | 0.639 | 0.891 |
| D5/7 | C3AR1 + IL1R2 + CD74 + IFNG + CX3CR1 + CIITA + CTLA4 | 0.767 | 0.64 | 0.894 |
| D5/7 | C3AR1 + IL1R2 + CD74 + IFNG + CX3CR1 + CIITA + TAP2 | 0.796 | 0.686 | 0.905 |
| D5/7 | C3AR1 + IL1R2 + CD74 + IFNG + CX3CR1 + CIITA + TAP2 + CD3D | 0.79 | 0.677 | 0.903 |
| D5/7 | C3AR1 + IL1R2 + CD74 + IFNG + CX3CR1 + CIITA + TAP2 + CD3D + CTLA4 | 0.785 | 0.669 | 0.901 |
| D5/7 | C3AR1 + IL1R2 + CD74 + IFNG + CX3CR1 + CIITA + TAP2 + CTLA4 | 0.789 | 0.673 | 0.904 |
| D5/7 | C3AR1 + IL1R2 + CD74 + IFNG + CX3CR1 + CTLA4 | 0.772 | 0.651 | 0.894 |
| D5/7 | C3AR1 + IL1R2 + CD74 + IFNG + CX3CR1 + TAP2 | 0.8 | 0.695 | 0.904 |
| D5/7 | C3AR1 + IL1R2 + CD74 + IFNG + CX3CR1 + TAP2 + CD3D | 0.792 | 0.684 | 0.9 |
| D5/7 | C3AR1 + IL1R2 + CD74 + IFNG + CX3CR1 + TAP2 + CD3D + CTLA4 | 0.787 | 0.675 | 0.899 |
| D5/7 | C3AR1 + IL1R2 + CD74 + IFNG + CX3CR1 + TAP2 + CTLA4 | 0.788 | 0.676 | 0.899 |
| D5/7 | C3AR1 + IL1R2 + CD74 + IFNG + S100A9 | 0.791 | 0.69 | 0.891 |
| D5/7 | C3AR1 + IL1R2 + CD74 + IFNG + S100A9 + CD3D | 0.799 | 0.701 | 0.898 |
| D5/7 | C3AR1 + IL1R2 + CD74 + IFNG + S100A9 + CD3D + CTLA4 | 0.797 | 0.698 | 0.896 |
| D5/7 | C3AR1 + IL1R2 + CD74 + IFNG + S100A9 + CIITA | 0.787 | 0.679 | 0.894 |
| D5/7 | C3AR1 + IL1R2 + CD74 + IFNG + S100A9 + CIITA + CD3D | 0.789 | 0.682 | 0.897 |
| D5/7 | C3AR1 + IL1R2 + CD74 + IFNG + S100A9 + CIITA + CD3D + CTLA4 | 0.788 | 0.679 | 0.897 |
| D5/7 | C3AR1 + IL1R2 + CD74 + IFNG + S100A9 + CIITA + CTLA4 | 0.78 | 0.663 | 0.897 |
| D5/7 | C3AR1 + IL1R2 + CD74 + IFNG + S100A9 + CIITA + TAP2 | 0.807 | 0.712 | 0.903 |
| D5/7 | C3AR1 + IL1R2 + CD74 + IFNG + S100A9 + CIITA + TAP2 + CD3D | 0.801 | 0.702 | 0.9 |
| D5/7 | C3AR1 + IL1R2 + CD74 + IFNG + S100A9 + CIITA + TAP2 + CD3D + CTLA4 | 0.801 | 0.702 | 0.901 |
| D5/7 | C3AR1 + IL1R2 + CD74 + IFNG + S100A9 + CIITA + TAP2 + CTLA4 | 0.804 | 0.702 | 0.905 |
| D5/7 | C3AR1 + IL1R2 + CD74 + IFNG + S100A9 + CTLA4 | 0.791 | 0.687 | 0.895 |
| D5/7 | C3AR1 + IL1R2 + CD74 + IFNG + S100A9 + CX3CR1 | 0.802 | 0.708 | 0.897 |
| D5/7 | C3AR1 + IL1R2 + CD74 + IFNG + S100A9 + CX3CR1 + CD3D | 0.802 | 0.703 | 0.9 |
| D5/7 | C3AR1 + IL1R2 + CD74 + IFNG + S100A9 + CX3CR1 + CD3D + CTLA4 | 0.798 | 0.696 | 0.901 |
| D5/7 | C3AR1 + IL1R2 + CD74 + IFNG + S100A9 + CX3CR1 + CIITA | 0.796 | 0.692 | 0.9 |
| D5/7 | C3AR1 + IL1R2 + CD74 + IFNG + S100A9 + CX3CR1 + CIITA + CD3D | 0.8 | 0.693 | 0.906 |
| D5/7 | C3AR1 + IL1R2 + CD74 + IFNG + S100A9 + CX3CR1 + CIITA + CD3D + CTLA4 | 0.801 | 0.693 | 0.91 |
| D5/7 | C3AR1 + IL1R2 + CD74 + IFNG + S100A9 + CX3CR1 + CIITA + CTLA4 | 0.794 | 0.684 | 0.905 |
| D5/7 | C3AR1 + IL1R2 + CD74 + IFNG + S100A9 + CX3CR1 + CIITA + TAP2 | 0.808 | 0.713 | 0.903 |
| D5/7 | C3AR1 + IL1R2 + CD74 + IFNG + S100A9 + CX3CR1 + CIITA + TAP2 + CD3D | 0.799 | 0.699 | 0.899 |
| D5/7 | C3AR1 + IL1R2 + CD74 + IFNG + S100A9 + CX3CR1 + CIITA + TAP2 + CD3D + | 0.796 | 0.695 | 0.898 |
| | CTLA4 | | | |
| D5/7 | C3AR1 + IL1R2 + CD74 + IFNG + S100A9 + CX3CR1 + CIITA + TAP2 + CTLA4 | 0.801 | 0.699 | 0.903 |
| D5/7 | C3AR1 + IL1R2 + CD74 + IFNG + S100A9 + CX3CR1 + CTLA4 | 0.798 | 0.695 | 0.901 |
| D5/7 | C3AR1 + IL1R2 + CD74 + IFNG + S100A9 + CX3CR1 + TAP2 | 0.812 | 0.723 | 0.901 |
| D5/7 | C3AR1 + IL1R2 + CD74 + IFNG + S100A9 + CX3CR1 + TAP2 + CD3D | 0.808 | 0.716 | 0.9 |
| D5/7 | C3AR1 + IL1R2 + CD74 + IFNG + S100A9 + CX3CR1 + TAP2 + CD3D + CTLA4 | 0.809 | 0.717 | 0.901 |
| D5/7 | C3AR1 + IL1R2 + CD74 + IFNG + S100A9 + CX3CR1 + TAP2 + CTLA4 | 0.807 | 0.712 | 0.901 |
| D5/7 | C3AR1 + IL1R2 + CD74 + IFNG + S100A9 + TAP2 | 0.815 | 0.726 | 0.903 |
| D5/7 | C3AR1 + IL1R2 + CD74 + IFNG + S100A9 + TAP2 + CD3D | 0.808 | 0.717 | 0.9 |
| D5/7 | C3AR1 + IL1R2 + CD74 + IFNG + S100A9 + TAP2 + CD3D + CTLA4 | 0.809 | 0.718 | 0.9 |
| D5/7 | C3AR1 + IL1R2 + CD74 + IFNG + S100A9 + TAP2 + CTLA4 | 0.812 | 0.722 | 0.903 |
| D5/7 | C3AR1 + IL1R2 + CD74 + IFNG + TAP2 | 0.8 | 0.696 | 0.905 |
| D5/7 | C3AR1 + IL1R2 + CD74 + IFNG + TAP2 + CD3D | 0.792 | 0.685 | 0.9 |
| D5/7 | C3AR1 + IL1R2 + CD74 + IFNG + TAP2 + CD3D + CTLA4 | 0.788 | 0.676 | 0.899 |
| D5/7 | C3AR1 + IL1R2 + CD74 + IFNG + TAP2 + CTLA4 | 0.79 | 0.679 | 0.9 |
| D5/7 | C3AR1 + IL1R2 + CD74 + S100A9 | 0.788 | 0.69 | 0.885 |
| D5/7 | C3AR1 + IL1R2 + CD74 + S100A9 + CD3D | 0.793 | 0.705 | 0.882 |
| D5/7 | C3AR1 + IL1R2 + CD74 + S100A9 + CD3D + CTLA4 | 0.788 | 0.694 | 0.881 |
| D5/7 | C3AR1 + IL1R2 + CD74 + S100A9 + CIITA | 0.783 | 0.672 | 0.894 |
| D5/7 | C3AR1 + IL1R2 + CD74 + S100A9 + CIITA + CD3D | 0.784 | 0.676 | 0.892 |
| D5/7 | C3AR1 + IL1R2 + CD74 + S100A9 + CIITA + CD3D + CTLA4 | 0.775 | 0.662 | 0.889 |
| D5/7 | C3AR1 + IL1R2 + CD74 + S100A9 + CIITA + CTLA4 | 0.77 | 0.648 | 0.892 |
| D5/7 | C3AR1 + IL1R2 + CD74 + S100A9 + CIITA + TAP2 | 0.796 | 0.696 | 0.895 |
| D5/7 | C3AR1 + IL1R2 + CD74 + S100A9 + CIITA + TAP2 + CD3D | 0.797 | 0.698 | 0.895 |
| D5/7 | C3AR1 + IL1R2 + CD74 + S100A9 + CIITA + TAP2 + CD3D + CTLA4 | 0.793 | 0.688 | 0.898 |
| D5/7 | C3AR1 + IL1R2 + CD74 + S100A9 + CIITA + TAP2 + CTLA4 | 0.793 | 0.687 | 0.898 |
| D5/7 | C3AR1 + IL1R2 + CD74 + S100A9 + CTLA4 | 0.789 | 0.689 | 0.888 |
| D5/7 | C3AR1 + IL1R2 + CD74 + S100A9 + CX3CR1 | 0.793 | 0.698 | 0.887 |
| D5/7 | C3AR1 + IL1R2 + CD74 + S100A9 + CX3CR1 + CD3D | 0.793 | 0.704 | 0.881 |
| D5/7 | C3AR1 + IL1R2 + CD74 + S100A9 + CX3CR1 + CD3D + CTLA4 | 0.789 | 0.695 | 0.882 |
| D5/7 | C3AR1 + IL1R2 + CD74 + S100A9 + CX3CR1 + CIITA | 0.788 | 0.677 | 0.898 |
| D5/7 | C3AR1 + IL1R2 + CD74 + S100A9 + CX3CR1 + CIITA + CD3D | 0.783 | 0.675 | 0.89 |
| D5/7 | C3AR1 + IL1R2 + CD74 + S100A9 + CX3CR1 + CIITA + CD3D + CTLA4 | 0.775 | 0.662 | 0.889 |
| D5/7 | C3AR1 + IL1R2 + CD74 + S100A9 + CX3CR1 + CIITA + CTLA4 | 0.772 | 0.652 | 0.893 |
| D5/7 | C3AR1 + IL1R2 + CD74 + S100A9 + CX3CR1 + CIITA + TAP2 | 0.798 | 0.698 | 0.897 |
| D5/7 | C3AR1 + IL1R2 + CD74 + S100A9 + CX3CR1 + CIITA + TAP2 + CD3D | 0.796 | 0.698 | 0.894 |
| D5/7 | C3AR1 + IL1R2 + CD74 + S100A9 + CX3CR1 + CIITA + TAP2 + CD3D + CTLA4 | 0.793 | 0.69 | 0.895 |
| D5/7 | C3AR1 + IL1R2 + CD74 + S100A9 + CX3CR1 + CIITA + TAP2 + CTLA4 | 0.793 | 0.688 | 0.897 |
| D5/7 | C3AR1 + IL1R2 + CD74 + S100A9 + CX3CR1 + CTLA4 | 0.787 | 0.687 | 0.886 |
| D5/7 | C3AR1 + IL1R2 + CD74 + S100A9 + CX3CR1 + TAP2 | 0.799 | 0.712 | 0.887 |
| D5/7 | C3AR1 + IL1R2 + CD74 + S100A9 + CX3CR1 + TAP2 + CD3D | 0.797 | 0.71 | 0.883 |
| D5/7 | C3AR1 + IL1R2 + CD74 + S100A9 + CX3CR1 + TAP2 + CD3D + CTLA4 | 0.796 | 0.708 | 0.885 |
| D5/7 | C3AR1 + IL1R2 + CD74 + S100A9 + CX3CR1 + TAP2 + CTLA4 | 0.796 | 0.706 | 0.887 |
| D5/7 | C3AR1 + IL1R2 + CD74 + S100A9 + TAP2 | 0.8 | 0.713 | 0.886 |
| D5/7 | C3AR1 + IL1R2 + CD74 + S100A9 + TAP2 + CD3D | 0.8 | 0.715 | 0.885 |
| D5/7 | C3AR1 + IL1R2 + CD74 + S100A9 + TAP2 + CD3D + CTLA4 | 0.795 | 0.705 | 0.884 |
| D5/7 | C3AR1 + IL1R2 + CD74 + S100A9 + TAP2 + CTLA4 | 0.794 | 0.705 | 0.884 |
| D5/7 | C3AR1 + IL1R2 + CD74 + TAP2 | 0.795 | 0.7 | 0.891 |
| D5/7 | C3AR1 + IL1R2 + CD74 + TAP2 + CD3D | 0.795 | 0.7 | 0.89 |
| D5/7 | C3AR1 + IL1R2 + CD74 + TAP2 + CD3D + CTLA4 | 0.793 | 0.691 | 0.895 |
| D5/7 | C3AR1 + IL1R2 + CD74 + TAP2 + CTLA4 | 0.792 | 0.692 | 0.891 |
| D5/7 | C3AR1 + IL1R2 + CIITA | 0.799 | 0.716 | 0.882 |
| D5/7 | C3AR1 + IL1R2 + CIITA + CD3D | 0.791 | 0.701 | 0.881 |
| D5/7 | C3AR1 + IL1R2 + CIITA + CD3D + CTLA4 | 0.772 | 0.668 | 0.876 |
| D5/7 | C3AR1 + IL1R2 + CIITA + CTLA4 | 0.77 | 0.665 | 0.876 |
| D5/7 | C3AR1 + IL1R2 + CIITA + TAP2 | 0.802 | 0.714 | 0.89 |
| D5/7 | C3AR1 + IL1R2 + CIITA + TAP2 + CD3D | 0.799 | 0.711 | 0.888 |
| D5/7 | C3AR1 + IL1R2 + CIITA + TAP2 + CD3D + CTLA4 | 0.797 | 0.7 | 0.894 |
| D5/7 | C3AR1 + IL1R2 + CIITA + TAP2 + CTLA4 | 0.796 | 0.703 | 0.889 |
| D5/7 | C3AR1 + IL1R2 + CTLA4 | 0.774 | 0.663 | 0.886 |
| D5/7 | C3AR1 + IL1R2 + CX3CR1 | 0.805 | 0.716 | 0.894 |
| D5/7 | C3AR1 + IL1R2 + CX3CR1 + CD3D | 0.787 | 0.683 | 0.891 |
| D5/7 | C3AR1 + IL1R2 + CX3CR1 + CD3D + CTLA4 | 0.774 | 0.66 | 0.889 |
| D5/7 | C3AR1 + IL1R2 + CX3CR1 + CIITA | 0.799 | 0.715 | 0.883 |
| D5/7 | C3AR1 + IL1R2 + CX3CR1 + CIITA + CD3D | 0.792 | 0.702 | 0.881 |
| D5/7 | C3AR1 + IL1R2 + CX3CR1 + CIITA + CD3D + CTLA4 | 0.773 | 0.665 | 0.88 |
| D5/7 | C3AR1 + IL1R2 + CX3CR1 + CIITA + CTLA4 | 0.772 | 0.665 | 0.88 |
| D5/7 | C3AR1 + IL1R2 + CX3CR1 + CIITA + TAP2 | 0.8 | 0.709 | 0.89 |
| D5/7 | C3AR1 + IL1R2 + CX3CR1 + CIITA + TAP2 + CD3D | 0.799 | 0.709 | 0.889 |
| D5/7 | C3AR1 + IL1R2 + CX3CR1 + CIITA + TAP2 + CD3D + CTLA4 | 0.799 | 0.705 | 0.893 |
| D5/ | C3AR1 + IL1R2 + CX3CR1 + CIITA + TAP2 + CTLA4 | 0.798 | 0.703 | 0.893 |
| D5/7 | C3AR1 + IL1R2 + CX3CR1 + CTLA4 | 0.774 | 0.66 | 0.887 |
| D5/7 | C3AR1 + IL1R2 + CX3CR1 + TAP2 | 0.799 | 0.709 | 0.89 |
| D5/7 | C3AR1 + IL1R2 + CX3CR1 + TAP2 + CD3D | 0.799 | 0.71 | 0.889 |
| D5/7 | C3AR1 + IL1R2 + CX3CR1 + TAP2 + CD3D + CTLA4 | 0.8 | 0.706 | 0.894 |
| D5/7 | C3AR1 + IL1R2 + CX3CR1 + TAP2 + CTLA4 | 0.8 | 0.706 | 0.894 |
| D5/7 | C3AR1 + IL1R2 + IFNG | 0.757 | 0.635 | 0.88 |
| D5/7 | C3AR1 + IL1R2 + IFNG + CD177 + CIITA | 0.753 | 0.648 | 0.858 |
| D5/7 | C3AR1 + IL1R2 + IFNG + CD177 + CIITA + CD3D | 0.751 | 0.632 | 0.869 |
| D5/7 | C3AR1 + IL1R2 + IFNG + CD177 + CIITA + CD3D + CTLA4 | 0.751 | 0.636 | 0.867 |
| D5/7 | C3AR1 + IL1R2 + IFNG + CD177 + CIITA + TAP2 | 0.789 | 0.687 | 0.89 |
| D5/7 | C3AR1 + IL1R2 + IFNG + CD177 + CIITA + TAP2 + CD3D | 0.78 | 0.675 | 0.886 |
| D5/7 | C3AR1 + IL1R2 + IFNG + CD177 + CIITA + TAP2 + CD3D + CTLA4 | 0.781 | 0.678 | 0.885 |
| D5/7 | C3AR1 + IL1R2 + IFNG + CD177 + CIITA + TAP2 + CTLA4 | 0.788 | 0.686 | 0.89 |
| D5/7 | C3AR1 + IL1R2 + IFNG + CD177 + CX3CR1 + CIITA | 0.765 | 0.663 | 0.867 |
| D5/7 | C3AR1 + IL1R2 + IFNG + CD177 + CX3CR1 + CIITA + CD3D | 0.765 | 0.647 | 0.883 |
| D5/7 | C3AR1 + IL1R2 + IFNG + CD177 + CX3CR1 + CIITA + CD3D + CTLA4 | 0.764 | 0.649 | 0.88 |
| D5/7 | C3AR1 + IL1R2 + IFNG + CD177 + CX3CR1 + CIITA + CTLA4 | 0.757 | 0.642 | 0.872 |
| D5/7 | C3AR1 + IL1R2 + IFNG + CD177 + CX3CR1 + CIITA + TAP2 | 0.789 | 0.689 | 0.889 |
| D5/7 | C3AR1 + IL1R2 + IFNG + CD177 + CX3CR1 + CIITA + TAP2 + CD3D | 0.783 | 0.679 | 0.886 |
| D5/7 | C3AR1 + IL1R2 + IFNG + CD177 + CX3CR1 + CIITA + TAP2 + CD3D + CTLA4 | 0.783 | 0.68 | 0.885 |
| D5/7 | C3AR1 + IL1R2 + IFNG + CD177 + CX3CR1 + CIITA + TAP2 + CTLA4 | 0.789 | 0.686 | 0.892 |
| D5/7 | C3AR1 + IL1R2 + IFNG + CD177 + CX3CR1 + TAP2 | 0.784 | 0.681 | 0.887 |
| D5/7 | C3AR1 + IL1R2 + IFNG + CD177 + CX3CR1 + TAP2 + CD3D | 0.769 | 0.657 | 0.882 |
| D5/7 | C3AR1 + IL1R2 + IFNG + CD177 + CX3CR1 + TAP2 + CD3D + CTLA4 | 0.772 | 0.661 | 0.882 |
| D5/7 | C3AR1 + IL1R2 + IFNG + CD177 + CX3CR1 + TAP2 + CTLA4 | 0.777 | 0.668 | 0.885 |
| D5/7 | C3AR1 + IL1R2 + IFNG + CD177 + S100A9 | 0.757 | 0.648 | 0.866 |
| D5/7 | C3AR1 + IL1R2 + IFNG + CD177 + S100A9 + CD3D | 0.77 | 0.652 | 0.888 |
| D5/7 | C3AR1 + IL1R2 + IFNG + CD177 + S100A9 + CD3D + CTLA4 | 0.77 | 0.651 | 0.889 |
| D5/7 | C3AR1 + IL1R2 + IFNG + CD177 + S100A9 + CIITA | 0.772 | 0.672 | 0.872 |
| D5/7 | C3AR1 + IL1R2 + IFNG + CD177 + S100A9 + CIITA + CD3D | 0.771 | 0.659 | 0.883 |
| D5/7 | C3AR1 + IL1R2 + IFNG + CD177 + S100A9 + CIITA + CD3D + CTLA4 | 0.769 | 0.657 | 0.882 |
| D5/7 | C3AR1 + IL1R2 + IFNG + CD177 + S100A9 + CIITA + CTLA4 | 0.758 | 0.644 | 0.873 |
| D5/7 | C3AR1 + IL1R2 + IFNG + CD177 + S100A9 + CIITA + TAP2 | 0.799 | 0.708 | 0.891 |
| D5/7 | C3AR1 + IL1R2 + IFNG + CD177 + S100A9 + CIITA + TAP2 + CD3D | 0.798 | 0.7 | 0.896 |
| D5/7 | C3AR1 + IL1R2 + IFNG + CD177 + S100A9 + CIITA + TAP2 + CD3D + CTLA4 | 0.8 | 0.705 | 0.895 |
| D5/7 | C3AR1 + IL1R2 + IFNG + CD177 + S100A9 + CIITA + TAP2 + CTLA4 | 0.798 | 0.705 | 0.891 |
| D5/7 | C3AR1 + IL1R2 + IFNG + CD177 + S100A9 + CTLA4 | 0.752 | 0.633 | 0.872 |
| D5/7 | C3AR1 + IL1R2 + IFNG + CD177 + S100A9 + CX3CR1 | 0.791 | 0.683 | 0.899 |
| D5/7 | C3AR1 + IL1R2 + IFNG + CD177 + S100A9 + CX3CR1 + CD3D | 0.778 | 0.657 | 0.899 |
| D5/7 | C3AR1 + IL1R2 + IFNG + CD177 + S100A9 + CX3CR1 + CD3D + CTLA4 | 0.78 | 0.657 | 0.904 |
| D5/7 | C3AR1 + IL1R2 + IFNG + CD177 + S100A9 + CX3CR1 + CIITA | 0.796 | 0.698 | 0.893 |
| D5/7 | C3AR1 + IL1R2 + IFNG + CD177 + S100A9 + CX3CR1 + CIITA + CD3D | 0.79 | 0.679 | 0.901 |
| D5/7 | C3AR1 + IL1R2 + IFNG + CD177 + S100A9 + CX3CR1 + CIITA + CD3D + CTLA4 | 0.789 | 0.676 | 0.902 |
| D5/7 | C3AR1 + IL1R2 + IFNG + CD177 + S100A9 + CX3CR1 + CIITA + CTLA4 | 0.792 | 0.683 | 0.902 |
| D5/7 | C3AR1 + IL1R2 + IFNG + CD177 + S100A9 + CX3CR1 + CIITA + TAP2 | 0.81 | 0.717 | 0.904 |
| D5/7 | C3AR1 + IL1R2 + IFNG + CD177 + S100A9 + CX3CR1 + CIITA + TAP2 + CD3D | 0.805 | 0.706 | 0.905 |
| D5/7 | C3AR1 + IL1R2 + IFNG + CD177 + S100A9 + CX3CR1 + CIITA + TAP2 + CD3D + | 0.807 | 0.708 | 0.905 |
| | CTLA4 | | | |
| D5/7 | C3AR1 + IL1R2 + IFNG + CD177 + S100A9 + CX3CR1 + CIITA + TAP2 + CTLA4 | 0.809 | 0.715 | 0.902 |
| D5/7 | C3AR1 + IL1R2 + IFNG + CD177 + S100A9 + CX3CR1 + CTLA4 | 0.78 | 0.662 | 0.899 |
| D5/7 | C3AR1 + IL1R2 + IFNG + CD177 + S100A9 + CX3CR1 + TAP2 | 0.81 | 0.716 | 0.904 |
| D5/7 | C3AR1 + IL1R2 + IFNG + CD177 + S100A9 + CX3CR1 + TAP2 + CD3D | 0.804 | 0.701 | 0.908 |
| D5/7 | C3AR1 + IL1R2 + IFNG + CD177 + S100A9 + CX3CR1 + TAP2 + CD3D + CTLA4 | 0.803 | 0.703 | 0.902 |
| D5/7 | C3AR1 + IL1R2 + IFNG + CD177 + S100A9 + CX3CR1 + TAP2 + CTLA4 | 0.806 | 0.711 | 0.901 |
| D5/7 | C3AR1 + IL1R2 + IFNG + CD177 + S100A9 + TAP2 | 0.802 | 0.711 | 0.893 |
| D5/7 | C3AR1 + IL1R2 + IFNG + CD177 + S100A9 + TAP2 + CD3D | 0.799 | 0.702 | 0.897 |
| D5/7 | C3AR1 + IL1R2 + IFNG + CD177 + S100A9 + TAP2 + CD3D + CTLA4 | 0.801 | 0.705 | 0.897 |
| D5/7 | C3AR1 + IL1R2 + IFNG + CD177 + S100A9 + TAP2 + CTLA4 | 0.799 | 0.708 | 0.891 |
| D5/7 | C3AR1 + IL1R2 + IFNG + CD177 + TAP2 | 0.776 | 0.67 | 0.882 |
| D5/7 | C3AR1 + IL1R2 + IFNG + CD177 + TAP2 + CD3D | 0.772 | 0.663 | 0.882 |
| D5/7 | C3AR1 + IL1R2 + IFNG + CD177 + TAP2 + CD3D + CTLA4 | 0.773 | 0.665 | 0.882 |
| D5/7 | C3AR1 + IL1R2 + IFNG + CD177 + TAP2 + CTLA4 | 0.774 | 0.667 | 0.881 |
| D5/7 | C3AR1 + IL1R2 + IFNG + CD3D | 0.754 | 0.62 | 0.887 |
| D5/7 | C3AR1 + IL1R2 + IFNG + CD3D + CTLA4 | 0.749 | 0.617 | 0.881 |
| D5/7 | C3AR1 + IL1R2 + IFNG + CIITA | 0.786 | 0.692 | 0.879 |
| D5/7 | C3AR1 + IL1R2 + IFNG + CIITA + CD3D | 0.78 | 0.672 | 0.888 |
| D5/7 | C3AR1 + IL1R2 + IFNG + CIITA + CD3D + CTLA4 | 0.769 | 0.658 | 0.881 |
| D5/7 | C3AR1 + IL1R2 + IFNG + CIITA + CTLA4 | 0.766 | 0.656 | 0.876 |
| D5/7 | C3AR1 + IL1R2 + IFNG + CIITA + TAP2 | 0.805 | 0.713 | 0.898 |
| D5/7 | C3AR1 + IL1R2 + IFNG + CIITA + TAP2 + CD3D | 0.798 | 0.701 | 0.895 |
| D5/7 | C3AR1 + IL1R2 + IFNG + CIITA + TAP2 + CD3D + CTLA4 | 0.795 | 0.694 | 0.897 |
| D5/7 | C3AR1 + IL1R2 + IFNG + CIITA + TAP2 + CTLA4 | 0.798 | 0.697 | 0.898 |
| D5/7 | C3AR1 + IL1R2 + IFNG + CTLA4 | 0.753 | 0.626 | 0.879 |
| D5/7 | C3AR1 + IL1R2 + IFNG + CX3CR1 | 0.779 | 0.663 | 0.895 |
| D5/7 | C3AR1 + IL1R2 + IFNG + CX3CR1 + CD3D | 0.765 | 0.633 | 0.897 |
| D5/7 | C3AR1 + IL1R2 + IFNG + CX3CR1 + CD3D + CTLA4 | 0.768 | 0.64 | 0.897 |
| D5/7 | C3AR1 + IL1R2 + IFNG + CX3CR1 + CIITA | 0.796 | 0.704 | 0.887 |
| D5/7 | C3AR1 + IL1R2 + IFNG + CX3CR1 + CIITA + CD3D | 0.784 | 0.677 | 0.89 |
| D5/7 | C3AR1 + IL1R2 + IFNG + CX3CR1 + CIITA + CD3D + CTLA4 | 0.778 | 0.67 | 0.886 |
| D5/7 | C3AR1 + IL1R2 + IFNG + CX3CR1 + CIITA + CTLA4 | 0.775 | 0.667 | 0.884 |
| D5/7 | C3AR1 + IL1R2 + IFNG + CX3CR1 + CIITA + TAP2 | 0.804 | 0.709 | 0.899 |
| D5/7 | C3AR1 + IL1R2 + IFNG + CX3CR1 + CIITA + TAP2 + CD3D | 0.8 | 0.703 | 0.896 |
| D5/7 | C3AR1 + IL1R2 + IFNG + CX3CR1 + CIITA + TAP2 + CD3D + CTLA4 | 0.795 | 0.695 | 0.895 |
| D5/7 | C3AR1 + IL1R2 + IFNG + CX3CR1 + CIITA + TAP2 + CTLA4 | 0.796 | 0.696 | 0.896 |
| D5/7 | C3AR1 + IL1R2 + IFNG + CX3CR1 + CTLA4 | 0.77 | 0.643 | 0.897 |
| D5/7 | C3AR1 + IL1R2 + IFNG + CX3CR1 + TAP2 | 0.802 | 0.703 | 0.901 |
| D5/7 | C3AR1 + IL1R2 + IFNG + CX3CR1 + TAP2 + CD3D | 0.795 | 0.694 | 0.897 |
| D5/7 | C3AR1 + IL1R2 + IFNG + CX3CR1 + TAP2 + CD3D + CTLA4 | 0.791 | 0.685 | 0.896 |
| D5/7 | C3AR1 + IL1R2 + IFNG + CX3CR1 + TAP2 + CTLA4 | 0.792 | 0.687 | 0.897 |
| D5/7 | C3AR1 + IL1R2 + IFNG + S100A9 | 0.784 | 0.684 | 0.883 |
| D5/7 | C3AR1 + IL1R2 + IFNG + S100A9 + CD3D | 0.783 | 0.674 | 0.892 |
| D5/7 | C3AR1 + IL1R2 + IFNG + S100A9 + CD3D + CTLA4 | 0.783 | 0.673 | 0.892 |
| D5/7 | C3AR1 + IL1R2 + IFNG + S100A9 + CIITA | 0.786 | 0.689 | 0.882 |
| D5/7 | C3AR1 + IL1R2 + IFNG + S100A9 + CIITA + CD3D | 0.782 | 0.677 | 0.886 |
| D5/7 | C3AR1 + IL1R2 + IFNG + S100A9 + CIITA + CD3D + CTLA4 | 0.783 | 0.675 | 0.891 |
| D5/7 | C3AR1 + IL1R2 + IFNG + S100A9 + CIITA + CTLA4 | 0.771 | 0.66 | 0.882 |
| D5/7 | C3AR1 + IL1R2 + IFNG + S100A9 + CIITA + TAP2 | 0.807 | 0.716 | 0.899 |
| D5/7 | C3AR1 + IL1R2 + IFNG + S100A9 + CIITA + TAP2 + CD3D | 0.807 | 0.712 | 0.902 |
| D5/7 | C3AR1 + IL1R2 + IFNG + S100A9 + CIITA + TAP2 + CD3D + CTLA4 | 0.806 | 0.712 | 0.901 |
| D5/7 | C3AR1 + IL1R2 + IFNG + S100A9 + CIITA + TAP2 + CTLA4 | 0.807 | 0.713 | 0.901 |
| D5/7 | C3AR1 + IL1R2 + IFNG + S100A9 + CTLA4 | 0.77 | 0.655 | 0.885 |
| D5/7 | C3AR1 + IL1R2 + IFNG + S100A9 + CX3CR1 | 0.8 | 0.702 | 0.899 |
| D5/7 | C3AR1 + IL1R2 + IFNG + S100A9 + CX3CR1 + CD3D | 0.789 | 0.681 | 0.898 |
| D5/7 | C3AR1 + IL1R2 + IFNG + S100A9 + CX3CR1 + CD3D + CTLA4 | 0.789 | 0.68 | 0.899 |
| D5/7 | C3AR1 + IL1R2 + IFNG + S100A9 + CX3CR1 + CIITA | 0.804 | 0.71 | 0.899 |
| D5/7 | C3AR1 + IL1R2 + IFNG + S100A9 + CX3CR1 + CIITA + CD3D | 0.794 | 0.691 | 0.897 |
| D5/7 | C3AR1 + IL1R2 + IFNG + S100A9 + CX3CR1 + CIITA + CD3D + CTLA4 | 0.792 | 0.683 | 0.9 |
| D5/7 | C3AR1 + IL1R2 + IFNG + S100A9 + CX3CR1 + CIITA + CTLA4 | 0.79 | 0.681 | 0.898 |
| D5/7 | C3AR1 + IL1R2 + IFNG + S100A9 + CX3CR1 + CIITA + TAP2 | 0.812 | 0.721 | 0.903 |
| D5/7 | C3AR1 + IL1R2 + IFNG + S100A9 + CX3CR1 + CIITA + TAP2 + CD3D | 0.808 | 0.712 | 0.904 |
| D5/7 | C3AR1 + IL1R2 + IFNG + S100A9 + CX3CR1 + CIITA + TAP2 + CD3D + CTLA4 | 0.806 | 0.709 | 0.903 |
| D5/7 | C3AR1 + IL1R2 + IFNG + S100A9 + CX3CR1 + CIITA + TAP2 + CTLA4 | 0.809 | 0.715 | 0.903 |
| D5/7 | C3AR1 + IL1R2 + IFNG + S100A9 + CX3CR1 + CTLA4 | 0.787 | 0.675 | 0.9 |
| D5/7 | C3AR1 + IL1R2 + IFNG + S100A9 + CX3CR1 + TAP2 | 0.813 | 0.724 | 0.902 |
| D5/7 | C3AR1 + IL1R2 + IFNG + S100A9 + CX3CR1 + TAP2 + CD3D | 0.81 | 0.717 | 0.904 |
| D5/7 | C3AR1 + IL1R2 + IFNG + S100A9 + CX3CR1 + TAP2 + CD3D + CTLA4 | 0.807 | 0.712 | 0.902 |
| D5/7 | C3AR1 + IL1R2 + IFNG + S100A9 + CX3CR1 + TAP2 + CTLA4 | 0.809 | 0.716 | 0.902 |
| D5/7 | C3AR1 + IL1R2 + IFNG + S100A9 + TAP2 | 0.813 | 0.727 | 0.899 |
| D5/7 | C3AR1 + IL1R2 + IFNG + S100A9 + TAP2 + CD3D | 0.807 | 0.713 | 0.901 |
| D5/7 | C3AR1 + IL1R2 + IFNG + S100A9 + TAP2 + CD3D + CTLA4 | 0.806 | 0.712 | 0.899 |
| D5/7 | C3AR1 + IL1R2 + IFNG + S100A9 + TAP2 + CTLA4 | 0.808 | 0.718 | 0.899 |
| D5/7 | C3AR1 + IL1R2 + IFNG + TAP2 | 0.794 | 0.695 | 0.894 |
| D5/7 | C3AR1 + IL1R2 + IFNG + TAP2 + CD3D | 0.791 | 0.688 | 0.893 |
| D5/7 | C3AR1 + IL1R2 + IFNG + TAP2 + CD3D + CTLA4 | 0.797 | 0.692 | 0.901 |
| D5/7 | C3AR1 + IL1R2 + IFNG + TAP2 + CTLA4 | 0.795 | 0.69 | 0.901 |
| D5/7 | C3AR1 + IL1R2 + S100A9 | 0.798 | 0.711 | 0.885 |
| D5/7 | C3AR1 + IL1R2 + S100A9 + CD3D | 0.788 | 0.694 | 0.881 |
| D5/7 | C3AR1 + IL1R2 + S100A9 + CD3D + CTLA4 | 0.775 | 0.67 | 0.88 |
| D5/7 | C3AR1 + IL1R2 + S100A9 + CIITA | 0.8 | 0.713 | 0.887 |
| D5/7 | C3AR1 + IL1R2 + S100A9 + CIITA + CD3D | 0.788 | 0.687 | 0.888 |
| D5/7 | C3AR1 + IL1R2 + S100A9 + CIITA + CD3D + CTLA4 | 0.775 | 0.665 | 0.886 |
| D5/7 | C3AR1 + IL1R2 + S100A9 + CIITA + CTLA4 | 0.774 | 0.661 | 0.888 |
| D5/7 | C3AR1 + IL1R2 + S100A9 + CIITA + TAP2 | 0.803 | 0.716 | 0.889 |
| D5/7 | C3AR1 + IL1R2 + S100A9 + CIITA + TAP2 + CD3D | 0.8 | 0.71 | 0.891 |
| D5/7 | C3AR1 + IL1R2 + S100A9 + CIITA + TAP2 + CD3D + CTLA4 | 0.797 | 0.703 | 0.891 |
| D5/7 | C3AR1 + IL1R2 + S100A9 + CIITA + TAP2 + CTLA4 | 0.799 | 0.706 | 0.892 |
| D5/7 | C3AR1 + IL1R2 + S100A9 + CTLA4 | 0.776 | 0.668 | 0.884 |
| D5/7 | C3AR1 + IL1R2 + S100A9 + CX3CR1 | 0.805 | 0.718 | 0.892 |
| D5/7 | C3AR1 + IL1R2 + S100A9 + CX3CR1 + CD3D | 0.794 | 0.701 | 0.887 |
| D5/7 | C3AR1 + IL1R2 + S100A9 + CX3CR1 + CD3D + CTLA4 | 0.779 | 0.673 | 0.886 |
| D5/7 | C3AR1 + IL1R2 + S100A9 + CX3CR1 + CIITA | 0.799 | 0.708 | 0.891 |
| D5/7 | C3AR1 + IL1R2 + S100A9 + CX3CR1 + CIITA + CD3D | 0.787 | 0.686 | 0.888 |
| D5/7 | C3AR1 + IL1R2 + S100A9 + CX3CR1 + CIITA + CD3D + CTLA4 | 0.78 | 0.669 | 0.892 |
| D5/7 | C3AR1 + IL1R2 + S100A9 + CX3CR1 + CIITA + CTLA4 | 0.783 | 0.671 | 0.894 |
| D5/7 | C3AR1 + IL1R2 + S100A9 + CX3CR1 + CIITA + TAP2 | 0.806 | 0.721 | 0.892 |
| D5/7 | C3AR1 + IL1R2 + S100A9 + CX3CR1 + CIITA + TAP2 + CD3D | 0.802 | 0.714 | 0.889 |
| D5/7 | C3AR1 + IL1R2 + S100A9 + CX3CR1 + CIITA + TAP2 + CD3D + CTLA4 | 0.801 | 0.708 | 0.894 |
| D5/7 | C3AR1 + IL1R2 + S100A9 + CX3CR1 + CIITA + TAP2 + CTLA4 | 0.801 | 0.709 | 0.893 |
| D5/7 | C3AR1 + IL1R2 + S100A9 + CX3CR1 + CTLA4 | 0.784 | 0.676 | 0.891 |
| D5/7 | C3AR1 + IL1R2 + S100A9 + CX3CR1 + TAP2 | 0.801 | 0.719 | 0.884 |
| D5/7 | C3AR1 + IL1R2 + S100A9 + CX3CR1 + TAP2 + CD3D | 0.8 | 0.718 | 0.882 |
| D5/7 | C3AR1 + IL1R2 + S100A9 + CX3CR1 + TAP2 + CD3D + CTLA4 | 0.797 | 0.714 | 0.881 |
| D5/7 | C3AR1 + IL1R2 + S100A9 + CX3CR1 + TAP2 + CTLA4 | 0.797 | 0.713 | 0.882 |
| D5/7 | C3AR1 + IL1R2 + S100A9 + TAP2 | 0.801 | 0.719 | 0.884 |
| D5/7 | C3AR1 + IL1R2 + S100A9 + TAP2 + CD3D | 0.799 | 0.716 | 0.881 |
| D5/7 | C3AR1 + IL1R2 + S100A9 + TAP2 + CD3D + CTLA4 | 0.798 | 0.713 | 0.884 |
| D5/7 | C3AR1 + IL1R2 + S100A9 + TAP2 + CTLA4 | 0.798 | 0.712 | 0.884 |
| D5/7 | C3AR1 + IL1R2 + TAP2 | 0.801 | 0.714 | 0.889 |
| D5/7 | C3AR1 + IL1R2 + TAP2 + CD3D | 0.8 | 0.711 | 0.888 |
| D5/7 | C3AR1 + IL1R2 + TAP2 + CD3D + CTLA4 | 0.797 | 0.701 | 0.893 |
| D5/7 | C3AR1 + IL1R2 + TAP2 + CTLA4 | 0.796 | 0.703 | 0.89 |
| D5/7 | C3AR1 + S100A9 | 0.77 | 0.678 | 0.862 |
| D5/7 | C3AR1 + S100A9 + CD3D | 0.776 | 0.683 | 0.87 |
| D5/7 | C3AR1 + S100A9 + CD3D + CTLA4 | 0.764 | 0.661 | 0.867 |
| D5/7 | C3AR1 + S100A9 + CIITA | 0.77 | 0.678 | 0.862 |
| D5/7 | C3AR1 + S100A9 + CIITA + CD3D | 0.774 | 0.676 | 0.872 |
| D5/7 | C3AR1 + S100A9 + CIITA + CD3D + CTLA4 | 0.763 | 0.658 | 0.869 |
| D5/7 | C3AR1 + S100A9 + CIITA + CTLA4 | 0.755 | 0.645 | 0.864 |
| D5/7 | C3AR1 + S100A9 + CIITA + TAP2 | 0.793 | 0.708 | 0.878 |
| D5/7 | C3AR1 + S100A9 + CIITA + TAP2 + CD3D | 0.789 | 0.7 | 0.878 |
| D5/7 | C3AR1 + S100A9 + CIITA + TAP2 + CD3D + CTLA4 | 0.79 | 0.698 | 0.881 |
| D5/7 | C3AR1 + S100A9 + CIITA + TAP2 + CTLA4 | 0.79 | 0.699 | 0.881 |
| D5/7 | C3AR1 + S100A9 + CTLA4 | 0.758 | 0.653 | 0.863 |
| D5/7 | C3AR1 + S100A9 + CX3CR1 | 0.79 | 0.701 | 0.88 |
| D5/7 | C3AR1 + S100A9 + CX3CR1 + CD3D | 0.785 | 0.693 | 0.877 |
| D5/7 | C3AR1 + S100A9 + CX3CR1 + CD3D + CTLA4 | 0.775 | 0.67 | 0.881 |
| D5/7 | C3AR1 + S100A9 + CX3CR1 + CIITA | 0.794 | 0.703 | 0.884 |
| D5/7 | C3AR1 + S100A9 + CX3CR1 + CIITA + CD3D | 0.782 | 0.682 | 0.881 |
| D5/7 | C3AR1 + S100A9 + CX3CR1 + CIITA + CD3D + CTLA4 | 0.773 | 0.664 | 0.883 |
| D5/7 | C3AR1 + S100A9 + CX3CR1 + CIITA + CTLA4 | 0.773 | 0.662 | 0.884 |
| D5/7 | C3AR1 + S100A9 + CX3CR1 + CIITA + TAP2 | 0.791 | 0.705 | 0.878 |
| D5/7 | C3AR1 + S100A9 + CX3CR1 + CIITA + TAP2 + CD3D | 0.789 | 0.7 | 0.878 |
| D5/7 | C3AR1 + S100A9 + CX3CR1 + CIITA + TAP2 + CD3D + CTLA4 | 0.789 | 0.697 | 0.88 |
| D5/7 | C3AR1 + S100A9 + CX3CR1 + CIITA + TAP2 + CTLA4 | 0.788 | 0.696 | 0.88 |
| D5/7 | C3AR1 + S100A9 + CX3CR1 + CTLA4 | 0.776 | 0.67 | 0.882 |
| D5/7 | C3AR1 + S100A9 + CX3CR1 + TAP2 | 0.789 | 0.706 | 0.871 |
| D5/7 | C3AR1 + S100A9 + CX3CR1 + TAP2 + CD3D | 0.791 | 0.71 | 0.873 |
| D5/7 | C3AR1 + S100A9 + CX3CR1 + TAP2 + CD3D + CTLA4 | 0.784 | 0.698 | 0.87 |
| D5/7 | C3AR1 + S100A9 + CX3CR1 + TAP2 + CTLA4 | 0.786 | 0.7 | 0.872 |
| D5/7 | C3AR1 + S100A9 + TAP2 | 0.788 | 0.705 | 0.871 |
| D5/7 | C3AR1 + S100A9 + TAP2 + CD3D | 0.791 | 0.708 | 0.873 |
| D5/7 | C3AR1 + S100A9 + TAP2 + CD3D + CTLA4 | 0.784 | 0.698 | 0.869 |
| D5/7 | C3AR1 + S100A9 + TAP2 + CTLA4 | 0.787 | 0.702 | 0.873 |
| D5/7 | C3AR1 + TAP2 | 0.785 | 0.694 | 0.875 |
| D5/7 | C3AR1 + TAP2 + CD3D | 0.783 | 0.692 | 0.873 |
| D5/7 | C3AR1 + TAP2 + CD3D + CTLA4 | 0.775 | 0.677 | 0.873 |
| D5/7 | C3AR1 + TAP2 + CTLA4 | 0.776 | 0.681 | 0.871 |

TABLE 7
Performance (AUC and 95% confidence interval, with AUC2.5 and AUC 97.5 limits) of the measurement of the expression
of C3AR1 in combination with one or more other biomarker(s) (multivariate analysis) in predicting the incidence
of a healthcare-associated infection in the 4 days or in the 7 days following the sample being taken.

| Maximum time | | | | |
|---|---|---|---|---|
| between sampling and | | | | |
| the incidence of | | | | |
| healthcare-associated | | | | |
| infection | Biomarkers | AUC | AUC.2.5 | AUC.97.5 |
| 4 days | C3AR1 + 130360601-HERV0808 | 0.661 | 0.546 | 0.776 |
| 4 days | C3AR1 + IL18RAP | 0.662 | 0.563 | 0.761 |
| 4 days | C3AR1 + IFIH1 | 0.663 | 0.535 | 0.79 |
| 4 days | C3AR1 + 121601801-HERV0492 | 0.664 | 0.563 | 0.765 |
| 4 days | C3AR1 + 050286701-HERV0513 | 0.665 | 0.567 | 0.764 |
| 4 days | C3AR1 + IL18 | 0.665 | 0.566 | 0.764 |
| 4 days | C3AR1 + CD74 | 0.666 | 0.568 | 0.763 |
| 4 days | C3AR1 + OAS3 | 0.666 | 0.567 | 0.765 |
| 4 days | C3AR1 + CD274 | 0.669 | 0.57 | 0.768 |
| 4 days | C3AR1 + IL18R1 | 0.671 | 0.573 | 0.769 |
| 4 days | C3AR1 + TNFSF4 | 0.673 | 0.574 | 0.772 |
| 4 days | C3AR1 + 011052202-HERV1033 | 0.676 | 0.578 | 0.773 |
| 4 days | C3AR1 + 011052702-MALR1044 | 0.677 | 0.579 | 0.776 |
| 4 days | C3AR1 + IL10 | 0.681 | 0.582 | 0.779 |
| 4 days | C3AR1 + IL1RN | 0.683 | 0.585 | 0.782 |
| 4 days | C3AR1 + 220247002-HERV0797 | 0.687 | 0.59 | 0.783 |
| 4 days | C3AR1 + CX3CR1 | 0.689 | 0.593 | 0.786 |
| 4 days | C3AR1 + 021456001-MALR1017 | 0.691 | 0.594 | 0.787 |
| 4 days | C3AR1 + IL7R | 0.693 | 0.596 | 0.79 |
| 4 days | C3AR1 + IL15 | 0.694 | 0.588 | 0.8 |
| 4 days | C3AR1 + IL1R2 | 0.695 | 0.599 | 0.791 |
| 4 days | C3AR1 + 170369402HE41 | 0.696 | 0.6 | 0.793 |
| 4 days | C3AR1 + HP | 0.715 | 0.622 | 0.808 |
| 4 days | C3AR1 + IFNG | 0.723 | 0.629 | 0.816 |
| 4 days | C3AR1 + CD177 | 0.68 | 0.489 | 0.87 |
| 4 days | C3AR1 + CD177 + CD3D + CTLA4 | 0.664 | 0.463 | 0.865 |
| 4 days | C3AR1 + CD177 + CIITA | 0.684 | 0.493 | 0.874 |
| 4 days | C3AR1 + CD177 + CIITA + CD3D | 0.668 | 0.472 | 0.864 |
| 4 days | C3AR1 + CD177 + CIITA + CD3D + CTLA4 | 0.68 | 0.486 | 0.873 |
| 4 days | C3AR1 + CD177 + CIITA + CTLA4 | 0.676 | 0.482 | 0.87 |
| 4 days | C3AR1 + CD177 + CIITA + TAP2 | 0.684 | 0.492 | 0.875 |
| 4 days | C3AR1 + CD177 + CIITA + TAP2 + CD3D | 0.684 | 0.492 | 0.876 |
| 4 days | C3AR1 + CD177 + CIITA + TAP2 + CD3D + CTLA4 | 0.707 | 0.518 | 0.896 |
| 4 days | C3AR1 + CD177 + CIITA + TAP2 + CTLA4 | 0.699 | 0.509 | 0.889 |
| 4 days | C3AR1 + CD177 + CX3CR1 | 0.703 | 0.515 | 0.891 |
| 4 days | C3AR1 + CD177 + CX3CR1 + CD3D | 0.695 | 0.508 | 0.883 |
| 4 days | C3AR1 + CD177 + CX3CR1 + CD3D + CTLA4 | 0.699 | 0.511 | 0.888 |
| 4 days | C3AR1 + CD177 + CX3CR1 + CIITA | 0.734 | 0.552 | 0.917 |
| 4 days | C3AR1 + CD177 + CX3CR1 + CIITA + CD3D | 0.719 | 0.531 | 0.907 |
| 4 days | C3AR1 + CD177 + CX3CR1 + CIITA + CD3D + CTLA4 | 0.715 | 0.525 | 0.905 |
| 4 days | C3AR1 + CD177 + CX3CR1 + CIITA + CTLA4 | 0.715 | 0.524 | 0.906 |
| 4 days | C3AR1 + CD177 + CX3CR1 + CIITA + TAP2 | 0.773 | 0.611 | 0.936 |
| 4 days | C3AR1 + CD177 + CX3CR1 + CIITA + TAP2 + CD3D | 0.773 | 0.61 | 0.937 |
| 4 days | C3AR1 + CD177 + CX3CR1 + CIITA + TAP2 + CD3D + CTLA4 | 0.77 | 0.605 | 0.934 |
| 4 days | C3AR1 + CD177 + CX3CR1 + CIITA + TAP2 + CTLA4 | 0.762 | 0.594 | 0.929 |
| 4 days | C3AR1 + CD177 + CX3CR1 + CTLA4 | 0.703 | 0.513 | 0.893 |
| 4 days | C3AR1 + CD177 + CX3CR1 + TAP2 | 0.73 | 0.553 | 0.908 |
| 4 days | C3AR1 + CD177 + CX3CR1 + TAP2 + CD3D | 0.746 | 0.575 | 0.917 |
| 4 days | C3AR1 + CD177 + CX3CR1 + TAP2 + CD3D + CTLA4 | 0.758 | 0.59 | 0.926 |
| 4 days | C3AR1 + CD177 + CX3CR1 + TAP2 + CTLA4 | 0.754 | 0.585 | 0.923 |
| 4 days | C3AR1 + CD177 + S100A9 | 0.781 | 0.614 | 0.948 |
| 4 days | C3AR1 + CD177 + S100A9 + CD3D | 0.781 | 0.617 | 0.946 |
| 4 days | C3AR1 + CD177 + S100A9 + CD3D + CTLA4 | 0.781 | 0.615 | 0.947 |
| 4 days | C3AR1 + CD177 + S100A9 + CIITA | 0.793 | 0.625 | 0.961 |
| 4 days | C3AR1 + CD177 + S100A9 + CIITA + CD3D | 0.781 | 0.615 | 0.948 |
| 4 days | C3AR1 + CD177 + S100A9 + CIITA + CD3D + CTLA4 | 0.801 | 0.64 | 0.961 |
| 4 days | C3AR1 + CD177 + S100A9 + CIITA + CTLA4 | 0.797 | 0.632 | 0.962 |
| 4 days | C3AR1 + CD177 + S100A9 + CIITA + TAP2 | 0.805 | 0.632 | 0.977 |
| 4 days | C3AR1 + CD177 + S100A9 + CIITA + TAP2 + CD3D | 0.809 | 0.647 | 0.971 |
| 4 days | C3AR1 + CD177 + S100A9 + CIITA + TAP2 + CD3D + CTLA4 | 0.805 | 0.642 | 0.967 |
| 4 days | C3AR1 + CD177 + S100A9 + CIITA + TAP2 + CTLA4 | 0.816 | 0.656 | 0.976 |
| 4 days | C3AR1 + CD177 + S100A9 + CTLA4 | 0.77 | 0.6 | 0.939 |
| 4 days | C3AR1 + CD177 + S100A9 + CX3CR1 | 0.859 | 0.711 | 1 |
| 4 days | C3AR1 + CD177 + S100A9 + CX3CR1 + CD3D | 0.887 | 0.755 | 1 |
| 4 days | C3AR1 + CD177 + S100A9 + CX3CR1 + CD3D + CTLA4 | 0.879 | 0.749 | 1 |
| 4 days | C3AR1 + CD177 + S100A9 + CX3CR1 + CIITA | 0.879 | 0.743 | 1 |
| 4 days | C3AR1 + CD177 + S100A9 + CX3CR1 + CIITA + CD3D | 0.887 | 0.755 | 1 |
| 4 days | C3AR1 + CD177 + S100A9 + CX3CR1 + CIITA + CD3D + CTLA4 | 0.887 | 0.762 | 1 |
| 4 days | C3AR1 + CD177 + S100A9 + CX3CR1 + CIITA + CTLA4 | 0.871 | 0.729 | 1 |
| 4 days | C3AR1 + CD177 + S100A9 + CX3CR1 + CIITA + TAP2 | 0.883 | 0.743 | 1 |
| 4 days | C3AR1 + CD177 + S100A9 + CX3CR1 + CIITA + TAP2 + CD3D | 0.887 | 0.755 | 1 |
| 4 days | C3AR1 + CD177 + S100A9 + CX3CR1 + CIITA + TAP2 + CD3D + CTLA4 | 0.887 | 0.76 | 1 |
| 4 days | C3AR1 + CD177 + S100A9 + CX3CR1 + CIITA + TAP2 + CTLA4 | 0.879 | 0.735 | 1 |
| 4 days | C3AR1 + CD177 + S100A9 + CX3CR1 + CTLA4 | 0.859 | 0.712 | 1 |
| 4 days | C3AR1 + CD177 + S100A9 + CX3CR1 + TAP2 | 0.883 | 0.743 | 1 |
| 4 days | C3AR1 + CD177 + S100A9 + CX3CR1 + TAP2 + CD3D | 0.887 | 0.755 | 1 |
| 4 days | C3AR1 + CD177 + S100A9 + CX3CR1 + TAP2 + CD3D + CTLA4 | 0.879 | 0.751 | 1 |
| 4 days | C3AR1 + CD177 + S100A9 + CX3CR1 + TAP2 + CTLA4 | 0.883 | 0.739 | 1 |
| 4 days | C3AR1 + CD177 + S100A9 + TAP2 | 0.801 | 0.635 | 0.967 |
| 4 days | C3AR1 + CD177 + S100A9 + TAP2 + CD3D | 0.801 | 0.642 | 0.959 |
| 4 days | C3AR1 + CD177 + S100A9 + TAP2 + CD3D + CTLA4 | 0.805 | 0.648 | 0.961 |
| 4 days | C3AR1 + CD177 + S100A9 + TAP2 + CTLA4 | 0.797 | 0.634 | 0.96 |
| 4 days | C3AR1 + CD177 + TAP2 | 0.699 | 0.511 | 0.888 |
| 4 days | C3AR1 + CD177 + TAP2 + CD3D | 0.695 | 0.504 | 0.886 |
| 4 days | C3AR1 + CD177 + TAP2 + CD3D + CTLA4 | 0.703 | 0.512 | 0.894 |
| 4 days | C3AR1 + CD177 + TAP2 + CTLA4 | 0.699 | 0.509 | 0.89 |
| 4 days | C3AR1 + CD74 + CD177 | 0.707 | 0.521 | 0.893 |
| 4 days | C3AR1 + CD74 + CD177 + CD3D | 0.719 | 0.536 | 0.902 |
| 4 days | C3AR1 + CD74 + CD177 + CD3D + CTLA4 | 0.73 | 0.551 | 0.91 |
| 4 days | C3AR1 + CD74 + CD177 + CIITA | 0.723 | 0.539 | 0.906 |
| 4 days | C3AR1 + CD74 + CD177 + CIITA + CD3D | 0.723 | 0.542 | 0.904 |
| 4 days | C3AR1 + CD74 + CD177 + CIITA + CD3D + CTLA4 | 0.727 | 0.546 | 0.907 |
| 4 days | C3AR1 + CD74 + CD177 + CIITA + CTLA4 | 0.727 | 0.546 | 0.907 |
| 4 days | C3AR1 + CD74 + CD177 + CIITA + TAP2 | 0.719 | 0.534 | 0.903 |
| 4 days | C3AR1 + CD74 + CD177 + CIITA + TAP2 + CD3D | 0.723 | 0.541 | 0.904 |
| 4 days | C3AR1 + CD74 + CD177 + CIITA + TAP2 + CD3D + CTLA4 | 0.742 | 0.564 | 0.92 |
| 4 days | C3AR1 + CD74 + CD177 + CIITA + TAP2 + CTLA4 | 0.738 | 0.558 | 0.919 |
| 4 days | C3AR1 + CD74 + CD177 + CTLA4 | 0.73 | 0.551 | 0.91 |
| 4 days | C3AR1 + CD74 + CD177 + CX3CR1 | 0.734 | 0.553 | 0.915 |
| 4 days | C3AR1 + CD74 + CD177 + CX3CR1 + CD3D | 0.742 | 0.567 | 0.917 |
| 4 days | C3AR1 + CD74 + CD177 + CX3CR1 + CD3D + CTLA4 | 0.75 | 0.576 | 0.924 |
| 4 days | C3AR1 + CD74 + CD177 + CX3CR1 + CIITA | 0.762 | 0.588 | 0.936 |
| 4 days | C3AR1 + CD74 + CD177 + CX3CR1 + CIITA + CD3D | 0.762 | 0.592 | 0.931 |
| 4 days | C3AR1 + CD74 + CD177 + CX3CR1 + CIITA + CD3D + CTLA4 | 0.758 | 0.586 | 0.929 |
| 4 days | C3AR1 + CD74 + CD177 + CX3CR1 + CIITA + CTLA4 | 0.754 | 0.579 | 0.929 |
| 4 days | C3AR1 + CD74 + CD177 + CX3CR1 + CIITA + TAP2 | 0.77 | 0.603 | 0.936 |
| 4 days | C3AR1 + CD74 + CD177 + CX3CR1 + CIITA + TAP2 + CD3D | 0.781 | 0.617 | 0.945 |
| 4 days | C3AR1 + CD74 + CD177 + CX3CR1 + CIITA + TAP2 + CD3D + CTLA4 | 0.785 | 0.622 | 0.948 |
| 4 days | C3AR1 + CD74 + CD177 + CX3CR1 + CIITA + TAP2 + CTLA4 | 0.77 | 0.601 | 0.938 |
| 4 days | C3AR1 + CD74 + CD177 + CX3CR1 + CTLA4 | 0.754 | 0.576 | 0.932 |
| 4 days | C3AR1 + CD74 + CD177 + CX3CR1 + TAP2 | 0.777 | 0.612 | 0.943 |
| 4 days | C3AR1 + CD74 + CD177 + CX3CR1 + TAP2 + CD3D | 0.777 | 0.61 | 0.945 |
| 4 days | C3AR1 + CD74 + CD177 + CX3CR1 + TAP2 + CD3D + CTLA4 | 0.809 | 0.649 | 0.968 |
| 4 days | C3AR1 + CD74 + CD177 + CX3CR1 + TAP2 + CTLA4 | 0.805 | 0.646 | 0.963 |
| 4 days | C3AR1 + CD74 + CD177 + S100A9 | 0.777 | 0.61 | 0.945 |
| 4 days | C3AR1 + CD74 + CD177 + S100A9 + CD3D | 0.785 | 0.622 | 0.948 |
| 4 days | C3AR1 + CD74 + CD177 + S100A9 + CD3D + CTLA4 | 0.781 | 0.615 | 0.947 |
| 4 days | C3AR1 + CD74 + CD177 + S100A9 + CIITA | 0.801 | 0.634 | 0.968 |
| 4 days | C3AR1 + CD74 + CD177 + S100A9 + CIITA + CD3D | 0.789 | 0.622 | 0.956 |
| 4 days | C3AR1 + CD74 + CD177 + S100A9 + CIITA + CD3D + CTLA4 | 0.805 | 0.644 | 0.965 |
| 4 days | C3AR1 + CD74 + CD177 + S100A9 + CIITA + CTLA4 | 0.797 | 0.632 | 0.962 |
| 4 days | C3AR1 + CD74 + CD177 + S100A9 + CIITA + TAP2 | 0.809 | 0.638 | 0.98 |
| 4 days | C3AR1 + CD74 + CD177 + S100A9 + CIITA + TAP2 + CD3D | 0.809 | 0.644 | 0.973 |
| 4 days | C3AR1 + CD74 + CD177 + S100A9 + CIITA + TAP2 + CD3D + CTLA4 | 0.805 | 0.637 | 0.972 |
| 4 days | C3AR1 + CD74 + CD177 + S100A9 + CIITA + TAP2 + CTLA4 | 0.816 | 0.652 | 0.981 |
| 4 days | C3AR1 + CD74 + CD177 + S100A9 + CTLA4 | 0.773 | 0.605 | 0.942 |
| 4 days | C3AR1 + CD74 + CD177 + S100A9 + CX3CR1 | 0.871 | 0.726 | 1 |
| 4 days | C3AR1 + CD74 + CD177 + S100A9 + CX3CR1 + CD3D | 0.887 | 0.75 | 1 |
| 4 days | C3AR1 + CD74 + CD177 + S100A9 + CX3CR1 + CD3D + CTLA4 | 0.887 | 0.759 | 1 |
| 4 days | C3AR1 + CD74 + CD177 + S100A9 + CX3CR1 + CIITA | 0.879 | 0.743 | 1 |
| 4 days | C3AR1 + CD74 + CD177 + S100A9 + CX3CR1 + CIITA + CD3D | 0.887 | 0.755 | 1 |
| 4 days | C3AR1 + CD74 + CD177 + S100A9 + CX3CR1 + CIITA + CD3D + CTLA4 | 0.883 | 0.754 | 1 |
| 4 days | C3AR1 + CD74 + CD177 + S100A9 + CX3CR1 + CIITA + CTLA4 | 0.875 | 0.738 | 1 |
| 4 days | C3AR1 + CD74 + CD177 + S100A9 + CX3CR1 + CIITA + TAP2 | 0.887 | 0.747 | 1 |
| 4 days | C3AR1 + CD74 + CD177 + S100A9 + CX3CR1 + CIITA + TAP2 + CD3D | 0.887 | 0.755 | 1 |
| 4 days | C3AR1 + CD74 + CD177 + S100A9 + CX3CR1 + CIITA + TAP2 + CD3D + CTLA4 | 0.891 | 0.767 | 1 |
| 4 days | C3AR1 + CD74 + CD177 + S100A9 + CX3CR1 + CIITA + TAP2 + CTLA4 | 0.883 | 0.739 | 1 |
| 4 days | C3AR1 + CD74 + CD177 + S100A9 + CX3CR1 + CTLA4 | 0.867 | 0.721 | 1 |
| 4 days | C3AR1 + CD74 + CD177 + S100A9 + CX3CR1 + TAP2 | 0.883 | 0.743 | 1 |
| 4 days | C3AR1 + CD74 + CD177 + S100A9 + CX3CR1 + TAP2 + CD3D | 0.887 | 0.75 | 1 |
| 4 days | C3AR1 + CD74 + CD177 + S100A9 + CX3CR1 + TAP2 + CD3D + CTLA4 | 0.879 | 0.749 | 1 |
| 4 days | C3AR1 + CD74 + CD177 + S100A9 + CX3CR1 + TAP2 + CTLA4 | 0.883 | 0.739 | 1 |
| 4 days | C3AR1 + CD74 + CD177 + S100A9 + TAP2 | 0.801 | 0.635 | 0.967 |
| 4 days | C3AR1 + CD74 + CD177 + S100A9 + TAP2 + CD3D | 0.805 | 0.647 | 0.963 |
| 4 days | C3AR1 + CD74 + CD177 + S100A9 + TAP2 + CD3D + CTLA4 | 0.805 | 0.648 | 0.961 |
| 4 days | C3AR1 + CD74 + CD177 + S100A9 + TAP2 + CTLA4 | 0.797 | 0.634 | 0.96 |
| 4 days | C3AR1 + CD74 + CD177 + TAP2 | 0.719 | 0.535 | 0.903 |
| 4 days | C3AR1 + CD74 + CD177 + TAP2 + CD3D | 0.719 | 0.535 | 0.903 |
| 4 days | C3AR1 + CD74 + CD177 + TAP2 + CD3D + CTLA4 | 0.742 | 0.564 | 0.92 |
| 4 days | C3AR1 + CD74 + CD177 + TAP2 + CTLA4 | 0.738 | 0.558 | 0.919 |
| 4 days | C3AR1 + CD74 + CD3D + CTLA4 | 0.668 | 0.474 | 0.862 |
| 4 days | C3AR1 + CD74 + CIITA + CD3D + CTLA4 | 0.672 | 0.479 | 0.865 |
| 4 days | C3AR1 + CD74 + CIITA + CTLA4 | 0.664 | 0.47 | 0.858 |
| 4 days | C3AR1 + CD74 + CIITA + TAP2 + CD3D + CTLA4 | 0.699 | 0.503 | 0.895 |
| 4 days | C3AR1 + CD74 + CIITA + TAP2 + CTLA4 | 0.695 | 0.505 | 0.885 |
| 4 days | C3AR1 + CD74 + CTLA4 | 0.668 | 0.475 | 0.861 |
| 4 days | C3AR1 + CD74 + CX3CR1 | 0.742 | 0.552 | 0.933 |
| 4 days | C3AR1 + CD74 + CX3CR1 + CD3D | 0.711 | 0.524 | 0.898 |
| 4 days | C3AR1 + CD74 + CX3CR1 + CD3D + CTLA4 | 0.734 | 0.552 | 0.916 |
| 4 days | C3AR1 + CD74 + CX3CR1 + CIITA | 0.754 | 0.568 | 0.939 |
| 4 days | C3AR1 + CD74 + CX3CR1 + CIITA + CD3D | 0.734 | 0.545 | 0.924 |
| 4 days | C3AR1 + CD74 + CX3CR1 + CIITA + CD3D + CTLA4 | 0.73 | 0.539 | 0.922 |
| 4 days | C3AR1 + CD74 + CX3CR1 + CIITA + CTLA4 | 0.73 | 0.539 | 0.922 |
| 4 days | C3AR1 + CD74 + CX3CR1 + CIITA + TAP2 | 0.777 | 0.606 | 0.949 |
| 4 days | C3AR1 + CD74 + CX3CR1 + CIITA + TAP2 + CD3D | 0.797 | 0.635 | 0.959 |
| 4 days | C3AR1 + CD74 + CX3CR1 + CIITA + TAP2 + CD3D + CTLA4 | 0.789 | 0.619 | 0.959 |
| 4 days | C3AR1 + CD74 + CX3CR1 + CIITA + TAP2 + CTLA4 | 0.793 | 0.626 | 0.96 |
| 4 days | C3AR1 + CD74 + CX3CR1 + CTLA4 | 0.734 | 0.551 | 0.918 |
| 4 days | C3AR1 + CD74 + CX3CR1 + TAP2 | 0.773 | 0.596 | 0.951 |
| 4 days | C3AR1 + CD74 + CX3CR1 + TAP2 + CD3D | 0.727 | 0.542 | 0.912 |
| 4 days | C3AR1 + CD74 + CX3CR1 + TAP2 + CD3D + CTLA4 | 0.785 | 0.622 | 0.948 |
| 4 days | C3AR1 + CD74 + CX3CR1 + TAP2 + CTLA4 | 0.781 | 0.614 | 0.948 |
| 4 days | C3AR1 + CD74 + IFNG + CD177 | 0.727 | 0.548 | 0.905 |
| 4 days | C3AR1 + CD74 + IFNG + CD177 + CD3D | 0.727 | 0.545 | 0.909 |
| 4 days | C3AR1 + CD74 + IFNG + CD177 + CD3D + CTLA4 | 0.723 | 0.541 | 0.905 |
| 4 days | C3AR1 + CD74 + IFNG + CD177 + CIITA | 0.727 | 0.548 | 0.905 |
| 4 days | C3AR1 + CD74 + IFNG + CD177 + CIITA + CD3D | 0.727 | 0.545 | 0.909 |
| 4 days | C3AR1 + CD74 + IFNG + CD177 + CIITA + CD3D + CTLA4 | 0.715 | 0.527 | 0.903 |
| 4 days | C3AR1 + CD74 + IFNG + CD177 + CIITA + CTLA4 | 0.723 | 0.537 | 0.909 |
| 4 days | C3AR1 + CD74 + IFNG + CD177 + CIITA + TAP2 | 0.723 | 0.543 | 0.902 |
| 4 days | C3AR1 + CD74 + IFNG + CD177 + CIITA + TAP2 + CD3D | 0.707 | 0.523 | 0.891 |
| 4 days | C3AR1 + CD74 + IFNG + CD177 + CIITA + TAP2 + CD3D + CTLA4 | 0.719 | 0.532 | 0.905 |
| 4 days | C3AR1 + CD74 + IFNG + CD177 + CIITA + TAP2 + CTLA4 | 0.73 | 0.548 | 0.913 |
| 4 days | C3AR1 + CD74 + IFNG + CD177 + CTLA4 | 0.723 | 0.538 | 0.907 |
| 4 days | C3AR1 + CD74 + IFNG + CD177 + CX3CR1 | 0.855 | 0.714 | 0.997 |
| 4 days | C3AR1 + CD74 + IFNG + CD177 + CX3CR1 + CD3D | 0.871 | 0.748 | 0.994 |
| 4 days | C3AR1 + CD74 + IFNG + CD177 + CX3CR1 + CD3D + CTLA4 | 0.891 | 0.778 | 1 |
| 4 days | C3AR1 + CD74 + IFNG + CD177 + CX3CR1 + CIITA | 0.879 | 0.754 | 1 |
| 4 days | C3AR1 + CD74 + IFNG + CD177 + CX3CR1 + CIITA + CD3D | 0.887 | 0.776 | 0.998 |
| 4 days | C3AR1 + CD74 + IFNG + CD177 + CX3CR1 + CIITA + CD3D + CTLA4 | 0.895 | 0.784 | 1 |
| 4 days | C3AR1 + CD74 + IFNG + CD177 + CX3CR1 + CIITA + CTLA4 | 0.895 | 0.784 | 1 |
| 4 days | C3AR1 + CD74 + IFNG + CD177 + CX3CR1 + CIITA + TAP2 | 0.871 | 0.744 | 0.998 |
| 4 days | C3AR1 + CD74 + IFNG + CD177 + CX3CR1 + CIITA + TAP2 + CD3D | 0.875 | 0.758 | 0.992 |
| 4 days | C3AR1 + CD74 + IFNG + CD177 + CX3CR1 + CIITA + TAP2 + CD3D + CTLA4 | 0.867 | 0.74 | 0.994 |
| 4 days | C3AR1 + CD74 + IFNG + CD177 + CX3CR1 + CIITA + TAP2 + CTLA4 | 0.867 | 0.742 | 0.992 |
| 4 days | C3AR1 + CD74 + IFNG + CD177 + CX3CR1 + CTLA4 | 0.898 | 0.79 | 1 |
| 4 days | C3AR1 + CD74 + IFNG + CD177 + CX3CR1 + TAP2 | 0.852 | 0.712 | 0.992 |
| 4 days | C3AR1 + CD74 + IFNG + CD177 + CX3CR1 + TAP2 + CD3D | 0.883 | 0.766 | 0.999 |
| 4 days | C3AR1 + CD74 + IFNG + CD177 + CX3CR1 + TAP2 + CD3D + CTLA4 | 0.867 | 0.74 | 0.994 |
| 4 days | C3AR1 + CD74 + IFNG + CD177 + CX3CR1 + TAP2 + CTLA4 | 0.871 | 0.748 | 0.995 |
| 4 days | C3AR1 + CD74 + IFNG + CD177 + S100A9 | 0.789 | 0.624 | 0.954 |
| 4 days | C3AR1 + CD74 + IFNG + CD177 + S100A9 + CD3D | 0.785 | 0.62 | 0.951 |
| 4 days | C3AR1 + CD74 + IFNG + CD177 + S100A9 + CD3D + CTLA4 | 0.785 | 0.62 | 0.95 |
| 4 days | C3AR1 + CD74 + IFNG + CD177 + S100A9 + CIITA | 0.793 | 0.625 | 0.961 |
| 4 days | C3AR1 + CD74 + IFNG + CD177 + S100A9 + CIITA + CD3D | 0.793 | 0.628 | 0.958 |
| 4 days | C3AR1 + CD74 + IFNG + CD177 + S100A9 + CIITA + CD3D + CTLA4 | 0.797 | 0.633 | 0.961 |
| 4 days | C3AR1 + CD74 + IFNG + CD177 + S100A9 + CIITA + CTLA4 | 0.785 | 0.617 | 0.953 |
| 4 days | C3AR1 + CD74 + IFNG + CD177 + S100A9 + CIITA + TAP2 | 0.801 | 0.628 | 0.974 |
| 4 days | C3AR1 + CD74 + IFNG + CD177 + S100A9 + CIITA + TAP2 + CD3D | 0.801 | 0.636 | 0.966 |
| 4 days | C3AR1 + CD74 + IFNG + CD177 + S100A9 + CIITA + TAP2 + CD3D + CTLA4 | 0.809 | 0.647 | 0.97 |
| 4 days | C3AR1 + CD74 + IFNG + CD177 + S100A9 + CIITA + TAP2 + CTLA4 | 0.797 | 0.631 | 0.963 |
| 4 days | C3AR1 + CD74 + IFNG + CD177 + S100A9 + CTLA4 | 0.773 | 0.605 | 0.942 |
| 4 days | C3AR1 + CD74 + IFNG + CD177 + S100A9 + CX3CR1 | 0.938 | 0.851 | 1 |
| 4 days | C3AR1 + CD74 + IFNG + CD177 + S100A9 + CX3CR1 + CD3D | 0.953 | 0.891 | 1 |
| 4 days | C3AR1 + CD74 + IFNG + CD177 + S100A9 + CX3CR1 + CD3D + CTLA4 | 0.953 | 0.891 | 1 |
| 4 days | C3AR1 + CD74 + IFNG + CD177 + S100A9 + CX3CR1 + CIITA | 0.941 | 0.857 | 1 |
| 4 days | C3AR1 + CD74 + IFNG + CD177 + S100A9 + CX3CR1 + CIITA + CD3D | 0.953 | 0.891 | 1 |
| 4 days | C3AR1 + CD74 + IFNG + CD177 + S100A9 + CX3CR1 + CIITA + CD3D + CTLA4 | 0.953 | 0.891 | 1 |
| 4 days | C3AR1 + CD74 + IFNG + CD177 + S100A9 + CX3CR1 + CIITA + CTLA4 | 0.961 | 0.905 | 1 |
| 4 days | C3AR1 + CD74 + IFNG + CD177 + S100A9 + CX3CR1 + CIITA + TAP2 | 0.934 | 0.845 | 1 |
| 4 days | C3AR1 + CD74 + IFNG + CD177 + S100A9 + CX3CR1 + CIITA + TAP2 + CD3D | 0.957 | 0.897 | 1 |
| 4 days | C3AR1 + CD74 + IFNG + CD177 + S100A9 + CX3CR1 + CIITA + TAP2 + CD3D + CTLA4 | 0.965 | 0.914 | 1 |
| 4 days | C3AR1 + CD74 + IFNG + CD177 + S100A9 + CX3CR1 + CIITA + TAP2 + CTLA4 | 0.953 | 0.889 | 1 |
| 4 days | C3AR1 + CD74 + IFNG + CD177 + S100A9 + CX3CR1 + CTLA4 | 0.945 | 0.871 | 1 |
| 4 days | C3AR1 + CD74 + IFNG + CD177 + S100A9 + CX3CR1 + TAP2 | 0.934 | 0.845 | 1 |
| 4 days | C3AR1 + CD74 + IFNG + CD177 + S100A9 + CX3CR1 + TAP2 + CD3D | 0.957 | 0.897 | 1 |
| 4 days | C3AR1 + CD74 + IFNG + CD177 + S100A9 + CX3CR1 + TAP2 + CD3D + CTLA4 | 0.965 | 0.914 | 1 |
| 4 days | C3AR1 + CD74 + IFNG + CD177 + S100A9 + CX3CR1 + TAP2 + CTLA4 | 0.938 | 0.849 | 1 |
| 4 days | C3AR1 + CD74 + IFNG + CD177 + S100A9 + TAP2 | 0.801 | 0.635 | 0.967 |
| 4 days | C3AR1 + CD74 + IFNG + CD177 + S100A9 + TAP2 + CD3D | 0.805 | 0.647 | 0.963 |
| 4 days | C3AR1 + CD74 + IFNG + CD177 + S100A9 + TAP2 + CD3D + CTLA4 | 0.805 | 0.648 | 0.961 |
| 4 days | C3AR1 + CD74 + IFNG + CD177 + S100A9 + TAP2 + CTLA4 | 0.789 | 0.626 | 0.952 |
| 4 days | C3AR1 + CD74 + IFNG + CD177 + TAP2 | 0.719 | 0.538 | 0.899 |
| 4 days | C3AR1 + CD74 + IFNG + CD177 + TAP2 + CD3D | 0.715 | 0.532 | 0.898 |
| 4 days | C3AR1 + CD74 + IFNG + CD177 + TAP2 + CD3D + CTLA4 | 0.719 | 0.536 | 0.901 |
| 4 days | C3AR1 + CD74 + IFNG + CD177 + TAP2 + CTLA4 | 0.715 | 0.53 | 0.9 |
| 4 days | C3AR1 + CD74 + IFNG + CD3D + CTLA4 | 0.684 | 0.49 | 0.877 |
| 4 days | C3AR1 + CD74 + IFNG + CIITA + CD3D + CTLA4 | 0.684 | 0.49 | 0.878 |
| 4 days | C3AR1 + CD74 + IFNG + CIITA + TAP2 + CD3D + CTLA4 | 0.68 | 0.484 | 0.875 |
| 4 days | C3AR1 + CD74 + IFNG + CTLA4 | 0.68 | 0.491 | 0.868 |
| 4 days | C3AR1 + CD74 + IFNG + CX3CR1 | 0.855 | 0.711 | 1 |
| 4 days | C3AR1 + CD74 + IFNG + CX3CR1 + CD3D | 0.871 | 0.748 | 0.994 |
| 4 days | C3AR1 + CD74 + IFNG + CX3CR1 + CD3D + CTLA4 | 0.891 | 0.783 | 0.998 |
| 4 days | C3AR1 + CD74 + IFNG + CX3CR1 + CIITA | 0.883 | 0.76 | 1 |
| 4 days | C3AR1 + CD74 + IFNG + CX3CR1 + CIITA + CD3D | 0.883 | 0.769 | 0.996 |
| 4 days | C3AR1 + CD74 + IFNG + CX3CR1 + CIITA + CD3D + CTLA4 | 0.898 | 0.795 | 1 |
| 4 days | C3AR1 + CD74 + IFNG + CX3CR1 + CIITA + CTLA4 | 0.887 | 0.776 | 0.998 |
| 4 days | C3AR1 + CD74 + IFNG + CX3CR1 + CIITA + TAP2 | 0.867 | 0.739 | 0.996 |
| 4 days | C3AR1 + CD74 + IFNG + CX3CR1 + CIITA + TAP2 + CD3D | 0.883 | 0.77 | 0.996 |
| 4 days | C3AR1 + CD74 + IFNG + CX3CR1 + CIITA + TAP2 + CD3D + CTLA4 | 0.891 | 0.783 | 0.998 |
| 4 days | C3AR1 + CD74 + IFNG + CX3CR1 + CIITA + TAP2 + CTLA4 | 0.887 | 0.776 | 0.997 |
| 4 days | C3AR1 + CD74 + IFNG + CX3CR1 + CTLA4 | 0.887 | 0.776 | 0.998 |
| 4 days | C3AR1 + CD74 + IFNG + CX3CR1 + TAP2 | 0.852 | 0.712 | 0.992 |
| 4 days | C3AR1 + CD74 + IFNG + CX3CR1 + TAP2 + CD3D | 0.883 | 0.766 | 0.999 |
| 4 days | C3AR1 + CD74 + IFNG + CX3CR1 + TAP2 + CD3D + CTLA4 | 0.887 | 0.777 | 0.997 |
| 4 days | C3AR1 + CD74 + IFNG + CX3CR1 + TAP2 + CTLA4 | 0.887 | 0.776 | 0.997 |
| 4 days | C3AR1 + CD74 + IFNG + S100A9 | 0.797 | 0.633 | 0.961 |
| 4 days | C3AR1 + CD74 + IFNG + S100A9 + CD3D | 0.789 | 0.626 | 0.953 |
| 4 days | C3AR1 + CD74 + IFNG + S100A9 + CD3D + CTLA4 | 0.789 | 0.628 | 0.95 |
| 4 days | C3AR1 + CD74 + IFNG + S100A9 + CIITA | 0.797 | 0.63 | 0.964 |
| 4 days | C3AR1 + CD74 + IFNG + S100A9 + CIITA + CD3D | 0.785 | 0.617 | 0.953 |
| 4 days | C3AR1 + CD74 + IFNG + S100A9 + CIITA + CD3D + CTLA4 | 0.793 | 0.629 | 0.957 |
| 4 days | C3AR1 + CD74 + IFNG + S100A9 + CIITA + CTLA4 | 0.793 | 0.628 | 0.958 |
| 4 days | C3AR1 + CD74 + IFNG + S100A9 + CIITA + TAP2 | 0.805 | 0.632 | 0.977 |
| 4 days | C3AR1 + CD74 + IFNG + S100A9 + CIITA + TAP2 + CD3D | 0.793 | 0.624 | 0.962 |
| 4 days | C3AR1 + CD74 + IFNG + S100A9 + CIITA + TAP2 + CD3D + CTLA4 | 0.805 | 0.642 | 0.967 |
| 4 days | C3AR1 + CD74 + IFNG + S100A9 + CIITA + TAP2 + CTLA4 | 0.805 | 0.64 | 0.97 |
| 4 days | C3AR1 + CD74 + IFNG + S100A9 + CTLA4 | 0.793 | 0.632 | 0.954 |
| 4 days | C3AR1 + CD74 + IFNG + S100A9 + CX3CR1 | 0.922 | 0.827 | 1 |
| 4 days | C3AR1 + CD74 + IFNG + S100A9 + CX3CR1 + CD3D | 0.938 | 0.862 | 1 |
| 4 days | C3AR1 + CD74 + IFNG + S100A9 + CX3CR1 + CD3D + CTLA4 | 0.941 | 0.867 | 1 |
| 4 days | C3AR1 + CD74 + IFNG + S100A9 + CX3CR1 + CIITA | 0.922 | 0.827 | 1 |
| 4 days | C3AR1 + CD74 + IFNG + S100A9 + CX3CR1 + CIITA + CD3D | 0.949 | 0.883 | 1 |
| 4 days | C3AR1 + CD74 + IFNG + S100A9 + CX3CR1 + CIITA + CD3D + CTLA4 | 0.945 | 0.876 | 1 |
| 4 days | C3AR1 + CD74 + IFNG + S100A9 + CX3CR1 + CIITA + CTLA4 | 0.922 | 0.832 | 1 |
| 4 days | C3AR1 + CD74 + IFNG + S100A9 + CX3CR1 + CIITA + TAP2 | 0.918 | 0.818 | 1 |
| 4 days | C3AR1 + CD74 + IFNG + S100A9 + CX3CR1 + CIITA + TAP2 + CD3D | 0.945 | 0.874 | 1 |
| 4 days | C3AR1 + CD74 + IFNG + S100A9 + CX3CR1 + CIITA + TAP2 + CD3D + CTLA4 | 0.949 | 0.877 | 1 |
| 4 days | C3AR1 + CD74 + IFNG + S100A9 + CX3CR1 + CIITA + TAP2 + CTLA4 | 0.918 | 0.82 | 1 |
| 4 days | C3AR1 + CD74 + IFNG + S100A9 + CX3CR1 + CTLA4 | 0.914 | 0.817 | 1 |
| 4 days | C3AR1 + CD74 + IFNG + S100A9 + CX3CR1 + TAP2 | 0.918 | 0.818 | 1 |
| 4 days | C3AR1 + CD74 + IFNG + S100A9 + CX3CR1 + TAP2 + CD3D | 0.941 | 0.866 | 1 |
| 4 days | C3AR1 + CD74 + IFNG + S100A9 + CX3CR1 + TAP2 + CD3D + CTLA4 | 0.953 | 0.883 | 1 |
| 4 days | C3AR1 + CD74 + IFNG + S100A9 + CX3CR1 + TAP2 + CTLA4 | 0.922 | 0.832 | 1 |
| 4 days | C3AR1 + CD74 + IFNG + S100A9 + TAP2 | 0.809 | 0.647 | 0.97 |
| 4 days | C3AR1 + CD74 + IFNG + S100A9 + TAP2 + CD3D | 0.797 | 0.633 | 0.961 |
| 4 days | C3AR1 + CD74 + IFNG + S100A9 + TAP2 + CD3D + CTLA4 | 0.809 | 0.649 | 0.968 |
| 4 days | C3AR1 + CD74 + IFNG + S100A9 + TAP2 + CTLA4 | 0.805 | 0.645 | 0.965 |
| 4 days | C3AR1 + CD74 + IFNG + TAP2 + CD3D + CTLA4 | 0.684 | 0.49 | 0.877 |
| 4 days | C3AR1 + CD74 + IFNG + TAP2 + CTLA4 | 0.684 | 0.496 | 0.871 |
| 4 days | C3AR1 + CD74 + S100A9 | 0.797 | 0.636 | 0.958 |
| 4 days | C3AR1 + CD74 + S100A9 + CD3D | 0.781 | 0.617 | 0.946 |
| 4 days | C3AR1 + CD74 + S100A9 + CD3D + CTLA4 | 0.785 | 0.622 | 0.948 |
| 4 days | C3AR1 + CD74 + S100A9 + CIITA | 0.805 | 0.639 | 0.97 |
| 4 days | C3AR1 + CD74 + S100A9 + CIITA + CD3D | 0.789 | 0.624 | 0.954 |
| 4 days | C3AR1 + CD74 + S100A9 + CIITA + CD3D + CTLA4 | 0.785 | 0.616 | 0.954 |
| 4 days | C3AR1 + CD74 + S100A9 + CIITA + CTLA4 | 0.797 | 0.631 | 0.962 |
| 4 days | C3AR1 + CD74 + S100A9 + CIITA + TAP2 | 0.809 | 0.634 | 0.983 |
| 4 days | C3AR1 + CD74 + S100A9 + CIITA + TAP2 + CD3D | 0.793 | 0.626 | 0.96 |
| 4 days | C3AR1 + CD74 + S100A9 + CIITA + TAP2 + CD3D + CTLA4 | 0.805 | 0.639 | 0.97 |
| 4 days | C3AR1 + CD74 + S100A9 + CIITA + TAP2 + CTLA4 | 0.816 | 0.653 | 0.98 |
| 4 days | C3AR1 + CD74 + S100A9 + CTLA4 | 0.789 | 0.627 | 0.951 |
| 4 days | C3AR1 + CD74 + S100A9 + CX3CR1 | 0.867 | 0.727 | 1 |
| 4 days | C3AR1 + CD74 + S100A9 + CX3CR1 + CD3D | 0.887 | 0.747 | 1 |
| 4 days | C3AR1 + CD74 + S100A9 + CX3CR1 + CD3D + CTLA4 | 0.879 | 0.744 | 1 |
| 4 days | C3AR1 + CD74 + S100A9 + CX3CR1 + CIITA | 0.871 | 0.736 | 1 |
| 4 days | C3AR1 + CD74 + S100A9 + CX3CR1 + CIITA + CD3D | 0.883 | 0.747 | 1 |
| 4 days | C3AR1 + CD74 + S100A9 + CX3CR1 + CIITA + CD3D + CTLA4 | 0.895 | 0.773 | 1 |
| 4 days | C3AR1 + CD74 + S100A9 + CX3CR1 + CIITA + CTLA4 | 0.875 | 0.741 | 1 |
| 4 days | C3AR1 + CD74 + S100A9 + CX3CR1 + CIITA + TAP2 | 0.879 | 0.743 | 1 |
| 4 days | C3AR1 + CD74 + S100A9 + CX3CR1 + CIITA + TAP2 + CD3D | 0.883 | 0.747 | 1 |
| 4 days | C3AR1 + CD74 + S100A9 + CX3CR1 + CIITA + TAP2 + CD3D + CTLA4 | 0.891 | 0.767 | 1 |
| 4 days | C3AR1 + CD74 + S100A9 + CX3CR1 + CIITA + TAP2 + CTLA4 | 0.879 | 0.739 | 1 |
| 4 days | C3AR1 + CD74 + S100A9 + CX3CR1 + CTLA4 | 0.875 | 0.737 | 1 |
| 4 days | C3AR1 + CD74 + S100A9 + CX3CR1 + TAP2 | 0.871 | 0.726 | 1 |
| 4 days | C3AR1 + CD74 + S100A9 + CX3CR1 + TAP2 + CD3D | 0.887 | 0.747 | 1 |
| 4 days | C3AR1 + CD74 + S100A9 + CX3CR1 + TAP2 + CD3D + CTLA4 | 0.883 | 0.753 | 1 |
| 4 days | C3AR1 + CD74 + S100A9 + CX3CR1 + TAP2 + CTLA4 | 0.875 | 0.73 | 1 |
| 4 days | C3AR1 + CD74 + S100A9 + TAP2 | 0.812 | 0.651 | 0.974 |
| 4 days | C3AR1 + CD74 + S100A9 + TAP2 + CD3D | 0.801 | 0.637 | 0.965 |
| 4 days | C3AR1 + CD74 + S100A9 + TAP2 + CD3D + CTLA4 | 0.805 | 0.642 | 0.967 |
| 4 days | C3AR1 + CD74 + S100A9 + TAP2 + CTLA4 | 0.816 | 0.66 | 0.973 |
| 4 days | C3AR1 + CD74 + TAP2 + CD3D + CTLA4 | 0.695 | 0.5 | 0.891 |
| 4 days | C3AR1 + CD74 + TAP2 + CTLA4 | 0.688 | 0.496 | 0.879 |
| 4 days | C3AR1 + CIITA + TAP2 + CD3D + CTLA4 | 0.688 | 0.498 | 0.877 |
| 4 days | C3AR1 + CIITA + TAP2 + CTLA4 | 0.664 | 0.469 | 0.859 |
| 4 days | C3AR1 + CX3CR1 + CD3D + CTLA4 | 0.668 | 0.474 | 0.862 |
| 4 days | C3AR1 + CX3CR1 + CIITA | 0.711 | 0.52 | 0.902 |
| 4 days | C3AR1 + CX3CR1 + CIITA + CD3D | 0.676 | 0.478 | 0.873 |
| 4 days | C3AR1 + CX3CR1 + CIITA + CD3D + CTLA4 | 0.688 | 0.492 | 0.883 |
| 4 days | C3AR1 + CX3CR1 + CIITA + CTLA4 | 0.719 | 0.528 | 0.909 |
| 4 days | C3AR1 + CX3CR1 + CIITA + TAP2 | 0.754 | 0.578 | 0.93 |
| 4 days | C3AR1 + CX3CR1 + CIITA + TAP2 + CD3D | 0.75 | 0.579 | 0.921 |
| 4 days | C3AR1 + CX3CR1 + CIITA + TAP2 + CD3D + CTLA4 | 0.75 | 0.578 | 0.922 |
| 4 days | C3AR1 + CX3CR1 + CIITA + TAP2 + CTLA4 | 0.762 | 0.595 | 0.928 |
| 4 days | C3AR1 + CX3CR1 + CTLA4 | 0.668 | 0.475 | 0.861 |
| 4 days | C3AR1 + CX3CR1 + TAP2 | 0.672 | 0.482 | 0.862 |
| 4 days | C3AR1 + CX3CR1 + TAP2 + CD3D | 0.676 | 0.484 | 0.868 |
| 4 days | C3AR1 + CX3CR1 + TAP2 + CD3D + CTLA4 | 0.695 | 0.509 | 0.881 |
| 4 days | C3AR1 + CX3CR1 + TAP2 + CTLA4 | 0.691 | 0.505 | 0.878 |
| 4 days | C3AR1 + IFNG + CD177 | 0.703 | 0.517 | 0.89 |
| 4 days | C3AR1 + IFNG + CD177 + CD3D | 0.707 | 0.523 | 0.891 |
| 4 days | C3AR1 + IFNG + CD177 + CD3D + CTLA4 | 0.711 | 0.526 | 0.896 |
| 4 days | C3AR1 + IFNG + CD177 + CIITA | 0.707 | 0.524 | 0.89 |
| 4 days | C3AR1 + IFNG + CD177 + CIITA + CD3D | 0.699 | 0.514 | 0.884 |
| 4 days | C3AR1 + IFNG + CD177 + CIITA + CD3D + CTLA4 | 0.699 | 0.509 | 0.889 |
| 4 days | C3AR1 + IFNG + CD177 + CIITA + CTLA4 | 0.734 | 0.552 | 0.916 |
| 4 days | C3AR1 + IFNG + CD177 + CIITA + TAP2 | 0.703 | 0.518 | 0.888 |
| 4 days | C3AR1 + IFNG + CD177 + CIITA + TAP2 + CD3D | 0.703 | 0.518 | 0.888 |
| 4 days | C3AR1 + IFNG + CD177 + CIITA + TAP2 + CD3D + CTLA4 | 0.703 | 0.512 | 0.895 |
| 4 days | C3AR1 + IFNG + CD177 + CIITA + TAP2 + CTLA4 | 0.734 | 0.552 | 0.916 |
| 4 days | C3AR1 + IFNG + CD177 + CTLA4 | 0.727 | 0.546 | 0.907 |
| 4 days | C3AR1 + IFNG + CD177 + CX3CR1 | 0.844 | 0.696 | 0.992 |
| 4 days | C3AR1 + IFNG + CD177 + CX3CR1 + CD3D | 0.852 | 0.716 | 0.987 |
| 4 days | C3AR1 + IFNG + CD177 + CX3CR1 + CD3D + CTLA4 | 0.895 | 0.778 | 1 |
| 4 days | C3AR1 + IFNG + CD177 + CX3CR1 + CIITA | 0.863 | 0.722 | 1 |
| 4 days | C3AR1 + IFNG + CD177 + CX3CR1 + CIITA + CD3D | 0.863 | 0.734 | 0.993 |
| 4 days | C3AR1 + IFNG + CD177 + CX3CR1 + CIITA + CD3D + CTLA4 | 0.883 | 0.757 | 1 |
| 4 days | C3AR1 + IFNG + CD177 + CX3CR1 + CIITA + CTLA4 | 0.895 | 0.781 | 1 |
| 4 days | C3AR1 + IFNG + CD177 + CX3CR1 + CIITA + TAP2 | 0.867 | 0.736 | 0.998 |
| 4 days | C3AR1 + IFNG + CD177 + CX3CR1 + CIITA + TAP2 + CD3D | 0.863 | 0.732 | 0.995 |
| 4 days | C3AR1 + IFNG + CD177 + CX3CR1 + CIITA + TAP2 + CD3D + CTLA4 | 0.879 | 0.75 | 1 |
| 4 days | C3AR1 + IFNG + CD177 + CX3CR1 + CIITA + TAP2 + CTLA4 | 0.875 | 0.746 | 1 |
| 4 days | C3AR1 + IFNG + CD177 + CX3CR1 + CTLA4 | 0.883 | 0.765 | 1 |
| 4 days | C3AR1 + IFNG + CD177 + CX3CR1 + TAP2 | 0.855 | 0.722 | 0.989 |
| 4 days | C3AR1 + IFNG + CD177 + CX3CR1 + TAP2 + CD3D | 0.859 | 0.731 | 0.987 |
| 4 days | C3AR1 + IFNG + CD177 + CX3CR1 + TAP2 + CD3D + CTLA4 | 0.883 | 0.757 | 1 |
| 4 days | C3AR1 + IFNG + CD177 + CX3CR1 + TAP2 + CTLA4 | 0.879 | 0.752 | 1 |
| 4 days | C3AR1 + IFNG + CD177 + S100A9 | 0.789 | 0.624 | 0.954 |
| 4 days | C3AR1 + IFNG + CD177 + S100A9 + CD3D | 0.781 | 0.615 | 0.947 |
| 4 days | C3AR1 + IFNG + CD177 + S100A9 + CD3D + CTLA4 | 0.785 | 0.62 | 0.95 |
| 4 days | C3AR1 + IFNG + CD177 + S100A9 + CIITA | 0.793 | 0.625 | 0.961 |
| 4 days | C3AR1 + IFNG + CD177 + S100A9 + CIITA + CD3D | 0.789 | 0.624 | 0.954 |
| 4 days | C3AR1 + IFNG + CD177 + S100A9 + CIITA + CD3D + CTLA4 | 0.785 | 0.62 | 0.951 |
| 4 days | C3AR1 + IFNG + CD177 + S100A9 + CIITA + CTLA4 | 0.785 | 0.619 | 0.952 |
| 4 days | C3AR1 + IFNG + CD177 + S100A9 + CIITA + TAP2 | 0.801 | 0.628 | 0.974 |
| 4 days | C3AR1 + IFNG + CD177 + S100A9 + CIITA + TAP2 + CD3D | 0.797 | 0.636 | 0.957 |
| 4 days | C3AR1 + IFNG + CD177 + S100A9 + CIITA + TAP2 + CD3D + CTLA4 | 0.797 | 0.635 | 0.959 |
| 4 days | C3AR1 + IFNG + CD177 + S100A9 + CIITA + TAP2 + CTLA4 | 0.812 | 0.653 | 0.972 |
| 4 days | C3AR1 + IFNG + CD177 + S100A9 + CTLA4 | 0.77 | 0.6 | 0.939 |
| 4 days | C3AR1 + IFNG + CD177 + S100A9 + CX3CR1 | 0.93 | 0.838 | 1 |
| 4 days | C3AR1 + IFNG + CD177 + S100A9 + CX3CR1 + CD3D | 0.941 | 0.866 | 1 |
| 4 days | C3AR1 + IFNG + CD177 + S100A9 + CX3CR1 + CD3D + CTLA4 | 0.957 | 0.897 | 1 |
| 4 days | C3AR1 + IFNG + CD177 + S100A9 + CX3CR1 + CIITA | 0.93 | 0.838 | 1 |
| 4 days | C3AR1 + IFNG + CD177 + S100A9 + CX3CR1 + CIITA + CD3D | 0.941 | 0.866 | 1 |
| 4 days | C3AR1 + IFNG + CD177 + S100A9 + CX3CR1 + CIITA + CD3D + CTLA4 | 0.957 | 0.897 | 1 |
| 4 days | C3AR1 + IFNG + CD177 + S100A9 + CX3CR1 + CIITA + CTLA4 | 0.945 | 0.871 | 1 |
| 4 days | C3AR1 + IFNG + CD177 + S100A9 + CX3CR1 + CIITA + TAP2 | 0.934 | 0.845 | 1 |
| 4 days | C3AR1 + IFNG + CD177 + S100A9 + CX3CR1 + CIITA + TAP2 + CD3D | 0.949 | 0.882 | 1 |
| 4 days | C3AR1 + IFNG + CD177 + S100A9 + CX3CR1 + CIITA + TAP2 + CD3D + CTLA4 | 0.953 | 0.889 | 1 |
| 4 days | C3AR1 + IFNG + CD177 + S100A9 + CX3CR1 + CIITA + TAP2 + CTLA4 | 0.941 | 0.863 | 1 |
| 4 days | C3AR1 + IFNG + CD177 + S100A9 + CX3CR1 + CTLA4 | 0.941 | 0.864 | 1 |
| 4 days | C3AR1 + IFNG + CD177 + S100A9 + CX3CR1 + TAP2 | 0.934 | 0.845 | 1 |
| 4 days | C3AR1 + IFNG + CD177 + S100A9 + CX3CR1 + TAP2 + CD3D | 0.949 | 0.882 | 1 |
| 4 days | C3AR1 + IFNG + CD177 + S100A9 + CX3CR1 + TAP2 + CD3D + CTLA4 | 0.953 | 0.889 | 1 |
| 4 days | C3AR1 + IFNG + CD177 + S100A9 + CX3CR1 + TAP2 + CTLA4 | 0.938 | 0.852 | 1 |
| 4 days | C3AR1 + IFNG + CD177 + S100A9 + TAP2 | 0.801 | 0.635 | 0.967 |
| 4 days | C3AR1 + IFNG + CD177 + S100A9 + TAP2 + CD3D | 0.801 | 0.642 | 0.959 |
| 4 days | C3AR1 + IFNG + CD177 + S100A9 + TAP2 + CD3D + CTLA4 | 0.805 | 0.648 | 0.961 |
| 4 days | C3AR1 + IFNG + CD177 + S100A9 + TAP2 + CTLA4 | 0.785 | 0.621 | 0.949 |
| 4 days | C3AR1 + IFNG + CD177 + TAP2 | 0.719 | 0.535 | 0.902 |
| 4 days | C3AR1 + IFNG + CD177 + TAP2 + CD3D | 0.719 | 0.536 | 0.902 |
| 4 days | C3AR1 + IFNG + CD177 + TAP2 + CD3D + CTLA4 | 0.707 | 0.521 | 0.893 |
| 4 days | C3AR1 + IFNG + CD177 + TAP2 + CTLA4 | 0.727 | 0.546 | 0.907 |
| 4 days | C3AR1 + IFNG + CD3D | 0.684 | 0.491 | 0.876 |
| 4 days | C3AR1 + IFNG + CIITA + CTLA4 | 0.684 | 0.495 | 0.873 |
| 4 days | C3AR1 + IFNG + CIITA + TAP2 | 0.668 | 0.471 | 0.865 |
| 4 days | C3AR1 + IFNG + CIITA + TAP2 + CD3D | 0.668 | 0.471 | 0.865 |
| 4 days | C3AR1 + IFNG + CIITA + TAP2 + CTLA4 | 0.691 | 0.503 | 0.88 |
| 4 days | C3AR1 + IFNG + CTLA4 | 0.668 | 0.477 | 0.859 |
| 4 days | C3AR1 + IFNG + CX3CR1 | 0.84 | 0.685 | 0.994 |
| 4 days | C3AR1 + IFNG + CX3CR1 + CD3D | 0.852 | 0.716 | 0.987 |
| 4 days | C3AR1 + IFNG + CX3CR1 + CD3D + CTLA4 | 0.879 | 0.762 | 0.996 |
| 4 days | C3AR1 + IFNG + CX3CR1 + CIITA | 0.863 | 0.723 | 1 |
| 4 days | C3AR1 + IFNG + CX3CR1 + CIITA + CD3D | 0.863 | 0.734 | 0.993 |
| 4 days | C3AR1 + IFNG + CX3CR1 + CIITA + CD3D + CTLA4 | 0.891 | 0.779 | 1 |
| 4 days | C3AR1 + IFNG + CX3CR1 + CIITA + CTLA4 | 0.883 | 0.767 | 0.998 |
| 4 days | C3AR1 + IFNG + CX3CR1 + CIITA + TAP2 | 0.867 | 0.736 | 0.998 |
| 4 days | C3AR1 + IFNG + CX3CR1 + CIITA + TAP2 + CD3D | 0.863 | 0.733 | 0.993 |
| 4 days | C3AR1 + IFNG + CX3CR1 + CIITA + TAP2 + CD3D + CTLA4 | 0.883 | 0.768 | 0.997 |
| 4 days | C3AR1 + IFNG + CX3CR1 + CIITA + TAP2 + CTLA4 | 0.887 | 0.774 | 1 |
| 4 days | C3AR1 + IFNG + CX3CR1 + CTLA4 | 0.867 | 0.745 | 0.99 |
| 4 days | C3AR1 + IFNG + CX3CR1 + TAP2 | 0.855 | 0.721 | 0.99 |
| 4 days | C3AR1 + IFNG + CX3CR1 + TAP2 + CD3D | 0.859 | 0.731 | 0.987 |
| 4 days | C3AR1 + IFNG + CX3CR1 + TAP2 + CD3D + CTLA4 | 0.879 | 0.76 | 0.998 |
| 4 days | C3AR1 + IFNG + CX3CR1 + TAP2 + CTLA4 | 0.875 | 0.753 | 0.997 |
| 4 days | C3AR1 + IFNG + S100A9 | 0.801 | 0.637 | 0.964 |
| 4 days | C3AR1 + IFNG + S100A9 + CD3D | 0.781 | 0.613 | 0.95 |
| 4 days | C3AR1 + IFNG + S100A9 + CD3D + CTLA4 | 0.797 | 0.639 | 0.955 |
| 4 days | C3AR1 + IFNG + S100A9 + CIITA | 0.797 | 0.629 | 0.965 |
| 4 days | C3AR1 + IFNG + S100A9 + CIITA + CD3D | 0.77 | 0.598 | 0.941 |
| 4 days | C3AR1 + IFNG + S100A9 + CIITA + CD3D + CTLA4 | 0.777 | 0.609 | 0.946 |
| 4 days | C3AR1 + IFNG + S100A9 + CIITA + CTLA4 | 0.777 | 0.606 | 0.948 |
| 4 days | C3AR1 + IFNG + S100A9 + CIITA + TAP2 | 0.809 | 0.639 | 0.979 |
| 4 days | C3AR1 + IFNG + S100A9 + CIITA + TAP2 + CD3D | 0.789 | 0.621 | 0.957 |
| 4 days | C3AR1 + IFNG + S100A9 + CIITA + TAP2 + CD3D + CTLA4 | 0.789 | 0.62 | 0.958 |
| 4 days | C3AR1 + IFNG + S100A9 + CIITA + TAP2 + CTLA4 | 0.797 | 0.632 | 0.962 |
| 4 days | C3AR1 + IFNG + S100A9 + CTLA4 | 0.793 | 0.632 | 0.954 |
| 4 days | C3AR1 + IFNG + S100A9 + CX3CR1 | 0.922 | 0.827 | 1 |
| 4 days | C3AR1 + IFNG + S100A9 + CX3CR1 + CD3D | 0.934 | 0.854 | 1 |
| 4 days | C3AR1 + IFNG + S100A9 + CX3CR1 + CD3D + CTLA4 | 0.938 | 0.861 | 1 |
| 4 days | C3AR1 + IFNG + S100A9 + CX3CR1 + CIITA | 0.918 | 0.82 | 1 |
| 4 days | C3AR1 + IFNG + S100A9 + CX3CR1 + CIITA + CD3D | 0.934 | 0.852 | 1 |
| 4 days | C3AR1 + IFNG + S100A9 + CX3CR1 + CIITA + CD3D + CTLA4 | 0.926 | 0.837 | 1 |
| 4 days | C3AR1 + IFNG + S100A9 + CX3CR1 + CIITA + CTLA4 | 0.922 | 0.832 | 1 |
| 4 days | C3AR1 + IFNG + S100A9 + CX3CR1 + CIITA + TAP2 | 0.918 | 0.82 | 1 |
| 4 days | C3AR1 + IFNG + S100A9 + CX3CR1 + CIITA + TAP2 + CD3D | 0.938 | 0.86 | 1 |
| 4 days | C3AR1 + IFNG + S100A9 + CX3CR1 + CIITA + TAP2 + CD3D + CTLA4 | 0.938 | 0.858 | 1 |
| 4 days | C3AR1 + IFNG + S100A9 + CX3CR1 + CIITA + TAP2 + CTLA4 | 0.922 | 0.827 | 1 |
| 4 days | C3AR1 + IFNG + S100A9 + CX3CR1 + CTLA4 | 0.918 | 0.825 | 1 |
| 4 days | C3AR1 + IFNG + S100A9 + CX3CR1 + TAP2 | 0.918 | 0.82 | 1 |
| 4 days | C3AR1 + IFNG + S100A9 + CX3CR1 + TAP2 + CD3D | 0.941 | 0.866 | 1 |
| 4 days | C3AR1 + IFNG + S100A9 + CX3CR1 + TAP2 + CD3D + CTLA4 | 0.941 | 0.865 | 1 |
| 4 days | C3AR1 + IFNG + S100A9 + CX3CR1 + TAP2 + CTLA4 | 0.922 | 0.832 | 1 |
| 4 days | C3AR1 + IFNG + S100A9 + TAP2 | 0.809 | 0.647 | 0.97 |
| 4 days | C3AR1 + IFNG + S100A9 + TAP2 + CD3D | 0.797 | 0.631 | 0.963 |
| 4 days | C3AR1 + IFNG + S100A9 + TAP2 + CD3D + CTLA4 | 0.801 | 0.637 | 0.965 |
| 4 days | C3AR1 + IFNG + S100A9 + TAP2 + CTLA4 | 0.805 | 0.645 | 0.965 |
| 4 days | C3AR1 + IFNG + TAP2 | 0.68 | 0.484 | 0.875 |
| 4 days | C3AR1 + IFNG + TAP2 + CD3D | 0.691 | 0.496 | 0.886 |
| 4 days | C3AR1 + IFNG + TAP2 + CTLA4 | 0.668 | 0.477 | 0.859 |
| 4 days | C3AR1 + IL1R2 + CD177 | 0.676 | 0.483 | 0.869 |
| 4 days | C3AR1 + IL1R2 + CD177 + CD3D | 0.691 | 0.501 | 0.882 |
| 4 days | C3AR1 + IL1R2 + CD177 + CD3D + CTLA4 | 0.699 | 0.508 | 0.89 |
| 4 days | C3AR1 + IL1R2 + CD177 + CIITA | 0.734 | 0.554 | 0.915 |
| 4 days | C3AR1 + IL1R2 + CD177 + CIITA + CD3D | 0.734 | 0.551 | 0.918 |
| 4 days | C3AR1 + IL1R2 + CD177 + CIITA + CD3D + CTLA4 | 0.719 | 0.535 | 0.902 |
| 4 days | C3AR1 + IL1R2 + CD177 + CIITA + CTLA4 | 0.703 | 0.515 | 0.891 |
| 4 days | C3AR1 + IL1R2 + CD177 + CIITA + TAP2 | 0.715 | 0.525 | 0.905 |
| 4 days | C3AR1 + IL1R2 + CD177 + CIITA + TAP2 + CD3D | 0.742 | 0.561 | 0.924 |
| 4 days | C3AR1 + IL1R2 + CD177 + CIITA + TAP2 + CD3D + CTLA4 | 0.723 | 0.539 | 0.907 |
| 4 days | C3AR1 + IL1R2 + CD177 + CIITA + TAP2 + CTLA4 | 0.727 | 0.544 | 0.91 |
| 4 days | C3AR1 + IL1R2 + CD177 + CTLA4 | 0.691 | 0.499 | 0.884 |
| 4 days | C3AR1 + IL1R2 + CD177 + CX3CR1 | 0.691 | 0.501 | 0.881 |
| 4 days | C3AR1 + IL1R2 + CD177 + CX3CR1 + CD3D | 0.699 | 0.51 | 0.888 |
| 4 days | C3AR1 + IL1R2 + CD177 + CX3CR1 + CD3D + CTLA4 | 0.688 | 0.493 | 0.882 |
| 4 days | C3AR1 + IL1R2 + CD177 + CX3CR1 + CIITA | 0.77 | 0.597 | 0.942 |
| 4 days | C3AR1 + IL1R2 + CD177 + CX3CR1 + CIITA + CD3D | 0.715 | 0.519 | 0.911 |
| 4 days | C3AR1 + IL1R2 + CD177 + CX3CR1 + CIITA + CD3D + CTLA4 | 0.715 | 0.519 | 0.911 |
| 4 days | C3AR1 + IL1R2 + CD177 + CX3CR1 + CIITA + CTLA4 | 0.711 | 0.52 | 0.902 |
| 4 days | C3AR1 + IL1R2 + CD177 + CX3CR1 + CIITA + TAP2 | 0.777 | 0.615 | 0.939 |
| 4 days | C3AR1 + IL1R2 + CD177 + CX3CR1 + CIITA + TAP2 + CD3D | 0.789 | 0.629 | 0.949 |
| 4 days | C3AR1 + IL1R2 + CD177 + CX3CR1 + CIITA + TAP2 + CD3D + CTLA4 | 0.777 | 0.612 | 0.942 |
| 4 days | C3AR1 + IL1R2 + CD177 + CX3CR1 + CIITA + TAP2 + CTLA4 | 0.781 | 0.62 | 0.943 |
| 4 days | C3AR1 + IL1R2 + CD177 + CX3CR1 + CTLA4 | 0.711 | 0.522 | 0.9 |
| 4 days | C3AR1 + IL1R2 + CD177 + CX3CR1 + TAP2 | 0.727 | 0.547 | 0.906 |
| 4 days | C3AR1 + IL1R2 + CD177 + CX3CR1 + TAP2 + CD3D | 0.734 | 0.559 | 0.909 |
| 4 days | C3AR1 + IL1R2 + CD177 + CX3CR1 + TAP2 + CD3D + CTLA4 | 0.734 | 0.56 | 0.908 |
| 4 days | C3AR1 + IL1R2 + CD177 + CX3CR1 + TAP2 + CTLA4 | 0.734 | 0.56 | 0.908 |
| 4 days | C3AR1 + IL1R2 + CD177 + S100A9 | 0.82 | 0.663 | 0.978 |
| 4 days | C3AR1 + IL1R2 + CD177 + S100A9 + CD3D | 0.824 | 0.676 | 0.972 |
| 4 days | C3AR1 + IL1R2 + CD177 + S100A9 + CD3D + CTLA4 | 0.82 | 0.67 | 0.971 |
| 4 days | C3AR1 + IL1R2 + CD177 + S100A9 + CIITA | 0.816 | 0.658 | 0.975 |
| 4 days | C3AR1 + IL1R2 + CD177 + S100A9 + CIITA + CD3D | 0.832 | 0.686 | 0.978 |
| 4 days | C3AR1 + IL1R2 + CD177 + S100A9 + CIITA + CD3D + CTLA4 | 0.836 | 0.692 | 0.979 |
| 4 days | C3AR1 + IL1R2 + CD177 + S100A9 + CIITA + CTLA4 | 0.828 | 0.677 | 0.979 |
| 4 days | C3AR1 + IL1R2 + CD177 + S100A9 + CIITA + TAP2 | 0.832 | 0.679 | 0.985 |
| 4 days | C3AR1 + IL1R2 + CD177 + S100A9 + CIITA + TAP2 + CD3D | 0.832 | 0.688 | 0.976 |
| 4 days | C3AR1 + IL1R2 + CD177 + S100A9 + CIITA + TAP2 + CD3D + CTLA4 | 0.844 | 0.705 | 0.983 |
| 4 days | C3AR1 + IL1R2 + CD177 + S100A9 + CIITA + TAP2 + CTLA4 | 0.828 | 0.677 | 0.979 |
| 4 days | C3AR1 + IL1R2 + CD177 + S100A9 + CTLA4 | 0.828 | 0.677 | 0.979 |
| 4 days | C3AR1 + IL1R2 + CD177 + S100A9 + CX3CR1 | 0.859 | 0.716 | 1 |
| 4 days | C3AR1 + IL1R2 + CD177 + S100A9 + CX3CR1 + CD3D | 0.891 | 0.766 | 1 |
| 4 days | C3AR1 + IL1R2 + CD177 + S100A9 + CX3CR1 + CD3D + CTLA4 | 0.887 | 0.764 | 1 |
| 4 days | C3AR1 + IL1R2 + CD177 + S100A9 + CX3CR1 + CIITA | 0.867 | 0.731 | 1 |
| 4 days | C3AR1 + IL1R2 + CD177 + S100A9 + CX3CR1 + CIITA + CD3D | 0.895 | 0.774 | 1 |
| 4 days | C3AR1 + IL1R2 + CD177 + S100A9 + CX3CR1 + CIITA + CD3D + CTLA4 | 0.895 | 0.779 | 1 |
| 4 days | C3AR1 + IL1R2 + CD177 + S100A9 + CX3CR1 + CIITA + CTLA4 | 0.867 | 0.728 | 1 |
| 4 days | C3AR1 + IL1R2 + CD177 + S100A9 + CX3CR1 + CIITA + TAP2 | 0.883 | 0.751 | 1 |
| 4 days | C3AR1 + IL1R2 + CD177 + S100A9 + CX3CR1 + CIITA + TAP2 + CD3D | 0.906 | 0.796 | 1 |
| 4 days | C3AR1 + IL1R2 + CD177 + S100A9 + CX3CR1 + CIITA + TAP2 + CD3D + CTLA4 | 0.898 | 0.79 | 1 |
| 4 days | C3AR1 + IL1R2 + CD177 + S100A9 + CX3CR1 + CIITA + TAP2 + CTLA4 | 0.883 | 0.751 | 1 |
| 4 days | C3AR1 + IL1R2 + CD177 + S100A9 + CX3CR1 + CTLA4 | 0.863 | 0.722 | 1 |
| 4 days | C3AR1 + IL1R2 + CD177 + S100A9 + CX3CR1 + TAP2 | 0.871 | 0.735 | 1 |
| 4 days | C3AR1 + IL1R2 + CD177 + S100A9 + CX3CR1 + TAP2 + CD3D | 0.898 | 0.781 | 1 |
| 4 days | C3AR1 + IL1R2 + CD177 + S100A9 + CX3CR1 + TAP2 + CD3D + CTLA4 | 0.895 | 0.782 | 1 |
| 4 days | C3AR1 + IL1R2 + CD177 + S100A9 + CX3CR1 + TAP2 + CTLA4 | 0.875 | 0.737 | 1 |
| 4 days | C3AR1 + IL1R2 + CD177 + S100A9 + TAP2 | 0.832 | 0.679 | 0.985 |
| 4 days | C3AR1 + IL1R2 + CD177 + S100A9 + TAP2 + CD3D | 0.832 | 0.69 | 0.975 |
| 4 days | C3AR1 + IL1R2 + CD177 + S100A9 + TAP2 + CD3D + CTLA4 | 0.848 | 0.712 | 0.983 |
| 4 days | C3AR1 + IL1R2 + CD177 + S100A9 + TAP2 + CTLA4 | 0.82 | 0.667 | 0.974 |
| 4 days | C3AR1 + IL1R2 + CD177 + TAP2 | 0.707 | 0.512 | 0.902 |
| 4 days | C3AR1 + IL1R2 + CD177 + TAP2 + CD3D | 0.707 | 0.513 | 0.902 |
| 4 days | C3AR1 + IL1R2 + CD177 + TAP2 + CD3D + CTLA4 | 0.73 | 0.551 | 0.91 |
| 4 days | C3AR1 + IL1R2 + CD177 + TAP2 + CTLA4 | 0.73 | 0.552 | 0.909 |
| 4 days | C3AR1 + IL1R2 + CD74 | 0.666 | 0.568 | 0.763 |
| 4 days | C3AR1 + IL1R2 + CD74 + CD177 | 0.723 | 0.541 | 0.904 |
| 4 days | C3AR1 + IL1R2 + CD74 + CD177 + CD3D | 0.75 | 0.579 | 0.921 |
| 4 days | C3AR1 + IL1R2 + CD74 + CD177 + CD3D + CTLA4 | 0.758 | 0.589 | 0.926 |
| 4 days | C3AR1 + IL1R2 + CD74 + CD177 + CIITA | 0.73 | 0.549 | 0.912 |
| 4 days | C3AR1 + IL1R2 + CD74 + CD177 + CIITA + CD3D | 0.738 | 0.565 | 0.912 |
| 4 days | C3AR1 + IL1R2 + CD74 + CD177 + CIITA + CD3D + CTLA4 | 0.742 | 0.569 | 0.915 |
| 4 days | C3AR1 + IL1R2 + CD74 + CD177 + CIITA + CTLA4 | 0.746 | 0.573 | 0.919 |
| 4 days | C3AR1 + IL1R2 + CD74 + CD177 + CIITA + TAP2 | 0.738 | 0.562 | 0.915 |
| 4 days | C3AR1 + IL1R2 + CD74 + CD177 + CIITA + TAP2 + CD3D | 0.742 | 0.568 | 0.916 |
| 4 days | C3AR1 + IL1R2 + CD74 + CD177 + CIITA + TAP2 + CD3D + CTLA4 | 0.754 | 0.585 | 0.922 |
| 4 days | C3AR1 + IL1R2 + CD74 + CD177 + CIITA + TAP2 + CTLA4 | 0.734 | 0.557 | 0.912 |
| 4 days | C3AR1 + IL1R2 + CD74 + CD177 + CTLA4 | 0.738 | 0.561 | 0.916 |
| 4 days | C3AR1 + IL1R2 + CD74 + CD177 + CX3CR1 | 0.738 | 0.561 | 0.915 |
| 4 days | C3AR1 + IL1R2 + CD74 + CD177 + CX3CR1 + CD3D | 0.758 | 0.588 | 0.927 |
| 4 days | C3AR1 + IL1R2 + CD74 + CD177 + CX3CR1 + CD3D + CTLA4 | 0.758 | 0.586 | 0.929 |
| 4 days | C3AR1 + IL1R2 + CD74 + CD177 + CX3CR1 + CIITA | 0.746 | 0.568 | 0.924 |
| 4 days | C3AR1 + IL1R2 + CD74 + CD177 + CX3CR1 + CIITA + CD3D | 0.762 | 0.593 | 0.931 |
| 4 days | C3AR1 + IL1R2 + CD74 + CD177 + CX3CR1 + CIITA + CD3D + CTLA4 | 0.766 | 0.601 | 0.93 |
| 4 days | C3AR1 + IL1R2 + CD74 + CD177 + CX3CR1 + CIITA + CTLA4 | 0.738 | 0.561 | 0.916 |
| 4 days | C3AR1 + IL1R2 + CD74 + CD177 + CX3CR1 + CIITA + TAP2 | 0.77 | 0.602 | 0.937 |
| 4 days | C3AR1 + IL1R2 + CD74 + CD177 + CX3CR1 + CIITA + TAP2 + CD3D | 0.789 | 0.625 | 0.953 |
| 4 days | C3AR1 + IL1R2 + CD74 + CD177 + CX3CR1 + CIITA + TAP2 + CD3D + CTLA4 | 0.789 | 0.625 | 0.953 |
| 4 days | C3AR1 + IL1R2 + CD74 + CD177 + CX3CR1 + CIITA + TAP2 + CTLA4 | 0.781 | 0.618 | 0.944 |
| 4 days | C3AR1 + IL1R2 + CD74 + CD177 + CX3CR1 + CTLA4 | 0.75 | 0.573 | 0.927 |
| 4 days | C3AR1 + IL1R2 + CD74 + CD177 + CX3CR1 + TAP2 | 0.777 | 0.611 | 0.944 |
| 4 days | C3AR1 + IL1R2 + CD74 + CD177 + CX3CR1 + TAP2 + CD3D | 0.777 | 0.614 | 0.941 |
| 4 days | C3AR1 + IL1R2 + CD74 + CD177 + CX3CR1 + TAP2 + CD3D + CTLA4 | 0.812 | 0.656 | 0.969 |
| 4 days | C3AR1 + IL1R2 + CD74 + CD177 + CX3CR1 + TAP2 + CTLA4 | 0.797 | 0.638 | 0.955 |
| 4 days | C3AR1 + IL1R2 + CD74 + CD177 + S100A9 | 0.828 | 0.674 | 0.982 |
| 4 days | C3AR1 + IL1R2 + CD74 + CD177 + S100A9 + CD3D | 0.828 | 0.683 | 0.973 |
| 4 days | C3AR1 + IL1R2 + CD74 + CD177 + S100A9 + CD3D + CTLA4 | 0.828 | 0.683 | 0.973 |
| 4 days | C3AR1 + IL1R2 + CD74 + CD177 + S100A9 + CIITA | 0.828 | 0.676 | 0.98 |
| 4 days | C3AR1 + IL1R2 + CD74 + CD177 + S100A9 + CIITA + CD3D | 0.824 | 0.676 | 0.972 |
| 4 days | C3AR1 + IL1R2 + CD74 + CD177 + S100A9 + CIITA + CD3D + CTLA4 | 0.824 | 0.676 | 0.972 |
| 4 days | C3AR1 + IL1R2 + CD74 + CD177 + S100A9 + CIITA + CTLA4 | 0.828 | 0.677 | 0.979 |
| 4 days | C3AR1 + IL1R2 + CD74 + CD177 + S100A9 + CIITA + TAP2 | 0.828 | 0.674 | 0.982 |
| 4 days | C3AR1 + IL1R2 + CD74 + CD177 + S100A9 + CIITA + TAP2 + CD3D | 0.836 | 0.694 | 0.978 |
| 4 days | C3AR1 + IL1R2 + CD74 + CD177 + S100A9 + CIITA + TAP2 + CD3D + CTLA4 | 0.844 | 0.705 | 0.983 |
| 4 days | C3AR1 + IL1R2 + CD74 + CD177 + S100A9 + CIITA + TAP2 + CTLA4 | 0.828 | 0.677 | 0.979 |
| 4 days | C3AR1 + IL1R2 + CD74 + CD177 + S100A9 + CTLA4 | 0.828 | 0.677 | 0.979 |
| 4 days | C3AR1 + IL1R2 + CD74 + CD177 + S100A9 + CX3CR1 | 0.859 | 0.713 | 1 |
| 4 days | C3AR1 + IL1R2 + CD74 + CD177 + S100A9 + CX3CR1 + CD3D | 0.891 | 0.766 | 1 |
| 4 days | C3AR1 + IL1R2 + CD74 + CD177 + S100A9 + CX3CR1 + CD3D + CTLA4 | 0.895 | 0.775 | 1 |
| 4 days | C3AR1 + IL1R2 + CD74 + CD177 + S100A9 + CX3CR1 + CIITA | 0.867 | 0.734 | 1 |
| 4 days | C3AR1 + IL1R2 + CD74 + CD177 + S100A9 + CX3CR1 + CIITA + CD3D | 0.902 | 0.788 | 1 |
| 4 days | C3AR1 + IL1R2 + CD74 + CD177 + S100A9 + CX3CR1 + CIITA + CD3D + CTLA4 | 0.895 | 0.782 | 1 |
| 4 days | C3AR1 + IL1R2 + CD74 + CD177 + S100A9 + CX3CR1 + CIITA + CTLA4 | 0.871 | 0.74 | 1 |
| 4 days | C3AR1 + IL1R2 + CD74 + CD177 + S100A9 + CX3CR1 + CIITA + TAP2 | 0.887 | 0.762 | 1 |
| 4 days | C3AR1 + IL1R2 + CD74 + CD177 + S100A9 + CX3CR1 + CIITA + TAP2 + CD3D | 0.902 | 0.792 | 1 |
| 4 days | C3AR1 + IL1R2 + CD74 + CD177 + S100A9 + CX3CR1 + CIITA + TAP2 + CD3D + | 0.906 | 0.804 | 1 |
| | CTLA4 | | | |
| 4 days | C3AR1 + IL1R2 + CD74 + CD177 + S100A9 + CX3CR1 + CIITA + TAP2 + CTLA4 | 0.887 | 0.759 | 1 |
| 4 days | C3AR1 + IL1R2 + CD74 + CD177 + S100A9 + CX3CR1 + CTLA4 | 0.863 | 0.722 | 1 |
| 4 days | C3AR1 + IL1R2 + CD74 + CD177 + S100A9 + CX3CR1 + TAP2 | 0.871 | 0.735 | 1 |
| 4 days | C3AR1 + IL1R2 + CD74 + CD177 + S100A9 + CX3CR1 + TAP2 + CD3D | 0.898 | 0.781 | 1 |
| 4 days | C3AR1 + IL1R2 + CD74 + CD177 + S100A9 + CX3CR1 + TAP2 + CD3D + CTLA4 | 0.891 | 0.772 | 1 |
| 4 days | C3AR1 + IL1R2 + CD74 + CD177 + S100A9 + CX3CR1 + TAP2 + CTLA4 | 0.875 | 0.737 | 1 |
| 4 days | C3AR1 + IL1R2 + CD74 + CD177 + S100A9 + TAP2 | 0.824 | 0.669 | 0.979 |
| 4 days | C3AR1 + IL1R2 + CD74 + CD177 + S100A9 + TAP2 + CD3D | 0.836 | 0.694 | 0.978 |
| 4 days | C3AR1 + IL1R2 + CD74 + CD177 + S100A9 + TAP2 + CD3D + CTLA4 | 0.848 | 0.712 | 0.983 |
| 4 days | C3AR1 + IL1R2 + CD74 + CD177 + S100A9 + TAP2 + CTLA4 | 0.828 | 0.677 | 0.979 |
| 4 days | C3AR1 + IL1R2 + CD74 + CD177 + TAP2 | 0.727 | 0.544 | 0.909 |
| 4 days | C3AR1 + IL1R2 + CD74 + CD177 + TAP2 + CD3D | 0.746 | 0.573 | 0.92 |
| 4 days | C3AR1 + IL1R2 + CD74 + CD177 + TAP2 + CD3D + CTLA4 | 0.734 | 0.556 | 0.913 |
| 4 days | C3AR1 + IL1R2 + CD74 + CD177 + TAP2 + CTLA4 | 0.738 | 0.56 | 0.916 |
| 4 days | C3AR1 + IL1R2 + CD74 + CD3D | 0.672 | 0.48 | 0.863 |
| 4 days | C3AR1 + IL1R2 + CD74 + CD3D + CTLA4 | 0.703 | 0.52 | 0.886 |
| 4 days | C3AR1 + IL1R2 + CD74 + CIITA | 0.691 | 0.503 | 0.88 |
| 4 days | C3AR1 + IL1R2 + CD74 + CIITA + CD3D | 0.684 | 0.495 | 0.873 |
| 4 days | C3AR1 + IL1R2 + CD74 + CIITA + CD3D + CTLA4 | 0.711 | 0.529 | 0.893 |
| 4 days | C3AR1 + IL1R2 + CD74 + CIITA + CTLA4 | 0.691 | 0.506 | 0.877 |
| 4 days | C3AR1 + IL1R2 + CD74 + CIITA + TAP2 | 0.707 | 0.522 | 0.892 |
| 4 days | C3AR1 + IL1R2 + CD74 + CIITA + TAP2 + CD3D | 0.711 | 0.528 | 0.894 |
| 4 days | C3AR1 + IL1R2 + CD74 + CIITA + TAP2 + CD3D + CTLA4 | 0.723 | 0.543 | 0.902 |
| 4 days | C3AR1 + IL1R2 + CD74 + CIITA + TAP2 + CTLA4 | 0.711 | 0.528 | 0.894 |
| 4 days | C3AR1 + IL1R2 + CD74 + CTLA4 | 0.68 | 0.49 | 0.869 |
| 4 days | C3AR1 + IL1R2 + CD74 + CX3CR1 | 0.711 | 0.514 | 0.908 |
| 4 days | C3AR1 + IL1R2 + CD74 + CX3CR1 + CD3D | 0.715 | 0.532 | 0.897 |
| 4 days | C3AR1 + IL1R2 + CD74 + CX3CR1 + CD3D + CTLA4 | 0.73 | 0.55 | 0.911 |
| 4 days | C3AR1 + IL1R2 + CD74 + CX3CR1 + CIITA | 0.75 | 0.56 | 0.94 |
| 4 days | C3AR1 + IL1R2 + CD74 + CX3CR1 + CIITA + CD3D | 0.75 | 0.571 | 0.929 |
| 4 days | C3AR1 + IL1R2 + CD74 + CX3CR1 + CIITA + CD3D + CTLA4 | 0.754 | 0.577 | 0.93 |
| 4 days | C3AR1 + IL1R2 + CD74 + CX3CR1 + CIITA + CTLA4 | 0.738 | 0.551 | 0.926 |
| 4 days | C3AR1 + IL1R2 + CD74 + CX3CR1 + CIITA + TAP2 | 0.781 | 0.611 | 0.952 |
| 4 days | C3AR1 + IL1R2 + CD74 + CX3CR1 + CIITA + TAP2 + CD3D | 0.77 | 0.598 | 0.941 |
| 4 days | C3AR1 + IL1R2 + CD74 + CX3CR1 + CIITA + TAP2 + CD3D + CTLA4 | 0.785 | 0.62 | 0.95 |
| 4 days | C3AR1 + IL1R2 + CD74 + CX3CR1 + CIITA + TAP2 + CTLA4 | 0.77 | 0.595 | 0.944 |
| 4 days | C3AR1 + IL1R2 + CD74 + CX3CR1 + CTLA4 | 0.734 | 0.551 | 0.918 |
| 4 days | C3AR1 + IL1R2 + CD74 + CX3CR1 + TAP2 | 0.754 | 0.579 | 0.929 |
| 4 days | C3AR1 + IL1R2 + CD74 + CX3CR1 + TAP2 + CD3D | 0.742 | 0.567 | 0.917 |
| 4 days | C3AR1 + IL1R2 + CD74 + CX3CR1 + TAP2 + CD3D + CTLA4 | 0.77 | 0.604 | 0.936 |
| 4 days | C3AR1 + IL1R2 + CD74 + CX3CR1 + TAP2 + CTLA4 | 0.781 | 0.615 | 0.947 |
| 4 days | C3AR1 + IL1R2 + CD74 + IFNG | 0.727 | 0.547 | 0.906 |
| 4 days | C3AR1 + IL1R2 + CD74 + IFNG + CD177 | 0.738 | 0.563 | 0.914 |
| 4 days | C3AR1 + IL1R2 + CD74 + IFNG + CD177 + CD3D | 0.75 | 0.577 | 0.923 |
| 4 days | C3AR1 + IL1R2 + CD74 + IFNG + CD177 + CD3D + CTLA4 | 0.762 | 0.594 | 0.929 |
| 4 days | C3AR1 + IL1R2 + CD74 + IFNG + CD177 + CIITA | 0.738 | 0.563 | 0.913 |
| 4 days | C3AR1 + IL1R2 + CD74 + IFNG + CD177 + CIITA + CD3D | 0.75 | 0.578 | 0.922 |
| 4 days | C3AR1 + IL1R2 + CD74 + IFNG + CD177 + CIITA + CD3D + CTLA4 | 0.758 | 0.584 | 0.932 |
| 4 days | C3AR1 + IL1R2 + CD74 + IFNG + CD177 + CIITA + CTLA4 | 0.754 | 0.58 | 0.928 |
| 4 days | C3AR1 + IL1R2 + CD74 + IFNG + CD177 + CIITA + TAP2 | 0.742 | 0.568 | 0.917 |
| 4 days | C3AR1 + IL1R2 + CD74 + IFNG + CD177 + CIITA + TAP2 + CD3D | 0.754 | 0.584 | 0.924 |
| 4 days | C3AR1 + IL1R2 + CD74 + IFNG + CD177 + CIITA + TAP2 + CD3D + CTLA4 | 0.758 | 0.584 | 0.932 |
| 4 days | C3AR1 + IL1R2 + CD74 + IFNG + CD177 + CIITA + TAP2 + CTLA4 | 0.754 | 0.58 | 0.928 |
| 4 days | C3AR1 + IL1R2 + CD74 + IFNG + CD177 + CTLA4 | 0.758 | 0.589 | 0.927 |
| 4 days | C3AR1 + IL1R2 + CD74 + IFNG + CD177 + CX3CR1 | 0.867 | 0.737 | 0.997 |
| 4 days | C3AR1 + IL1R2 + CD74 + IFNG + CD177 + CX3CR1 + CD3D | 0.871 | 0.752 | 0.99 |
| 4 days | C3AR1 + IL1R2 + CD74 + IFNG + CD177 + CX3CR1 + CD3D + CTLA4 | 0.914 | 0.821 | 1 |
| 4 days | C3AR1 + IL1R2 + CD74 + IFNG + CD177 + CX3CR1 + CIITA | 0.875 | 0.753 | 0.997 |
| 4 days | C3AR1 + IL1R2 + CD74 + IFNG + CD177 + CX3CR1 + CIITA + CD3D | 0.871 | 0.753 | 0.989 |
| 4 days | C3AR1 + IL1R2 + CD74 + IFNG + CD177 + CX3CR1 + CIITA + CD3D + CTLA4 | 0.918 | 0.828 | 1 |
| 4 days | C3AR1 + IL1R2 + CD74 + IFNG + CD177 + CX3CR1 + CIITA + CTLA4 | 0.914 | 0.82 | 1 |
| 4 days | C3AR1 + IL1R2 + CD74 + IFNG + CD177 + CX3CR1 + CIITA + TAP2 | 0.855 | 0.725 | 0.986 |
| 4 days | C3AR1 + IL1R2 + CD74 + IFNG + CD177 + CX3CR1 + CIITA + TAP2 + CD3D | 0.871 | 0.752 | 0.99 |
| 4 days | C3AR1 + IL1R2 + CD74 + IFNG + CD177 + CX3CR1 + CIITA + TAP2 + CD3D + CTLA4 | 0.891 | 0.782 | 1 |
| 4 days | C3AR1 + IL1R2 + CD74 + IFNG + CD177 + CX3CR1 + CIITA + TAP2 + CTLA4 | 0.895 | 0.788 | 1 |
| 4 days | C3AR1 + IL1R2 + CD74 + IFNG + CD177 + CX3CR1 + CTLA4 | 0.902 | 0.801 | 1 |
| 4 days | C3AR1 + IL1R2 + CD74 + IFNG + CD177 + CX3CR1 + TAP2 | 0.863 | 0.738 | 0.989 |
| 4 days | C3AR1 + IL1R2 + CD74 + IFNG + CD177 + CX3CR1 + TAP2 + CD3D | 0.887 | 0.777 | 0.997 |
| 4 days | C3AR1 + IL1R2 + CD74 + IFNG + CD177 + CX3CR1 + TAP2 + CD3D + CTLA4 | 0.891 | 0.78 | 1 |
| 4 days | C3AR1 + IL1R2 + CD74 + IFNG + CD177 + CX3CR1 + TAP2 + CTLA4 | 0.891 | 0.78 | 1 |
| 4 days | C3AR1 + IL1R2 + CD74 + IFNG + CD177 + S100A9 | 0.828 | 0.683 | 0.973 |
| 4 days | C3AR1 + IL1R2 + CD74 + IFNG + CD177 + S100A9 + CD3D | 0.836 | 0.697 | 0.975 |
| 4 days | C3AR1 + IL1R2 + CD74 + IFNG + CD177 + S100A9 + CD3D + CTLA4 | 0.844 | 0.71 | 0.977 |
| 4 days | C3AR1 + IL1R2 + CD74 + IFNG + CD177 + S100A9 + CIITA | 0.824 | 0.673 | 0.975 |
| 4 days | C3AR1 + IL1R2 + CD74 + IFNG + CD177 + S100A9 + CIITA + CD3D | 0.844 | 0.71 | 0.978 |
| 4 days | C3AR1 + IL1R2 + CD74 + IFNG + CD177 + S100A9 + CIITA + CD3D + CTLA4 | 0.84 | 0.705 | 0.975 |
| 4 days | C3AR1 + IL1R2 + CD74 + IFNG + CD177 + S100A9 + CIITA + CTLA4 | 0.82 | 0.675 | 0.966 |
| 4 days | C3AR1 + IL1R2 + CD74 + IFNG + CD177 + S100A9 + CIITA + TAP2 | 0.855 | 0.718 | 0.993 |
| 4 days | C3AR1 + IL1R2 + CD74 + IFNG + CD177 + S100A9 + CIITA + TAP2 + CD3D | 0.855 | 0.726 | 0.985 |
| 4 days | C3AR1 + IL1R2 + CD74 + IFNG + CD177 + S100A9 + CIITA + TAP2 + CD3D + CTLA4 | 0.848 | 0.713 | 0.982 |
| 4 days | C3AR1 + IL1R2 + CD74 + IFNG + CD177 + S100A9 + CIITA + TAP2 + CTLA4 | 0.84 | 0.701 | 0.979 |
| 4 days | C3AR1 + IL1R2 + CD74 + IFNG + CD177 + S100A9 + CTLA4 | 0.828 | 0.688 | 0.969 |
| 4 days | C3AR1 + IL1R2 + CD74 + IFNG + CD177 + S100A9 + CX3CR1 | 0.941 | 0.864 | 1 |
| 4 days | C3AR1 + IL1R2 + CD74 + IFNG + CD177 + S100A9 + CX3CR1 + CD3D | 0.977 | 0.928 | 1 |
| 4 days | C3AR1 + IL1R2 + CD74 + IFNG + CD177 + S100A9 + CX3CR1 + CIITA | 0.945 | 0.871 | 1 |
| 4 days | C3AR1 + IL1R2 + CD74 + IFNG + CD177 + S100A9 + CX3CR1 + CIITA + CD3D | 0.984 | 0.951 | 1 |
| 4 days | C3AR1 + IL1R2 + CD74 + IFNG + CD177 + S100A9 + CX3CR1 + CIITA + TAP2 | 0.945 | 0.871 | 1 |
| 4 days | C3AR1 + IL1R2 + CD74 + IFNG + CD177 + S100A9 + CX3CR1 + TAP2 | 0.941 | 0.864 | 1 |
| 4 days | C3AR1 + IL1R2 + CD74 + IFNG + CD177 + S100A9 + TAP2 | 0.84 | 0.7 | 0.98 |
| 4 days | C3AR1 + IL1R2 + CD74 + IFNG + CD177 + S100A9 + TAP2 + CD3D | 0.848 | 0.714 | 0.981 |
| 4 days | C3AR1 + IL1R2 + CD74 + IFNG + CD177 + S100A9 + TAP2 + CD3D + CTLA4 | 0.844 | 0.709 | 0.979 |
| 4 days | C3AR1 + IL1R2 + CD74 + IFNG + CD177 + S100A9 + TAP2 + CTLA4 | 0.824 | 0.68 | 0.969 |
| 4 days | C3AR1 + IL1R2 + CD74 + IFNG + CD177 + TAP2 | 0.738 | 0.563 | 0.914 |
| 4 days | C3AR1 + IL1R2 + CD74 + IFNG + CD177 + TAP2 + CD3D | 0.746 | 0.572 | 0.92 |
| 4 days | C3AR1 + IL1R2 + CD74 + IFNG + CD177 + TAP2 + CD3D + CTLA4 | 0.762 | 0.593 | 0.93 |
| 4 days | C3AR1 + IL1R2 + CD74 + IFNG + CD177 + TAP2 + CTLA4 | 0.758 | 0.589 | 0.927 |
| 4 days | C3AR1 + IL1R2 + CD74 + IFNG + CD3D | 0.727 | 0.546 | 0.907 |
| 4 days | C3AR1 + IL1R2 + CD74 + IFNG + CD3D + CTLA4 | 0.762 | 0.593 | 0.93 |
| 4 days | C3AR1 + IL1R2 + CD74 + IFNG + CIITA | 0.727 | 0.548 | 0.905 |
| 4 days | C3AR1 + IL1R2 + CD74 + IFNG + CIITA + CD3D | 0.734 | 0.558 | 0.911 |
| 4 days | C3AR1 + IL1R2 + CD74 + IFNG + CIITA + CD3D + CTLA4 | 0.758 | 0.584 | 0.932 |
| 4 days | C3AR1 + IL1R2 + CD74 + IFNG + CIITA + CTLA4 | 0.762 | 0.59 | 0.934 |
| 4 days | C3AR1 + IL1R2 + CD74 + IFNG + CIITA + TAP2 | 0.719 | 0.538 | 0.9 |
| 4 days | C3AR1 + IL1R2 + CD74 + IFNG + CIITA + TAP2 + CD3D | 0.73 | 0.553 | 0.908 |
| 4 days | C3AR1 + IL1R2 + CD74 + IFNG + CIITA + TAP2 + CD3D + CTLA4 | 0.758 | 0.584 | 0.932 |
| 4 days | C3AR1 + IL1R2 + CD74 + IFNG + CIITA + TAP2 + CTLA4 | 0.762 | 0.59 | 0.934 |
| 4 days | C3AR1 + IL1R2 + CD74 + IFNG + CTLA4 | 0.758 | 0.587 | 0.928 |
| 4 days | C3AR1 + IL1R2 + CD74 + IFNG + CX3CR1 | 0.867 | 0.737 | 0.997 |
| 4 days | C3AR1 + IL1R2 + CD74 + IFNG + CX3CR1 + CD3D | 0.875 | 0.758 | 0.992 |
| 4 days | C3AR1 + IL1R2 + CD74 + IFNG + CX3CR1 + CD3D + CTLA4 | 0.887 | 0.776 | 0.997 |
| 4 days | C3AR1 + IL1R2 + CD74 + IFNG + CX3CR1 + CIITA | 0.875 | 0.753 | 0.997 |
| 4 days | C3AR1 + IL1R2 + CD74 + IFNG + CX3CR1 + CIITA + CD3D | 0.875 | 0.758 | 0.992 |
| 4 days | C3AR1 + IL1R2 + CD74 + IFNG + CX3CR1 + CIITA + CD3D + CTLA4 | 0.883 | 0.769 | 0.996 |
| 4 days | C3AR1 + IL1R2 + CD74 + IFNG + CX3CR1 + CIITA + CTLA4 | 0.879 | 0.764 | 0.993 |
| 4 days | C3AR1 + IL1R2 + CD74 + IFNG + CX3CR1 + CIITA + TAP2 | 0.867 | 0.742 | 0.993 |
| 4 days | C3AR1 + IL1R2 + CD74 + IFNG + CX3CR1 + CIITA + TAP2 + CD3D | 0.879 | 0.763 | 0.995 |
| 4 days | C3AR1 + IL1R2 + CD74 + IFNG + CX3CR1 + CIITA + TAP2 + CD3D + CTLA4 | 0.879 | 0.763 | 0.995 |
| 4 days | C3AR1 + IL1R2 + CD74 + IFNG + CX3CR1 + CIITA + TAP2 + CTLA4 | 0.887 | 0.776 | 0.998 |
| 4 days | C3AR1 + IL1R2 + CD74 + IFNG + CX3CR1 + CTLA4 | 0.879 | 0.764 | 0.993 |
| 4 days | C3AR1 + IL1R2 + CD74 + IFNG + CX3CR1 + TAP2 | 0.859 | 0.732 | 0.986 |
| 4 days | C3AR1 + IL1R2 + CD74 + IFNG + CX3CR1 + TAP2 + CD3D | 0.883 | 0.771 | 0.995 |
| 4 days | C3AR1 + IL1R2 + CD74 + IFNG + CX3CR1 + TAP2 + CD3D + CTLA4 | 0.875 | 0.757 | 0.993 |
| 4 days | C3AR1 + IL1R2 + CD74 + IFNG + CX3CR1 + TAP2 + CTLA4 | 0.887 | 0.776 | 0.998 |
| 4 days | C3AR1 + IL1R2 + CD74 + IFNG + S100A9 | 0.816 | 0.666 | 0.967 |
| 4 days | C3AR1 + IL1R2 + CD74 + IFNG + S100A9 + CD3D | 0.852 | 0.719 | 0.984 |
| 4 days | C3AR1 + IL1R2 + CD74 + IFNG + S100A9 + CD3D + CTLA4 | 0.848 | 0.713 | 0.982 |
| 4 days | C3AR1 + IL1R2 + CD74 + IFNG + S100A9 + CIITA | 0.828 | 0.681 | 0.975 |
| 4 days | C3AR1 + IL1R2 + CD74 + IFNG + S100A9 + CIITA + CD3D | 0.848 | 0.712 | 0.983 |
| 4 days | C3AR1 + IL1R2 + CD74 + IFNG + S100A9 + CIITA + CD3D + CTLA4 | 0.844 | 0.706 | 0.982 |
| 4 days | C3AR1 + IL1R2 + CD74 + IFNG + S100A9 + CIITA + CTLA4 | 0.816 | 0.668 | 0.965 |
| 4 days | C3AR1 + IL1R2 + CD74 + IFNG + S100A9 + CIITA + TAP2 | 0.844 | 0.699 | 0.989 |
| 4 days | C3AR1 + IL1R2 + CD74 + IFNG + S100A9 + CIITA + TAP2 + CD3D | 0.852 | 0.716 | 0.987 |
| 4 days | C3AR1 + IL1R2 + CD74 + IFNG + S100A9 + CIITA + TAP2 + CD3D + CTLA4 | 0.848 | 0.711 | 0.984 |
| 4 days | C3AR1 + IL1R2 + CD74 + IFNG + S100A9 + CIITA + TAP2 + CTLA4 | 0.828 | 0.681 | 0.975 |
| 4 days | C3AR1 + IL1R2 + CD74 + IFNG + S100A9 + CTLA4 | 0.816 | 0.669 | 0.963 |
| 4 days | C3AR1 + IL1R2 + CD74 + IFNG + S100A9 + CX3CR1 | 0.926 | 0.838 | 1 |
| 4 days | C3AR1 + IL1R2 + CD74 + IFNG + S100A9 + CX3CR1 + CD3D | 0.945 | 0.876 | 1 |
| 4 days | C3AR1 + IL1R2 + CD74 + IFNG + S100A9 + CX3CR1 + CD3D + CTLA4 | 0.945 | 0.876 | 1 |
| 4 days | C3AR1 + IL1R2 + CD74 + IFNG + S100A9 + CX3CR1 + CIITA | 0.926 | 0.838 | 1 |
| 4 days | C3AR1 + IL1R2 + CD74 + IFNG + S100A9 + CX3CR1 + CIITA + CD3D | 0.945 | 0.873 | 1 |
| 4 days | C3AR1 + IL1R2 + CD74 + IFNG + S100A9 + CX3CR1 + CIITA + CD3D + CTLA4 | 0.949 | 0.882 | 1 |
| 4 days | C3AR1 + IL1R2 + CD74 + IFNG + S100A9 + CX3CR1 + CIITA + CTLA4 | 0.918 | 0.825 | 1 |
| 4 days | C3AR1 + IL1R2 + CD74 + IFNG + S100A9 + CX3CR1 + CIITA + TAP2 | 0.93 | 0.843 | 1 |
| 4 days | C3AR1 + IL1R2 + CD74 + IFNG + S100A9 + CX3CR1 + CIITA + TAP2 + CD3D | 0.949 | 0.883 | 1 |
| 4 days | C3AR1 + IL1R2 + CD74 + IFNG + S100A9 + CX3CR1 + CIITA + TAP2 + CD3D + CTLA4 | 0.949 | 0.881 | 1 |
| 4 days | C3AR1 + IL1R2 + CD74 + IFNG + S100A9 + CX3CR1 + CIITA + TAP2 + CTLA4 | 0.914 | 0.815 | 1 |
| 4 days | C3AR1 + IL1R2 + CD74 + IFNG + S100A9 + CX3CR1 + CTLA4 | 0.922 | 0.833 | 1 |
| 4 days | C3AR1 + IL1R2 + CD74 + IFNG + S100A9 + CX3CR1 + TAP2 | 0.93 | 0.843 | 1 |
| 4 days | C3AR1 + IL1R2 + CD74 + IFNG + S100A9 + CX3CR1 + TAP2 + CD3D | 0.938 | 0.862 | 1 |
| 4 days | C3AR1 + IL1R2 + CD74 + IFNG + S100A9 + CX3CR1 + TAP2 + CD3D + CTLA4 | 0.953 | 0.887 | 1 |
| 4 days | C3AR1 + IL1R2 + CD74 + IFNG + S100A9 + CX3CR1 + TAP2 + CTLA4 | 0.918 | 0.825 | 1 |
| 4 days | C3AR1 + IL1R2 + CD74 + IFNG + S100A9 + TAP2 | 0.832 | 0.685 | 0.979 |
| 4 days | C3AR1 + IL1R2 + CD74 + IFNG + S100A9 + TAP2 + CD3D | 0.848 | 0.709 | 0.986 |
| 4 days | C3AR1 + IL1R2 + CD74 + IFNG + S100A9 + TAP2 + CD3D + CTLA4 | 0.852 | 0.717 | 0.986 |
| 4 days | C3AR1 + IL1R2 + CD74 + IFNG + S100A9 + TAP2 + CTLA4 | 0.812 | 0.661 | 0.964 |
| 4 days | C3AR1 + IL1R2 + CD74 + IFNG + TAP2 | 0.719 | 0.537 | 0.9 |
| 4 days | C3AR1 + IL1R2 + CD74 + IFNG + TAP2 + CD3D | 0.73 | 0.552 | 0.909 |
| 4 days | C3AR1 + IL1R2 + CD74 + IFNG + TAP2 + CD3D + CTLA4 | 0.762 | 0.592 | 0.932 |
| 4 days | C3AR1 + IL1R2 + CD74 + IFNG + TAP2 + CTLA4 | 0.758 | 0.587 | 0.928 |
| 4 days | C3AR1 + IL1R2 + CD74 + S100A9 | 0.824 | 0.67 | 0.979 |
| 4 days | C3AR1 + IL1R2 + CD74 + S100A9 + CD3D | 0.844 | 0.708 | 0.979 |
| 4 days | C3AR1 + IL1R2 + CD74 + S100A9 + CD3D + CTLA4 | 0.844 | 0.708 | 0.979 |
| 4 days | C3AR1 + IL1R2 + CD74 + S100A9 + CIITA | 0.824 | 0.67 | 0.979 |
| 4 days | C3AR1 + IL1R2 + CD74 + S100A9 + CIITA + CD3D | 0.844 | 0.708 | 0.979 |
| 4 days | C3AR1 + IL1R2 + CD74 + S100A9 + CIITA + CD3D + CTLA4 | 0.84 | 0.703 | 0.977 |
| 4 days | C3AR1 + IL1R2 + CD74 + S100A9 + CIITA + CTLA4 | 0.816 | 0.661 | 0.972 |
| 4 days | C3AR1 + IL1R2 + CD74 + S100A9 + CIITA + TAP2 | 0.824 | 0.672 | 0.977 |
| 4 days | C3AR1 + IL1R2 + CD74 + S100A9 + CIITA + TAP2 + CD3D | 0.844 | 0.706 | 0.981 |
| 4 days | C3AR1 + IL1R2 + CD74 + S100A9 + CIITA + TAP2 + CD3D + CTLA4 | 0.844 | 0.706 | 0.982 |
| 4 days | C3AR1 + IL1R2 + CD74 + S100A9 + CIITA + TAP2 + CTLA4 | 0.816 | 0.661 | 0.972 |
| 4 days | C3AR1 + IL1R2 + CD74 + S100A9 + CTLA4 | 0.816 | 0.661 | 0.972 |
| 4 days | C3AR1 + IL1R2 + CD74 + S100A9 + CX3CR1 | 0.871 | 0.733 | 1 |
| 4 days | C3AR1 + IL1R2 + CD74 + S100A9 + CX3CR1 + CD3D | 0.898 | 0.781 | 1 |
| 4 days | C3AR1 + IL1R2 + CD74 + S100A9 + CX3CR1 + CD3D + CTLA4 | 0.891 | 0.766 | 1 |
| 4 days | C3AR1 + IL1R2 + CD74 + S100A9 + CX3CR1 + CIITA | 0.867 | 0.732 | 1 |
| 4 days | C3AR1 + IL1R2 + CD74 + S100A9 + CX3CR1 + CIITA + CD3D | 0.898 | 0.786 | 1 |
| 4 days | C3AR1 + IL1R2 + CD74 + S100A9 + CX3CR1 + CIITA + CD3D + CTLA4 | 0.902 | 0.791 | 1 |
| 4 days | C3AR1 + IL1R2 + CD74 + S100A9 + CX3CR1 + CIITA + CTLA4 | 0.875 | 0.742 | 1 |
| 4 days | C3AR1 + IL1R2 + CD74 + S100A9 + CX3CR1 + CIITA + TAP2 | 0.879 | 0.746 | 1 |
| 4 days | C3AR1 + IL1R2 + CD74 + S100A9 + CX3CR1 + CIITA + TAP2 + CD3D | 0.898 | 0.786 | 1 |
| 4 days | C3AR1 + IL1R2 + CD74 + S100A9 + CX3CR1 + CIITA + TAP2 + CD3D + CTLA4 | 0.922 | 0.826 | 1 |
| 4 days | C3AR1 + IL1R2 + CD74 + S100A9 + CX3CR1 + CIITA + TAP2 + CTLA4 | 0.883 | 0.754 | 1 |
| 4 days | C3AR1 + IL1R2 + CD74 + S100A9 + CX3CR1 + CTLA4 | 0.879 | 0.747 | 1 |
| 4 days | C3AR1 + IL1R2 + CD74 + S100A9 + CX3CR1 + TAP2 | 0.871 | 0.736 | 1 |
| 4 days | C3AR1 + IL1R2 + CD74 + S100A9 + CX3CR1 + TAP2 + CD3D | 0.898 | 0.781 | 1 |
| 4 days | C3AR1 + IL1R2 + CD74 + S100A9 + CX3CR1 + TAP2 + CD3D + CTLA4 | 0.891 | 0.768 | 1 |
| 4 days | C3AR1 + IL1R2 + CD74 + S100A9 + CX3CR1 + TAP2 + CTLA4 | 0.883 | 0.751 | 1 |
| 4 days | C3AR1 + IL1R2 + CD74 + S100A9 + TAP2 | 0.816 | 0.658 | 0.975 |
| 4 days | C3AR1 + IL1R2 + CD74 + S100A9 + TAP2 + CD3D | 0.844 | 0.706 | 0.981 |
| 4 days | C3AR1 + IL1R2 + CD74 + S100A9 + TAP2 + CD3D + CTLA4 | 0.848 | 0.713 | 0.983 |
| 4 days | C3AR1 + IL1R2 + CD74 + S100A9 + TAP2 + CTLA4 | 0.816 | 0.661 | 0.972 |
| 4 days | C3AR1 + IL1R2 + CD74 + TAP2 | 0.703 | 0.52 | 0.886 |
| 4 days | C3AR1 + IL1R2 + CD74 + TAP2 + CD3D | 0.711 | 0.529 | 0.893 |
| 4 days | C3AR1 + IL1R2 + CD74 + TAP2 + CD3D + CTLA4 | 0.715 | 0.531 | 0.898 |
| 4 days | C3AR1 + IL1R2 + CD74 + TAP2 + CTLA4 | 0.707 | 0.521 | 0.893 |
| 4 days | C3AR1 + IL1R2 + CIITA | 0.723 | 0.538 | 0.907 |
| 4 days | C3AR1 + IL1R2 + CIITA + CD3D | 0.707 | 0.52 | 0.894 |
| 4 days | C3AR1 + IL1R2 + CIITA + CD3D + CTLA4 | 0.676 | 0.481 | 0.871 |
| 4 days | C3AR1 + IL1R2 + CIITA + CTLA4 | 0.668 | 0.474 | 0.862 |
| 4 days | C3AR1 + IL1R2 + CIITA + TAP2 | 0.715 | 0.531 | 0.899 |
| 4 days | C3AR1 + IL1R2 + CIITA + TAP2 + CD3D | 0.723 | 0.543 | 0.903 |
| 4 days | C3AR1 + IL1R2 + CIITA + TAP2 + CD3D + CTLA4 | 0.707 | 0.523 | 0.891 |
| 4 days | C3AR1 + IL1R2 + CIITA + TAP2 + CTLA4 | 0.707 | 0.523 | 0.891 |
| 4 days | C3AR1 + IL1R2 + CX3CR1 | 0.676 | 0.479 | 0.872 |
| 4 days | C3AR1 + IL1R2 + CX3CR1 + CD3D | 0.664 | 0.465 | 0.863 |
| 4 days | C3AR1 + IL1R2 + CX3CR1 + CD3D + CTLA4 | 0.672 | 0.48 | 0.864 |
| 4 days | C3AR1 + IL1R2 + CX3CR1 + CIITA | 0.73 | 0.542 | 0.919 |
| 4 days | C3AR1 + IL1R2 + CX3CR1 + CIITA + CD3D | 0.703 | 0.51 | 0.896 |
| 4 days | C3AR1 + IL1R2 + CX3CR1 + CIITA + CD3D + CTLA4 | 0.711 | 0.52 | 0.902 |
| 4 days | C3AR1 + IL1R2 + CX3CR1 + CIITA + CTLA4 | 0.723 | 0.53 | 0.915 |
| 4 days | C3AR1 + IL1R2 + CX3CR1 + CIITA + TAP2 | 0.758 | 0.584 | 0.932 |
| 4 days | C3AR1 + IL1R2 + CX3CR1 + CIITA + TAP2 + CD3D | 0.777 | 0.615 | 0.94 |
| 4 days | C3AR1 + IL1R2 + CX3CR1 + CIITA + TAP2 + CD3D + CTLA4 | 0.766 | 0.599 | 0.933 |
| 4 days | C3AR1 + IL1R2 + CX3CR1 + CIITA + TAP2 + CTLA4 | 0.754 | 0.584 | 0.924 |
| 4 days | C3AR1 + IL1R2 + CX3CR1 + CTLA4 | 0.672 | 0.479 | 0.864 |
| 4 days | C3AR1 + IL1R2 + CX3CR1 + TAP2 | 0.707 | 0.52 | 0.894 |
| 4 days | C3AR1 + IL1R2 + CX3CR1 + TAP2 + CD3D | 0.699 | 0.515 | 0.883 |
| 4 days | C3AR1 + IL1R2 + CX3CR1 + TAP2 + CD3D + CTLA4 | 0.715 | 0.535 | 0.894 |
| 4 days | C3AR1 + IL1R2 + CX3CR1 + TAP2 + CTLA4 | 0.719 | 0.54 | 0.897 |
| 4 days | C3AR1 + IL1R2 + IFNG | 0.715 | 0.527 | 0.902 |
| 4 days | C3AR1 + IL1R2 + IFNG + CD177 | 0.73 | 0.548 | 0.913 |
| 4 days | C3AR1 + IL1R2 + IFNG + CD177 + CD3D | 0.754 | 0.577 | 0.931 |
| 4 days | C3AR1 + IL1R2 + IFNG + CD177 + CD3D + CTLA4 | 0.75 | 0.579 | 0.921 |
| 4 days | C3AR1 + IL1R2 + IFNG + CD177 + CIITA | 0.734 | 0.556 | 0.913 |
| 4 days | C3AR1 + IL1R2 + IFNG + CD177 + CIITA + CD3D | 0.746 | 0.567 | 0.925 |
| 4 days | C3AR1 + IL1R2 + IFNG + CD177 + CIITA + CD3D + CTLA4 | 0.75 | 0.579 | 0.921 |
| 4 days | C3AR1 + IL1R2 + IFNG + CD177 + CIITA + CTLA4 | 0.75 | 0.58 | 0.92 |
| 4 days | C3AR1 + IL1R2 + IFNG + CD177 + CIITA + TAP2 | 0.746 | 0.568 | 0.924 |
| 4 days | C3AR1 + IL1R2 + IFNG + CD177 + CIITA + TAP2 + CD3D | 0.75 | 0.574 | 0.926 |
| 4 days | C3AR1 + IL1R2 + IFNG + CD177 + CIITA + TAP2 + CD3D + CTLA4 | 0.75 | 0.578 | 0.922 |
| 4 days | C3AR1 + IL1R2 + IFNG + CD177 + CIITA + TAP2 + CTLA4 | 0.766 | 0.597 | 0.934 |
| 4 days | C3AR1 + IL1R2 + IFNG + CD177 + CTLA4 | 0.75 | 0.58 | 0.92 |
| 4 days | C3AR1 + IL1R2 + IFNG + CD177 + CX3CR1 | 0.844 | 0.699 | 0.988 |
| 4 days | C3AR1 + IL1R2 + IFNG + CD177 + CX3CR1 + CD3D | 0.863 | 0.74 | 0.987 |
| 4 days | C3AR1 + IL1R2 + IFNG + CD177 + CX3CR1 + CD3D + CTLA4 | 0.895 | 0.784 | 1 |
| 4 days | C3AR1 + IL1R2 + IFNG + CD177 + CX3CR1 + CIITA | 0.863 | 0.727 | 1 |
| 4 days | C3AR1 + IL1R2 + IFNG + CD177 + CX3CR1 + CIITA + CD3D | 0.875 | 0.754 | 0.996 |
| 4 days | C3AR1 + IL1R2 + IFNG + CD177 + CX3CR1 + CIITA + CD3D + CTLA4 | 0.891 | 0.778 | 1 |
| 4 days | C3AR1 + IL1R2 + IFNG + CD177 + CX3CR1 + CIITA + CTLA4 | 0.891 | 0.778 | 1 |
| 4 days | C3AR1 + IL1R2 + IFNG + CD177 + CX3CR1 + CIITA + TAP2 | 0.871 | 0.746 | 0.996 |
| 4 days | C3AR1 + IL1R2 + IFNG + CD177 + CX3CR1 + CIITA + TAP2 + CD3D | 0.871 | 0.749 | 0.994 |
| 4 days | C3AR1 + IL1R2 + IFNG + CD177 + CX3CR1 + CIITA + TAP2 + CD3D + CTLA4 | 0.895 | 0.779 | 1 |
| 4 days | C3AR1 + IL1R2 + IFNG + CD177 + CX3CR1 + CIITA + TAP2 + CTLA4 | 0.895 | 0.781 | 1 |
| 4 days | C3AR1 + IL1R2 + IFNG + CD177 + CX3CR1 + CTLA4 | 0.895 | 0.784 | 1 |
| 4 days | C3AR1 + IL1R2 + IFNG + CD177 + CX3CR1 + TAP2 | 0.852 | 0.72 | 0.983 |
| 4 days | C3AR1 + IL1R2 + IFNG + CD177 + CX3CR1 + TAP2 + CD3D | 0.871 | 0.749 | 0.994 |
| 4 days | C3AR1 + IL1R2 + IFNG + CD177 + CX3CR1 + TAP2 + CD3D + CTLA4 | 0.895 | 0.779 | 1 |
| 4 days | C3AR1 + IL1R2 + IFNG + CD177 + CX3CR1 + TAP2 + CTLA4 | 0.906 | 0.797 | 1 |
| 4 days | C3AR1 + IL1R2 + IFNG + CD177 + S100A9 | 0.828 | 0.681 | 0.976 |
| 4 days | C3AR1 + IL1R2 + IFNG + CD177 + S100A9 + CD3D | 0.836 | 0.698 | 0.974 |
| 4 days | C3AR1 + IL1R2 + IFNG + CD177 + S100A9 + CD3D + CTLA4 | 0.836 | 0.699 | 0.973 |
| 4 days | C3AR1 + IL1R2 + IFNG + CD177 + S100A9 + CIITA | 0.82 | 0.668 | 0.972 |
| 4 days | C3AR1 + IL1R2 + IFNG + CD177 + S100A9 + CIITA + CD3D | 0.844 | 0.71 | 0.978 |
| 4 days | C3AR1 + IL1R2 + IFNG + CD177 + S100A9 + CIITA + CD3D + CTLA4 | 0.84 | 0.705 | 0.975 |
| 4 days | C3AR1 + IL1R2 + IFNG + CD177 + S100A9 + CIITA + CTLA4 | 0.82 | 0.675 | 0.966 |
| 4 days | C3AR1 + IL1R2 + IFNG + CD177 + S100A9 + CIITA + TAP2 | 0.852 | 0.713 | 0.99 |
| 4 days | C3AR1 + IL1R2 + IFNG + CD177 + S100A9 + CIITA + TAP2 + CD3D | 0.855 | 0.726 | 0.985 |
| 4 days | C3AR1 + IL1R2 + IFNG + CD177 + S100A9 + CIITA + TAP2 + CD3D + CTLA4 | 0.848 | 0.713 | 0.982 |
| 4 days | C3AR1 + IL1R2 + IFNG + CD177 + S100A9 + CIITA + TAP2 + CTLA4 | 0.84 | 0.701 | 0.979 |
| 4 days | C3AR1 + IL1R2 + IFNG + CD177 + S100A9 + CTLA4 | 0.824 | 0.681 | 0.968 |
| 4 days | C3AR1 + IL1R2 + IFNG + CD177 + S100A9 + CX3CR1 | 0.941 | 0.864 | 1 |
| 4 days | C3AR1 + IL1R2 + IFNG + CD177 + S100A9 + CX3CR1 + CD3D | 0.977 | 0.928 | 1 |
| 4 days | C3AR1 + IL1R2 + IFNG + CD177 + S100A9 + CX3CR1 + CIITA | 0.938 | 0.857 | 1 |
| 4 days | C3AR1 + IL1R2 + IFNG + CD177 + S100A9 + CX3CR1 + CIITA + CD3D | 0.98 | 0.939 | 1 |
| 4 days | C3AR1 + IL1R2 + IFNG + CD177 + S100A9 + CX3CR1 + CIITA + TAP2 | 0.938 | 0.857 | 1 |
| 4 days | C3AR1 + IL1R2 + IFNG + CD177 + S100A9 + CX3CR1 + TAP2 | 0.941 | 0.864 | 1 |
| 4 days | C3AR1 + IL1R2 + IFNG + CD177 + S100A9 + CX3CR1 + TAP2 + CD3D | 0.984 | 0.951 | 1 |
| 4 days | C3AR1 + IL1R2 + IFNG + CD177 + S100A9 + TAP2 | 0.84 | 0.696 | 0.984 |
| 4 days | C3AR1 + IL1R2 + IFNG + CD177 + S100A9 + TAP2 + CD3D | 0.84 | 0.703 | 0.976 |
| 4 days | C3AR1 + IL1R2 + IFNG + CD177 + S100A9 + TAP2 + CD3D + CTLA4 | 0.84 | 0.703 | 0.976 |
| 4 days | C3AR1 + IL1R2 + IFNG + CD177 + S100A9 + TAP2 + CTLA4 | 0.824 | 0.68 | 0.969 |
| 4 days | C3AR1 + IL1R2 + IFNG + CD177 + TAP2 | 0.742 | 0.562 | 0.923 |
| 4 days | C3AR1 + IL1R2 + IFNG + CD177 + TAP2 + CD3D | 0.75 | 0.571 | 0.929 |
| 4 days | C3AR1 + IL1R2 + IFNG + CD177 + TAP2 + CD3D + CTLA4 | 0.758 | 0.588 | 0.928 |
| 4 days | C3AR1 + IL1R2 + IFNG + CD177 + TAP2 + CTLA4 | 0.766 | 0.597 | 0.934 |
| 4 days | C3AR1 + IL1R2 + IFNG + CD3D | 0.73 | 0.552 | 0.909 |
| 4 days | C3AR1 + IL1R2 + IFNG + CD3D + CTLA4 | 0.742 | 0.57 | 0.915 |
| 4 days | C3AR1 + IL1R2 + IFNG + CIITA | 0.715 | 0.528 | 0.901 |
| 4 days | C3AR1 + IL1R2 + IFNG + CIITA + CD3D | 0.738 | 0.561 | 0.916 |
| 4 days | C3AR1 + IL1R2 + IFNG + CIITA + CD3D + CTLA4 | 0.734 | 0.56 | 0.909 |
| 4 days | C3AR1 + IL1R2 + IFNG + CIITA + CTLA4 | 0.742 | 0.57 | 0.915 |
| 4 days | C3AR1 + IL1R2 + IFNG + CIITA + TAP2 | 0.734 | 0.555 | 0.914 |
| 4 days | C3AR1 + IL1R2 + IFNG + CIITA + TAP2 + CD3D | 0.746 | 0.57 | 0.923 |
| 4 days | C3AR1 + IL1R2 + IFNG + CIITA + TAP2 + CD3D + CTLA4 | 0.742 | 0.567 | 0.917 |
| 4 days | C3AR1 + IL1R2 + IFNG + CIITA + TAP2 + CTLA4 | 0.754 | 0.583 | 0.925 |
| 4 days | C3AR1 + IL1R2 + IFNG + CTLA4 | 0.742 | 0.57 | 0.915 |
| 4 days | C3AR1 + IL1R2 + IFNG + CX3CR1 | 0.844 | 0.701 | 0.986 |
| 4 days | C3AR1 + IL1R2 + IFNG + CX3CR1 + CD3D | 0.844 | 0.71 | 0.977 |
| 4 days | C3AR1 + IL1R2 + IFNG + CX3CR1 + CD3D + CTLA4 | 0.867 | 0.746 | 0.988 |
| 4 days | C3AR1 + IL1R2 + IFNG + CX3CR1 + CIITA | 0.859 | 0.722 | 0.997 |
| 4 days | C3AR1 + IL1R2 + IFNG + CX3CR1 + CIITA + CD3D | 0.883 | 0.769 | 0.996 |
| 4 days | C3AR1 + IL1R2 + IFNG + CX3CR1 + CIITA + CD3D + CTLA4 | 0.883 | 0.771 | 0.994 |
| 4 days | C3AR1 + IL1R2 + IFNG + CX3CR1 + CIITA + CTLA4 | 0.883 | 0.771 | 0.994 |
| 4 days | C3AR1 + IL1R2 + IFNG + CX3CR1 + CIITA + TAP2 | 0.875 | 0.751 | 0.999 |
| 4 days | C3AR1 + IL1R2 + IFNG + CX3CR1 + CIITA + TAP2 + CD3D | 0.871 | 0.751 | 0.992 |
| 4 days | C3AR1 + IL1R2 + IFNG + CX3CR1 + CIITA + TAP2 + CD3D + CTLA4 | 0.879 | 0.764 | 0.993 |
| 4 days | C3AR1 + IL1R2 + IFNG + CX3CR1 + CIITA + TAP2 + CTLA4 | 0.879 | 0.764 | 0.993 |
| 4 days | C3AR1 + IL1R2 + IFNG + CX3CR1 + CTLA4 | 0.871 | 0.752 | 0.99 |
| 4 days | C3AR1 + IL1R2 + IFNG + CX3CR1 + TAP2 | 0.852 | 0.722 | 0.981 |
| 4 days | C3AR1 + IL1R2 + IFNG + CX3CR1 + TAP2 + CD3D | 0.867 | 0.743 | 0.992 |
| 4 days | C3AR1 + IL1R2 + IFNG + CX3CR1 + TAP2 + CD3D + CTLA4 | 0.875 | 0.758 | 0.992 |
| 4 days | C3AR1 + IL1R2 + IFNG + CX3CR1 + TAP2 + CTLA4 | 0.863 | 0.739 | 0.988 |
| 4 days | C3AR1 + IL1R2 + IFNG + S100A9 | 0.816 | 0.666 | 0.967 |
| 4 days | C3AR1 + IL1R2 + IFNG + S100A9 + CD3D | 0.852 | 0.718 | 0.985 |
| 4 days | C3AR1 + IL1R2 + IFNG + S100A9 + CD3D + CTLA4 | 0.852 | 0.718 | 0.985 |
| 4 days | C3AR1 + IL1R2 + IFNG + S100A9 + CIITA | 0.824 | 0.675 | 0.973 |
| 4 days | C3AR1 + IL1R2 + IFNG + S100A9 + CIITA + CD3D | 0.848 | 0.712 | 0.983 |
| 4 days | C3AR1 + IL1R2 + IFNG + S100A9 + CIITA + CD3D + CTLA4 | 0.848 | 0.712 | 0.983 |
| 4 days | C3AR1 + IL1R2 + IFNG + S100A9 + CIITA + CTLA4 | 0.816 | 0.668 | 0.965 |
| 4 days | C3AR1 + IL1R2 + IFNG + S100A9 + CIITA + TAP2 | 0.844 | 0.699 | 0.989 |
| 4 days | C3AR1 + IL1R2 + IFNG + S100A9 + CIITA + TAP2 + CD3D | 0.844 | 0.704 | 0.983 |
| 4 days | C3AR1 + IL1R2 + IFNG + S100A9 + CIITA + TAP2 + CD3D + CTLA4 | 0.848 | 0.711 | 0.984 |
| 4 days | C3AR1 + IL1R2 + IFNG + S100A9 + CIITA + TAP2 + CTLA4 | 0.828 | 0.681 | 0.975 |
| 4 days | C3AR1 + IL1R2 + IFNG + S100A9 + CTLA4 | 0.816 | 0.669 | 0.964 |
| 4 days | C3AR1 + IL1R2 + IFNG + S100A9 + CX3CR1 | 0.918 | 0.82 | 1 |
| 4 days | C3AR1 + IL1R2 + IFNG + S100A9 + CX3CR1 + CD3D | 0.941 | 0.867 | 1 |
| 4 days | C3AR1 + IL1R2 + IFNG + S100A9 + CX3CR1 + CD3D + CTLA4 | 0.938 | 0.861 | 1 |
| 4 days | C3AR1 + IL1R2 + IFNG + S100A9 + CX3CR1 + CIITA | 0.918 | 0.82 | 1 |
| 4 days | C3AR1 + IL1R2 + IFNG + S100A9 + CX3CR1 + CIITA + CD3D | 0.941 | 0.867 | 1 |
| 4 days | C3AR1 + IL1R2 + IFNG + S100A9 + CX3CR1 + CIITA + CD3D + CTLA4 | 0.941 | 0.867 | 1 |
| 4 days | C3AR1 + IL1R2 + IFNG + S100A9 + CX3CR1 + CIITA + CTLA4 | 0.922 | 0.832 | 1 |
| 4 days | C3AR1 + IL1R2 + IFNG + S100A9 + CX3CR1 + CIITA + TAP2 | 0.918 | 0.82 | 1 |
| 4 days | C3AR1 + IL1R2 + IFNG + S100A9 + CX3CR1 + CIITA + TAP2 + CD3D | 0.941 | 0.868 | 1 |
| 4 days | C3AR1 + IL1R2 + IFNG + S100A9 + CX3CR1 + CIITA + TAP2 + CD3D + CTLA4 | 0.938 | 0.858 | 1 |
| 4 days | C3AR1 + IL1R2 + IFNG + S100A9 + CX3CR1 + CIITA + TAP2 + CTLA4 | 0.918 | 0.82 | 1 |
| 4 days | C3AR1 + IL1R2 + IFNG + S100A9 + CX3CR1 + CTLA4 | 0.922 | 0.833 | 1 |
| 4 days | C3AR1 + IL1R2 + IFNG + S100A9 + CX3CR1 + TAP2 | 0.918 | 0.82 | 1 |
| 4 days | C3AR1 + IL1R2 + IFNG + S100A9 + CX3CR1 + TAP2 + CD3D | 0.941 | 0.868 | 1 |
| 4 days | C3AR1 + IL1R2 + IFNG + S100A9 + CX3CR1 + TAP2 + CD3D + CTLA4 | 0.941 | 0.867 | 1 |
| 4 days | C3AR1 + IL1R2 + IFNG + S100A9 + CX3CR1 + TAP2 + CTLA4 | 0.914 | 0.817 | 1 |
| 4 days | C3AR1 + IL1R2 + IFNG + S100A9 + TAP2 | 0.832 | 0.685 | 0.979 |
| 4 days | C3AR1 + IL1R2 + IFNG + S100A9 + TAP2 + CD3D | 0.852 | 0.718 | 0.985 |
| 4 days | C3AR1 + IL1R2 + IFNG + S100A9 + TAP2 + CD3D + CTLA4 | 0.848 | 0.713 | 0.983 |
| 4 days | C3AR1 + IL1R2 + IFNG + S100A9 + TAP2 + CTLA4 | 0.812 | 0.661 | 0.964 |
| 4 days | C3AR1 + IL1R2 + IFNG + TAP2 | 0.711 | 0.524 | 0.898 |
| 4 days | C3AR1 + IL1R2 + IFNG + TAP2 + CD3D | 0.723 | 0.538 | 0.907 |
| 4 days | C3AR1 + IL1R2 + IFNG + TAP2 + CD3D + CTLA4 | 0.742 | 0.567 | 0.917 |
| 4 days | C3AR1 + IL1R2 + IFNG + TAP2 + CTLA4 | 0.754 | 0.583 | 0.925 |
| 4 days | C3AR1 + IL1R2 + S100A9 | 0.824 | 0.67 | 0.979 |
| 4 days | C3AR1 + IL1R2 + S100A9 + CD3D | 0.84 | 0.7 | 0.979 |
| 4 days | C3AR1 + IL1R2 + S100A9 + CD3D + CTLA4 | 0.844 | 0.705 | 0.982 |
| 4 days | C3AR1 + IL1R2 + S100A9 + CIITA | 0.824 | 0.67 | 0.979 |
| 4 days | C3AR1 + IL1R2 + S100A9 + CIITA + CD3D | 0.848 | 0.715 | 0.98 |
| 4 days | C3AR1 + IL1R2 + S100A9 + CIITA + CD3D + CTLA4 | 0.832 | 0.689 | 0.975 |
| 4 days | C3AR1 + IL1R2 + S100A9 + CIITA + CTLA4 | 0.816 | 0.661 | 0.972 |
| 4 days | C3AR1 + IL1R2 + S100A9 + CIITA + TAP2 | 0.82 | 0.665 | 0.976 |
| 4 days | C3AR1 + IL1R2 + S100A9 + CIITA + TAP2 + CD3D | 0.844 | 0.706 | 0.981 |
| 4 days | C3AR1 + IL1R2 + S100A9 + CIITA + TAP2 + CD3D + CTLA4 | 0.844 | 0.706 | 0.982 |
| 4 days | C3AR1 + IL1R2 + S100A9 + CIITA + TAP2 + CTLA4 | 0.816 | 0.661 | 0.972 |
| 4 days | C3AR1 + IL1R2 + S100A9 + CTLA4 | 0.82 | 0.667 | 0.974 |
| 4 days | C3AR1 + IL1R2 + S100A9 + CX3CR1 | 0.852 | 0.71 | 0.993 |
| 4 days | C3AR1 + IL1R2 + S100A9 + CX3CR1 + CD3D | 0.898 | 0.781 | 1 |
| 4 days | C3AR1 + IL1R2 + S100A9 + CX3CR1 + CD3D + CTLA4 | 0.887 | 0.762 | 1 |
| 4 days | C3AR1 + IL1R2 + S100A9 + CX3CR1 + CIITA | 0.867 | 0.732 | 1 |
| 4 days | C3AR1 + IL1R2 + S100A9 + CX3CR1 + CIITA + CD3D | 0.895 | 0.779 | 1 |
| 4 days | C3AR1 + IL1R2 + S100A9 + CX3CR1 + CIITA + CD3D + CTLA4 | 0.902 | 0.795 | 1 |
| 4 days | C3AR1 + IL1R2 + S100A9 + CX3CR1 + CIITA + CTLA4 | 0.883 | 0.751 | 1 |
| 4 days | C3AR1 + IL1R2 + S100A9 + CX3CR1 + CIITA + TAP2 | 0.883 | 0.751 | 1 |
| 4 days | C3AR1 + IL1R2 + S100A9 + CX3CR1 + CIITA + TAP2 + CD3D | 0.898 | 0.786 | 1 |
| 4 days | C3AR1 + IL1R2 + S100A9 + CX3CR1 + CIITA + TAP2 + CD3D + CTLA4 | 0.914 | 0.815 | 1 |
| 4 days | C3AR1 + IL1R2 + S100A9 + CX3CR1 + CIITA + TAP2 + CTLA4 | 0.883 | 0.751 | 1 |
| 4 days | C3AR1 + IL1R2 + S100A9 + CX3CR1 + CTLA4 | 0.863 | 0.727 | 1 |
| 4 days | C3AR1 + IL1R2 + S100A9 + CX3CR1 + TAP2 | 0.875 | 0.745 | 1 |
| 4 days | C3AR1 + IL1R2 + S100A9 + CX3CR1 + TAP2 + CD3D | 0.895 | 0.773 | 1 |
| 4 days | C3AR1 + IL1R2 + S100A9 + CX3CR1 + TAP2 + CD3D + CTLA4 | 0.875 | 0.746 | 1 |
| 4 days | C3AR1 + IL1R2 + S100A9 + CX3CR1 + TAP2 + CTLA4 | 0.879 | 0.747 | 1 |
| 4 days | C3AR1 + IL1R2 + S100A9 + TAP2 | 0.816 | 0.658 | 0.975 |
| 4 days | C3AR1 + IL1R2 + S100A9 + TAP2 + CD3D | 0.84 | 0.701 | 0.979 |
| 4 days | C3AR1 + IL1R2 + S100A9 + TAP2 + CD3D + CTLA4 | 0.848 | 0.713 | 0.983 |
| 4 days | C3AR1 + IL1R2 + S100A9 + TAP2 + CTLA4 | 0.816 | 0.661 | 0.972 |
| 4 days | C3AR1 + IL1R2 + TAP2 | 0.68 | 0.488 | 0.871 |
| 4 days | C3AR1 + IL1R2 + TAP2 + CD3D | 0.715 | 0.523 | 0.907 |
| 4 days | C3AR1 + IL1R2 + TAP2 + CD3D + CTLA4 | 0.715 | 0.535 | 0.895 |
| 4 days | C3AR1 + IL1R2 + TAP2 + CTLA4 | 0.711 | 0.53 | 0.892 |
| 4 days | C3AR1 + S100A9 | 0.793 | 0.63 | 0.956 |
| 4 days | C3AR1 + S100A9 + CD3D | 0.781 | 0.617 | 0.946 |
| 4 days | C3AR1 + S100A9 + CD3D + CTLA4 | 0.789 | 0.628 | 0.95 |
| 4 days | C3AR1 + S100A9 + CIITA | 0.801 | 0.637 | 0.965 |
| 4 days | C3AR1 + S100A9 + CIITA + CD3D | 0.773 | 0.603 | 0.944 |
| 4 days | C3AR1 + S100A9 + CIITA + CD3D + CTLA4 | 0.785 | 0.618 | 0.952 |
| 4 days | C3AR1 + S100A9 + CIITA + CTLA4 | 0.785 | 0.616 | 0.954 |
| 4 days | C3AR1 + S100A9 + CIITA + TAP2 | 0.805 | 0.63 | 0.979 |
| 4 days | C3AR1 + S100A9 + CIITA + TAP2 + CD3D | 0.785 | 0.616 | 0.954 |
| 4 days | C3AR1 + S100A9 + CIITA + TAP2 + CD3D + CTLA4 | 0.797 | 0.631 | 0.963 |
| 4 days | C3AR1 + S100A9 + CIITA + TAP2 + CTLA4 | 0.812 | 0.649 | 0.976 |
| 4 days | C3AR1 + S100A9 + CTLA4 | 0.789 | 0.627 | 0.951 |
| 4 days | C3AR1 + S100A9 + CX3CR1 | 0.863 | 0.719 | 1 |
| 4 days | C3AR1 + S100A9 + CX3CR1 + CD3D | 0.887 | 0.756 | 1 |
| 4 days | C3AR1 + S100A9 + CX3CR1 + CD3D + CTLA4 | 0.879 | 0.748 | 1 |
| 4 days | C3AR1 + S100A9 + CX3CR1 + CIITA | 0.871 | 0.736 | 1 |
| 4 days | C3AR1 + S100A9 + CX3CR1 + CIITA + CD3D | 0.879 | 0.742 | 1 |
| 4 days | C3AR1 + S100A9 + CX3CR1 + CIITA + CD3D + CTLA4 | 0.891 | 0.768 | 1 |
| 4 days | C3AR1 + S100A9 + CX3CR1 + CIITA + CTLA4 | 0.875 | 0.741 | 1 |
| 4 days | C3AR1 + S100A9 + CX3CR1 + CIITA + TAP2 | 0.875 | 0.734 | 1 |
| 4 days | C3AR1 + S100A9 + CX3CR1 + CIITA + TAP2 + CD3D | 0.883 | 0.747 | 1 |
| 4 days | C3AR1 + S100A9 + CX3CR1 + CIITA + TAP2 + CD3D + CTLA4 | 0.887 | 0.76 | 1 |
| 4 days | C3AR1 + S100A9 + CX3CR1 + CIITA + TAP2 + CTLA4 | 0.875 | 0.73 | 1 |
| 4 days | C3AR1 + S100A9 + CX3CR1 + CTLA4 | 0.871 | 0.729 | 1 |
| 4 days | C3AR1 + S100A9 + CX3CR1 + TAP2 | 0.871 | 0.726 | 1 |
| 4 days | C3AR1 + S100A9 + CX3CR1 + TAP2 + CD3D | 0.887 | 0.756 | 1 |
| 4 days | C3AR1 + S100A9 + CX3CR1 + TAP2 + CD3D + CTLA4 | 0.875 | 0.743 | 1 |
| 4 days | C3AR1 + S100A9 + CX3CR1 + TAP2 + CTLA4 | 0.875 | 0.73 | 1 |
| 4 days | C3AR1 + S100A9 + TAP2 | 0.812 | 0.651 | 0.974 |
| 4 days | C3AR1 + S100A9 + TAP2 + CD3D | 0.797 | 0.631 | 0.963 |
| 4 days | C3AR1 + S100A9 + TAP2 + CD3D + CTLA4 | 0.801 | 0.637 | 0.965 |
| 4 days | C3AR1 + S100A9 + TAP2 + CTLA4 | 0.816 | 0.66 | 0.973 |
| 4 days | C3AR1 + TAP2 + CD3D + CTLA4 | 0.695 | 0.505 | 0.885 |
| 7 days | C3AR1 + 121601801-HERV0492 | 0.633 | 0.54 | 0.726 |
| 7 days | C3AR1 + IL18RAP | 0.633 | 0.541 | 0.725 |
| 7 days | C3AR1 + IL18 | 0.634 | 0.542 | 0.726 |
| 7 days | C3AR1 + BTLA | 0.635 | 0.543 | 0.727 |
| 7 days | C3AR1 + 050286701-HERV0513 | 0.635 | 0.544 | 0.727 |
| 7 days | C3AR1 + OAS3 | 0.636 | 0.544 | 0.728 |
| 7 days | C3AR1 + 011052202-HERV1033 | 0.643 | 0.552 | 0.734 |
| 7 days | C3AR1 + 011052702-MALR1044 | 0.644 | 0.552 | 0.736 |
| 7 days | C3AR1 + CD3D | 0.646 | 0.555 | 0.737 |
| 7 days | C3AR1 + IL7R | 0.646 | 0.555 | 0.738 |
| 7 days | C3AR1 + STAT4 | 0.649 | 0.558 | 0.741 |
| 7 days | C3AR1 + CD274 | 0.651 | 0.559 | 0.742 |
| 7 days | C3AR1 + CD40LG | 0.655 | 0.565 | 0.746 |
| 7 days | C3AR1 + 190665001-HERV0376 | 0.658 | 0.568 | 0.748 |
| 7 days | C3AR1 + IL10 | 0.665 | 0.572 | 0.757 |
| 7 days | C3AR1 + 170369402HE41 | 0.666 | 0.575 | 0.757 |
| 7 days | C3AR1 + 220247002-HERV0797 | 0.667 | 0.577 | 0.757 |
| 7 days | C3AR1 + IL1RN | 0.667 | 0.576 | 0.758 |
| 7 days | C3AR1 + 130360601-HERV0808 | 0.677 | 0.579 | 0.774 |
| 7 days | C3AR1 + CD74 | 0.682 | 0.595 | 0.769 |
| 7 days | C3AR1 + S100A9 | 0.686 | 0.598 | 0.773 |
| 7 days | C3AR1 + CX3CR1 | 0.688 | 0.601 | 0.776 |
| 7 days | C3AR1 + 021456001-MALR1017 | 0.69 | 0.602 | 0.778 |
| 7 days | C3AR1 + IL18R1 | 0.69 | 0.603 | 0.777 |
| 7 days | C3AR1 + IL15 | 0.691 | 0.597 | 0.785 |
| 7 days | C3AR1 + IFNG | 0.692 | 0.603 | 0.781 |
| 7 days | C3AR1 + IFIH1 | 0.7 | 0.589 | 0.811 |
| 7 days | C3AR1 + IL1R2 | 0.706 | 0.62 | 0.793 |
| 7 days | C3AR1 + HP | 0.709 | 0.624 | 0.794 |
| 7 days | C3AR1 + CD177 | 0.723 | 0.561 | 0.885 |
| 7 days | C3AR1 + CD177 + CD3D | 0.726 | 0.565 | 0.887 |
| 7 days | C3AR1 + CD177 + CD3D + CTLA4 | 0.731 | 0.571 | 0.892 |
| 7 days | C3AR1 + CD177 + CIITA | 0.85 | 0.725 | 0.976 |
| 7 days | C3AR1 + CD177 + CIITA + CD3D | 0.856 | 0.733 | 0.979 |
| 7 days | C3AR1 + CD177 + CIITA + CD3D + CTLA4 | 0.886 | 0.783 | 0.989 |
| 7 days | C3AR1 + CD177 + CIITA + CTLA4 | 0.875 | 0.764 | 0.987 |
| 7 days | C3AR1 + CD177 + CIITA + TAP2 | 0.85 | 0.724 | 0.977 |
| 7 days | C3AR1 + CD177 + CIITA + TAP2 + CD3D | 0.859 | 0.737 | 0.98 |
| 7 days | C3AR1 + CD177 + CIITA + TAP2 + CD3D + CTLA4 | 0.881 | 0.774 | 0.988 |
| 7 days | C3AR1 + CD177 + CIITA + TAP2 + CTLA4 | 0.875 | 0.764 | 0.987 |
| 7 days | C3AR1 + CD177 + CTLA4 | 0.717 | 0.552 | 0.883 |
| 7 days | C3AR1 + CD177 + CX3CR1 | 0.734 | 0.573 | 0.895 |
| 7 days | C3AR1 + CD177 + CX3CR1 + CD3D | 0.734 | 0.575 | 0.893 |
| 7 days | C3AR1 + CD177 + CX3CR1 + CD3D + CTLA4 | 0.723 | 0.56 | 0.886 |
| 7 days | C3AR1 + CD177 + CX3CR1 + CIITA | 0.853 | 0.727 | 0.979 |
| 7 days | C3AR1 + CD177 + CX3CR1 + CIITA + CD3D | 0.853 | 0.729 | 0.977 |
| 7 days | C3AR1 + CD177 + CX3CR1 + CIITA + CD3D + CTLA4 | 0.889 | 0.786 | 0.992 |
| 7 days | C3AR1 + CD177 + CX3CR1 + CIITA + CTLA4 | 0.87 | 0.756 | 0.984 |
| 7 days | C3AR1 + CD177 + CX3CR1 + CIITA + TAP2 | 0.856 | 0.731 | 0.981 |
| 7 days | C3AR1 + CD177 + CX3CR1 + CIITA + TAP2 + CD3D | 0.853 | 0.727 | 0.979 |
| 7 days | C3AR1 + CD177 + CX3CR1 + CIITA + TAP2 + CD3D + CTLA4 | 0.892 | 0.79 | 0.994 |
| 7 days | C3AR1 + CD177 + CX3CR1 + CIITA + TAP2 + CTLA4 | 0.873 | 0.759 | 0.986 |
| 7 days | C3AR1 + CD177 + CX3CR1 + CTLA4 | 0.731 | 0.569 | 0.894 |
| 7 days | C3AR1 + CD177 + CX3CR1 + TAP2 | 0.77 | 0.614 | 0.926 |
| 7 days | C3AR1 + CD177 + CX3CR1 + TAP2 + CD3D | 0.767 | 0.613 | 0.922 |
| 7 days | C3AR1 + CD177 + CX3CR1 + TAP2 + CD3D + CTLA4 | 0.77 | 0.614 | 0.926 |
| 7 days | C3AR1 + CD177 + CX3CR1 + TAP2 + CTLA4 | 0.767 | 0.609 | 0.926 |
| 7 days | C3AR1 + CD177 + S100A9 | 0.762 | 0.605 | 0.918 |
| 7 days | C3AR1 + CD177 + S100A9 + CD3D | 0.767 | 0.615 | 0.92 |
| 7 days | C3AR1 + CD177 + S100A9 + CD3D + CTLA4 | 0.756 | 0.602 | 0.91 |
| 7 days | C3AR1 + CD177 + S100A9 + CIITA | 0.853 | 0.729 | 0.977 |
| 7 days | C3AR1 + CD177 + S100A9 + CIITA + CD3D | 0.859 | 0.736 | 0.981 |
| 7 days | C3AR1 + CD177 + S100A9 + CIITA + CD3D + CTLA4 | 0.878 | 0.77 | 0.986 |
| 7 days | C3AR1 + CD177 + S100A9 + CIITA + CTLA4 | 0.875 | 0.764 | 0.987 |
| 7 days | C3AR1 + CD177 + S100A9 + CIITA + TAP2 | 0.856 | 0.734 | 0.978 |
| 7 days | C3AR1 + CD177 + S100A9 + CIITA + TAP2 + CD3D | 0.859 | 0.736 | 0.981 |
| 7 days | C3AR1 + CD177 + S100A9 + CIITA + TAP2 + CD3D + CTLA4 | 0.886 | 0.783 | 0.99 |
| 7 days | C3AR1 + CD177 + S100A9 + CIITA + TAP2 + CTLA4 | 0.878 | 0.768 | 0.988 |
| 7 days | C3AR1 + CD177 + S100A9 + CTLA4 | 0.765 | 0.608 | 0.921 |
| 7 days | C3AR1 + CD177 + S100A9 + CX3CR1 | 0.77 | 0.616 | 0.924 |
| 7 days | C3AR1 + CD177 + S100A9 + CX3CR1 + CD3D | 0.784 | 0.636 | 0.932 |
| 7 days | C3AR1 + CD177 + S100A9 + CX3CR1 + CD3D + CTLA4 | 0.773 | 0.621 | 0.925 |
| 7 days | C3AR1 + CD177 + S100A9 + CX3CR1 + CIITA | 0.856 | 0.733 | 0.979 |
| 7 days | C3AR1 + CD177 + S100A9 + CX3CR1 + CIITA + CD3D | 0.867 | 0.747 | 0.987 |
| 7 days | C3AR1 + CD177 + S100A9 + CX3CR1 + CIITA + CD3D + CTLA4 | 0.898 | 0.799 | 0.996 |
| 7 days | C3AR1 + CD177 + S100A9 + CX3CR1 + CIITA + CTLA4 | 0.873 | 0.761 | 0.985 |
| 7 days | C3AR1 + CD177 + S100A9 + CX3CR1 + CIITA + TAP2 | 0.859 | 0.735 | 0.982 |
| 7 days | C3AR1 + CD177 + S100A9 + CX3CR1 + CIITA + TAP2 + CD3D | 0.856 | 0.733 | 0.979 |
| 7 days | C3AR1 + CD177 + S100A9 + CX3CR1 + CIITA + TAP2 + CD3D + CTLA4 | 0.898 | 0.799 | 0.996 |
| 7 days | C3AR1 + CD177 + S100A9 + CX3CR1 + CIITA + TAP2 + CTLA4 | 0.881 | 0.774 | 0.988 |
| 7 days | C3AR1 + CD177 + S100A9 + CX3CR1 + CTLA4 | 0.77 | 0.616 | 0.924 |
| 7 days | C3AR1 + CD177 + S100A9 + CX3CR1 + TAP2 | 0.781 | 0.629 | 0.933 |
| 7 days | C3AR1 + CD177 + S100A9 + CX3CR1 + TAP2 + CD3D | 0.781 | 0.631 | 0.932 |
| 7 days | C3AR1 + CD177 + S100A9 + CX3CR1 + TAP2 + CD3D + CTLA4 | 0.784 | 0.637 | 0.931 |
| 7 days | C3AR1 + CD177 + S100A9 + CX3CR1 + TAP2 + CTLA4 | 0.789 | 0.64 | 0.939 |
| 7 days | C3AR1 + CD177 + S100A9 + TAP2 | 0.792 | 0.643 | 0.941 |
| 7 days | C3AR1 + CD177 + S100A9 + TAP2 + CD3D | 0.789 | 0.644 | 0.935 |
| 7 days | C3AR1 + CD177 + S100A9 + TAP2 + CD3D + CTLA4 | 0.789 | 0.644 | 0.935 |
| 7 days | C3AR1 + CD177 + S100A9 + TAP2 + CTLA4 | 0.792 | 0.643 | 0.941 |
| 7 days | C3AR1 + CD177 + TAP2 | 0.767 | 0.611 | 0.924 |
| 7 days | C3AR1 + CD177 + TAP2 + CD3D | 0.762 | 0.606 | 0.918 |
| 7 days | C3AR1 + CD177 + TAP2 + CD3D + CTLA4 | 0.77 | 0.615 | 0.925 |
| 7 days | C3AR1 + CD177 + TAP2 + CTLA4 | 0.767 | 0.612 | 0.923 |
| 7 days | C3AR1 + CD74 + CD177 | 0.812 | 0.669 | 0.954 |
| 7 days | C3AR1 + CD74 + CD177 + CD3D | 0.803 | 0.661 | 0.946 |
| 7 days | C3AR1 + CD74 + CD177 + CD3D + CTLA4 | 0.803 | 0.661 | 0.946 |
| 7 days | C3AR1 + CD74 + CD177 + CIITA | 0.87 | 0.752 | 0.987 |
| 7 days | C3AR1 + CD74 + CD177 + CIITA + CD3D | 0.875 | 0.76 | 0.991 |
| 7 days | C3AR1 + CD74 + CD177 + CIITA + CD3D + CTLA4 | 0.889 | 0.787 | 0.992 |
| 7 days | C3AR1 + CD74 + CD177 + CIITA + CTLA4 | 0.875 | 0.765 | 0.985 |
| 7 days | C3AR1 + CD74 + CD177 + CIITA + TAP2 | 0.87 | 0.752 | 0.987 |
| 7 days | C3AR1 + CD74 + CD177 + CIITA + TAP2 + CD3D | 0.87 | 0.753 | 0.987 |
| 7 days | C3AR1 + CD74 + CD177 + CIITA + TAP2 + CD3D + CTLA4 | 0.898 | 0.8 | 0.995 |
| 7 days | C3AR1 + CD74 + CD177 + CIITA + TAP2 + CTLA4 | 0.873 | 0.761 | 0.985 |
| 7 days | C3AR1 + CD74 + CD177 + CTLA4 | 0.817 | 0.678 | 0.956 |
| 7 days | C3AR1 + CD74 + CD177 + CX3CR1 | 0.823 | 0.684 | 0.961 |
| 7 days | C3AR1 + CD74 + CD177 + CX3CR1 + CD3D | 0.812 | 0.671 | 0.952 |
| 7 days | C3AR1 + CD74 + CD177 + CX3CR1 + CD3D + CTLA4 | 0.812 | 0.672 | 0.951 |
| 7 days | C3AR1 + CD74 + CD177 + CX3CR1 + CIITA | 0.878 | 0.767 | 0.99 |
| 7 days | C3AR1 + CD74 + CD177 + CX3CR1 + CIITA + CD3D | 0.878 | 0.767 | 0.989 |
| 7 days | C3AR1 + CD74 + CD177 + CX3CR1 + CIITA + CD3D + CTLA4 | 0.892 | 0.792 | 0.992 |
| 7 days | C3AR1 + CD74 + CD177 + CX3CR1 + CIITA + CTLA4 | 0.875 | 0.763 | 0.988 |
| 7 days | C3AR1 + CD74 + CD177 + CX3CR1 + CIITA + TAP2 | 0.875 | 0.762 | 0.989 |
| 7 days | C3AR1 + CD74 + CD177 + CX3CR1 + CIITA + TAP2 + CD3D | 0.873 | 0.758 | 0.987 |
| 7 days | C3AR1 + CD74 + CD177 + CX3CR1 + CIITA + TAP2 + CD3D + CTLA4 | 0.898 | 0.801 | 0.994 |
| 7 days | C3AR1 + CD74 + CD177 + CX3CR1 + CIITA + TAP2 + CTLA4 | 0.875 | 0.763 | 0.987 |
| 7 days | C3AR1 + CD74 + CD177 + CX3CR1 + CTLA4 | 0.823 | 0.683 | 0.962 |
| 7 days | C3AR1 + CD74 + CD177 + CX3CR1 + TAP2 | 0.817 | 0.675 | 0.96 |
| 7 days | C3AR1 + CD74 + CD177 + CX3CR1 + TAP2 + CD3D | 0.82 | 0.684 | 0.956 |
| 7 days | C3AR1 + CD74 + CD177 + CX3CR1 + TAP2 + CD3D + CTLA4 | 0.82 | 0.684 | 0.956 |
| 7 days | C3AR1 + CD74 + CD177 + CX3CR1 + TAP2 + CTLA4 | 0.825 | 0.685 | 0.966 |
| 7 days | C3AR1 + CD74 + CD177 + S100A9 | 0.825 | 0.685 | 0.966 |
| 7 days | C3AR1 + CD74 + CD177 + S100A9 + CD3D | 0.831 | 0.696 | 0.966 |
| 7 days | C3AR1 + CD74 + CD177 + S100A9 + CD3D + CTLA4 | 0.828 | 0.692 | 0.964 |
| 7 days | C3AR1 + CD74 + CD177 + S100A9 + CIITA | 0.873 | 0.757 | 0.988 |
| 7 days | C3AR1 + CD74 + CD177 + S100A9 + CIITA + CD3D | 0.87 | 0.753 | 0.987 |
| 7 days | C3AR1 + CD74 + CD177 + S100A9 + CIITA + CD3D + CTLA4 | 0.889 | 0.787 | 0.992 |
| 7 days | C3AR1 + CD74 + CD177 + S100A9 + CIITA + CTLA4 | 0.878 | 0.77 | 0.987 |
| 7 days | C3AR1 + CD74 + CD177 + S100A9 + CIITA + TAP2 | 0.878 | 0.765 | 0.991 |
| 7 days | C3AR1 + CD74 + CD177 + S100A9 + CIITA + TAP2 + CD3D | 0.875 | 0.761 | 0.99 |
| 7 days | C3AR1 + CD74 + CD177 + S100A9 + CIITA + TAP2 + CD3D + CTLA4 | 0.9 | 0.802 | 0.998 |
| 7 days | C3AR1 + CD74 + CD177 + S100A9 + CIITA + TAP2 + CTLA4 | 0.878 | 0.77 | 0.987 |
| 7 days | C3AR1 + CD74 + CD177 + S100A9 + CTLA4 | 0.825 | 0.685 | 0.966 |
| 7 days | C3AR1 + CD74 + CD177 + S100A9 + CX3CR1 | 0.853 | 0.718 | 0.989 |
| 7 days | C3AR1 + CD74 + CD177 + S100A9 + CX3CR1 + CD3D | 0.845 | 0.708 | 0.981 |
| 7 days | C3AR1 + CD74 + CD177 + S100A9 + CX3CR1 + CD3D + CTLA4 | 0.85 | 0.715 | 0.986 |
| 7 days | C3AR1 + CD74 + CD177 + S100A9 + CX3CR1 + CIITA | 0.881 | 0.766 | 0.995 |
| 7 days | C3AR1 + CD74 + CD177 + S100A9 + CX3CR1 + CIITA + CD3D | 0.875 | 0.758 | 0.993 |
| 7 days | C3AR1 + CD74 + CD177 + S100A9 + CX3CR1 + CIITA + CD3D + CTLA4 | 0.889 | 0.784 | 0.994 |
| 7 days | C3AR1 + CD74 + CD177 + S100A9 + CX3CR1 + CIITA + CTLA4 | 0.875 | 0.763 | 0.988 |
| 7 days | C3AR1 + CD74 + CD177 + S100A9 + CX3CR1 + CIITA + TAP2 | 0.881 | 0.769 | 0.993 |
| 7 days | C3AR1 + CD74 + CD177 + S100A9 + CX3CR1 + CIITA + TAP2 + CD3D | 0.878 | 0.761 | 0.995 |
| 7 days | C3AR1 + CD74 + CD177 + S100A9 + CX3CR1 + CIITA + TAP2 + CD3D + CTLA4 | 0.898 | 0.8 | 0.995 |
| 7 days | C3AR1 + CD74 + CD177 + S100A9 + CX3CR1 + CIITA + TAP2 + CTLA4 | 0.884 | 0.774 | 0.993 |
| 7 days | C3AR1 + CD74 + CD177 + S100A9 + CX3CR1 + CTLA4 | 0.856 | 0.722 | 0.99 |
| 7 days | C3AR1 + CD74 + CD177 + S100A9 + CX3CR1 + TAP2 | 0.856 | 0.72 | 0.992 |
| 7 days | C3AR1 + CD74 + CD177 + S100A9 + CX3CR1 + TAP2 + CD3D | 0.853 | 0.719 | 0.987 |
| 7 days | C3AR1 + CD74 + CD177 + S100A9 + CX3CR1 + TAP2 + CD3D + CTLA4 | 0.85 | 0.715 | 0.986 |
| 7 days | C3AR1 + CD74 + CD177 + S100A9 + CX3CR1 + TAP2 + CTLA4 | 0.859 | 0.723 | 0.994 |
| 7 days | C3AR1 + CD74 + CD177 + S100A9 + TAP2 | 0.823 | 0.68 | 0.966 |
| 7 days | C3AR1 + CD74 + CD177 + S100A9 + TAP2 + CD3D | 0.831 | 0.696 | 0.966 |
| 7 days | C3AR1 + CD74 + CD177 + S100A9 + TAP2 + CD3D + CTLA4 | 0.828 | 0.693 | 0.964 |
| 7 days | C3AR1 + CD74 + CD177 + S100A9 + TAP2 + CTLA4 | 0.823 | 0.68 | 0.966 |
| 7 days | C3AR1 + CD74 + CD177 + TAP2 | 0.82 | 0.682 | 0.958 |
| 7 days | C3AR1 + CD74 + CD177 + TAP2 + CD3D | 0.814 | 0.675 | 0.954 |
| 7 days | C3AR1 + CD74 + CD177 + TAP2 + CD3D + CTLA4 | 0.809 | 0.667 | 0.95 |
| 7 days | C3AR1 + CD74 + CD177 + TAP2 + CTLA4 | 0.817 | 0.677 | 0.958 |
| 7 days | C3AR1 + CD74 + CD3D | 0.745 | 0.586 | 0.905 |
| 7 days | C3AR1 + CD74 + CD3D + CTLA4 | 0.745 | 0.586 | 0.904 |
| 7 days | C3AR1 + CD74 + CIITA | 0.861 | 0.74 | 0.983 |
| 7 days | C3AR1 + CD74 + CIITA + CD3D | 0.853 | 0.728 | 0.979 |
| 7 days | C3AR1 + CD74 + CIITA + CD3D + CTLA4 | 0.867 | 0.753 | 0.981 |
| 7 days | C3AR1 + CD74 + CIITA + CTLA4 | 0.878 | 0.766 | 0.99 |
| 7 days | C3AR1 + CD74 + CIITA + TAP2 | 0.859 | 0.736 | 0.981 |
| 7 days | C3AR1 + CD74 + CIITA + TAP2 + CD3D | 0.853 | 0.728 | 0.979 |
| 7 days | C3AR1 + CD74 + CIITA + TAP2 + CD3D + CTLA4 | 0.87 | 0.757 | 0.983 |
| 7 days | C3AR1 + CD74 + CIITA + TAP2 + CTLA4 | 0.881 | 0.769 | 0.992 |
| 7 days | C3AR1 + CD74 + CTLA4 | 0.765 | 0.61 | 0.919 |
| 7 days | C3AR1 + CD74 + CX3CR1 | 0.776 | 0.619 | 0.932 |
| 7 days | C3AR1 + CD74 + CX3CR1 + CD3D | 0.767 | 0.616 | 0.919 |
| 7 days | C3AR1 + CD74 + CX3CR1 + CD3D + CTLA4 | 0.767 | 0.616 | 0.919 |
| 7 days | C3AR1 + CD74 + CX3CR1 + CIITA | 0.867 | 0.744 | 0.99 |
| 7 days | C3AR1 + CD74 + CX3CR1 + CIITA + CD3D | 0.867 | 0.747 | 0.987 |
| 7 days | C3AR1 + CD74 + CX3CR1 + CIITA + CD3D + CTLA4 | 0.87 | 0.756 | 0.984 |
| 7 days | C3AR1 + CD74 + CX3CR1 + CIITA + CTLA4 | 0.873 | 0.754 | 0.991 |
| 7 days | C3AR1 + CD74 + CX3CR1 + CIITA + TAP2 | 0.864 | 0.74 | 0.989 |
| 7 days | C3AR1 + CD74 + CX3CR1 + CIITA + TAP2 + CD3D | 0.864 | 0.743 | 0.986 |
| 7 days | C3AR1 + CD74 + CX3CR1 + CIITA + TAP2 + CD3D + CTLA4 | 0.87 | 0.756 | 0.984 |
| 7 days | C3AR1 + CD74 + CX3CR1 + CIITA + TAP2 + CTLA4 | 0.87 | 0.749 | 0.991 |
| 7 days | C3AR1 + CD74 + CX3CR1 + CTLA4 | 0.776 | 0.623 | 0.929 |
| 7 days | C3AR1 + CD74 + CX3CR1 + TAP2 | 0.781 | 0.627 | 0.936 |
| 7 days | C3AR1 + CD74 + CX3CR1 + TAP2 + CD3D | 0.778 | 0.626 | 0.931 |
| 7 days | C3AR1 + CD74 + CX3CR1 + TAP2 + CD3D + CTLA4 | 0.792 | 0.644 | 0.94 |
| 7 days | C3AR1 + CD74 + CX3CR1 + TAP2 + CTLA4 | 0.798 | 0.647 | 0.948 |
| 7 days | C3AR1 + CD74 + IFNG | 0.823 | 0.692 | 0.954 |
| 7 days | C3AR1 + CD74 + IFNG + CD177 | 0.842 | 0.719 | 0.965 |
| 7 days | C3AR1 + CD74 + IFNG + CD177 + CD3D | 0.839 | 0.715 | 0.964 |
| 7 days | C3AR1 + CD74 + IFNG + CD177 + CD3D + CTLA4 | 0.845 | 0.723 | 0.967 |
| 7 days | C3AR1 + CD74 + IFNG + CD177 + CIITA | 0.892 | 0.791 | 0.993 |
| 7 days | C3AR1 + CD74 + IFNG + CD177 + CIITA + CD3D | 0.892 | 0.791 | 0.993 |
| 7 days | C3AR1 + CD74 + IFNG + CD177 + CIITA + CD3D + CTLA4 | 0.892 | 0.795 | 0.989 |
| 7 days | C3AR1 + CD74 + IFNG + CD177 + CIITA + CTLA4 | 0.886 | 0.786 | 0.987 |
| 7 days | C3AR1 + CD74 + IFNG + CD177 + CIITA + TAP2 | 0.892 | 0.792 | 0.992 |
| 7 days | C3AR1 + CD74 + IFNG + CD177 + CIITA + TAP2 + CD3D | 0.892 | 0.792 | 0.992 |
| 7 days | C3AR1 + CD74 + IFNG + CD177 + CIITA + TAP2 + CD3D + CTLA4 | 0.892 | 0.794 | 0.99 |
| 7 days | C3AR1 + CD74 + IFNG + CD177 + CIITA + TAP2 + CTLA4 | 0.9 | 0.807 | 0.994 |
| 7 days | C3AR1 + CD74 + IFNG + CD177 + CTLA4 | 0.839 | 0.715 | 0.963 |
| 7 days | C3AR1 + CD74 + IFNG + CD177 + CX3CR1 | 0.881 | 0.775 | 0.986 |
| 7 days | C3AR1 + CD74 + IFNG + CD177 + CX3CR1 + CD3D | 0.886 | 0.784 | 0.989 |
| 7 days | C3AR1 + CD74 + IFNG + CD177 + CX3CR1 + CD3D + CTLA4 | 0.886 | 0.782 | 0.991 |
| 7 days | C3AR1 + CD74 + IFNG + CD177 + CX3CR1 + CIITA | 0.917 | 0.827 | 1 |
| 7 days | C3AR1 + CD74 + IFNG + CD177 + CX3CR1 + CIITA + CD3D | 0.906 | 0.808 | 1 |
| 7 days | C3AR1 + CD74 + IFNG + CD177 + CX3CR1 + CIITA + CD3D + CTLA4 | 0.911 | 0.817 | 1 |
| 7 days | C3AR1 + CD74 + IFNG + CD177 + CX3CR1 + CIITA + CTLA4 | 0.917 | 0.827 | 1 |
| 7 days | C3AR1 + CD74 + IFNG + CD177 + CX3CR1 + CIITA + TAP2 | 0.917 | 0.827 | 1 |
| 7 days | C3AR1 + CD74 + IFNG + CD177 + CX3CR1 + CIITA + TAP2 + CD3D | 0.914 | 0.822 | 1 |
| 7 days | C3AR1 + CD74 + IFNG + CD177 + CX3CR1 + CIITA + TAP2 + CD3D + CTLA4 | 0.928 | 0.844 | 1 |
| 7 days | C3AR1 + CD74 + IFNG + CD177 + CX3CR1 + CIITA + TAP2 + CTLA4 | 0.914 | 0.823 | 1 |
| 7 days | C3AR1 + CD74 + IFNG + CD177 + CX3CR1 + CTLA4 | 0.881 | 0.774 | 0.988 |
| 7 days | C3AR1 + CD74 + IFNG + CD177 + CX3CR1 + TAP2 | 0.881 | 0.775 | 0.986 |
| 7 days | C3AR1 + CD74 + IFNG + CD177 + CX3CR1 + TAP2 + CD3D | 0.892 | 0.79 | 0.994 |
| 7 days | C3AR1 + CD74 + IFNG + CD177 + CX3CR1 + TAP2 + CD3D + CTLA4 | 0.889 | 0.786 | 0.992 |
| 7 days | C3AR1 + CD74 + IFNG + CD177 + CX3CR1 + TAP2 + CTLA4 | 0.878 | 0.768 | 0.988 |
| 7 days | C3AR1 + CD74 + IFNG + CD177 + S100A9 | 0.842 | 0.72 | 0.965 |
| 7 days | C3AR1 + CD74 + IFNG + CD177 + S100A9 + CD3D | 0.845 | 0.723 | 0.967 |
| 7 days | C3AR1 + CD74 + IFNG + CD177 + S100A9 + CD3D + CTLA4 | 0.842 | 0.719 | 0.965 |
| 7 days | C3AR1 + CD74 + IFNG + CD177 + S100A9 + CIITA | 0.889 | 0.789 | 0.989 |
| 7 days | C3AR1 + CD74 + IFNG + CD177 + S100A9 + CIITA + CD3D | 0.884 | 0.781 | 0.986 |
| 7 days | C3AR1 + CD74 + IFNG + CD177 + S100A9 + CIITA + CD3D + CTLA4 | 0.886 | 0.786 | 0.987 |
| 7 days | C3AR1 + CD74 + IFNG + CD177 + S100A9 + CIITA + CTLA4 | 0.889 | 0.79 | 0.988 |
| 7 days | C3AR1 + CD74 + IFNG + CD177 + S100A9 + CIITA + TAP2 | 0.906 | 0.814 | 0.997 |
| 7 days | C3AR1 + CD74 + IFNG + CD177 + S100A9 + CIITA + TAP2 + CD3D | 0.906 | 0.814 | 0.997 |
| 7 days | C3AR1 + CD74 + IFNG + CD177 + S100A9 + CIITA + TAP2 + CD3D + CTLA4 | 0.892 | 0.794 | 0.99 |
| 7 days | C3AR1 + CD74 + IFNG + CD177 + S100A9 + CIITA + TAP2 + CTLA4 | 0.895 | 0.798 | 0.992 |
| 7 days | C3AR1 + CD74 + IFNG + CD177 + S100A9 + CTLA4 | 0.842 | 0.72 | 0.965 |
| 7 days | C3AR1 + CD74 + IFNG + CD177 + S100A9 + CX3CR1 | 0.881 | 0.777 | 0.985 |
| 7 days | C3AR1 + CD74 + IFNG + CD177 + S100A9 + CX3CR1 + CD3D | 0.881 | 0.776 | 0.986 |
| 7 days | C3AR1 + CD74 + IFNG + CD177 + S100A9 + CX3CR1 + CD3D + CTLA4 | 0.886 | 0.784 | 0.989 |
| 7 days | C3AR1 + CD74 + IFNG + CD177 + S100A9 + CX3CR1 + CIITA | 0.92 | 0.831 | 1 |
| 7 days | C3AR1 + CD74 + IFNG + CD177 + S100A9 + CX3CR1 + CIITA + CD3D | 0.914 | 0.821 | 1 |
| 7 days | C3AR1 + CD74 + IFNG + CD177 + S100A9 + CX3CR1 + CIITA + CD3D + CTLA4 | 0.909 | 0.813 | 1 |
| 7 days | C3AR1 + CD74 + IFNG + CD177 + S100A9 + CX3CR1 + CIITA + CTLA4 | 0.922 | 0.835 | 1 |
| 7 days | C3AR1 + CD74 + IFNG + CD177 + S100A9 + CX3CR1 + CIITA + TAP2 | 0.922 | 0.834 | 1 |
| 7 days | C3AR1 + CD74 + IFNG + CD177 + S100A9 + CX3CR1 + CIITA + TAP2 + CD3D | 0.922 | 0.834 | 1 |
| 7 days | C3AR1 + CD74 + IFNG + CD177 + S100A9 + CX3CR1 + CIITA + TAP2 + CD3D + CTLA4 | 0.925 | 0.84 | 1 |
| 7 days | C3AR1 + CD74 + IFNG + CD177 + S100A9 + CX3CR1 + CIITA + TAP2 + CTLA4 | 0.922 | 0.835 | 1 |
| 7 days | C3AR1 + CD74 + IFNG + CD177 + S100A9 + CX3CR1 + CTLA4 | 0.884 | 0.778 | 0.989 |
| 7 days | C3AR1 + CD74 + IFNG + CD177 + S100A9 + CX3CR1 + TAP2 | 0.881 | 0.777 | 0.985 |
| 7 days | C3AR1 + CD74 + IFNG + CD177 + S100A9 + CX3CR1 + TAP2 + CD3D | 0.878 | 0.771 | 0.985 |
| 7 days | C3AR1 + CD74 + IFNG + CD177 + S100A9 + CX3CR1 + TAP2 + CD3D + CTLA4 | 0.881 | 0.775 | 0.987 |
| 7 days | C3AR1 + CD74 + IFNG + CD177 + S100A9 + CX3CR1 + TAP2 + CTLA4 | 0.878 | 0.769 | 0.987 |
| 7 days | C3AR1 + CD74 + IFNG + CD177 + S100A9 + TAP2 | 0.842 | 0.718 | 0.967 |
| 7 days | C3AR1 + CD74 + IFNG + CD177 + S100A9 + TAP2 + CD3D | 0.834 | 0.707 | 0.96 |
| 7 days | C3AR1 + CD74 + IFNG + CD177 + S100A9 + TAP2 + CD3D + CTLA4 | 0.839 | 0.715 | 0.964 |
| 7 days | C3AR1 + CD74 + IFNG + CD177 + S100A9 + TAP2 + CTLA4 | 0.839 | 0.714 | 0.964 |
| 7 days | C3AR1 + CD74 + IFNG + CD177 + TAP2 | 0.845 | 0.722 | 0.968 |
| 7 days | C3AR1 + CD74 + IFNG + CD177 + TAP2 + CD3D | 0.839 | 0.715 | 0.964 |
| 7 days | C3AR1 + CD74 + IFNG + CD177 + TAP2 + CD3D + CTLA4 | 0.839 | 0.715 | 0.964 |
| 7 days | C3AR1 + CD74 + IFNG + CD177 + TAP2 + CTLA4 | 0.839 | 0.714 | 0.964 |
| 7 days | C3AR1 + CD74 + IFNG + CD3D | 0.823 | 0.692 | 0.954 |
| 7 days | C3AR1 + CD74 + IFNG + CD3D + CTLA4 | 0.831 | 0.703 | 0.959 |
| 7 days | C3AR1 + CD74 + IFNG + CIITA | 0.87 | 0.758 | 0.981 |
| 7 days | C3AR1 + CD74 + IFNG + CIITA + CD3D | 0.878 | 0.769 | 0.987 |
| 7 days | C3AR1 + CD74 + IFNG + CIITA + CD3D + CTLA4 | 0.875 | 0.765 | 0.985 |
| 7 days | C3AR1 + CD74 + IFNG + CIITA + CTLA4 | 0.881 | 0.775 | 0.987 |
| 7 days | C3AR1 + CD74 + IFNG + CIITA + TAP2 | 0.878 | 0.769 | 0.987 |
| 7 days | C3AR1 + CD74 + IFNG + CIITA + TAP2 + CD3D | 0.878 | 0.769 | 0.987 |
| 7 days | C3AR1 + CD74 + IFNG + CIITA + TAP2 + CD3D + CTLA4 | 0.884 | 0.777 | 0.99 |
| 7 days | C3AR1 + CD74 + IFNG + CIITA + TAP2 + CTLA4 | 0.884 | 0.777 | 0.991 |
| 7 days | C3AR1 + CD74 + IFNG + CTLA4 | 0.825 | 0.695 | 0.956 |
| 7 days | C3AR1 + CD74 + IFNG + CX3CR1 | 0.878 | 0.767 | 0.99 |
| 7 days | C3AR1 + CD74 + IFNG + CX3CR1 + CD3D | 0.873 | 0.758 | 0.987 |
| 7 days | C3AR1 + CD74 + IFNG + CX3CR1 + CD3D + CTLA4 | 0.873 | 0.756 | 0.989 |
| 7 days | C3AR1 + CD74 + IFNG + CX3CR1 + CIITA | 0.917 | 0.824 | 1 |
| 7 days | C3AR1 + CD74 + IFNG + CX3CR1 + CIITA + CD3D | 0.914 | 0.819 | 1 |
| 7 days | C3AR1 + CD74 + IFNG + CX3CR1 + CIITA + CD3D + CTLA4 | 0.925 | 0.835 | 1 |
| 7 days | C3AR1 + CD74 + IFNG + CX3CR1 + CIITA + CTLA4 | 0.92 | 0.83 | 1 |
| 7 days | C3AR1 + CD74 + IFNG + CX3CR1 + CIITA + TAP2 | 0.92 | 0.83 | 1 |
| 7 days | C3AR1 + CD74 + IFNG + CX3CR1 + CIITA + TAP2 + CD3D | 0.911 | 0.814 | 1 |
| 7 days | C3AR1 + CD74 + IFNG + CX3CR1 + CIITA + TAP2 + CD3D + CTLA4 | 0.931 | 0.844 | 1 |
| 7 days | C3AR1 + CD74 + IFNG + CX3CR1 + CIITA + TAP2 + CTLA4 | 0.917 | 0.826 | 1 |
| 7 days | C3AR1 + CD74 + IFNG + CX3CR1 + CTLA4 | 0.873 | 0.758 | 0.987 |
| 7 days | C3AR1 + CD74 + IFNG + CX3CR1 + TAP2 | 0.878 | 0.766 | 0.99 |
| 7 days | C3AR1 + CD74 + IFNG + CX3CR1 + TAP2 + CD3D | 0.873 | 0.757 | 0.988 |
| 7 days | C3AR1 + CD74 + IFNG + CX3CR1 + TAP2 + CD3D + CTLA4 | 0.87 | 0.752 | 0.988 |
| 7 days | C3AR1 + CD74 + IFNG + CX3CR1 + TAP2 + CTLA4 | 0.873 | 0.758 | 0.987 |
| 7 days | C3AR1 + CD74 + IFNG + S100A9 | 0.828 | 0.699 | 0.957 |
| 7 days | C3AR1 + CD74 + IFNG + S100A9 + CD3D | 0.828 | 0.699 | 0.957 |
| 7 days | C3AR1 + CD74 + IFNG + S100A9 + CD3D + CTLA4 | 0.823 | 0.691 | 0.955 |
| 7 days | C3AR1 + CD74 + IFNG + S100A9 + CIITA | 0.867 | 0.754 | 0.98 |
| 7 days | C3AR1 + CD74 + IFNG + S100A9 + CIITA + CD3D | 0.867 | 0.753 | 0.981 |
| 7 days | C3AR1 + CD74 + IFNG + S100A9 + CIITA + CD3D + CTLA4 | 0.87 | 0.758 | 0.982 |
| 7 days | C3AR1 + CD74 + IFNG + S100A9 + CIITA + CTLA4 | 0.884 | 0.779 | 0.989 |
| 7 days | C3AR1 + CD74 + IFNG + S100A9 + CIITA + TAP2 | 0.878 | 0.769 | 0.987 |
| 7 days | C3AR1 + CD74 + IFNG + S100A9 + CIITA + TAP2 + CD3D | 0.875 | 0.765 | 0.986 |
| 7 days | C3AR1 + CD74 + IFNG + S100A9 + CIITA + TAP2 + CD3D + CTLA4 | 0.881 | 0.773 | 0.989 |
| 7 days | C3AR1 + CD74 + IFNG + S100A9 + CIITA + TAP2 + CTLA4 | 0.884 | 0.777 | 0.991 |
| 7 days | C3AR1 + CD74 + IFNG + S100A9 + CTLA4 | 0.823 | 0.691 | 0.955 |
| 7 days | C3AR1 + CD74 + IFNG + S100A9 + CX3CR1 | 0.859 | 0.74 | 0.977 |
| 7 days | C3AR1 + CD74 + IFNG + S100A9 + CX3CR1 + CD3D | 0.875 | 0.766 | 0.985 |
| 7 days | C3AR1 + CD74 + IFNG + S100A9 + CX3CR1 + CD3D + CTLA4 | 0.878 | 0.767 | 0.99 |
| 7 days | C3AR1 + CD74 + IFNG + S100A9 + CX3CR1 + CIITA | 0.917 | 0.824 | 1 |
| 7 days | C3AR1 + CD74 + IFNG + S100A9 + CX3CR1 + CIITA + CD3D | 0.914 | 0.819 | 1 |
| 7 days | C3AR1 + CD74 + IFNG + S100A9 + CX3CR1 + CIITA + CD3D + CTLA4 | 0.92 | 0.828 | 1 |
| 7 days | C3AR1 + CD74 + IFNG + S100A9 + CX3CR1 + CIITA + CTLA4 | 0.922 | 0.832 | 1 |
| 7 days | C3AR1 + CD74 + IFNG + S100A9 + CX3CR1 + CIITA + TAP2 | 0.922 | 0.832 | 1 |
| 7 days | C3AR1 + CD74 + IFNG + S100A9 + CX3CR1 + CIITA + TAP2 + CD3D | 0.911 | 0.814 | 1 |
| 7 days | C3AR1 + CD74 + IFNG + S100A9 + CX3CR1 + CIITA + TAP2 + CD3D + CTLA4 | 0.925 | 0.838 | 1 |
| 7 days | C3AR1 + CD74 + IFNG + S100A9 + CX3CR1 + CIITA + TAP2 + CTLA4 | 0.925 | 0.839 | 1 |
| 7 days | C3AR1 + CD74 + IFNG + S100A9 + CX3CR1 + CTLA4 | 0.881 | 0.772 | 0.99 |
| 7 days | C3AR1 + CD74 + IFNG + S100A9 + CX3CR1 + TAP2 | 0.861 | 0.744 | 0.979 |
| 7 days | C3AR1 + CD74 + IFNG + S100A9 + CX3CR1 + TAP2 + CD3D | 0.878 | 0.77 | 0.987 |
| 7 days | C3AR1 + CD74 + IFNG + S100A9 + CX3CR1 + TAP2 + CD3D + CTLA4 | 0.873 | 0.759 | 0.986 |
| 7 days | C3AR1 + CD74 + IFNG + S100A9 + CX3CR1 + TAP2 + CTLA4 | 0.881 | 0.772 | 0.99 |
| 7 days | C3AR1 + CD74 + IFNG + S100A9 + TAP2 | 0.845 | 0.721 | 0.969 |
| 7 days | C3AR1 + CD74 + IFNG + S100A9 + TAP2 + CD3D | 0.839 | 0.714 | 0.965 |
| 7 days | C3AR1 + CD74 + IFNG + S100A9 + TAP2 + CD3D + CTLA4 | 0.839 | 0.714 | 0.965 |
| 7 days | C3AR1 + CD74 + IFNG + S100A9 + TAP2 + CTLA4 | 0.831 | 0.702 | 0.96 |
| 7 days | C3AR1 + CD74 + IFNG + TAP2 | 0.828 | 0.699 | 0.957 |
| 7 days | C3AR1 + CD74 + IFNG + TAP2 + CD3D | 0.825 | 0.696 | 0.955 |
| 7 days | C3AR1 + CD74 + IFNG + TAP2 + CD3D + CTLA4 | 0.834 | 0.706 | 0.962 |
| 7 days | C3AR1 + CD74 + IFNG + TAP2 + CTLA4 | 0.831 | 0.702 | 0.96 |
| 7 days | C3AR1 + CD74 + S100A9 | 0.828 | 0.687 | 0.97 |
| 7 days | C3AR1 + CD74 + S100A9 + CD3D | 0.814 | 0.672 | 0.957 |
| 7 days | C3AR1 + CD74 + S100A9 + CD3D + CTLA4 | 0.823 | 0.684 | 0.962 |
| 7 days | C3AR1 + CD74 + S100A9 + CIITA | 0.886 | 0.778 | 0.995 |
| 7 days | C3AR1 + CD74 + S100A9 + CIITA + CD3D | 0.878 | 0.766 | 0.99 |
| 7 days | C3AR1 + CD74 + S100A9 + CIITA + CD3D + CTLA4 | 0.867 | 0.754 | 0.98 |
| 7 days | C3AR1 + CD74 + S100A9 + CIITA + CTLA4 | 0.878 | 0.765 | 0.992 |
| 7 days | C3AR1 + CD74 + S100A9 + CIITA + TAP2 | 0.88€ | 0.778 | 0.995 |
| 7 days | C3AR1 + CD74 + S100A9 + CIITA + TAP2 + CD3D | 0.886 | 0.779 | 0.994 |
| 7 days | C3AR1 + CD74 + S100A9 + CIITA + TAP2 + CD3D + CTLA4 | 0.884 | 0.778 | 0.99 |
| 7 days | C3AR1 + CD74 + S100A9 + CIITA + TAP2 + CTLA4 | 0.881 | 0.768 | 0.994 |
| 7 days | C3AR1 + CD74 + S100A9 + CTLA4 | 0.828 | 0.687 | 0.97 |
| 7 days | C3AR1 + CD74 + S100A9 + CX3CR1 | 0.848 | 0.711 | 0.984 |
| 7 days | C3AR1 + CD74 + S100A9 + CX3CR1 + CD3D | 0.856 | 0.722 | 0.99 |
| 7 days | C3AR1 + CD74 + S100A9 + CX3CR1 + CD3D + CTLA4 | 0.856 | 0.722 | 0.99 |
| 7 days | C3AR1 + CD74 + S100A9 + CX3CR1 + CIITA | 0.881 | 0.764 | 0.998 |
| 7 days | C3AR1 + CD74 + S100A9 + CX3CR1 + CIITA + CD3D | 0.884 | 0.769 | 0.998 |
| 7 days | C3AR1 + CD74 + S100A9 + CX3CR1 + CIITA + CD3D + CTLA4 | 0.889 | 0.785 | 0.994 |
| 7 days | C3AR1 + CD74 + S100A9 + CX3CR1 + CIITA + CTLA4 | 0.886 | 0.773 | 1 |
| 7 days | C3AR1 + CD74 + S100A9 + CX3CR1 + CIITA + TAP2 | 0.881 | 0.763 | 0.998 |
| 7 days | C3AR1 + CD74 + S100A9 + CX3CR1 + CIITA + TAP2 + CD3D | 0.884 | 0.769 | 0.998 |
| 7 days | C3AR1 + CD74 + S100A9 + CX3CR1 + CIITA + TAP2 + CD3D + CTLA4 | 0.892 | 0.79 | 0.994 |
| 7 days | C3AR1 + CD74 + S100A9 + CX3CR1 + CIITA + TAP2 + CTLA4 | 0.884 | 0.768 | 0.999 |
| 7 days | C3AR1 + CD74 + S100A9 + CX3CR1 + CTLA4 | 0.856 | 0.724 | 0.988 |
| 7 days | C3AR1 + CD74 + S100A9 + CX3CR1 + TAP2 | 0.853 | 0.714 | 0.992 |
| 7 days | C3AR1 + CD74 + S100A9 + CX3CR1 + TAP2 + CD3D | 0.856 | 0.722 | 0.99 |
| 7 days | C3AR1 + CD74 + S100A9 + CX3CR1 + TAP2 + CD3D + CTLA4 | 0.853 | 0.717 | 0.99 |
| 7 days | C3AR1 + CD74 + S100A9 + CX3CR1 + TAP2 + CTLA4 | 0.85 | 0.711 | 0.989 |
| 7 days | C3AR1 + CD74 + S100A9 + TAP2 | 0.825 | 0.684 | 0.967 |
| 7 days | C3AR1 + CD74 + S100A9 + TAP2 + CD3D | 0.823 | 0.682 | 0.963 |
| 7 days | C3AR1 + CD74 + S100A9 + TAP2 + CD3D + CTLA4 | 0.831 | 0.695 | 0.967 |
| 7 days | C3AR1 + CD74 + S100A9 + TAP2 + CTLA4 | 0.825 | 0.684 | 0.967 |
| 7 days | C3AR1 + CD74 + TAP2 | 0.773 | 0.621 | 0.925 |
| 7 days | C3AR1 + CD74 + TAP2 + CD3D | 0.773 | 0.623 | 0.922 |
| 7 days | C3AR1 + CD74 + TAP2 + CD3D + CTLA4 | 0.767 | 0.615 | 0.919 |
| 7 days | C3AR1 + CD74 + TAP2 + CTLA4 | 0.789 | 0.641 | 0.938 |
| 7 days | C3AR1 + CIITA | 0.825 | 0.694 | 0.957 |
| 7 days | C3AR1 + CIITA + CD3D | 0.825 | 0.692 | 0.959 |
| 7 days | C3AR1 + CIITA + CD3D + CTLA4 | 0.839 | 0.716 | 0.963 |
| 7 days | C3AR1 + CIITA + CTLA4 | 0.839 | 0.715 | 0.964 |
| 7 days | C3AR1 + CIITA + TAP2 | 0.834 | 0.705 | 0.963 |
| 7 days | C3AR1 + CIITA + TAP2 + CD3D | 0.828 | 0.698 | 0.959 |
| 7 days | C3AR1 + CIITA + TAP2 + CD3D + CTLA4 | 0.831 | 0.704 | 0.958 |
| 7 days | C3AR1 + CIITA + TAP2 + CTLA4 | 0.842 | 0.719 | 0.965 |
| 7 days | C3AR1 + CX3CR1 + CD3D | 0.64 | 0.459 | 0.821 |
| 7 days | C3AR1 + CX3CR1 + CD3D + CTLA4 | 0.632 | 0.449 | 0.815 |
| 7 days | C3AR1 + CX3CR1 + CIITA | 0.817 | 0.682 | 0.952 |
| 7 days | C3AR1 + CX3CR1 + CIITA + CD3D | 0.837 | 0.711 | 0.962 |
| 7 days | C3AR1 + CX3CR1 + CIITA + CD3D + CTLA4 | 0.831 | 0.704 | 0.958 |
| 7 days | C3AR1 + CX3CR1 + CIITA + CTLA4 | 0.837 | 0.712 | 0.961 |
| 7 days | C3AR1 + CX3CR1 + CIITA + TAP2 | 0.831 | 0.699 | 0.963 |
| 7 days | C3AR1 + CX3CR1 + CIITA + TAP2 + CD3D | 0.839 | 0.714 | 0.965 |
| 7 days | C3AR1 + CX3CR1 + CIITA + TAP2 + CD3D + CTLA4 | 0.853 | 0.736 | 0.97 |
| 7 days | C3AR1 + CX3CR1 + CIITA + TAP2 + CTLA4 | 0.845 | 0.72 | 0.969 |
| 7 days | C3AR1 + CX3CR1 + TAP2 | 0.693 | 0.516 | 0.869 |
| 7 days | C3AR1 + CX3CR1 + TAP2 + CD3D | 0.693 | 0.516 | 0.87 |
| 7 days | C3AR1 + CX3CR1 + TAP2 + CD3D + CTLA4 | 0.695 | 0.518 | 0.873 |
| 7 days | C3AR1 + CX3CR1 + TAP2 + CTLA4 | 0.693 | 0.515 | 0.87 |
| 7 days | C3AR1 + IFNG | 0.734 | 0.571 | 0.897 |
| 7 days | C3AR1 + IFNG + CD177 | 0.806 | 0.665 | 0.947 |
| 7 days | C3AR1 + IFNG + CD177 + CD3D | 0.812 | 0.674 | 0.949 |
| 7 days | C3AR1 + IFNG + CD177 + CD3D + CTLA4 | 0.809 | 0.671 | 0.947 |
| 7 days | C3AR1 + IFNG + CD177 + CIITA | 0.875 | 0.766 | 0.985 |
| 7 days | C3AR1 + IFNG + CD177 + CIITA + CD3D | 0.873 | 0.761 | 0.984 |
| 7 days | C3AR1 + IFNG + CD177 + CIITA + CD3D + CTLA4 | 0.881 | 0.776 | 0.985 |
| 7 days | C3AR1 + IFNG + CD177 + CIITA + CTLA4 | 0.878 | 0.774 | 0.983 |
| 7 days | C3AR1 + IFNG + CD177 + CIITA + TAP2 | 0.878 | 0.77 | 0.987 |
| 7 days | C3AR1 + IFNG + CD177 + CIITA + TAP2 + CD3D | 0.878 | 0.77 | 0.987 |
| 7 days | C3AR1 + IFNG + CD177 + CIITA + TAP2 + CD3D + CTLA4 | 0.892 | 0.795 | 0.989 |
| 7 days | C3AR1 + IFNG + CD177 + CIITA + TAP2 + CTLA4 | 0.881 | 0.778 | 0.984 |
| 7 days | C3AR1 + IFNG + CD177 + CTLA4 | 0.809 | 0.671 | 0.947 |
| 7 days | C3AR1 + IFNG + CD177 + CX3CR1 | 0.803 | 0.66 | 0.947 |
| 7 days | C3AR1 + IFNG + CD177 + CX3CR1 + CD3D | 0.814 | 0.676 | 0.953 |
| 7 days | C3AR1 + IFNG + CD177 + CX3CR1 + CD3D + CTLA4 | 0.792 | 0.648 | 0.937 |
| 7 days | C3AR1 + IFNG + CD177 + CX3CR1 + CIITA | 0.884 | 0.78 | 0.987 |
| 7 days | C3AR1 + IFNG + CD177 + CX3CR1 + CIITA + CD3D | 0.881 | 0.775 | 0.987 |
| 7 days | C3AR1 + IFNG + CD177 + CX3CR1 + CIITA + CD3D + CTLA4 | 0.9 | 0.805 | 0.996 |
| 7 days | C3AR1 + IFNG + CD177 + CX3CR1 + CIITA + CTLA4 | 0.889 | 0.79 | 0.988 |
| 7 days | C3AR1 + IFNG + CD177 + CX3CR1 + CIITA + TAP2 | 0.889 | 0.79 | 0.988 |
| 7 days | C3AR1 + IFNG + CD177 + CX3CR1 + CIITA + TAP2 + CD3D | 0.884 | 0.781 | 0.987 |
| 7 days | C3AR1 + IFNG + CD177 + CX3CR1 + CIITA + TAP2 + CD3D + CTLA4 | 0.9 | 0.803 | 0.997 |
| 7 days | C3AR1 + IFNG + CD177 + CX3CR1 + CIITA + TAP2 + CTLA4 | 0.886 | 0.784 | 0.989 |
| 7 days | C3AR1 + IFNG + CD177 + CX3CR1 + CTLA4 | 0.801 | 0.659 | 0.942 |
| 7 days | C3AR1 + IFNG + CD177 + CX3CR1 + TAP2 | 0.817 | 0.68 | 0.954 |
| 7 days | C3AR1 + IFNG + CD177 + CX3CR1 + TAP2 + CD3D | 0.82 | 0.684 | 0.956 |
| 7 days | C3AR1 + IFNG + CD177 + CX3CR1 + TAP2 + CD3D + CTLA4 | 0.825 | 0.693 | 0.958 |
| 7 days | C3AR1 + IFNG + CD177 + CX3CR1 + TAP2 + CTLA4 | 0.825 | 0.693 | 0.958 |
| 7 days | C3AR1 + IFNG + CD177 + S100A9 | 0.798 | 0.655 | 0.94 |
| 7 days | C3AR1 + IFNG + CD177 + S100A9 + CD3D | 0.795 | 0.651 | 0.939 |
| 7 days | C3AR1 + IFNG + CD177 + S100A9 + CD3D + CTLA4 | 0.801 | 0.659 | 0.942 |
| 7 days | C3AR1 + IFNG + CD177 + S100A9 + CIITA | 0.873 | 0.762 | 0.983 |
| 7 days | C3AR1 + IFNG + CD177 + S100A9 + CIITA + CD3D | 0.875 | 0.766 | 0.985 |
| 7 days | C3AR1 + IFNG + CD177 + S100A9 + CIITA + CD3D + CTLA4 | 0.881 | 0.777 | 0.985 |
| 7 days | C3AR1 + IFNG + CD177 + S100A9 + CIITA + CTLA4 | 0.873 | 0.765 | 0.98 |
| 7 days | C3AR1 + IFNG + CD177 + S100A9 + CIITA + TAP2 | 0.873 | 0.763 | 0.982 |
| 7 days | C3AR1 + IFNG + CD177 + S100A9 + CIITA + TAP2 + CD3D | 0.873 | 0.762 | 0.983 |
| 7 days | C3AR1 + IFNG + CD177 + S100A9 + CIITA + TAP2 + CD3D + CTLA4 | 0.892 | 0.795 | 0.989 |
| 7 days | C3AR1 + IFNG + CD177 + S100A9 + CIITA + TAP2 + CTLA4 | 0.881 | 0.777 | 0.985 |
| 7 days | C3AR1 + IFNG + CD177 + S100A9 + CTLA4 | 0.801 | 0.659 | 0.942 |
| 7 days | C3AR1 + IFNG + CD177 + S100A9 + CX3CR1 | 0.795 | 0.652 | 0.938 |
| 7 days | C3AR1 + IFNG + CD177 + S100A9 + CX3CR1 + CD3D | 0.789 | 0.646 | 0.933 |
| 7 days | C3AR1 + IFNG + CD177 + S100A9 + CX3CR1 + CD3D + CTLA4 | 0.789 | 0.646 | 0.933 |
| 7 days | C3AR1 + IFNG + CD177 + S100A9 + CX3CR1 + CIITA | 0.884 | 0.778 | 0.989 |
| 7 days | C3AR1 + IFNG + CD177 + S100A9 + CX3CR1 + CIITA + CD3D | 0.881 | 0.775 | 0.987 |
| 7 days | C3AR1 + IFNG + CD177 + S100A9 + CX3CR1 + CIITA + CD3D + CTLA4 | 0.895 | 0.797 | 0.993 |
| 7 days | C3AR1 + IFNG + CD177 + S100A9 + CX3CR1 + CIITA + CTLA4 | 0.889 | 0.788 | 0.991 |
| 7 days | C3AR1 + IFNG + CD177 + S100A9 + CX3CR1 + CIITA + TAP2 | 0.903 | 0.807 | 0.999 |
| 7 days | C3AR1 + IFNG + CD177 + S100A9 + CX3CR1 + CIITA + TAP2 + CD3D | 0.9 | 0.803 | 0.997 |
| 7 days | C3AR1 + IFNG + CD177 + S100A9 + CX3CR1 + CIITA + TAP2 + CD3D + CTLA4 | 0.892 | 0.792 | 0.992 |
| 7 days | C3AR1 + IFNG + CD177 + S100A9 + CX3CR1 + CIITA + TAP2 + CTLA4 | 0.892 | 0.789 | 0.995 |
| 7 days | C3AR1 + IFNG + CD177 + S100A9 + CX3CR1 + CTLA4 | 0.806 | 0.667 | 0.945 |
| 7 days | C3AR1 + IFNG + CD177 + S100A9 + CX3CR1 + TAP2 | 0.82 | 0.688 | 0.952 |
| 7 days | C3AR1 + IFNG + CD177 + S100A9 + CX3CR1 + TAP2 + CD3D | 0.809 | 0.672 | 0.945 |
| 7 days | C3AR1 + IFNG + CD177 + S100A9 + CX3CR1 + TAP2 + CD3D + CTLA4 | 0.817 | 0.684 | 0.951 |
| 7 days | C3AR1 + IFNG + CD177 + S100A9 + CX3CR1 + TAP2 + CTLA4 | 0.823 | 0.691 | 0.954 |
| 7 days | C3AR1 + IFNG + CD177 + S100A9 + TAP2 | 0.801 | 0.66 | 0.941 |
| 7 days | C3AR1 + IFNG + CD177 + S100A9 + TAP2 + CD3D | 0.798 | 0.656 | 0.94 |
| 7 days | C3AR1 + IFNG + CD177 + S100A9 + TAP2 + CD3D + CTLA4 | 0.806 | 0.667 | 0.945 |
| 7 days | C3AR1 + IFNG + CD177 + S100A9 + TAP2 + CTLA4 | 0.798 | 0.656 | 0.94 |
| 7 days | C3AR1 + IFNG + CD177 + TAP2 | 0.809 | 0.669 | 0.949 |
| 7 days | C3AR1 + IFNG + CD177 + TAP2 + CD3D | 0.812 | 0.673 | 0.95 |
| 7 days | C3AR1 + IFNG + CD177 + TAP2 + CD3D + CTLA4 | 0.806 | 0.666 | 0.947 |
| 7 days | C3AR1 + IFNG + CD177 + TAP2 + CTLA4 | 0.812 | 0.673 | 0.95 |
| 7 days | C3AR1 + IFNG + CD3D | 0.748 | 0.591 | 0.905 |
| 7 days | C3AR1 + IFNG + CD3D + CTLA4 | 0.745 | 0.586 | 0.904 |
| 7 days | C3AR1 + IFNG + CIITA | 0.85 | 0.73 | 0.97 |
| 7 days | C3AR1 + IFNG + CIITA + CD3D | 0.85 | 0.73 | 0.97 |
| 7 days | C3AR1 + IFNG + CIITA + CD3D + CTLA4 | 0.859 | 0.742 | 0.975 |
| 7 days | C3AR1 + IFNG + CIITA + CTLA4 | 0.859 | 0.743 | 0.975 |
| 7 days | C3AR1 + IFNG + CIITA + TAP2 | 0.848 | 0.727 | 0.969 |
| 7 days | C3AR1 + IFNG + CIITA + TAP2 + CD3D | 0.845 | 0.723 | 0.966 |
| 7 days | C3AR1 + IFNG + CIITA + TAP2 + CD3D + CTLA4 | 0.856 | 0.738 | 0.974 |
| 7 days | C3AR1 + IFNG + CIITA + TAP2 + CTLA4 | 0.856 | 0.738 | 0.974 |
| 7 days | C3AR1 + IFNG + CTLA4 | 0.748 | 0.589 | 0.907 |
| 7 days | C3AR1 + IFNG + CX3CR1 | 0.742 | 0.583 | 0.901 |
| 7 days | C3AR1 + IFNG + CX3CR1 + CD3D | 0.748 | 0.588 | 0.908 |
| 7 days | C3AR1 + IFNG + CX3CR1 + CD3D + CTLA4 | 0.77 | 0.616 | 0.924 |
| 7 days | C3AR1 + IFNG + CX3CR1 + CIITA | 0.861 | 0.745 | 0.978 |
| 7 days | C3AR1 + IFNG + CX3CR1 + CIITA + CD3D | 0.864 | 0.749 | 0.98 |
| 7 days | C3AR1 + IFNG + CX3CR1 + CIITA + CD3D + CTLA4 | 0.867 | 0.751 | 0.983 |
| 7 days | C3AR1 + IFNG + CX3CR1 + CIITA + CTLA4 | 0.859 | 0.738 | 0.979 |
| 7 days | C3AR1 + IFNG + CX3CR1 + CIITA + TAP2 | 0.875 | 0.766 | 0.984 |
| 7 days | C3AR1 + IFNG + CX3CR1 + CIITA + TAP2 + CD3D | 0.878 | 0.771 | 0.986 |
| 7 days | C3AR1 + IFNG + CX3CR1 + CIITA + TAP2 + CD3D + CTLA4 | 0.889 | 0.787 | 0.992 |
| 7 days | C3AR1 + IFNG + CX3CR1 + CIITA + TAP2 + CTLA4 | 0.886 | 0.779 | 0.994 |
| 7 days | C3AR1 + IFNG + CX3CR1 + CTLA4 | 0.77 | 0.616 | 0.924 |
| 7 days | C3AR1 + IFNG + CX3CR1 + TAP2 | 0.776 | 0.622 | 0.929 |
| 7 days | C3AR1 + IFNG + CX3CR1 + TAP2 + CD3D | 0.784 | 0.635 | 0.933 |
| 7 days | C3AR1 + IFNG + CX3CR1 + TAP2 + CD3D + CTLA4 | 0.787 | 0.641 | 0.932 |
| 7 days | C3AR1 + IFNG + CX3CR1 + TAP2 + CTLA4 | 0.784 | 0.637 | 0.931 |
| 7 days | C3AR1 + IFNG + S100A9 | 0.762 | 0.609 | 0.915 |
| 7 days | C3AR1 + IFNG + S100A9 + CD3D | 0.762 | 0.609 | 0.914 |
| 7 days | C3AR1 + IFNG + S100A9 + CD3D + CTLA4 | 0.759 | 0.606 | 0.912 |
| 7 days | C3AR1 + IFNG + S100A9 + CIITA | 0.859 | 0.743 | 0.974 |
| 7 days | C3AR1 + IFNG + S100A9 + CIITA + CD3D | 0.859 | 0.743 | 0.974 |
| 7 days | C3AR1 + IFNG + S100A9 + CIITA + CD3D + CTLA4 | 0.853 | 0.736 | 0.971 |
| 7 days | C3AR1 + IFNG + S100A9 + CIITA + CTLA4 | 0.859 | 0.744 | 0.974 |
| 7 days | C3AR1 + IFNG + S100A9 + CIITA + TAP2 | 0.856 | 0.739 | 0.973 |
| 7 days | C3AR1 + IFNG + S100A9 + CIITA + TAP2 + CD3D | 0.856 | 0.739 | 0.973 |
| 7 days | C3AR1 + IFNG + S100A9 + CIITA + TAP2 + CD3D + CTLA4 | 0.853 | 0.736 | 0.971 |
| 7 days | C3AR1 + IFNG + S100A9 + CIITA + TAP2 + CTLA4 | 0.85 | 0.731 | 0.97 |
| 7 days | C3AR1 + IFNG + S100A9 + CTLA4 | 0.745 | 0.587 | 0.903 |
| 7 days | C3AR1 + IFNG + S100A9 + CX3CR1 | 0.781 | 0.633 | 0.929 |
| 7 days | C3AR1 + IFNG + S100A9 + CX3CR1 + CD3D | 0.789 | 0.643 | 0.936 |
| 7 days | C3AR1 + IFNG + S100A9 + CX3CR1 + CD3D + CTLA4 | 0.787 | 0.639 | 0.934 |
| 7 days | C3AR1 + IFNG + S100A9 + CX3CR1 + CIITA | 0.873 | 0.762 | 0.983 |
| 7 days | C3AR1 + IFNG + S100A9 + CX3CR1 + CIITA + CD3D | 0.875 | 0.765 | 0.986 |
| 7 days | C3AR1 + IFNG + S100A9 + CX3CR1 + CIITA + CD3D + CTLA4 | 0.884 | 0.78 | 0.987 |
| 7 days | C3AR1 + IFNG + S100A9 + CX3CR1 + CIITA + CTLA4 | 0.87 | 0.759 | 0.981 |
| 7 days | C3AR1 + IFNG + S100A9 + CX3CR1 + CIITA + TAP2 | 0.884 | 0.78 | 0.987 |
| 7 days | C3AR1 + IFNG + S100A9 + CX3CR1 + CIITA + TAP2 + CD3D | 0.889 | 0.789 | 0.989 |
| 7 days | C3AR1 + IFNG + S100A9 + CX3CR1 + CIITA + TAP2 + CD3D + CTLA4 | 0.881 | 0.777 | 0.985 |
| 7 days | C3AR1 + IFNG + S100A9 + CX3CR1 + CIITA + TAP2 + CTLA4 | 0.886 | 0.784 | 0.989 |
| 7 days | C3AR1 + IFNG + S100A9 + CX3CR1 + CTLA4 | 0.787 | 0.64 | 0.934 |
| 7 days | C3AR1 + IFNG + S100A9 + CX3CR1 + TAP2 | 0.787 | 0.642 | 0.932 |
| 7 days | C3AR1 + IFNG + S100A9 + CX3CR1 + TAP2 + CD3D | 0.787 | 0.641 | 0.932 |
| 7 days | C3AR1 + IFNG + S100A9 + CX3CR1 + TAP2 + CD3D + CTLA4 | 0.792 | 0.649 | 0.936 |
| 7 days | C3AR1 + IFNG + S100A9 + CX3CR1 + TAP2 + CTLA4 | 0.781 | 0.633 | 0.929 |
| 7 days | C3AR1 + IFNG + S100A9 + TAP2 | 0.762 | 0.608 | 0.915 |
| 7 days | C3AR1 + IFNG + S100A9 + TAP2 + CD3D | 0.762 | 0.608 | 0.916 |
| 7 days | C3AR1 + IFNG + S100A9 + TAP2 + CD3D + CTLA4 | 0.762 | 0.608 | 0.915 |
| 7 days | C3AR1 + IFNG + S100A9 + TAP2 + CTLA4 | 0.762 | 0.608 | 0.915 |
| 7 days | C3AR1 + IFNG + TAP2 | 0.753 | 0.594 | 0.913 |
| 7 days | C3AR1 + IFNG + TAP2 + CD3D | 0.748 | 0.587 | 0.908 |
| 7 days | C3AR1 + IFNG + TAP2 + CD3D + CTLA4 | 0.748 | 0.588 | 0.907 |
| 7 days | C3AR1 + IFNG + TAP2 + CTLA4 | 0.748 | 0.587 | 0.909 |
| 7 days | C3AR1 + IL1R2 + CD177 | 0.726 | 0.564 | 0.887 |
| 7 days | C3AR1 + IL1R2 + CD177 + CD3D | 0.726 | 0.564 | 0.887 |
| 7 days | C3AR1 + IL1R2 + CD177 + CD3D + CTLA4 | 0.726 | 0.564 | 0.887 |
| 7 days | C3AR1 + IL1R2 + CD177 + CIITA | 0.853 | 0.729 | 0.977 |
| 7 days | C3AR1 + IL1R2 + CD177 + CIITA + CD3D | 0.859 | 0.736 | 0.981 |
| 7 days | C3AR1 + IL1R2 + CD177 + CIITA + CD3D + CTLA4 | 0.878 | 0.771 | 0.985 |
| 7 days | C3AR1 + IL1R2 + CD177 + CIITA + CTLA4 | 0.875 | 0.764 | 0.987 |
| 7 days | C3AR1 + IL1R2 + CD177 + CIITA + TAP2 | 0.856 | 0.732 | 0.98 |
| 7 days | C3AR1 + IL1R2 + CD177 + CIITA + TAP2 + CD3D | 0.861 | 0.74 | 0.983 |
| 7 days | C3AR1 + IL1R2 + CD177 + CIITA + TAP2 + CD3D + CTLA4 | 0.875 | 0.765 | 0.986 |
| 7 days | C3AR1 + IL1R2 + CD177 + CIITA + TAP2 + CTLA4 | 0.878 | 0.769 | 0.987 |
| 7 days | C3AR1 + IL1R2 + CD177 + CTLA4 | 0.726 | 0.564 | 0.887 |
| 7 days | C3AR1 + IL1R2 + CD177 + CX3CR1 | 0.723 | 0.561 | 0.885 |
| 7 days | C3AR1 + IL1R2 + CD177 + CX3CR1 + CD3D | 0.72 | 0.557 | 0.883 |
| 7 days | C3AR1 + IL1R2 + CD177 + CX3CR1 + CD3D + CTLA4 | 0.723 | 0.561 | 0.885 |
| 7 days | C3AR1 + IL1R2 + CD177 + CX3CR1 + CIITA | 0.856 | 0.732 | 0.98 |
| 7 days | C3AR1 + IL1R2 + CD177 + CX3CR1 + CIITA + CD3D | 0.853 | 0.729 | 0.977 |
| 7 days | C3AR1 + IL1R2 + CD177 + CX3CR1 + CIITA + CD3D + CTLA4 | 0.884 | 0.776 | 0.991 |
| 7 days | C3AR1 + IL1R2 + CD177 + CX3CR1 + CIITA + CTLA4 | 0.87 | 0.756 | 0.984 |
| 7 days | C3AR1 + IL1R2 + CD177 + CX3CR1 + CIITA + TAP2 | 0.856 | 0.731 | 0.981 |
| 7 days | C3AR1 + IL1R2 + CD177 + CX3CR1 + CIITA + TAP2 + CD3D | 0.856 | 0.732 | 0.98 |
| 7 days | C3AR1 + IL1R2 + CD177 + CX3CR1 + CIITA + TAP2 + CD3D + CTLA4 | 0.884 | 0.776 | 0.991 |
| 7 days | C3AR1 + IL1R2 + CD177 + CX3CR1 + CIITA + TAP2 + CTLA4 | 0.873 | 0.759 | 0.986 |
| 7 days | C3AR1 + IL1R2 + CD177 + CX3CR1 + CTLA4 | 0.731 | 0.571 | 0.892 |
| 7 days | C3AR1 + IL1R2 + CD177 + CX3CR1 + TAP2 | 0.767 | 0.612 | 0.922 |
| 7 days | C3AR1 + IL1R2 + CD177 + CX3CR1 + TAP2 + CD3D | 0.767 | 0.614 | 0.921 |
| 7 days | C3AR1 + IL1R2 + CD177 + CX3CR1 + TAP2 + CD3D + CTLA4 | 0.77 | 0.613 | 0.927 |
| 7 days | C3AR1 + IL1R2 + CD177 + CX3CR1 + TAP2 + CTLA4 | 0.767 | 0.609 | 0.926 |
| 7 days | C3AR1 + IL1R2 + CD177 + S100A9 | 0.767 | 0.615 | 0.92 |
| 7 days | C3AR1 + IL1R2 + CD177 + S100A9 + CD3D | 0.776 | 0.626 | 0.926 |
| 7 days | C3AR1 + IL1R2 + CD177 + S100A9 + CD3D + CTLA4 | 0.767 | 0.616 | 0.918 |
| 7 days | C3AR1 + IL1R2 + CD177 + S100A9 + CIITA | 0.859 | 0.739 | 0.979 |
| 7 days | C3AR1 + IL1R2 + CD177 + S100A9 + CIITA + CD3D | 0.864 | 0.747 | 0.982 |
| 7 days | C3AR1 + IL1R2 + CD177 + S100A9 + CIITA + CD3D + CTLA4 | 0.892 | 0.792 | 0.992 |
| 7 days | C3AR1 + IL1R2 + CD177 + S100A9 + CIITA + CTLA4 | 0.884 | 0.778 | 0.989 |
| 7 days | C3AR1 + IL1R2 + CD177 + S100A9 + CIITA + TAP2 | 0.861 | 0.743 | 0.98 |
| 7 days | C3AR1 + IL1R2 + CD177 + S100A9 + CIITA + TAP2 + CD3D | 0.864 | 0.747 | 0.982 |
| 7 days | C3AR1 + IL1R2 + CD177 + S100A9 + CIITA + TAP2 + CD3D + CTLA4 | 0.889 | 0.789 | 0.989 |
| 7 days | C3AR1 + IL1R2 + CD177 + S100A9 + CIITA + TAP2 + CTLA4 | 0.881 | 0.773 | 0.988 |
| 7 days | C3AR1 + IL1R2 + CD177 + S100A9 + CTLA4 | 0.767 | 0.614 | 0.92 |
| 7 days | C3AR1 + IL1R2 + CD177 + S100A9 + CX3CR1 | 0.773 | 0.622 | 0.924 |
| 7 days | C3AR1 + IL1R2 + CD177 + S100A9 + CX3CR1 + CD3D | 0.789 | 0.642 | 0.937 |
| 7 days | C3AR1 + IL1R2 + CD177 + S100A9 + CX3CR1 + CD3D + CTLA4 | 0.767 | 0.613 | 0.921 |
| 7 days | C3AR1 + IL1R2 + CD177 + S100A9 + CX3CR1 + CIITA | 0.856 | 0.733 | 0.979 |
| 7 days | C3AR1 + IL1R2 + CD177 + S100A9 + CX3CR1 + CIITA + CD3D | 0.861 | 0.742 | 0.981 |
| 7 days | C3AR1 + IL1R2 + CD177 + S100A9 + CX3CR1 + CIITA + CD3D + CTLA4 | 0.9 | 0.804 | 0.996 |
| 7 days | C3AR1 + IL1R2 + CD177 + S100A9 + CX3CR1 + CIITA + CTLA4 | 0.873 | 0.761 | 0.984 |
| 7 days | C3AR1 + IL1R2 + CD177 + S100A9 + CX3CR1 + CIITA + TAP2 | 0.861 | 0.74 | 0.983 |
| 7 days | C3AR1 + IL1R2 + CD177 + S100A9 + CX3CR1 + CIITA + TAP2 + CD3D | 0.859 | 0.737 | 0.98 |
| 7 days | C3AR1 + IL1R2 + CD177 + S100A9 + CX3CR1 + CIITA + TAP2 + CD3D + CTLA4 | 0.9 | 0.806 | 0.995 |
| 7 days | C3AR1 + IL1R2 + CD177 + S100A9 + CX3CR1 + CIITA + TAP2 + CTLA4 | 0.875 | 0.765 | 0.985 |
| 7 days | C3AR1 + IL1R2 + CD177 + S100A9 + CX3CR1 + CTLA4 | 0.776 | 0.625 | 0.926 |
| 7 days | C3AR1 + IL1R2 + CD177 + S100A9 + CX3CR1 + TAP2 | 0.778 | 0.626 | 0.93 |
| 7 days | C3AR1 + IL1R2 + CD177 + S100A9 + CX3CR1 + TAP2 + CD3D | 0.781 | 0.631 | 0.932 |
| 7 days | C3AR1 + IL1R2 + CD177 + S100A9 + CX3CR1 + TAP2 + CD3D + CTLA4 | 0.784 | 0.637 | 0.931 |
| 7 days | C3AR1 + IL1R2 + CD177 + S100A9 + CX3CR1 + TAP2 + CTLA4 | 0.781 | 0.63 | 0.932 |
| 7 days | C3AR1 + IL1R2 + CD177 + S100A9 + TAP2 | 0.795 | 0.649 | 0.941 |
| 7 days | C3AR1 + IL1R2 + CD177 + S100A9 + TAP2 + CD3D | 0.787 | 0.639 | 0.934 |
| 7 days | C3AR1 + IL1R2 + CD177 + S100A9 + TAP2 + CD3D + CTLA4 | 0.787 | 0.64 | 0.933 |
| 7 days | C3AR1 + IL1R2 + CD177 + S100A9 + TAP2 + CTLA4 | 0.795 | 0.649 | 0.941 |
| 7 days | C3AR1 + IL1R2 + CD177 + TAP2 | 0.767 | 0.612 | 0.922 |
| 7 days | C3AR1 + IL1R2 + CD177 + TAP2 + CD3D | 0.765 | 0.609 | 0.92 |
| 7 days | C3AR1 + IL1R2 + CD177 + TAP2 + CD3D + CTLA4 | 0.773 | 0.62 | 0.926 |
| 7 days | C3AR1 + IL1R2 + CD177 + TAP2 + CTLA4 | 0.765 | 0.61 | 0.919 |
| 7 days | C3AR1 + IL1R2 + CD74 | 0.688 | 0.601 | 0.775 |
| 7 days | C3AR1 + IL1R2 + CD74 + CD177 | 0.812 | 0.67 | 0.953 |
| 7 days | C3AR1 + IL1R2 + CD74 + CD177 + CD3D | 0.809 | 0.668 | 0.95 |
| 7 days | C3AR1 + IL1R2 + CD74 + CD177 + CD3D + CTLA4 | 0.806 | 0.664 | 0.948 |
| 7 days | C3AR1 + IL1R2 + CD74 + CD177 + CIITA | 0.875 | 0.763 | 0.988 |
| 7 days | C3AR1 + IL1R2 + CD74 + CD177 + CIITA + CD3D | 0.878 | 0.768 | 0.988 |
| 7 days | C3AR1 + IL1R2 + CD74 + CD177 + CIITA + CD3D + CTLA4 | 0.881 | 0.775 | 0.986 |
| 7 days | C3AR1 + IL1R2 + CD74 + CD177 + CIITA + CTLA4 | 0.873 | 0.761 | 0.985 |
| 7 days | C3AR1 + IL1R2 + CD74 + CD177 + CIITA + TAP2 | 0.875 | 0.763 | 0.988 |
| 7 days | C3AR1 + IL1R2 + CD74 + CD177 + CIITA + TAP2 + CD3D | 0.875 | 0.764 | 0.986 |
| 7 days | C3AR1 + IL1R2 + CD74 + CD177 + CIITA + TAP2 + CD3D + CTLA4 | 0.881 | 0.775 | 0.987 |
| 7 days | C3AR1 + IL1R2 + CD74 + CD177 + CIITA + TAP2 + CTLA4 | 0.873 | 0.761 | 0.985 |
| 7 days | C3AR1 + IL1R2 + CD74 + CD177 + CTLA4 | 0.817 | 0.678 | 0.957 |
| 7 days | C3AR1 + IL1R2 + CD74 + CD177 + CX3CR1 | 0.828 | 0.688 | 0.969 |
| 7 days | C3AR1 + IL1R2 + CD74 + CD177 + CX3CR1 + CD3D | 0.814 | 0.674 | 0.955 |
| 7 days | C3AR1 + IL1R2 + CD74 + CD177 + CX3CR1 + CD3D + CTLA4 | 0.817 | 0.678 | 0.957 |
| 7 days | C3AR1 + IL1R2 + CD74 + CD177 + CX3CR1 + CIITA | 0.881 | 0.771 | 0.991 |
| 7 days | C3AR1 + IL1R2 + CD74 + CD177 + CX3CR1 + CIITA + CD3D | 0.875 | 0.763 | 0.988 |
| 7 days | C3AR1 + IL1R2 + CD74 + CD177 + CX3CR1 + CIITA + CD3D + CTLA4 | 0.889 | 0.787 | 0.991 |
| 7 days | C3AR1 + IL1R2 + CD74 + CD177 + CX3CR1 + CIITA + CTLA4 | 0.881 | 0.771 | 0.991 |
| 7 days | C3AR1 + IL1R2 + CD74 + CD177 + CX3CR1 + CIITA + TAP2 | 0.875 | 0.762 | 0.989 |
| 7 days | C3AR1 + IL1R2 + CD74 + CD177 + CX3CR1 + CIITA + TAP2 + CD3D | 0.873 | 0.758 | 0.987 |
| 7 days | C3AR1 + IL1R2 + CD74 + CD177 + CX3CR1 + CIITA + TAP2 + CD3D + CTLA4 | 0.898 | 0.801 | 0.994 |
| 7 days | C3AR1 + IL1R2 + CD74 + CD177 + CX3CR1 + CIITA + TAP2 + CTLA4 | 0.884 | 0.775 | 0.992 |
| 7 days | C3AR1 + IL1R2 + CD74 + CD177 + CX3CR1 + CTLA4 | 0.834 | 0.697 | 0.97 |
| 7 days | C3AR1 + IL1R2 + CD74 + CD177 + CX3CR1 + TAP2 | 0.82 | 0.676 | 0.964 |
| 7 days | C3AR1 + IL1R2 + CD74 + CD177 + CX3CR1 + TAP2 + CD3D | 0.823 | 0.682 | 0.963 |
| 7 days | C3AR1 + IL1R2 + CD74 + CD177 + CX3CR1 + TAP2 + CD3D + CTLA4 | 0.823 | 0.682 | 0.963 |
| 7 days | C3AR1 + IL1R2 + CD74 + CD177 + CX3CR1 + TAP2 + CTLA4 | 0.831 | 0.692 | 0.97 |
| 7 days | C3AR1 + IL1R2 + CD74 + CD177 + S100A9 | 0.828 | 0.691 | 0.965 |
| 7 days | C3AR1 + IL1R2 + CD74 + CD177 + S100A9 + CD3D | 0.839 | 0.706 | 0.973 |
| 7 days | C3AR1 + IL1R2 + CD74 + CD177 + S100A9 + CD3D + CTLA4 | 0.837 | 0.706 | 0.967 |
| 7 days | C3AR1 + IL1R2 + CD74 + CD177 + S100A9 + CIITA | 0.884 | 0.775 | 0.992 |
| 7 days | C3AR1 + IL1R2 + CD74 + CD177 + S100A9 + CIITA + CD3D | 0.884 | 0.776 | 0.992 |
| 7 days | C3AR1 + IL1R2 + CD74 + CD177 + S100A9 + CIITA + CD3D + CTLA4 | 0.892 | 0.791 | 0.993 |
| 7 days | C3AR1 + IL1R2 + CD74 + CD177 + S100A9 + CIITA + CTLA4 | 0.875 | 0.766 | 0.985 |
| 7 days | C3AR1 + IL1R2 + CD74 + CD177 + S100A9 + CIITA + TAP2 | 0.881 | 0.771 | 0.99 |
| 7 days | C3AR1 + IL1R2 + CD74 + CD177 + S100A9 + CIITA + TAP2 + CD3D | 0.884 | 0.775 | 0.992 |
| 7 days | C3AR1 + IL1R2 + CD74 + CD177 + S100A9 + CIITA + TAP2 + CD3D + CTLA4 | 0.903 | 0.809 | 0.997 |
| 7 days | C3AR1 + IL1R2 + CD74 + CD177 + S100A9 + CIITA + TAP2 + CTLA4 | 0.875 | 0.766 | 0.985 |
| 7 days | C3AR1 + IL1R2 + CD74 + CD177 + S100A9 + CTLA4 | 0.828 | 0.691 | 0.965 |
| 7 days | C3AR1 + IL1R2 + CD74 + CD177 + S100A9 + CX3CR1 | 0.859 | 0.726 | 0.992 |
| 7 days | C3AR1 + IL1R2 + CD74 + CD177 + S100A9 + CX3CR1 + CD3D | 0.848 | 0.712 | 0.983 |
| 7 days | C3AR1 + IL1R2 + CD74 + CD177 + S100A9 + CX3CR1 + CD3D + CTLA4 | 0.845 | 0.708 | 0.981 |
| 7 days | C3AR1 + IL1R2 + CD74 + CD177 + S100A9 + CX3CR1 + CIITA | 0.881 | 0.766 | 0.995 |
| 7 days | C3AR1 + IL1R2 + CD74 + CD177 + S100A9 + CX3CR1 + CIITA + CD3D | 0.884 | 0.771 | 0.996 |
| 7 days | C3AR1 + IL1R2 + CD74 + CD177 + S100A9 + CX3CR1 + CIITA + CD3D + CTLA4 | 0.9 | 0.802 | 0.999 |
| 7 days | C3AR1 + IL1R2 + CD74 + CD177 + S100A9 + CX3CR1 + CIITA + CTLA4 | 0.878 | 0.767 | 0.989 |
| 7 days | C3AR1 + IL1R2 + CD74 + CD177 + S100A9 + CX3CR1 + CIITA + TAP2 | 0.881 | 0.769 | 0.993 |
| 7 days | C3AR1 + IL1R2 + CD74 + CD177 + S100A9 + CX3CR1 + CIITA + TAP2 + CD3D | 0.881 | 0.768 | 0.993 |
| 7 days | C3AR1 + IL1R2 + CD74 + CD177 + S100A9 + CX3CR1 + CIITA + TAP2 + CD3D + | 0.909 | 0.814 | 1 |
| | CTLA4 | | | |
| 7 days | C3AR1 + IL1R2 + CD74 + CD177 + S100A9 + CX3CR1 + CIITA + TAP2 + CTLA4 | 0.884 | 0.774 | 0.993 |
| 7 days | C3AR1 + IL1R2 + CD74 + CD177 + S100A9 + CX3CR1 + CTLA4 | 0.861 | 0.731 | 0.992 |
| 7 days | C3AR1 + IL1R2 + CD74 + CD177 + S100A9 + CX3CR1 + TAP2 | 0.859 | 0.725 | 0.992 |
| 7 days | C3AR1 + IL1R2 + CD74 + CD177 + S100A9 + CX3CR1 + TAP2 + CD3D | 0.85 | 0.716 | 0.985 |
| 7 days | C3AR1 + IL1R2 + CD74 + CD177 + S100A9 + CX3CR1 + TAP2 + CD3D + CTLA4 | 0.845 | 0.708 | 0.981 |
| 7 days | C3AR1 + IL1R2 + CD74 + CD177 + S100A9 + CX3CR1 + TAP2 + CTLA4 | 0.859 | 0.725 | 0.992 |
| 7 days | C3AR1 + IL1R2 + CD74 + CD177 + S100A9 + TAP2 | 0.828 | 0.69 | 0.966 |
| 7 days | C3AR1 + IL1R2 + CD74 + CD177 + S100A9 + TAP2 + CD3D | 0.831 | 0.696 | 0.966 |
| 7 days | C3AR1 + IL1R2 + CD74 + CD177 + S100A9 + TAP2 + CD3D + CTLA4 | 0.839 | 0.709 | 0.969 |
| 7 days | C3AR1 + IL1R2 + CD74 + CD177 + S100A9 + TAP2 + CTLA4 | 0.828 | 0.69 | 0.966 |
| 7 days | C3AR1 + IL1R2 + CD74 + CD177 + TAP2 | 0.817 | 0.677 | 0.958 |
| 7 days | C3AR1 + IL1R2 + CD74 + CD177 + TAP2 + CD3D | 0.814 | 0.674 | 0.955 |
| 7 days | C3AR1 + IL1R2 + CD74 + CD177 + TAP2 + CD3D + CTLA4 | 0.812 | 0.67 | 0.953 |
| 7 days | C3AR1 + IL1R2 + CD74 + CD177 + TAP2 + CTLA4 | 0.814 | 0.672 | 0.956 |
| 7 days | C3AR1 + IL1R2 + CD74 + CD3D | 0.759 | 0.607 | 0.911 |
| 7 days | C3AR1 + IL1R2 + CD74 + CD3D + CTLA4 | 0.756 | 0.602 | 0.91 |
| 7 days | C3AR1 + IL1R2 + CD74 + CIITA | 0.873 | 0.758 | 0.987 |
| 7 days | C3AR1 + IL1R2 + CD74 + CIITA + CD3D | 0.861 | 0.743 | 0.98 |
| 7 days | C3AR1 + IL1R2 + CD74 + CIITA + CD3D + CTLA4 | 0.856 | 0.737 | 0.975 |
| 7 days | C3AR1 + IL1R2 + CD74 + CIITA + CTLA4 | 0.878 | 0.766 | 0.99 |
| 7 days | C3AR1 + IL1R2 + CD74 + CIITA + TAP2 | 0.864 | 0.745 | 0.983 |
| 7 days | C3AR1 + IL1R2 + CD74 + CIITA + TAP2 + CD3D | 0.861 | 0.743 | 0.98 |
| 7 days | C3AR1 + IL1R2 + CD74 + CIITA + TAP2 + CD3D + CTLA4 | 0.856 | 0.737 | 0.975 |
| 7 days | C3AR1 + IL1R2 + CD74 + CIITA + TAP2 + CTLA4 | 0.878 | 0.766 | 0.991 |
| 7 days | C3AR1 + IL1R2 + CD74 + CTLA4 | 0.767 | 0.614 | 0.921 |
| 7 days | C3AR1 + IL1R2 + CD74 + CX3CR1 | 0.776 | 0.616 | 0.935 |
| 7 days | C3AR1 + IL1R2 + CD74 + CX3CR1 + CD3D | 0.773 | 0.622 | 0.924 |
| 7 days | C3AR1 + IL1R2 + CD74 + CX3CR1 + CD3D + CTLA4 | 0.776 | 0.627 | 0.924 |
| 7 days | C3AR1 + IL1R2 + CD74 + CX3CR1 + CIITA | 0.873 | 0.756 | 0.989 |
| 7 days | C3AR1 + IL1R2 + CD74 + CX3CR1 + CIITA + CD3D | 0.867 | 0.75 | 0.984 |
| 7 days | C3AR1 + IL1R2 + CD74 + CX3CR1 + CIITA + CD3D + CTLA4 | 0.867 | 0.753 | 0.981 |
| 7 days | C3AR1 + IL1R2 + CD74 + CX3CR1 + CIITA + CTLA4 | 0.884 | 0.773 | 0.995 |
| 7 days | C3AR1 + IL1R2 + CD74 + CX3CR1 + CIITA + TAP2 | 0.873 | 0.753 | 0.992 |
| 7 days | C3AR1 + IL1R2 + CD74 + CX3CR1 + CIITA + TAP2 + CD3D | 0.87 | 0.754 | 0.986 |
| 7 days | C3AR1 + IL1R2 + CD74 + CX3CR1 + CIITA + TAP2 + CD3D + CTLA4 | 0.864 | 0.749 | 0.979 |
| 7 days | C3AR1 + IL1R2 + CD74 + CX3CR1 + CIITA + TAP2 + CTLA4 | 0.87 | 0.752 | 0.988 |
| 7 days | C3AR1 + IL1R2 + CD74 + CX3CR1 + CTLA4 | 0.776 | 0.623 | 0.929 |
| 7 days | C3AR1 + IL1R2 + CD74 + CX3CR1 + TAP2 | 0.781 | 0.626 | 0.936 |
| 7 days | C3AR1 + IL1R2 + CD74 + CX3CR1 + TAP2 + CD3D | 0.784 | 0.634 | 0.934 |
| 7 days | C3AR1 + IL1R2 + CD74 + CX3CR1 + TAP2 + CD3D + CTLA4 | 0.798 | 0.652 | 0.944 |
| 7 days | C3AR1 + IL1R2 + CD74 + CX3CR1 + TAP2 + CTLA4 | 0.798 | 0.647 | 0.948 |
| 7 days | C3AR1 + IL1R2 + CD74 + IFNG | 0.823 | 0.689 | 0.956 |
| 7 days | C3AR1 + IL1R2 + CD74 + IFNG + CD177 | 0.842 | 0.719 | 0.965 |
| 7 days | C3AR1 + IL1R2 + CD74 + IFNG + CD177 + CD3D | 0.839 | 0.715 | 0.963 |
| 7 days | C3AR1 + IL1R2 + CD74 + IFNG + CD177 + CD3D + CTLA4 | 0.839 | 0.715 | 0.963 |
| 7 days | C3AR1 + IL1R2 + CD74 + IFNG + CD177 + CIITA | 0.895 | 0.798 | 0.991 |
| 7 days | C3AR1 + IL1R2 + CD74 + IFNG + CD177 + CIITA + CD3D | 0.898 | 0.803 | 0.992 |
| 7 days | C3AR1 + IL1R2 + CD74 + IFNG + CD177 + CIITA + CD3D + CTLA4 | 0.9 | 0.807 | 0.993 |
| 7 days | C3AR1 + IL1R2 + CD74 + IFNG + CD177 + CIITA + CTLA4 | 0.898 | 0.803 | 0.992 |
| 7 days | C3AR1 + IL1R2 + CD74 + IFNG + CD177 + CIITA + TAP2 | 0.895 | 0.796 | 0.994 |
| 7 days | C3AR1 + IL1R2 + CD74 + IFNG + CD177 + CIITA + TAP2 + CD3D | 0.895 | 0.797 | 0.992 |
| 7 days | C3AR1 + IL1R2 + CD74 + IFNG + CD177 + CIITA + TAP2 + CD3D + CTLA4 | 0.9 | 0.806 | 0.994 |
| 7 days | C3AR1 + IL1R2 + CD74 + IFNG + CD177 + CIITA + TAP2 + CTLA4 | 0.889 | 0.788 | 0.991 |
| 7 days | C3AR1 + IL1R2 + CD74 + IFNG + CD177 + CTLA4 | 0.839 | 0.715 | 0.963 |
| 7 days | C3AR1 + IL1R2 + CD74 + IFNG + CD177 + CX3CR1 | 0.881 | 0.775 | 0.987 |
| 7 days | C3AR1 + IL1R2 + CD74 + IFNG + CD177 + CX3CR1 + CD3D | 0.892 | 0.792 | 0.992 |
| 7 days | C3AR1 + IL1R2 + CD74 + IFNG + CD177 + CX3CR1 + CD3D + CTLA4 | 0.889 | 0.786 | 0.993 |
| 7 days | C3AR1 + IL1R2 + CD74 + IFNG + CD177 + CX3CR1 + CIITA | 0.917 | 0.827 | 1 |
| 7 days | C3AR1 + IL1R2 + CD74 + IFNG + CD177 + CX3CR1 + CIITA + CD3D | 0.928 | 0.84 | 1 |
| 7 days | C3AR1 + IL1R2 + CD74 + IFNG + CD177 + CX3CR1 + CIITA + CD3D + CTLA4 | 0.925 | 0.836 | 1 |
| 7 days | C3AR1 + IL1R2 + CD74 + IFNG + CD177 + CX3CR1 + CIITA + CTLA4 | 0.92 | 0.831 | 1 |
| 7 days | C3AR1 + IL1R2 + CD74 + IFNG + CD177 + CX3CR1 + CIITA + TAP2 | 0.917 | 0.827 | 1 |
| 7 days | C3AR1 + IL1R2 + CD74 + IFNG + CD177 + CX3CR1 + CIITA + TAP2 + CD3D | 0.934 | 0.848 | 1 |
| 7 days | C3AR1 + IL1R2 + CD74 + IFNG + CD177 + CX3CR1 + CIITA + TAP2 + CD3D + CTLA4 | 0.939 | 0.86 | 1 |
| 7 days | C3AR1 + IL1R2 + CD74 + IFNG + CD177 + CX3CR1 + CIITA + TAP2 + CTLA4 | 0.922 | 0.835 | 1 |
| 7 days | C3AR1 + IL1R2 + CD74 + IFNG + CD177 + CX3CR1 + CTLA4 | 0.886 | 0.783 | 0.99 |
| 7 days | C3AR1 + IL1R2 + CD74 + IFNG + CD177 + CX3CR1 + TAP2 | 0.881 | 0.775 | 0.987 |
| 7 days | C3AR1 + IL1R2 + CD74 + IFNG + CD177 + CX3CR1 + TAP2 + CD3D | 0.892 | 0.79 | 0.994 |
| 7 days | C3AR1 + IL1R2 + CD74 + IFNG + CD177 + CX3CR1 + TAP2 + CD3D + CTLA4 | 0.889 | 0.786 | 0.992 |
| 7 days | C3AR1 + IL1R2 + CD74 + IFNG + CD177 + CX3CR1 + TAP2 + CTLA4 | 0.881 | 0.774 | 0.988 |
| 7 days | C3AR1 + IL1R2 + CD74 + IFNG + CD177 + S100A9 | 0.839 | 0.715 | 0.963 |
| 7 days | C3AR1 + IL1R2 + CD74 + IFNG + CD177 + S100A9 + CD3D | 0.839 | 0.715 | 0.963 |
| 7 days | C3AR1 + IL1R2 + CD74 + IFNG + CD177 + S100A9 + CD3D + CTLA4 | 0.837 | 0.711 | 0.962 |
| 7 days | C3AR1 + IL1R2 + CD74 + IFNG + CD177 + S100A9 + CIITA | 0.889 | 0.79 | 0.989 |
| 7 days | C3AR1 + IL1R2 + CD74 + IFNG + CD177 + S100A9 + CIITA + CD3D | 0.892 | 0.794 | 0.99 |
| 7 days | C3AR1 + IL1R2 + CD74 + IFNG + CD177 + S100A9 + CIITA + CD3D + CTLA4 | 0.889 | 0.79 | 0.988 |
| 7 days | C3AR1 + IL1R2 + CD74 + IFNG + CD177 + S100A9 + CIITA + CTLA4 | 0.9 | 0.808 | 0.993 |
| 7 days | C3AR1 + IL1R2 + CD74 + IFNG + CD177 + S100A9 + CIITA + TAP2 | 0.898 | 0.801 | 0.994 |
| 7 days | C3AR1 + IL1R2 + CD74 + IFNG + CD177 + S100A9 + CIITA + TAP2 + CD3D | 0.898 | 0.802 | 0.993 |
| 7 days | C3AR1 + IL1R2 + CD74 + IFNG + CD177 + S100A9 + CIITA + TAP2 + CD3D + CTLA4 | 0.909 | 0.819 | 0.998 |
| 7 days | C3AR1 + IL1R2 + CD74 + IFNG + CD177 + S100A9 + CIITA + TAP2 + CTLA4 | 0.886 | 0.784 | 0.989 |
| 7 days | C3AR1 + IL1R2 + CD74 + IFNG + CD177 + S100A9 + CTLA4 | 0.837 | 0.712 | 0.961 |
| 7 days | C3AR1 + IL1R2 + CD74 + IFNG + CD177 + S100A9 + CX3CR1 | 0.878 | 0.77 | 0.986 |
| 7 days | C3AR1 + IL1R2 + CD74 + IFNG + CD177 + S100A9 + CX3CR1 + CD3D | 0.881 | 0.776 | 0.986 |
| 7 days | C3AR1 + IL1R2 + CD74 + IFNG + CD177 + S100A9 + CX3CR1 + CD3D + CTLA4 | 0.886 | 0.784 | 0.989 |
| 7 days | C3AR1 + IL1R2 + CD74 + IFNG + CD177 + S100A9 + CX3CR1 + CIITA | 0.925 | 0.839 | 1 |
| 7 days | C3AR1 + IL1R2 + CD74 + IFNG + CD177 + S100A9 + CX3CR1 + CIITA + CD3D | 0.928 | 0.84 | 1 |
| 7 days | C3AR1 + IL1R2 + CD74 + IFNG + CD177 + S100A9 + CX3CR1 + CIITA + CD3D + | 0.925 | 0.84 | 1 |
| | CTLA4 | | | |
| 7 days | C3AR1 + IL1R2 + CD74 + IFNG + CD177 + S100A9 + CX3CR1 + CIITA + CTLA4 | 0.925 | 0.839 | 1 |
| 7 days | C3AR1 + IL1R2 + CD74 + IFNG + CD177 + S100A9 + CX3CR1 + CIITA + TAP2 | 0.928 | 0.842 | 1 |
| 7 days | C3AR1 + IL1R2 + CD74 + IFNG + CD177 + S100A9 + CX3CR1 + CIITA + TAP2 + CD3D | 0.934 | 0.849 | 1 |
| 7 days | C3AR1 + IL1R2 + CD74 + IFNG + CD177 + S100A9 + CX3CR1 + CIITA + TAP2 + CD3D + | 0.936 | 0.855 | 1 |
| | CTLA4 | | | |
| 7 days | C3AR1 + IL1R2 + CD74 + IFNG + CD177 + S100A9 + CX3CR1 + CIITA + TAP2 + CTLA4 | 0.928 | 0.844 | 1 |
| 7 days | C3AR1 + IL1R2 + CD74 + IFNG + CD177 + S100A9 + CX3CR1 + CTLA4 | 0.886 | 0.783 | 0.99 |
| 7 days | C3AR1 + IL1R2 + CD74 + IFNG + CD177 + S100A9 + CX3CR1 + TAP2 | 0.878 | 0.77 | 0.986 |
| 7 days | C3AR1 + IL1R2 + CD74 + IFNG + CD177 + S100A9 + CX3CR1 + TAP2 + CD3D | 0.878 | 0.771 | 0.985 |
| 7 days | C3AR1 + IL1R2 + CD74 + IFNG + CD177 + S100A9 + CX3CR1 + TAP2 + CD3D + CTLA4 | 0.881 | 0.775 | 0.987 |
| 7 days | C3AR1 + IL1R2 + CD74 + IFNG + CD177 + S100A9 + CX3CR1 + TAP2 + CTLA4 | 0.881 | 0.774 | 0.988 |
| 7 days | C3AR1 + IL1R2 + CD74 + IFNG + CD177 + S100A9 + TAP2 | 0.842 | 0.717 | 0.967 |
| 7 days | C3AR1 + IL1R2 + CD74 + IFNG + CD177 + S100A9 + TAP2 + CD3D | 0.842 | 0.718 | 0.966 |
| 7 days | C3AR1 + IL1R2 + CD74 + IFNG + CD177 + S100A9 + TAP2 + CD3D + CTLA4 | 0.842 | 0.718 | 0.966 |
| 7 days | C3AR1 + IL1R2 + CD74 + IFNG + CD177 + S100A9 + TAP2 + CTLA4 | 0.839 | 0.714 | 0.965 |
| 7 days | C3AR1 + IL1R2 + CD74 + IFNG + CD177 + TAP2 | 0.848 | 0.725 | 0.97 |
| 7 days | C3AR1 + IL1R2 + CD74 + IFNG + CD177 + TAP2 + CD3D | 0.839 | 0.714 | 0.964 |
| 7 days | C3AR1 + IL1R2 + CD74 + IFNG + CD177 + TAP2 + CD3D + CTLA4 | 0.839 | 0.714 | 0.964 |
| 7 days | C3AR1 + IL1R2 + CD74 + IFNG + CD177 + TAP2 + CTLA4 | 0.837 | 0.709 | 0.964 |
| 7 days | C3AR1 + IL1R2 + CD74 + IFNG + CD3D | 0.823 | 0.69 | 0.955 |
| 7 days | C3AR1 + IL1R2 + CD74 + IFNG + CD3D + CTLA4 | 0.828 | 0.698 | 0.958 |
| 7 days | C3AR1 + IL1R2 + CD74 + IFNG + CIITA | 0.886 | 0.784 | 0.989 |
| 7 days | C3AR1 + IL1R2 + CD74 + IFNG + CIITA + CD3D | 0.886 | 0.785 | 0.988 |
| 7 days | C3AR1 + IL1R2 + CD74 + IFNG + CIITA + CD3D + CTLA4 | 0.884 | 0.779 | 0.988 |
| 7 days | C3AR1 + IL1R2 + CD74 + IFNG + CIITA + CTLA4 | 0.881 | 0.775 | 0.987 |
| 7 days | C3AR1 + IL1R2 + CD74 + IFNG + CIITA + TAP2 | 0.898 | 0.799 | 0.996 |
| 7 days | C3AR1 + IL1R2 + CD74 + IFNG + CIITA + TAP2 + CD3D | 0.889 | 0.787 | 0.991 |
| 7 days | C3AR1 + IL1R2 + CD74 + IFNG + CIITA + TAP2 + CD3D + CTLA4 | 0.895 | 0.791 | 0.998 |
| 7 days | C3AR1 + IL1R2 + CD74 + IFNG + CIITA + TAP2 + CTLA4 | 0.892 | 0.787 | 0.997 |
| 7 days | C3AR1 + IL1R2 + CD74 + IFNG + CTLA4 | 0.828 | 0.698 | 0.958 |
| 7 days | C3AR1 + IL1R2 + CD74 + IFNG + CX3CR1 | 0.875 | 0.761 | 0.989 |
| 7 days | C3AR1 + IL1R2 + CD74 + IFNG + CX3CR1 + CD3D | 0.87 | 0.754 | 0.986 |
| 7 days | C3AR1 + IL1R2 + CD74 + IFNG + CX3CR1 + CD3D + CTLA4 | 0.886 | 0.779 | 0.994 |
| 7 days | C3AR1 + IL1R2 + CD74 + IFNG + CX3CR1 + CIITA | 0.925 | 0.836 | 1 |
| 7 days | C3AR1 + IL1R2 + CD74 + IFNG + CX3CR1 + CIITA + CD3D | 0.928 | 0.84 | 1 |
| 7 days | C3AR1 + IL1R2 + CD74 + IFNG + CX3CR1 + CIITA + CD3D + CTLA4 | 0.939 | 0.857 | 1 |
| 7 days | C3AR1 + IL1R2 + CD74 + IFNG + CX3CR1 + CIITA + CTLA4 | 0.925 | 0.838 | 1 |
| 7 days | C3AR1 + IL1R2 + CD74 + IFNG + CX3CR1 + CIITA + TAP2 | 0.922 | 0.834 | 1 |
| 7 days | C3AR1 + IL1R2 + CD74 + IFNG + CX3CR1 + CIITA + TAP2 + CD3D | 0.934 | 0.848 | 1 |
| 7 days | C3AR1 + IL1R2 + CD74 + IFNG + CX3CR1 + CIITA + TAP2 + CD3D + CTLA4 | 0.945 | 0.868 | 1 |
| 7 days | C3AR1 + IL1R2 + CD74 + IFNG + CX3CR1 + CIITA + TAP2 + CTLA4 | 0.925 | 0.838 | 1 |
| 7 days | C3AR1 + IL1R2 + CD74 + IFNG + CX3CR1 + CTLA4 | 0.878 | 0.767 | 0.99 |
| 7 days | C3AR1 + IL1R2 + CD74 + IFNG + CX3CR1 + TAP2 | 0.878 | 0.766 | 0.99 |
| 7 days | C3AR1 + IL1R2 + CD74 + IFNG + CX3CR1 + TAP2 + CD3D | 0.881 | 0.771 | 0.991 |
| 7 days | C3AR1 + IL1R2 + CD74 + IFNG + CX3CR1 + TAP2 + CD3D + CTLA4 | 0.881 | 0.77 | 0.992 |
| 7 days | C3AR1 + IL1R2 + CD74 + IFNG + CX3CR1 + TAP2 + CTLA4 | 0.878 | 0.767 | 0.99 |
| 7 days | C3AR1 + IL1R2 + CD74 + IFNG + S100A9 | 0.842 | 0.716 | 0.968 |
| 7 days | C3AR1 + IL1R2 + CD74 + IFNG + S100A9 + CD3D | 0.842 | 0.718 | 0.966 |
| 7 days | C3AR1 + IL1R2 + CD74 + IFNG + S100A9 + CD3D + CTLA4 | 0.845 | 0.722 | 0.968 |
| 7 days | C3AR1 + IL1R2 + CD74 + IFNG + S100A9 + CIITA | 0.886 | 0.785 | 0.988 |
| 7 days | C3AR1 + IL1R2 + CD74 + IFNG + S100A9 + CIITA + CD3D | 0.886 | 0.785 | 0.988 |
| 7 days | C3AR1 + IL1R2 + CD74 + IFNG + S100A9 + CIITA + CD3D + CTLA4 | 0.884 | 0.78 | 0.987 |
| 7 days | C3AR1 + IL1R2 + CD74 + IFNG + S100A9 + CIITA + CTLA4 | 0.875 | 0.767 | 0.984 |
| 7 days | C3AR1 + IL1R2 + CD74 + IFNG + S100A9 + CIITA + TAP2 | 0.895 | 0.795 | 0.994 |
| 7 days | C3AR1 + IL1R2 + CD74 + IFNG + S100A9 + CIITA + TAP2 + CD3D | 0.895 | 0.797 | 0.992 |
| 7 days | C3AR1 + IL1R2 + CD74 + IFNG + S100A9 + CIITA + TAP2 + CD3D + CTLA4 | 0.906 | 0.814 | 0.998 |
| 7 days | C3AR1 + IL1R2 + CD74 + IFNG + S100A9 + CIITA + TAP2 + CTLA4 | 0.889 | 0.785 | 0.994 |
| 7 days | C3AR1 + IL1R2 + CD74 + IFNG + S100A9 + CTLA4 | 0.839 | 0.713 | 0.965 |
| 7 days | C3AR1 + IL1R2 + CD74 + IFNG + S100A9 + CX3CR1 | 0.861 | 0.745 | 0.978 |
| 7 days | C3AR1 + IL1R2 + CD74 + IFNG + S100A9 + CX3CR1 + CD3D | 0.878 | 0.772 | 0.985 |
| 7 days | C3AR1 + IL1R2 + CD74 + IFNG + S100A9 + CX3CR1 + CD3D + CTLA4 | 0.884 | 0.776 | 0.991 |
| 7 days | C3AR1 + IL1R2 + CD74 + IFNG + S100A9 + CX3CR1 + CIITA | 0.93 | 0.844 | 1 |
| 7 days | C3AR1 + IL1R2 + CD74 + IFNG + S100A9 + CX3CR1 + CIITA + CD3D | 0.928 | 0.84 | 1 |
| 7 days | C3AR1 + IL1R2 + CD74 + IFNG + S100A9 + CX3CR1 + CIITA + CD3D + CTLA4 | 0.925 | 0.84 | 1 |
| 7 days | C3AR1 + IL1R2 + CD74 + IFNG + S100A9 + CX3CR1 + CIITA + CTLA4 | 0.934 | 0.851 | 1 |
| 7 days | C3AR1 + IL1R2 + CD74 + IFNG + S100A9 + CX3CR1 + CIITA + TAP2 | 0.925 | 0.836 | 1 |
| 7 days | C3AR1 + IL1R2 + CD74 + IFNG + S100A9 + CX3CR1 + CIITA + TAP2 + CD3D | 0.931 | 0.845 | 1 |
| 7 days | C3AR1 + IL1R2 + CD74 + IFNG + S100A9 + CX3CR1 + CIITA + TAP2 + CD3D + CTLA4 | 0.936 | 0.856 | 1 |
| 7 days | C3AR1 + IL1R2 + CD74 + IFNG + S100A9 + CX3CR1 + CIITA + TAP2 + CTLA4 | 0.931 | 0.847 | 1 |
| 7 days | C3AR1 + IL1R2 + CD74 + IFNG + S100A9 + CX3CR1 + CTLA4 | 0.878 | 0.769 | 0.987 |
| 7 days | C3AR1 + IL1R2 + CD74 + IFNG + S100A9 + CX3CR1 + TAP2 | 0.867 | 0.753 | 0.982 |
| 7 days | C3AR1 + IL1R2 + CD74 + IFNG + S100A9 + CX3CR1 + TAP2 + CD3D | 0.886 | 0.783 | 0.99 |
| 7 days | C3AR1 + IL1R2 + CD74 + IFNG + S100A9 + CX3CR1 + TAP2 + CD3D + CTLA4 | 0.886 | 0.783 | 0.99 |
| 7 days | C3AR1 + IL1R2 + CD74 + IFNG + S100A9 + CX3CR1 + TAP2 + CTLA4 | 0.881 | 0.771 | 0.991 |
| 7 days | C3AR1 + IL1R2 + CD74 + IFNG + S100A9 + TAP2 | 0.845 | 0.717 | 0.972 |
| 7 days | C3AR1 + IL1R2 + CD74 + IFNG + S100A9 + TAP2 + CD3D | 0.842 | 0.716 | 0.968 |
| 7 days | C3AR1 + IL1R2 + CD74 + IFNG + S100A9 + TAP2 + CD3D + CTLA4 | 0.845 | 0.721 | 0.969 |
| 7 days | C3AR1 + IL1R2 + CD74 + IFNG + S100A9 + TAP2 + CTLA4 | 0.848 | 0.722 | 0.973 |
| 7 days | C3AR1 + IL1R2 + CD74 + IFNG + TAP2 | 0.839 | 0.712 | 0.966 |
| 7 days | C3AR1 + IL1R2 + CD74 + IFNG + TAP2 + CD3D | 0.837 | 0.711 | 0.962 |
| 7 days | C3AR1 + IL1R2 + CD74 + IFNG + TAP2 + CD3D + CTLA4 | 0.845 | 0.721 | 0.969 |
| 7 days | C3AR1 + IL1R2 + CD74 + IFNG + TAP2 + CTLA4 | 0.85 | 0.727 | 0.974 |
| 7 days | C3AR1 + IL1R2 + CD74 + S100A9 | 0.831 | 0.69 | 0.972 |
| 7 days | C3AR1 + IL1R2 + CD74 + S100A9 + CD3D | 0.825 | 0.685 | 0.966 |
| 7 days | C3AR1 + IL1R2 + CD74 + S100A9 + CD3D + CTLA4 | 0.828 | 0.694 | 0.963 |
| 7 days | C3AR1 + IL1R2 + CD74 + S100A9 + CIITA | 0.886 | 0.776 | 0.997 |
| 7 days | C3AR1 + IL1R2 + CD74 + S100A9 + CIITA + CD3D | 0.898 | 0.798 | 0.997 |
| 7 days | C3AR1 + IL1R2 + CD74 + S100A9 + CIITA + CD3D + CTLA4 | 0.892 | 0.791 | 0.993 |
| 7 days | C3AR1 + IL1R2 + CD74 + S100A9 + CIITA + CTLA4 | 0.884 | 0.772 | 0.995 |
| 7 days | C3AR1 + IL1R2 + CD74 + S100A9 + CIITA + TAP2 | 0.884 | 0.772 | 0.995 |
| 7 days | C3AR1 + IL1R2 + CD74 + S100A9 + CIITA + TAP2 + CD3D | 0.895 | 0.792 | 0.997 |
| 7 days | C3AR1 + IL1R2 + CD74 + S100A9 + CIITA + TAP2 + CD3D + CTLA4 | 0.9 | 0.804 | 0.996 |
| 7 days | C3AR1 + IL1R2 + CD74 + S100A9 + CIITA + TAP2 + CTLA4 | 0.881 | 0.771 | 0.991 |
| 7 days | C3AR1 + IL1R2 + CD74 + S100A9 + CTLA4 | 0.831 | 0.69 | 0.972 |
| 7 days | C3AR1 + IL1R2 + CD74 + S100A9 + CX3CR1 | 0.848 | 0.711 | 0.984 |
| 7 days | C3AR1 + IL1R2 + CD74 + S100A9 + CX3CR1 + CD3D | 0.848 | 0.712 | 0.983 |
| 7 days | C3AR1 + IL1R2 + CD74 + S100A9 + CX3CR1 + CD3D + CTLA4 | 0.842 | 0.703 | 0.981 |
| 7 days | C3AR1 + IL1R2 + CD74 + S100A9 + CX3CR1 + CIITA | 0.881 | 0.764 | 0.997 |
| 7 days | C3AR1 + IL1R2 + CD74 + S100A9 + CX3CR1 + CIITA + CD3D | 0.881 | 0.767 | 0.995 |
| 7 days | C3AR1 + IL1R2 + CD74 + S100A9 + CX3CR1 + CIITA + CD3D + CTLA4 | 0.892 | 0.79 | 0.994 |
| 7 days | C3AR1 + IL1R2 + CD74 + S100A9 + CX3CR1 + CIITA + CTLA4 | 0.889 | 0.78 | 0.998 |
| 7 days | C3AR1 + IL1R2 + CD74 + S100A9 + CX3CR1 + CIITA + TAP2 | 0.878 | 0.762 | 0.994 |
| 7 days | C3AR1 + IL1R2 + CD74 + S100A9 + CX3CR1 + CIITA + TAP2 + CD3D | 0.886 | 0.776 | 0.997 |
| 7 days | C3AR1 + IL1R2 + CD74 + S100A9 + CX3CR1 + CIITA + TAP2 + CD3D + CTLA4 | 0.906 | 0.81 | 1 |
| 7 days | C3AR1 + IL1R2 + CD74 + S100A9 + CX3CR1 + CIITA + TAP2 + CTLA4 | 0.884 | 0.769 | 0.999 |
| 7 days | C3AR1 + IL1R2 + CD74 + S100A9 + CX3CR1 + CTLA4 | 0.853 | 0.72 | 0.986 |
| 7 days | C3AR1 + IL1R2 + CD74 + S100A9 + CX3CR1 + TAP2 | 0.856 | 0.717 | 0.995 |
| 7 days | C3AR1 + IL1R2 + CD74 + S100A9 + CX3CR1 + TAP2 + CD3D | 0.842 | 0.704 | 0.98 |
| 7 days | C3AR1 + IL1R2 + CD74 + S100A9 + CX3CR1 + TAP2 + CD3D + CTLA4 | 0.845 | 0.708 | 0.982 |
| 7 days | C3AR1 + IL1R2 + CD74 + S100A9 + CX3CR1 + TAP2 + CTLA4 | 0.85 | 0.711 | 0.989 |
| 7 days | C3AR1 + IL1R2 + CD74 + S100A9 + TAP2 | 0.828 | 0.687 | 0.969 |
| 7 days | C3AR1 + IL1R2 + CD74 + S100A9 + TAP2 + CD3D | 0.839 | 0.705 | 0.974 |
| 7 days | C3AR1 + IL1R2 + CD74 + S100A9 + TAP2 + CD3D + CTLA4 | 0.839 | 0.708 | 0.97 |
| 7 days | C3AR1 + IL1R2 + CD74 + S100A9 + TAP2 + CTLA4 | 0.828 | 0.687 | 0.969 |
| 7 days | C3AR1 + IL1R2 + CD74 + TAP2 | 0.767 | 0.614 | 0.921 |
| 7 days | C3AR1 + IL1R2 + CD74 + TAP2 + CD3D | 0.776 | 0.627 | 0.924 |
| 7 days | C3AR1 + IL1R2 + CD74 + TAP2 + CD3D + CTLA4 | 0.778 | 0.629 | 0.928 |
| 7 days | C3AR1 + IL1R2 + CD74 + TAP2 + CTLA4 | 0.789 | 0.64 | 0.939 |
| 7 days | C3AR1 + IL1R2 + CIITA | 0.837 | 0.707 | 0.966 |
| 7 days | C3AR1 + IL1R2 + CIITA + CD3D | 0.839 | 0.713 | 0.966 |
| 7 days | C3AR1 + IL1R2 + CIITA + CD3D + CTLA4 | 0.853 | 0.736 | 0.97 |
| 7 days | C3AR1 + IL1R2 + CIITA + CTLA4 | 0.848 | 0.725 | 0.97 |
| 7 days | C3AR1 + IL1R2 + CIITA + TAP2 | 0.845 | 0.719 | 0.971 |
| 7 days | C3AR1 + IL1R2 + CIITA + TAP2 + CD3D | 0.845 | 0.722 | 0.968 |
| 7 days | C3AR1 + IL1R2 + CIITA + TAP2 + CD3D + CTLA4 | 0.848 | 0.728 | 0.967 |
| 7 days | C3AR1 + IL1R2 + CIITA + TAP2 + CTLA4 | 0.842 | 0.716 | 0.968 |
| 7 days | C3AR1 + IL1R2 + CX3CR1 + CIITA | 0.839 | 0.711 | 0.967 |
| 7 days | C3AR1 + IL1R2 + CX3CR1 + CIITA + CD3D | 0.834 | 0.706 | 0.962 |
| 7 days | C3AR1 + IL1R2 + CX3CR1 + CIITA + CD3D + CTLA4 | 0.853 | 0.736 | 0.97 |
| 7 days | C3AR1 + IL1R2 + CX3CR1 + CIITA + CTLA4 | 0.842 | 0.718 | 0.966 |
| 7 days | C3AR1 + IL1R2 + CX3CR1 + CIITA + TAP2 | 0.853 | 0.732 | 0.974 |
| 7 days | C3AR1 + IL1R2 + CX3CR1 + CIITA + TAP2 + CD3D | 0.842 | 0.718 | 0.966 |
| 7 days | C3AR1 + IL1R2 + CX3CR1 + CIITA + TAP2 + CD3D + CTLA4 | 0.856 | 0.74 | 0.972 |
| 7 days | C3AR1 + IL1R2 + CX3CR1 + CIITA + TAP2 + CTLA4 | 0.85 | 0.727 | 0.974 |
| 7 days | C3AR1 + IL1R2 + CX3CR1 + TAP2 | 0.695 | 0.52 | 0.871 |
| 7 days | C3AR1 + IL1R2 + CX3CR1 + TAP2 + CD3D | 0.69 | 0.512 | 0.868 |
| 7 days | C3AR1 + IL1R2 + CX3CR1 + TAP2 + CD3D + CTLA4 | 0.695 | 0.518 | 0.873 |
| 7 days | C3AR1 + IL1R2 + CX3CR1 + TAP2 + CTLA4 | 0.695 | 0.518 | 0.873 |
| 7 days | C3AR1 + IL1R2 + IFNG | 0.726 | 0.562 | 0.89 |
| 7 days | C3AR1 + IL1R2 + IFNG + CD177 | 0.801 | 0.658 | 0.943 |
| 7 days | C3AR1 + IL1R2 + IFNG + CD177 + CD3D | 0.803 | 0.663 | 0.943 |
| 7 days | C3AR1 + IL1R2 + IFNG + CD177 + CD3D + CTLA4 | 0.812 | 0.675 | 0.948 |
| 7 days | C3AR1 + IL1R2 + IFNG + CD177 + CIITA | 0.881 | 0.777 | 0.985 |
| 7 days | C3AR1 + IL1R2 + IFNG + CD177 + CIITA + CD3D | 0.875 | 0.768 | 0.982 |
| 7 days | C3AR1 + IL1R2 + IFNG + CD177 + CIITA + CD3D + CTLA4 | 0.884 | 0.781 | 0.986 |
| 7 days | C3AR1 + IL1R2 + IFNG + CD177 + CIITA + CTLA4 | 0.878 | 0.773 | 0.983 |
| 7 days | C3AR1 + IL1R2 + IFNG + CD177 + CIITA + TAP2 | 0.878 | 0.773 | 0.983 |
| 7 days | C3AR1 + IL1R2 + IFNG + CD177 + CIITA + TAP2 + CD3D | 0.878 | 0.772 | 0.984 |
| 7 days | C3AR1 + IL1R2 + IFNG + CD177 + CIITA + TAP2 + CD3D + CTLA4 | 0.889 | 0.789 | 0.989 |
| 7 days | C3AR1 + IL1R2 + IFNG + CD177 + CIITA + TAP2 + CTLA4 | 0.878 | 0.773 | 0.983 |
| 7 days | C3AR1 + IL1R2 + IFNG + CD177 + CTLA4 | 0.809 | 0.671 | 0.947 |
| 7 days | C3AR1 + IL1R2 + IFNG + CD177 + CX3CR1 | 0.806 | 0.664 | 0.949 |
| 7 days | C3AR1 + IL1R2 + IFNG + CD177 + CX3CR1 + CD3D | 0.812 | 0.673 | 0.951 |
| 7 days | C3AR1 + IL1R2 + IFNG + CD177 + CX3CR1 + CD3D + CTLA4 | 0.806 | 0.665 | 0.947 |
| 7 days | C3AR1 + IL1R2 + IFNG + CD177 + CX3CR1 + CIITA | 0.875 | 0.768 | 0.982 |
| 7 days | C3AR1 + IL1R2 + IFNG + CD177 + CX3CR1 + CIITA + CD3D | 0.881 | 0.775 | 0.987 |
| 7 days | C3AR1 + IL1R2 + IFNG + CD177 + CX3CR1 + CIITA + CD3D + CTLA4 | 0.903 | 0.808 | 0.999 |
| 7 days | C3AR1 + IL1R2 + IFNG + CD177 + CX3CR1 + CIITA + CTLA4 | 0.886 | 0.786 | 0.987 |
| 7 days | C3AR1 + IL1R2 + IFNG + CD177 + CX3CR1 + CIITA + TAP2 | 0.878 | 0.772 | 0.984 |
| 7 days | C3AR1 + IL1R2 + IFNG + CD177 + CX3CR1 + CIITA + TAP2 + CD3D | 0.892 | 0.793 | 0.991 |
| 7 days | C3AR1 + IL1R2 + IFNG + CD177 + CX3CR1 + CIITA + TAP2 + CD3D + CTLA4 | 0.903 | 0.808 | 0.999 |
| 7 days | C3AR1 + IL1R2 + IFNG + CD177 + CX3CR1 + CIITA + TAP2 + CTLA4 | 0.892 | 0.793 | 0.991 |
| 7 days | C3AR1 + IL1R2 + IFNG + CD177 + CX3CR1 + CTLA4 | 0.806 | 0.665 | 0.948 |
| 7 days | C3AR1 + IL1R2 + IFNG + CD177 + CX3CR1 + TAP2 | 0.82 | 0.684 | 0.956 |
| 7 days | C3AR1 + IL1R2 + IFNG + CD177 + CX3CR1 + TAP2 + CD3D | 0.817 | 0.68 | 0.954 |
| 7 days | C3AR1 + IL1R2 + IFNG + CD177 + CX3CR1 + TAP2 + CD3D + CTLA4 | 0.82 | 0.684 | 0.956 |
| 7 days | C3AR1 + IL1R2 + IFNG + CD177 + CX3CR1 + TAP2 + CTLA4 | 0.82 | 0.685 | 0.955 |
| 7 days | C3AR1 + IL1R2 + IFNG + CD177 + S100A9 | 0.795 | 0.651 | 0.939 |
| 7 days | C3AR1 + IL1R2 + IFNG + CD177 + S100A9 + CD3D | 0.795 | 0.651 | 0.939 |
| 7 days | C3AR1 + IL1R2 + IFNG + CD177 + S100A9 + CD3D + CTLA4 | 0.801 | 0.659 | 0.942 |
| 7 days | C3AR1 + IL1R2 + IFNG + CD177 + S100A9 + CIITA | 0.875 | 0.768 | 0.982 |
| 7 days | C3AR1 + IL1R2 + IFNG + CD177 + S100A9 + CIITA + CD3D | 0.875 | 0.768 | 0.982 |
| 7 days | C3AR1 + IL1R2 + IFNG + CD177 + S100A9 + CIITA + CD3D + CTLA4 | 0.875 | 0.769 | 0.982 |
| 7 days | C3AR1 + IL1R2 + IFNG + CD177 + S100A9 + CIITA + CTLA4 | 0.878 | 0.773 | 0.983 |
| 7 days | C3AR1 + IL1R2 + IFNG + CD177 + S100A9 + CIITA + TAP2 | 0.878 | 0.772 | 0.984 |
| 7 days | C3AR1 + IL1R2 + IFNG + CD177 + S100A9 + CIITA + TAP2 + CD3D | 0.878 | 0.772 | 0.984 |
| 7 days | C3AR1 + IL1R2 + IFNG + CD177 + S100A9 + CIITA + TAP2 + CD3D + CTLA4 | 0.884 | 0.782 | 0.985 |
| 7 days | C3AR1 + IL1R2 + IFNG + CD177 + S100A9 + CIITA + TAP2 + CTLA4 | 0.875 | 0.769 | 0.982 |
| 7 days | C3AR1 + IL1R2 + IFNG + CD177 + S100A9 + CTLA4 | 0.801 | 0.659 | 0.942 |
| 7 days | C3AR1 + IL1R2 + IFNG + CD177 + S100A9 + CX3CR1 | 0.789 | 0.644 | 0.935 |
| 7 days | C3AR1 + IL1R2 + IFNG + CD177 + S100A9 + CX3CR1 + CD3D | 0.798 | 0.657 | 0.939 |
| 7 days | C3AR1 + IL1R2 + IFNG + CD177 + S100A9 + CX3CR1 + CD3D + CTLA4 | 0.792 | 0.65 | 0.935 |
| 7 days | C3AR1 + IL1R2 + IFNG + CD177 + S100A9 + CX3CR1 + CIITA | 0.881 | 0.775 | 0.986 |
| 7 days | C3AR1 + IL1R2 + IFNG + CD177 + S100A9 + CX3CR1 + CIITA + CD3D | 0.884 | 0.78 | 0.988 |
| 7 days | C3AR1 + IL1R2 + IFNG + CD177 + S100A9 + CX3CR1 + CIITA + CD3D + CTLA4 | 0.889 | 0.788 | 0.991 |
| 7 days | C3AR1 + IL1R2 + IFNG + CD177 + S100A9 + CX3CR1 + CIITA + CTLA4 | 0.889 | 0.789 | 0.989 |
| 7 days | C3AR1 + IL1R2 + IFNG + CD177 + S100A9 + CX3CR1 + CIITA + TAP2 | 0.898 | 0.798 | 0.997 |
| 7 days | C3AR1 + IL1R2 + IFNG + CD177 + S100A9 + CX3CR1 + CIITA + TAP2 + CD3D | 0.892 | 0.793 | 0.991 |
| 7 days | C3AR1 + IL1R2 + IFNG + CD177 + S100A9 + CX3CR1 + CIITA + TAP2 + CD3D + CTLA4 | 0.9 | 0.806 | 0.994 |
| 7 days | C3AR1 + IL1R2 + IFNG + CD177 + S100A9 + CX3CR1 + CIITA + TAP2 + CTLA4 | 0.889 | 0.787 | 0.992 |
| 7 days | C3AR1 + IL1R2 + IFNG + CD177 + S100A9 + CX3CR1 + CTLA4 | 0.806 | 0.666 | 0.946 |
| 7 days | C3AR1 + IL1R2 + IFNG + CD177 + S100A9 + CX3CR1 + TAP2 | 0.814 | 0.679 | 0.95 |
| 7 days | C3AR1 + IL1R2 + IFNG + CD177 + S100A9 + CX3CR1 + TAP2 + CD3D | 0.809 | 0.672 | 0.946 |
| 7 days | C3AR1 + IL1R2 + IFNG + CD177 + S100A9 + CX3CR1 + TAP2 + CD3D + CTLA4 | 0.814 | 0.679 | 0.95 |
| 7 days | C3AR1 + IL1R2 + IFNG + CD177 + S100A9 + CX3CR1 + TAP2 + CTLA4 | 0.814 | 0.679 | 0.95 |
| 7 days | C3AR1 + IL1R2 + IFNG + CD177 + S100A9 + TAP2 | 0.801 | 0.66 | 0.941 |
| 7 days | C3AR1 + IL1R2 + IFNG + CD177 + S100A9 + TAP2 + CD3D | 0.801 | 0.659 | 0.942 |
| 7 days | C3AR1 + IL1R2 + IFNG + CD177 + S100A9 + TAP2 + CD3D + CTLA4 | 0.803 | 0.662 | 0.945 |
| 7 days | C3AR1 + IL1R2 + IFNG + CD177 + S100A9 + TAP2 + CTLA4 | 0.798 | 0.656 | 0.94 |
| 7 days | C3AR1 + IL1R2 + IFNG + CD177 + TAP2 | 0.817 | 0.68 | 0.954 |
| 7 days | C3AR1 + IL1R2 + IFNG + CD177 + TAP2 + CD3D | 0.812 | 0.673 | 0.95 |
| 7 days | C3AR1 + IL1R2 + IFNG + CD177 + TAP2 + CD3D + CTLA4 | 0.803 | 0.662 | 0.945 |
| 7 days | C3AR1 + IL1R2 + IFNG + CD177 + TAP2 + CTLA4 | 0.809 | 0.669 | 0.948 |
| 7 days | C3AR1 + IL1R2 + IFNG + CD3D | 0.734 | 0.573 | 0.895 |
| 7 days | C3AR1 + IL1R2 + IFNG + CD3D + CTLA4 | 0.726 | 0.563 | 0.888 |
| 7 days | C3AR1 + IL1R2 + IFNG + CIITA | 0.853 | 0.734 | 0.972 |
| 7 days | C3AR1 + IL1R2 + IFNG + CIITA + CD3D | 0.853 | 0.734 | 0.973 |
| 7 days | C3AR1 + IL1R2 + IFNG + CIITA + CD3D + CTLA4 | 0.867 | 0.753 | 0.981 |
| 7 days | C3AR1 + IL1R2 + IFNG + CIITA + CTLA4 | 0.859 | 0.741 | 0.977 |
| 7 days | C3AR1 + IL1R2 + IFNG + CIITA + TAP2 | 0.856 | 0.739 | 0.973 |
| 7 days | C3AR1 + IL1R2 + IFNG + CIITA + TAP2 + CD3D | 0.853 | 0.734 | 0.973 |
| 7 days | C3AR1 + IL1R2 + IFNG + CIITA + TAP2 + CD3D + CTLA4 | 0.87 | 0.757 | 0.983 |
| 7 days | C3AR1 + IL1R2 + IFNG + CIITA + TAP2 + CTLA4 | 0.853 | 0.732 | 0.974 |
| 7 days | C3AR1 + IL1R2 + IFNG + CTLA4 | 0.72 | 0.557 | 0.884 |
| 7 days | C3AR1 + IL1R2 + IFNG + CX3CR1 | 0.756 | 0.6 | 0.913 |
| 7 days | C3AR1 + IL1R2 + IFNG + CX3CR1 + CD3D | 0.745 | 0.585 | 0.906 |
| 7 days | C3AR1 + IL1R2 + IFNG + CX3CR1 + CD3D + CTLA4 | 0.77 | 0.616 | 0.924 |
| 7 days | C3AR1 + IL1R2 + IFNG + CX3CR1 + CIITA | 0.864 | 0.749 | 0.98 |
| 7 days | C3AR1 + IL1R2 + IFNG + CX3CR1 + CIITA + CD3D | 0.881 | 0.772 | 0.99 |
| 7 days | C3AR1 + IL1R2 + IFNG + CX3CR1 + CIITA + CD3D + CTLA4 | 0.884 | 0.777 | 0.99 |
| 7 days | C3AR1 + IL1R2 + IFNG + CX3CR1 + CIITA + CTLA4 | 0.87 | 0.759 | 0.981 |
| 7 days | C3AR1 + IL1R2 + IFNG + CX3CR1 + CIITA + TAP2 | 0.873 | 0.763 | 0.982 |
| 7 days | C3AR1 + IL1R2 + IFNG + CX3CR1 + CIITA + TAP2 + CD3D | 0.873 | 0.763 | 0.982 |
| 7 days | C3AR1 + IL1R2 + IFNG + CX3CR1 + CIITA + TAP2 + CD3D + CTLA4 | 0.886 | 0.783 | 0.99 |
| 7 days | C3AR1 + IL1R2 + IFNG + CX3CR1 + CIITA + TAP2 + CTLA4 | 0.875 | 0.765 | 0.985 |
| 7 days | C3AR1 + IL1R2 + IFNG + CX3CR1 + CTLA4 | 0.77 | 0.616 | 0.924 |
| 7 days | C3AR1 + IL1R2 + IFNG + CX3CR1 + TAP2 | 0.77 | 0.614 | 0.926 |
| 7 days | C3AR1 + IL1R2 + IFNG + CX3CR1 + TAP2 + CD3D | 0.778 | 0.626 | 0.931 |
| 7 days | C3AR1 + IL1R2 + IFNG + CX3CR1 + TAP2 + CD3D + CTLA4 | 0.778 | 0.629 | 0.927 |
| 7 days | C3AR1 + IL1R2 + IFNG + CX3CR1 + TAP2 + CTLA4 | 0.784 | 0.637 | 0.931 |
| 7 days | C3AR1 + IL1R2 + IFNG + S100A9 | 0.751 | 0.595 | 0.906 |
| 7 days | C3AR1 + IL1R2 + IFNG + S100A9 + CD3D | 0.759 | 0.606 | 0.912 |
| 7 days | C3AR1 + IL1R2 + IFNG + S100A9 + CD3D + CTLA4 | 0.753 | 0.599 | 0.908 |
| 7 days | C3AR1 + IL1R2 + IFNG + S100A9 + CIITA | 0.856 | 0.74 | 0.972 |
| 7 days | C3AR1 + IL1R2 + IFNG + S100A9 + CIITA + CD3D | 0.87 | 0.76 | 0.979 |
| 7 days | C3AR1 + IL1R2 + IFNG + S100A9 + CIITA + CD3D + CTLA4 | 0.87 | 0.759 | 0.98 |
| 7 days | C3AR1 + IL1R2 + IFNG + S100A9 + CIITA + CTLA4 | 0.864 | 0.752 | 0.977 |
| 7 days | C3AR1 + IL1R2 + IFNG + S100A9 + CIITA + TAP2 | 0.859 | 0.744 | 0.974 |
| 7 days | C3AR1 + IL1R2 + IFNG + S100A9 + CIITA + TAP2 + CD3D | 0.87 | 0.76 | 0.979 |
| 7 days | C3AR1 + IL1R2 + IFNG + S100A9 + CIITA + TAP2 + CD3D + CTLA4 | 0.881 | 0.777 | 0.985 |
| 7 days | C3AR1 + IL1R2 + IFNG + S100A9 + CIITA + TAP2 + CTLA4 | 0.864 | 0.752 | 0.976 |
| 7 days | C3AR1 + IL1R2 + IFNG + S100A9 + CTLA4 | 0.74 | 0.582 | 0.898 |
| 7 days | C3AR1 + IL1R2 + IFNG + S100A9 + CX3CR1 | 0.778 | 0.63 | 0.927 |
| 7 days | C3AR1 + IL1R2 + IFNG + S100A9 + CX3CR1 + CD3D | 0.787 | 0.639 | 0.934 |
| 7 days | C3AR1 + IL1R2 + IFNG + S100A9 + CX3CR1 + CD3D + CTLA4 | 0.787 | 0.639 | 0.934 |
| 7 days | C3AR1 + IL1R2 + IFNG + S100A9 + CX3CR1 + CIITA | 0.873 | 0.764 | 0.981 |
| 7 days | C3AR1 + IL1R2 + IFNG + S100A9 + CX3CR1 + CIITA + CD3D | 0.881 | 0.777 | 0.985 |
| 7 days | C3AR1 + IL1R2 + IFNG + S100A9 + CX3CR1 + CIITA + CD3D + CTLA4 | 0.9 | 0.807 | 0.993 |
| 7 days | C3AR1 + IL1R2 + IFNG + S100A9 + CX3CR1 + CIITA + CTLA4 | 0.875 | 0.766 | 0.985 |
| 7 days | C3AR1 + IL1R2 + IFNG + S100A9 + CX3CR1 + CIITA + TAP2 | 0.875 | 0.768 | 0.983 |
| 7 days | C3AR1 + IL1R2 + IFNG + S100A9 + CX3CR1 + CIITA + TAP2 + CD3D | 0.886 | 0.786 | 0.987 |
| 7 days | C3AR1 + IL1R2 + IFNG + S100A9 + CX3CR1 + CIITA + TAP2 + CD3D + CTLA4 | 0.911 | 0.824 | 0.999 |
| 7 days | C3AR1 + IL1R2 + IFNG + S100A9 + CX3CR1 + CIITA + TAP2 + CTLA4 | 0.881 | 0.776 | 0.986 |
| 7 days | C3AR1 + IL1R2 + IFNG + S100A9 + CX3CR1 + CTLA4 | 0.789 | 0.643 | 0.936 |
| 7 days | C3AR1 + IL1R2 + IFNG + S100A9 + CX3CR1 + TAP2 | 0.787 | 0.642 | 0.932 |
| 7 days | C3AR1 + IL1R2 + IFNG + S100A9 + CX3CR1 + TAP2 + CD3D | 0.784 | 0.638 | 0.93 |
| 7 days | C3AR1 + IL1R2 + IFNG + S100A9 + CX3CR1 + TAP2 + CD3D + CTLA4 | 0.787 | 0.641 | 0.932 |
| 7 days | C3AR1 + IL1R2 + IFNG + S100A9 + CX3CR1 + TAP2 + CTLA4 | 0.784 | 0.637 | 0.931 |
| 7 days | C3AR1 + IL1R2 + IFNG + S100A9 + TAP2 | 0.759 | 0.605 | 0.913 |
| 7 days | C3AR1 + IL1R2 + IFNG + S100A9 + TAP2 + CD3D | 0.765 | 0.613 | 0.916 |
| 7 days | C3AR1 + IL1R2 + IFNG + S100A9 + TAP2 + CD3D + CTLA4 | 0.765 | 0.613 | 0.916 |
| 7 days | C3AR1 + IL1R2 + IFNG + S100A9 + TAP2 + CTLA4 | 0.759 | 0.605 | 0.913 |
| 7 days | C3AR1 + IL1R2 + IFNG + TAP2 | 0.753 | 0.596 | 0.91 |
| 7 days | C3AR1 + IL1R2 + IFNG + TAP2 + CD3D | 0.753 | 0.596 | 0.91 |
| 7 days | C3AR1 + IL1R2 + IFNG + TAP2 + CD3D + CTLA4 | 0.734 | 0.572 | 0.896 |
| 7 days | C3AR1 + IL1R2 + IFNG + TAP2 + CTLA4 | 0.731 | 0.569 | 0.893 |
| 7 days | C3AR1 + IL1R2 + S100A9 | 0.748 | 0.582 | 0.914 |
| 7 days | C3AR1 + IL1R2 + S100A9 + CD3D | 0.753 | 0.594 | 0.913 |
| 7 days | C3AR1 + IL1R2 + S100A9 + CD3D + CTLA4 | 0.751 | 0.594 | 0.907 |
| 7 days | C3AR1 + IL1R2 + S100A9 + CIITA | 0.878 | 0.768 | 0.988 |
| 7 days | C3AR1 + IL1R2 + S100A9 + CIITA + CD3D | 0.87 | 0.756 | 0.983 |
| 7 days | C3AR1 + IL1R2 + S100A9 + CIITA + CD3D + CTLA4 | 0.892 | 0.792 | 0.992 |
| 7 days | C3AR1 + IL1R2 + S100A9 + CIITA + CTLA4 | 0.87 | 0.757 | 0.983 |
| 7 days | C3AR1 + IL1R2 + S100A9 + CIITA + TAP2 | 0.87 | 0.757 | 0.983 |
| 7 days | C3AR1 + IL1R2 + S100A9 + CIITA + TAP2 + CD3D | 0.861 | 0.743 | 0.98 |
| 7 days | C3AR1 + IL1R2 + S100A9 + CIITA + TAP2 + CD3D + CTLA4 | 0.875 | 0.768 | 0.983 |
| 7 days | C3AR1 + IL1R2 + S100A9 + CIITA + TAP2 + CTLA4 | 0.87 | 0.755 | 0.985 |
| 7 days | C3AR1 + IL1R2 + S100A9 + CTLA4 | 0.748 | 0.584 | 0.912 |
| 7 days | C3AR1 + IL1R2 + S100A9 + CX3CR1 | 0.745 | 0.578 | 0.912 |
| 7 days | C3AR1 + IL1R2 + S100A9 + CX3CR1 + CD3D | 0.759 | 0.603 | 0.915 |
| 7 days | C3AR1 + IL1R2 + S100A9 + CX3CR1 + CD3D + CTLA4 | 0.762 | 0.609 | 0.915 |
| 7 days | C3AR1 + IL1R2 + S100A9 + CX3CR1 + CIITA | 0.881 | 0.772 | 0.99 |
| 7 days | C3AR1 + IL1R2 + S100A9 + CX3CR1 + CIITA + CD3D | 0.864 | 0.748 | 0.981 |
| 7 days | C3AR1 + IL1R2 + S100A9 + CX3CR1 + CIITA + CD3D + CTLA4 | 0.886 | 0.784 | 0.989 |
| 7 days | C3AR1 + IL1R2 + S100A9 + CX3CR1 + CIITA + CTLA4 | 0.873 | 0.76 | 0.985 |
| 7 days | C3AR1 + IL1R2 + S100A9 + CX3CR1 + CIITA + TAP2 | 0.864 | 0.747 | 0.982 |
| 7 days | C3AR1 + IL1R2 + S100A9 + CX3CR1 + CIITA + TAP2 + CD3D | 0.864 | 0.747 | 0.981 |
| 7 days | C3AR1 + IL1R2 + S100A9 + CX3CR1 + CIITA + TAP2 + CD3D + CTLA4 | 0.875 | 0.768 | 0.982 |
| 7 days | C3AR1 + IL1R2 + S100A9 + CX3CR1 + CIITA + TAP2 + CTLA4 | 0.873 | 0.759 | 0.986 |
| 7 days | C3AR1 + IL1R2 + S100A9 + CX3CR1 + CTLA4 | 0.742 | 0.575 | 0.91 |
| 7 days | C3AR1 + IL1R2 + S100A9 + CX3CR1 + TAP2 | 0.781 | 0.627 | 0.935 |
| 7 days | C3AR1 + IL1R2 + S100A9 + CX3CR1 + TAP2 + CD3D | 0.765 | 0.607 | 0.922 |
| 7 days | C3AR1 + IL1R2 + S100A9 + CX3CR1 + TAP2 + CD3D + CTLA4 | 0.77 | 0.618 | 0.923 |
| 7 days | C3AR1 + IL1R2 + S100A9 + CX3CR1 + TAP2 + CTLA4 | 0.784 | 0.631 | 0.937 |
| 7 days | C3AR1 + IL1R2 + S100A9 + TAP2 | 0.776 | 0.622 | 0.929 |
| 7 days | C3AR1 + IL1R2 + S100A9 + TAP2 + CD3D | 0.756 | 0.598 | 0.914 |
| 7 days | C3AR1 + IL1R2 + S100A9 + TAP2 + CD3D + CTLA4 | 0.77 | 0.618 | 0.922 |
| 7 days | C3AR1 + IL1R2 + S100A9 + TAP2 + CTLA4 | 0.773 | 0.619 | 0.927 |
| 7 days | C3AR1 + IL1R2 + TAP2 | 0.676 | 0.497 | 0.854 |
| 7 days | C3AR1 + IL1R2 + TAP2 + CD3D | 0.679 | 0.501 | 0.856 |
| 7 days | C3AR1 + IL1R2 + TAP2 + CD3D + CTLA4 | 0.698 | 0.525 | 0.871 |
| 7 days | C3AR1 + IL1R2 + TAP2 + CTLA4 | 0.704 | 0.531 | 0.876 |
| 7 days | C3AR1 + S100A9 | 0.742 | 0.577 | 0.908 |
| 7 days | C3AR1 + S100A9 + CD3D | 0.74 | 0.58 | 0.9 |
| 7 days | C3AR1 + S100A9 + CD3D + CTLA4 | 0.753 | 0.597 | 0.91 |
| 7 days | C3AR1 + S100A9 + CIITA | 0.85 | 0.729 | 0.972 |
| 7 days | C3AR1 + S100A9 + CIITA + CD3D | 0.856 | 0.736 | 0.976 |
| 7 days | C3AR1 + S100A9 + CIITA + CD3D + CTLA4 | 0.859 | 0.743 | 0.974 |
| 7 days | C3AR1 + S100A9 + CIITA + CTLA4 | 0.85 | 0.729 | 0.972 |
| 7 days | C3AR1 + S100A9 + CIITA + TAP2 | 0.856 | 0.737 | 0.975 |
| 7 days | C3AR1 + S100A9 + CIITA + TAP2 + CD3D | 0.85 | 0.729 | 0.972 |
| 7 days | C3AR1 + S100A9 + CIITA + TAP2 + CD3D + CTLA4 | 0.864 | 0.751 | 0.977 |
| 7 days | C3AR1 + S100A9 + CIITA + TAP2 + CTLA4 | 0.845 | 0.723 | 0.967 |
| 7 days | C3AR1 + S100A9 + CTLA4 | 0.742 | 0.579 | 0.905 |
| 7 days | C3AR1 + S100A9 + CX3CR1 | 0.737 | 0.57 | 0.904 |
| 7 days | C3AR1 + S100A9 + CX3CR1 + CD3D | 0.756 | 0.594 | 0.919 |
| 7 days | C3AR1 + S100A9 + CX3CR1 + CD3D + CTLA4 | 0.762 | 0.609 | 0.915 |
| 7 days | C3AR1 + S100A9 + CX3CR1 + CIITA | 0.856 | 0.737 | 0.974 |
| 7 days | C3AR1 + S100A9 + CX3CR1 + CIITA + CD3D | 0.875 | 0.765 | 0.985 |
| 7 days | C3AR1 + S100A9 + CX3CR1 + CIITA + CD3D + CTLA4 | 0.87 | 0.759 | 0.981 |
| 7 days | C3AR1 + S100A9 + CX3CR1 + CIITA + CTLA4 | 0.853 | 0.732 | 0.974 |
| 7 days | C3AR1 + S100A9 + CX3CR1 + CIITA + TAP2 | 0.864 | 0.747 | 0.982 |
| 7 days | C3AR1 + S100A9 + CX3CR1 + CIITA + TAP2 + CD3D | 0.867 | 0.751 | 0.983 |
| 7 days | C3AR1 + S100A9 + CX3CR1 + CIITA + TAP2 + CD3D + CTLA4 | 0.867 | 0.756 | 0.978 |
| 7 days | C3AR1 + S100A9 + CX3CR1 + CIITA + TAP2 + CTLA4 | 0.861 | 0.742 | 0.981 |
| 7 days | C3AR1 + S100A9 + CX3CR1 + CTLA4 | 0.737 | 0.569 | 0.905 |
| 7 days | C3AR1 + S100A9 + CX3CR1 + TAP2 | 0.789 | 0.638 | 0.941 |
| 7 days | C3AR1 + S100A9 + CX3CR1 + TAP2 + CD3D | 0.778 | 0.625 | 0.932 |
| 7 days | C3AR1 + S100A9 + CX3CR1 + TAP2 + CD3D + CTLA4 | 0.762 | 0.605 | 0.919 |
| 7 days | C3AR1 + S100A9 + CX3CR1 + TAP2 + CTLA4 | 0.789 | 0.637 | 0.942 |
| 7 days | C3AR1 + S100A9 + TAP2 | 0.776 | 0.623 | 0.929 |
| 7 days | C3AR1 + S100A9 + TAP2 + CD3D | 0.759 | 0.601 | 0.917 |
| 7 days | C3AR1 + S100A9 + TAP2 + CD3D + CTLA4 | 0.767 | 0.611 | 0.924 |
| 7 days | C3AR1 + S100A9 + TAP2 + CTLA4 | 0.778 | 0.625 | 0.932 |
| 7 days | C3AR1 + TAP2 | 0.676 | 0.497 | 0.855 |
| 7 days | C3AR1 + TAP2 + CD3D | 0.69 | 0.512 | 0.867 |
| 7 days | C3AR1 + TAP2 + CD3D + CTLA4 | 0.704 | 0.53 | 0.877 |
| 7 days | C3AR1 + TAP2 + CTLA4 | 0.701 | 0.527 | 0.875 |

1. An in vitro or ex vivo method for determining the risk of complication, in a patient, comprising a step of measuring the expression of C3AR1 in a biological sample from said patient.

2. The method as claimed in claim 1, wherein the method additionally comprises a step of measuring, in the biological sample from the patient, the expression:
of at least one gene selected from the family of genes encoding molecules involved in the innate immune system, and/or
of at least one gene selected from the family of genes encoding molecules of the cell cycle, and/or
of at least one gene selected from the family of genes encoding cytokines, and/or
of at least one gene selected from the family of genes encoding anti-inflammatory cytokines, and/or
of at least one gene selected from the family of genes encoding pro-inflammatory cytokine receptors, located on chromosome 2 in the region 2q11-2q12, and/or
of at least one gene selected from the family of genes encoding pro-inflammatory cytokines, and/or
of at least one gene selected from the family of genes encoding molecules involved in cytoskeleton formation, and/or
of at least one gene selected from the family of genes encoding molecules involved in gene expression and/or transcription, and/or
of at least one gene selected from the family of genes encoding growth factors, and/or
of at least one gene selected from the family of genes encoding molecules of the metabolism, and/or
of at least one gene selected from the family of genes encoding molecules involved in the adaptive immune system, and/or
of at least one gene selected from the family of genes encoding molecules involved in signal transduction, and/or
of at least one gene selected from the family of genes encoding molecules involved in modulation of the acute phase, and/or
of at least one human endogenous retrovirus (HERV).

3. The method as claimed in claim 1, wherein the method comprises a step of measuring, in the biological sample from the patient, the expression:
of at least one gene selected from the following genes: GNLY, S100A9, ADGRE3, CD177, CX3CR1, IFIH1, OAS2, OAS3, and/or
of the CCNB1IP1 gene, and/or
of at least one gene selected from the following genes: IL15, IL2, MCP1(CCL2), CXCL10, and/or
of at least one gene selected from the following genes: IL10, IL1RN, and/or
of at least one gene selected from the following genes: IL18R1, IL1R2, IL1R1 and IL18RAP, and/or
of at least one gene selected from the following genes: IFNG, IL1B, IL17A, IL18, IL6, TNF, and/or
of at least one gene selected from the following genes: ARL14EP, GSN, and/or
of at least one gene selected from the following genes: CIITA, DYRK2, GATA3, MDC1, NFKB1, RORC, STAT4, TBX21, TDRD9, and/or
of the CSF2 gene, and/or
of at least one gene selected from the following genes: ALOX5, BPGM, TRAP1, and/or
of at least one gene selected from the following genes: CD40LG, CD3D, BTLA, CD274, CTLA4, ICOS, PDCD1, TNFSF4, CD74, FCGR1A, LILRB2, TAP2, and/or
of at least one gene selected from the following genes: FLT1, HAVCR2, IL7R, ZAP70, and/or
of the HP gene, and/or
of at least one HERV selected from the following HERVs: 190665001-HERV0376, 220247002-HERV0797, 050286701-HERV0513, 130360601-HERV0808, 021456001-MALR1017, 170369402HE41, 121601801-HERV0492, 011052202-HERV1033, 011052702-MALR1044.

4. The method as claimed in claim 1, wherein the method comprises a step of measuring, in the biological sample from the patient, the expression of at least one gene selected from the following genes: GNLY, S100A9, ADGRE3, CD177, CX3CR1, OAS2, CCNB1IP1, IL10, IL1RN, IL1R2, IFNG, TNF, ARL14EP, CIITA, GATA3, MDC1, TDRD9, BPGM, CD3D, CD274, CTLA4, CD74, TAP2, IL7R, ZAP70, HP.

5. The method as claimed in claim 4, wherein the method comprises a step of measuring, in the biological sample from the patient, the expression of at least one gene selected from the following genes: S100A9, CD177, CX3CR1, IL1R2, IFNG, CIITA, CD3D, CTLA4, CD74, TAP2, HP.

6. The method as claimed in claim 1, wherein the biological sample is a blood sample.

7. The method as claimed in claim 1, wherein the expression is measured at the messenger RNA (mRNA) level.

8. The method as claimed in claim 1, wherein the expression is measured by RT-PCR.

9. The method as claimed in claim 1, wherein the expression is measured by sequencing.

10. The method as claimed in claim 1, wherein the expression is measured by hybridization.

11. The method as claimed in claim 7, wherein the expression is normalized in relation to the expression of one or more housekeeping genes.

12. A kit comprising means for amplifying and/or means for detecting the expression of C3AR1 and of at least one other biomarker, selected from the biomarkers of claim 2, said kit wherein all the amplification and/or detection means of said kit enable the detection and/or amplification of at most 100 biomarkers in total.

13. A method comprising using the in vitro or ex vivo:
of means for amplifying and/or means for detecting the expression of C3AR1, or
of a kit comprising such amplification and/or detection means,
for determining the risk of incidence of complication in a patient.
